(19) United States
(12) Patent Application Publication
(10) Pub. No.: US 2026/0031218 A2
(43) Pub. Date: Jan. 29, 2026

(54) EVALUATION OF TOPOLOGICAL COMPLEXITY AND GENERATION OF QUANTITATIVE MARKERS IN MEDICAL IMAGES

(71) Applicant: QT IMAGING, INC., Novato, CA (US)

(72) Inventors: James W. Wiskin, Novato, CA (US); Bilal Hameed Malik, Novato, CA (US)

(21) Appl. No.: 18/955,499

(22) Filed: Nov. 21, 2024

Related U.S. Application Data
(63) Continuation-in-part of application No. 18/888,547, filed on Sep. 18, 2024.
(60) Provisional application No. 63/552,191, filed on Feb. 11, 2024.

(51) Int. Cl. G16H 30/40; G06T 5/30; G06T 7/00; G06T 7/155; G16H 50/30

(52) U.S. Cl. CPC G16H 30/40; G06T 5/30; G06T 7/0012; G06T 7/155; G16H 50/30; G06T 2207/20036; G06T 2207/30068

(57) ABSTRACT

A method can include receiving, at a computing system, a reconstruction image of a breast, the reconstruction image comprising image data corresponding to a plurality of transmission frequencies used by a transmitter of an imaging system; generating, by the computing system, a ductal and/or glandular image from the reconstruction image of the breast; and determining, by the computing system, a quantitative measure of topological complexity of the breast from the ductal and/or glandular image. In certain applications, a quantitative measure of topological complexity can be used as a correction factor to estimates such as a Volpara estimate. In certain applications, a quantitative measure of topological complexity can be used as an input to a risk assessment model.

### CROSS-REFERENCE TO RELATED APPLICATION

This application is a continuation-in-part of U.S. application Ser. No. 18/888,547, filed Sep. 18, 2024, which claims the benefit of U.S. Provisional Application No. 63/552,191, filed Feb. 11, 2024.

### BACKGROUND

Elastic, acoustic, and electromagnetic waves in homogeneous and layered environments in the frequency range of a fraction of a cycle per second up to hundreds of millions of cycles per second and higher can be propagated through many solids and liquids. Elastic waves are waves that propagate through solids and have components of particle motion both parallel (longitudinal, or pressure, wave) and perpendicular (shear wave) to the direction of propagation of the wave energy itself. Acoustic waves are those waves that generate particle motion that is exclusively parallel to the propagation of wave energy. Electromagnetic waves have components of variation of field strength solely in the direction perpendicular to the direction of propagation. All of these types of waves may be used to image the acoustic longitudinal wavespeed and absorption, the electromagnetic wavespeed and absorption, the shear wavespeed, and the density of the material through which the wave energy has travelled.

Scattering is produced not only by spatial fluctuations in acoustic impedance, which is the product of mass density times wavespeed, but also by independent fluctuations in electromagnetic permeability, permittivity and conductivity, elastic compressibility, shear modulus, density, and absorption. These lead to variations in phase speed (which is the speed of propagation of fronts of constant phase) and in impedance (for the electromagnetic case, the ratio of the electric to the magnetic field strength). The net property of an object which describes the phenomenon of scattering in a given modality, is called the “scattering potential”.

There are numerous techniques to image scattering bodies within a given medium. The direct or forward scattering problem is concerned with a determination of the scattered energy or fields when the elastic or electromagnetic properties of the scattering potential are known. The inverse scattering problem uses scattered electromagnetic, elastic, and/or acoustic waves to determine the internal material properties of objects embedded in a known (ambient) medium. An incident wave field is imposed upon the ambient medium and the scatterer. The scattered field is measured at detectors placed a finite distance from the scattering objects. The material parameters of the scatterer are then reconstructed from the information contained in the incident and scattered fields. In other words, acoustic or electromagnetic imaging using inverse scattering techniques involves electronic or optical reconstruction and display of the size, shape, and unique distribution of material elastic or electromagnetic and viscous properties of an object scanned with elastic, electromagnetic, or acoustic energy (e.g., reconstruction of that scattering potential which, for a given incident field and for a given wave equation, would replicate a given measurement of the scattered field for any source location).

Medical imaging involves acquisition of data and image reconstruction from the data. Image reconstruction from acquired data can be treated as an inverse problem that can be solved via iterative methods. Often, an approximate solution—an assumed image or an initial estimate—is first obtained and then a better reconstruction is generated using multiple iteration steps—utilizing all the acquired projections data, the difference between the measured and the modeled fields is calculated, and updating the image based on comparison, resulting in progressive improvement of image quality and also reduction in reconstruction image artifacts. Image reconstruction may be carried out for imaging modalities such as magnetic resonance imaging (MRI), positron emission tomography (PET), computed tomography (CT), and Ultrasound.

### BRIEF SUMMARY

Evaluation of topological complexity and generation of quantitative markers in medical images are described. Through the described techniques, it is possible to obtain quantitative estimates of complexity and use these quantitative estimates of complexity as markers for assisting a variety of diagnostic and predictive applications. Examples of applications of the quantitative estimates of complexity include generation of correction factors and inputs to risk assessment models. The quantitative estimates of complexity, among other features that can be observed from the medical images, can be used clinical decision-making, tailored therapeutic approaches, and real-time response monitoring, all of which contribute to more personalized and effective patient management. For quantitative estimates of complexity, medical images can be processed to generate segmented images of fibroglandular tissue including in the form of glandular or ductal tissue separately or in combination, which are evaluated using graph theory and/or algebraic topology. The processed medical images can be generated through adaptive reconstruction, which involve the adaptability of the algorithms and operations for imaging an object based on specified criteria and/or tests. Imaging techniques incorporating adaptive reconstruction can be referred to as adaptive imaging.

In some aspects, the techniques described herein relate to a computer-implemented method for adaptive image reconstruction, including: generating, by a processor, a preliminary reconstruction image using a preliminary reconstruction configuration; automatically adjusting the preliminary reconstruction configuration to an updated reconstruction configuration, by, at least: obtaining, by the processor, preliminary information from the preliminary reconstruction image; accessing, by the processor, a database of reconstruction configurations, the database providing a mapping of characteristics of images and objects in the images to reconstruction configurations; and performing, by the processor, a lookup operation to identify the updated reconstruction configuration based on the preliminary information; and generating, by the processor, a reconstruction image using the updated reconstruction configuration. Reconstruction information can be obtained from the reconstruction image. For example, topological complexity of a breast can be determined from the reconstruction image.

In some aspects, the techniques described herein relate to receiving, at a computing system, a reconstruction image of a breast, the reconstruction image comprising image data corresponding to a plurality of transmission frequencies used by a transmitter of an imaging system; generating, by the computing system, a ductal and/or glandular image from the reconstruction image of the breast; and determining, by the computing system, a quantitative measure of topological complexity of the breast from the ductal and/or glandular image. In certain applications, a quantitative measure of topological complexity can be used as a correction factor to estimates such as a surrogate estimate of breast density similar to that provided by Volpara software for mammograms. In certain applications, a quantitative measure of topological complexity can be used as an input to a risk assessment model.

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter.

### BRIEF DESCRIPTION OF THE DRAWINGS

FIG. 1 illustrates an ultrasound-based tomographic system that may be used to collect data used in the described image reconstruction and analysis techniques.

FIG. 2 illustrates a process flow diagram of a process that can be carried out by an acquisition control system.

FIG. 3 illustrates an example architecture for adaptive imaging.

FIGS. 4A and 4B illustrate an example computer-implemented method for adaptive image reconstruction.

FIG. 5 illustrates an example adaptive imaging process for generating transmission/attenuation image(s).

FIGS. 6A-6D illustrate results of a cylindrical coordinate system-based algorithm.

FIG. 7 shows reflection images of a breast, including the fusing of a processed attenuation image and reflection image.

FIGS. 8A and 8B illustrate light wave propagation for diffraction-less beams.

FIG. 9 shows a log plot of attenuation coefficient vs frequency from Bamber, 1986, Attenuation and absorption. Physical Principles of Medical Ultrasonics, ed. C R Hill.

FIGS. 10A-10D illustrate identification of tissue type through the power law variation of attenuation with frequency.

FIG. 11 illustrates propagation between a transmitter and receiver.

FIG. 12 shows a plot of Cs vs compressional speed.

FIGS. 13A-13D provide images showing application of porosity estimation using Biot as described herein.

FIG. 14A shows a method of generating a quantitative measure of topological complexity.

FIG. 14B shows an example process of a graph-based approach for determining topological complexity.

FIGS. 15A and 15B show example images of ductal structures.

FIG. 16 shows a plot of VolparaDensity to mammographic density estimated by techniques described herein.

FIG. 17 is schematic showing the Reflection coefficients relevant to the layered medium Green's function approach.

FIG. 18 illustrates a rectangular scattering matrix according to an embodiment of the present invention.

FIG. 19 illustrates a N-by-N scattering region according to an embodiment of the present invention.

FIG. 20 illustrates a N-by-N scattering coalesced region according to the present invention.

FIG. 21 shows a transducer coupling according to an embodiment of the present invention.

FIG. 22 shows the flowchart for the solution of the forward problem by means of the Parabolic FFT Marching method.

FIGS. 23A/B/C show the flowchart for the solution of the Jacobian of the forward problem for the Parabolic FFT Marching method.

FIGS. 24A/B/C show the flowchart for the action of the Hermitian conjugate of the Jacobian for the Parabolic FFT Marching method.

FIGS. 25A/B/C/D show the flowchart for the application of the conjugate gradient method applied to the generalization of the Propagation-Backpropagation method of Inverse Scattering.

FIG. 26 is the flowchart for the original Propagation-Backpropagation Method described in the paper “A Propagation-Backpropagation Method for Ultrasound Tomography”, Frank Natterer, which is included herein as reference.

FIGS. 27A/B/C/D/E show the flowchart for the brightness maximization based phase aberration correction algorithm.

FIG. 28 shows the basic Geometry for the paper “A Propagation-Backpropagation Method for Ultrasound Tomography”, Frank Natterer, which is included herein as reference. This is referred to as FIG. 1 in this paper. The square Qⱼ is the square of sidelength 2ρ whose boundary is made up of Γⱼ (side-scatter directions),

$${Γ}_{j}^{-}$$

(Backscatter direction), and

$${Γ}_{j}^{+}$$

(forward scatter direction). It encompasses the region Ω, which contains the support of the object function γ. θⱼ is the direction of the incident wavefield propagation.

FIGS. 29A and 29B show an elongated rectangle Qⱼ enclosing the region Ω, with boundaries Γⱼ (side-scatter directions),

$${Γ}_{j}^{-}$$

(Backscatter direction), and

$${Γ}_{j}^{+}$$

(forward scatter direction). θⱼ is the direction of the incident wavefield propagation (for FIG. 29A).

FIG. 30 shows the geometry of an acoustic transducer array illuminating an anatomical region through an aberrating layer of fat. A region of interest (ROI), selected by the user, is also shown. The transducer elements that contribute to the image in the ROI are denoted eₘ₁ to eₘ₂.

FIG. 31 shows the method for solving for the object function γ by means of a SQUARE nonlinear map, iteratively, in the scattered field domain.

FIG. 32 shows the iterative method for solving for the object function by means of a SQUARE nonlinear map in the object function (γ) domain.

FIG. 33 shows the geometric set up for a typical calibration procedure.

FIG. 34 shows a comparison of the normalized total fields' (predicted and measured) magnitude versus receiver position.

FIG. 35 shows the comparison of the normalized total fields' phase. Here, the two fields are the predicted field using the starting values for the scattering parameters, and the measured field.

FIG. 36 shows the detailed model used for the receiver in the acoustic 2D case, in a typical calibration setup.

FIG. 37 shows the geometric setup used for the calibration of the receiver and transmitter.

FIG. 38 shows the scattering parameters used in the 2D acoustic calibration procedure typical of the calibration procedure used in inverse scattering, both for acoustic and EM modalities.

FIG. 39 illustrates a generic imaging algorithm, according to the present invention.

FIGS. 40A-40D detail an expanded imaging algorithm with respect to that shown in FIG. 39.

FIG. 41 shows the generic solution to the forward problem according to the present invention.

FIGS. 42A and 42B show the generic solution to the forward problem (or any square system) using a biconjugate gradient algorithm according to an embodiment of the present invention.

FIGS. 43A and 43B show the generic solution to the forward problem using the stabilized conjugate gradient algorithm, according to an embodiment of the present invention

FIG. 44 illustrates the application of the Lippmann-Schwinger Operator to the internal field estimate according to an embodiment of the present invention.

FIG. 45 illustrates the application of the Lippmann-Schwinger Operator in the presence of layering to the internal field estimate according to an embodiment of the present invention.

FIG. 46 illustrates the application of the Hermitian conjugate of the Lippmann-Schwinger operator.

FIG. 47 illustrates the scattering subroutine, according to an embodiment of the present invention.

FIG. 48 illustrates the propagation or transportation of fields from image space to detector position.

FIGS. 49A/B/C illustrate application of the Jacobian Matrix in the generic case (free space—or no layering), according to an embodiment of the present invention

FIGS. 50A/B/C illustrates application of the Hermitian Jacobian Matrix in the generic case according to an embodiment of the present invention.

FIGS. 51A/B illustrates application of the Jacobian Matrix with correlations according to an embodiment of the present invention.

FIG. 52 illustrates the application of the second Lippmann-Schwinger operator in the generic case.

FIG. 53 illustrates the application of Hermitian conjugate of the second Lippmann Schwinger Operators

FIG. 54 the illustrates application the Hermitian conjugate of transportation of fields from image space to detector position.

FIGS. 55A/B illustrate application of the Hermitian conjugate of the Jacobian Matrix in the presence of layering according to an embodiment of the present invention.

FIG. 56 shows an example computing system through which X may be carried out.

### DETAILED DESCRIPTION

Evaluation of topological complexity and generation of quantitative markers in medical images are described. Through the described techniques, it is possible to obtain quantitative estimates of complexity and use these quantitative estimates of complexity as markers for assisting a variety of diagnostic and predictive applications. Examples of applications of the quantitative estimates of complexity include generation of correction factors and inputs to risk assessment models. The quantitative estimates of complexity, among other features that can be observed from the medical images, can be used clinical decision-making, tailored therapeutic approaches, and real-time response monitoring, all of which contribute to more personalized and effective patient management. For quantitative estimates of complexity, medical images can be processed to generate segmented images of fibroglandular tissue in the form of glandular or ductal tissue separately or in combination, which are evaluated using graph theory and/or algebraic topology.

Medical imaging techniques including adaptive reconstruction are provided herein. Adaptive reconstruction involves the adaptability of the algorithms and operations for imaging an object based on specified criteria and/or tests. Imaging techniques incorporating adaptive reconstruction can be referred to as adaptive imaging.

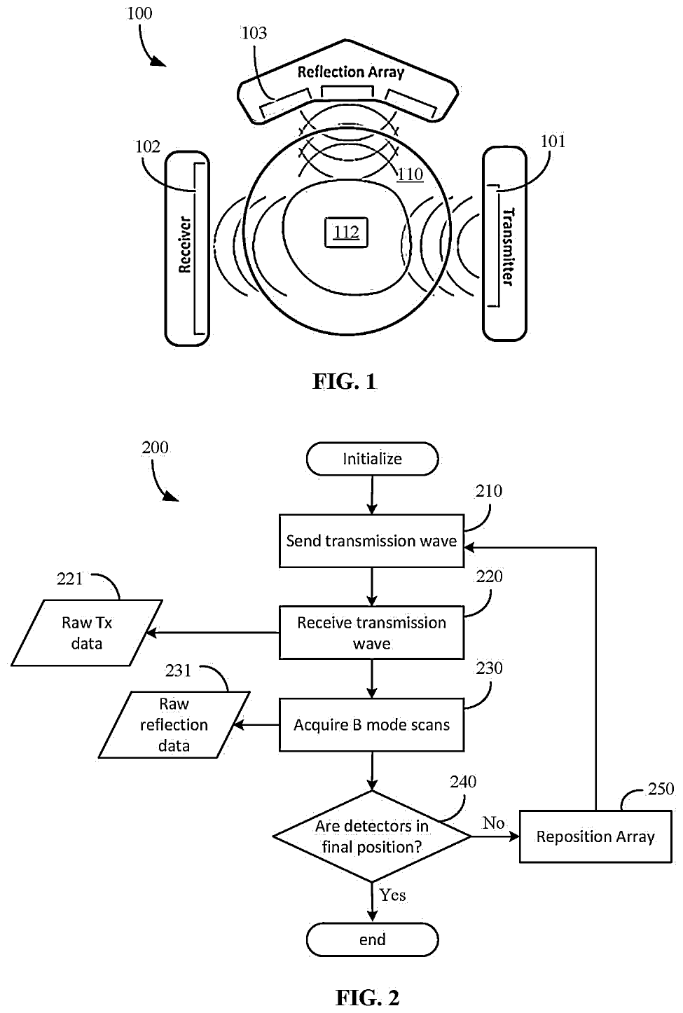

FIG. 1 illustrates an ultrasound-based tomographic system that may be used to collect data used in the described image reconstruction and analysis techniques. Referring to FIG. 1, imaging system 100 can include a transmitter 101, receiver 102, and transceiver 103. When operating in ultrasound frequencies, imaging system 100 can perform both reflection and transmission ultrasound methods to gather data. The reflection portion (e.g., transceiver 103) directs pulses of sound wave energy into tissues and receives the reflected energy from those pulses—hence it is referred to as “reflection ultrasound.” Detection of the sound pulse energies on the opposite side of a tissue after it has passed through the tissue is referred to as “transmission ultrasound.”

The transmitter 101 and a receiver 102 are provided on opposite sides to enable the performing of transmission ultrasound. The transmitter 101 and the receiver 102 may be in the form of an array of transmitters and receivers. The transmitter array can emit broad-band plane pulses (e.g., 0.3-2 MHz) while the receiver array includes elements that digitize the time signal. The transceiver 103, which can include a set of reflection transducers, can be used to perform reflection measurements. The reflection transducers of the transceiver 103 can include transducers of varying focal lengths, providing a large depth of focus when combined. The focus can be fixed or variable in either or both directions. For example, in some cases, the focus may be in the vertical direction only, and the horizontal focus may be dynamic as controlled by a beamformer. In another embodiment, beamforming is included for the vertical direction.

The transmitter 101 and/or transceiver 103 can include a waveform generator that can produce repetitive narrow bandwidth signals or a wide bandwidth signal. The advantages of using a suitable wide bandwidth signal is that in one transmit-receive event, information at multiple frequencies of interest may be collected. Indeed, by using multiple frequencies, it will typically be possible to obtain more data for use in reconstructing the image. Accordingly, a computing system performing image reconstruction can receive data captured by an imaging procedure using a multifrequency data collection method, which includes transmitting a pulse containing multiple frequencies towards an object being imaged.

The lowest frequency used for the signal is selected such that the wavelength of the signal is not significantly smaller than the object to be imaged. In one embodiment, for example, the wavelength may be approximately twice the relevant characteristic length of the object (e.g., breast or other anatomical body part such as abdomen, extremities, etc.) or other smaller length. As a non-limiting example, a selected lowest frequency for imaging a breast may be 0.300 MHz. The highest frequency is selected such that the acoustic signal may be propagated through the object without being absorbed to such an extent as to render detection of the scattered signal impossible or impractical. Thus, depending upon the absorption properties and size of the object which is to be scanned, use of multiple frequencies within this range constraint will typically enhance the ability to more accurately reconstruct the image of the object. The use of multiple frequencies or signals containing many frequencies also has the advantage of obtaining data that may be used to accurately reconstruct frequency dependent material properties.

A coupling medium (e.g., in the form of a liquid or gel for matching of refractive indices) can be used between the object of interest and the imaging system 100. For example, a receptacle 110 can be provided to present a water (or other liquid or gel) bath in which a patient may rest at least the region of interest (e.g., the part 112 being imaged). Because the motion artifacts associated with patient movement can affect the image quality, mechanisms can be provided to facilitate retention and positioning of the breast or other body part. For example, in the case of breast tissue, an adhesive pad with a magnet can be placed near the nipple region of the breast and docked to a magnetized retention rod that gently holds the breast in a consistent position during the scan. As another example, a membrane over the bath between the breast and the liquid is used to hold the breast (and allow for alternative liquids in the bath).

360° of data can be obtained through rotation of the system. The system (particularly arms containing the transmitter 101 and the receiver 102) may rotate 360° to acquire measurements from effectively all the angles (e.g., data sufficient to provide a 360° view even if not taken at every angle between 0° and 360°) and collect tomographic views of ultrasound wave data. The reflection transducer data can be collected with one or more horizontal reflection transducers of transceiver 103 that acquire data in steps or continuously as they rotate 360° along with the transmitter 101 and receiver 102. The transducers may also be tilted with a non-zero polar or azimuth angle.

In a specific implementation, the system rotates around the patient while both transmission and reflection information are captured. It is not necessary to acquire an entire 360° scan; images can be reconstructed with limited information. For example, a patient can lie prone with their breast pendent in a controlled temperature water bath (e.g., 31° C.) within the field of view of the transmitter 101, receiver 102, and transceiver 103 as the transmitter 101, receiver 102, and transceiver 103 rotate 360° around the patient. Then, in one example case 180 projections of ultrasound wave data may be obtained. In another example case, 200 to up to 360 projections or beyond of the ultrasound wave data may be obtained. After performing a rotation, an array chassis holding the transmitter 101, receiver 102, and transceiver 103 can be raised or lowered in a desired sequence to acquire data at another level. The sequence of levels for acquisition may be monotonic or non-monotonic, depending on implementation

Other detector configurations may be used. For example, additional detectors in a continuous or discontinuous ring or polygon configurations may be used. Of course, any configuration selected will have tradeoffs in speed and cost. In addition, in some cases, reflection arrays (the transducers for the reflection measurements) can do double-duty and perform independent transmission and receiver functions as well as reflection measurements. In another embodiment, the reflection array consists of a matrix of elements with multiple rows and columns.

An acquisition control system can be used to operate the various active components (e.g., the transducers) and can control their physical motion (when system 100 is arranged in a rotating configuration). An acquisition control system can automate a scan in response to a start signal from an operator. This automated acquisition process does not require operator interaction during the scanning procedure. Once the scan is complete, the acquisition control system (or other computing system having access to the data) can compute the reflection, speed of sound, and attenuation results from the collected data.

Accordingly, the data captured by system 100 can be used to reconstruct an image using inverse scattering techniques. Applications describing techniques for inverse scattering include U.S. Pat. Nos. 4,662,222; 5,339,282; 6,005,916; 5,588,032; 6,587,540; 6,636,584; 7,570,742; 7,684,846; 7,699,713; 7,771,360; 7,841,982; 8,246,543; and 8,366,617, which are hereby incorporated by reference in their entirety—except for that which is inconsistent with the apparatus and techniques described herein. In addition, the techniques described in “Three-dimensional nonlinear inverse scattering: Quantitative transmission algorithms, refraction corrected reflection, scanner design and clinical results,” Wiskin et al., Proceedings of Meetings on Acoustics, Vol. 19, 075001 (2013), are hereby incorporated by reference in their entirety.

Results can be provided to various computing systems including a viewing station and/or a picture archival and communication system (PACS). Thus, images can be automatically acquired, stored for processing, and available for physician review and interpretation at a review workstation.

FIG. 2 illustrates a process flow diagram of a process that can be carried out by an acquisition control system. Referring to FIG. 2, operation of imaging system 100 of FIG. 1 can be carried out using an acquisition control system performing process 200. Here, in response to receiving an indication to initiate automated scanning (e.g., from an operator or from some programmatic trigger), an acquisition control system can initialize and send a transmission wave from a specified angle about a patient (210), for example from one or more transmitters (such as transmitter 101). As the receiver(s) 102 sense the signal transmitting through the patient (220), raw transmission data 221 is captured. Then, spatially compounded extended depth of focus B mode scans, for example using transceiver(s) 103, are acquired (230) to obtain raw reflection data 231. Of course, in some cases, the B mode scans may be performed before the transmission ones. Additionally, in some embodiments, reflection transducers may have different focal lengths to extend the overall depth of focus within the imaging volume. In one embodiment the focal length is in the vertical direction. Of course, as mentioned above, the focus can be fixed or variable in either or both directions. In some cases, the transmitters (and transceivers) can be used to transmit a pulse containing multiple frequencies towards an object being imaged.

The acquisition control system determines whether the detectors are in the final position (240). For a rotating system, the acquisition control system can communicate with a motor control of the platform on which the active components are provided so that a current and/or next position of the platform is known and able to be actuated. For a fixed system, the acquisition control system determines the selection of the active arrays according to an activation program. In some implementations, the system may be a combination of rotating and fixed. For example, the system may be partially rotating and/or allow for vertical movement only. Accordingly, the “detection” of final position may be based on information provided by the motor control, position sensors, and/or a position program (e.g., using counter to determine whether appropriate number of scans have been carried out or following a predetermined pattern for activating transceivers). If the detectors are not in final position, the acquisition control system causes the array to be repositioned (250), for example, by causing the platform to rotate or by selecting an appropriate array of transceivers of a fixed platform configuration. After the array is repositioned, the transmission wave is sent (210) and received (220) so that the raw transmission data 221 is collected and the B mode scans can be acquired (230) for raw reflection data 231. This repeats until the detectors are determined to be in the final position.

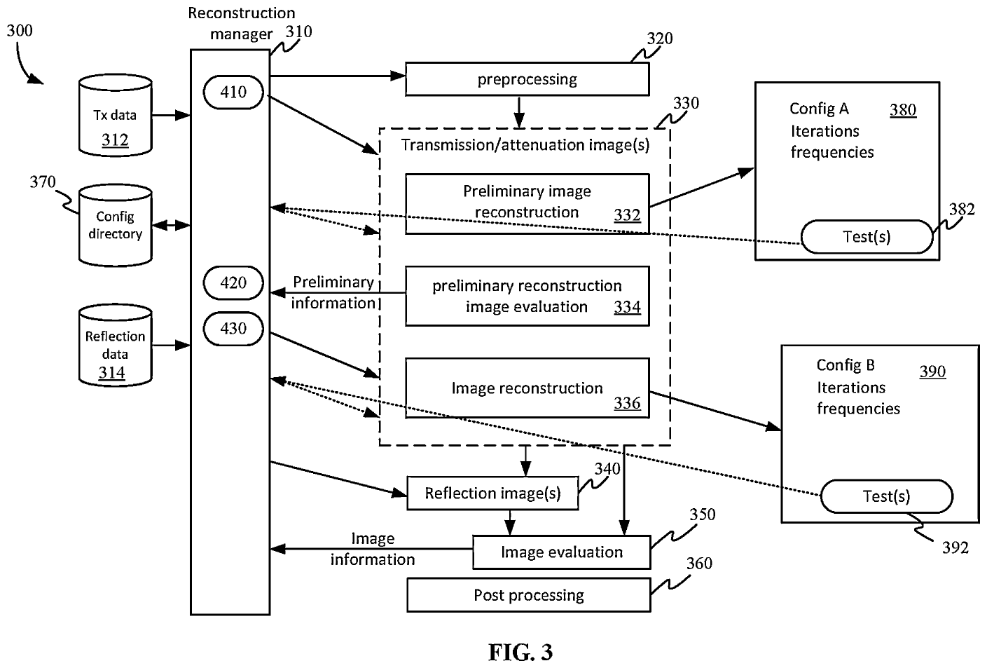

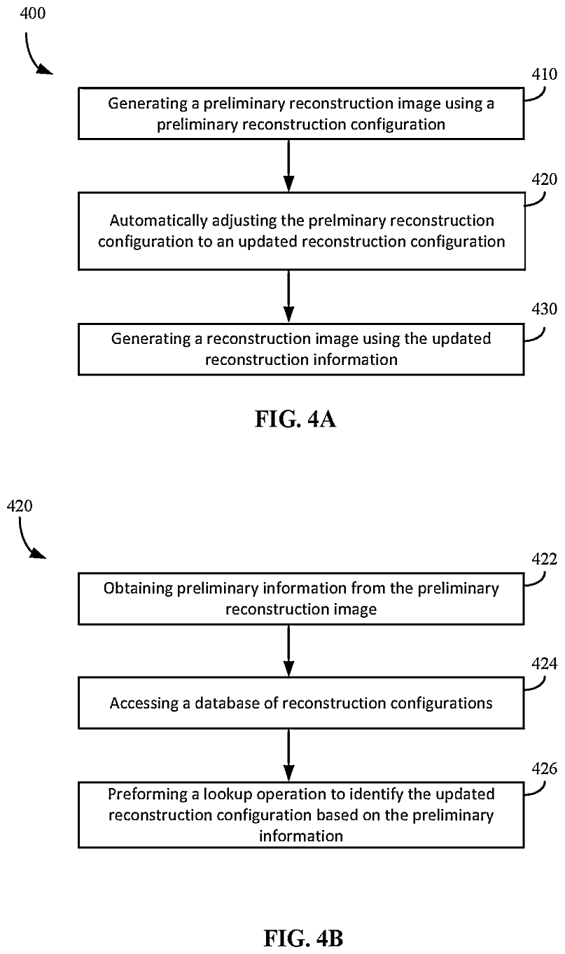

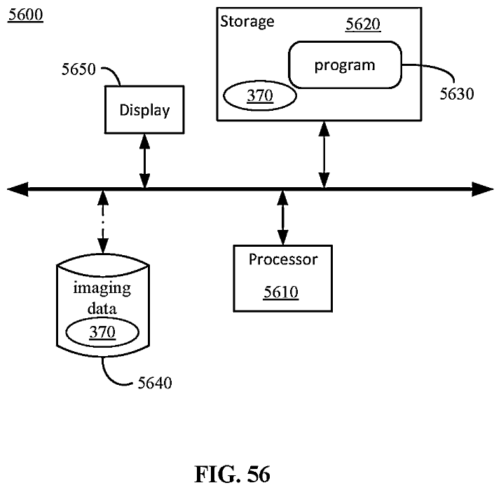

FIG. 3 illustrates an example architecture for adaptive imaging. Referring to FIG. 3, an adaptive imaging architecture 300, which may be implemented on a computing system such as described with respect to FIG. 56, includes a reconstruction manager 310. Reconstruction manager 310 includes the instructions for controlling an adaptive imaging algorithm. For example, reconstruction manager 310 initiates appropriate preprocessing 320 of imaging data such as transmission data 312 and/or reflection data 314, directs generation of transmission/speed of sound/attenuation image(s) 330, directs generation of reflection image(s) 340, applies appropriate image evaluation processes 350 with respect to the generated images, and applies appropriate post processing 360 for the generated images. Reconstruction manager 310 enables automatic adjustments to the applied algorithms based on various aspects of the object being imaged in part based on the image evaluation processes 350 and the image itself such that each process can be adapted, allowing for dynamic selection of optimal parameters. As an illustrative example, as part of generating transmission/attenuation image(s) 330, the reconstruction manager provides an initial reconstruction configuration (e.g., from configuration directory 370) for generating an image and then uses information obtained from the image to update the reconstruction configuration so as to generate a better image. This process can include preliminary image reconstruction 332, preliminary reconstruction image evaluation 334, and image reconstruction. For example, reconstruction manager 310 can perform operations 410, 420, and 430 described with respect to FIGS. 4A and 4B. Within each reconstruction process (e.g., including preliminary image reconstruction 332 and image reconstruction 336), aspects of the reconstruction can be dynamic and adaptable based on various characteristics identified, for example, using specified tests. For example, reconstruction manager 310 can provide a particular reconstruction configuration to use for generating an image along with the particular tests to apply during the reconstruction that can cause changes to certain parameters and/or cause the process to stop and be restarted with the same or different reconstruction configuration. In the illustrated example, the preliminary image reconstruction 332 involves reconstruction configuration “Config A” 380 and test(s) 382; and the image reconstruction 33 involves reconstruction configuration “Config B” 390 and test(s) 392.

FIGS. 4A and 4B illustrate an example computer-implemented method for adaptive image reconstruction. Referring to FIG. 4A, a method 400 for adaptive image reconstruction can include generating (410), by a processor, a preliminary reconstruction image using a preliminary reconstruction configuration; automatically adjusting (420) the preliminary reconstruction configuration to an updated reconstruction configuration; and generating (430), by the processor, a reconstruction image using the updated reconstruction configuration.

Referring to FIG. 4B, automatically adjusting (420) the preliminary reconstruction configuration to an updated reconstruction configuration can include obtaining (422), by the processor, preliminary information from the preliminary reconstruction image; accessing (424), by the processor, a database of reconstruction configurations, the database providing a mapping of characteristics of images and objects in the images to reconstruction configurations; and performing (426), by the processor, a lookup operation to identify the updated reconstruction configuration based on the preliminary information.

Generating (430) a reconstruction image using the updated reconstruction configuration can include applying an inverse scattering algorithm based on propagation through freespace (which can be modeled as empty water); and applying a phase mask at each propagation step of the inverse scattering algorithm to give a total field comprising an incident field plus a scattered field. This method for generating the reconstruction image is applicable for generating (410) the preliminary reconstruction image as well. As will be described in more detail herein, the inverse scattering algorithm can be adapted to a particular coordinate system. In addition, various aspects of the inverse scattering algorithm can be adapted in accordance with the adaptive imaging processes described herein.

After the reconstruction image is generated, reconstruction information can be obtained from the reconstruction image; and the reconstruction image and reconstruction information can be output, for example, to a display.

Returning to FIG. 3, as an illustrative example implementing adaptive imaging architecture 300, the process may be started by command prompts from a technician. Certain preselected parameters may be selected to provide a starting point for operations and/or provide user input to the adaptive imaging processes. A preliminary configuration is selected from the configuration directory 370 by default, based on characteristics of the data, and/or based on input received from the technician. The configuration directory 370 contains all possible recipes. Recipes are sets of frequencies to be used in reconstruction of images from the transmission data. The recipe can include a range of frequencies and the intervals between each frequency. The recipe can also include the iterations at each frequency and each data level. Other aspects can be part of the recipes including parameters such as sections, anisotropic pixel selection, coordinate system, and stopping criteria.

Based at least in part on the recipes that may be selected for the preliminary configuration, preprocessing for the raw transmission data 312 is carried out. For example, preprocessing can include performing a Fourier transform. The Fourier transform is a mathematical operation that transforms the data from the time domain to the frequency domain. After the Fourier transform, magnitude is the y-axis and frequency is the x-axis for the data for operations carried out using the cartesian coordinate system. In another embodiment, the order of the data is changed to allow for quicker sequential access in memory. For a cylindrical or spherical coordinate system, additional preprocessing may be performed to transform coordinates to cylindrical or spherical. In some cases, the data is collected on a system that has the geometric shape corresponding to cylindrical or spherical or ellipsoidal coordinate surfaces.

After preprocessing, the generation of a preliminary image is carried out. The preliminary configuration enables an initial image to be generated quickly. The preliminary image is a relatively fast image, formed using relatively fewer frequencies, for relatively fewer iterations, and is generally blurry. In one embodiment, the image is formed using all levels of data. To form the preliminary image: all data levels may be used. “Data levels” refers to one particular level of data collection. In one embodiment, the array chassis (see e.g., discussion of system of FIG. 1) rotates through 360 degrees collecting transmission and/or reflection data at various azimuthal angles. In another embodiment, the rotational and vertical movement are combined so the concept of ‘data level’ refers to one 360 degree rotation before the array changes azimuthal direction. In another embodiment, the arrays change direction before all 360 degrees are traversed. “All data levels” means every cross-section from the raw data, whether the cross-sections intercept the object or not. Taking all data levels will provide quantitative data regarding the size of the object, which can be included in the preliminary information obtained during the preliminary reconstruction image evaluation 334.

Some of the magnitude and frequency data from the preprocessed data are selected for each image, based on the recipe from preliminary configuration (e.g., Config A 380). In some cases, the preliminary reconstruction configuration includes a set of instructions involving capturing an entire volume of a 3-dimensional image space; using a maximum distance between cross-sections of the 3-dimensional image space; using a limited range of frequencies to generate the preliminary reconstruction image; and using a limited number of iterations to generate the preliminary reconstruction image. Other intermediate preliminary reconstruction images may be included.

As an illustrative example, the recipe can include first generating an initial attenuation image (time of flight), then generating a 2D image (e.g., based on the initial attenuation image), then generating a 3D coarse image (e.g., based on the 2D image), and then generating a 3D fine image (e.g., based on the 3D coarse image), where the 2D image is generated for each frequency of a set of frequencies selected between 0.3 MHz and 0.8 MHz, the 3D coarse image is generated for each frequency of a set of frequencies selected between 0.4 MHz and 0.8 MHz, and the 3D fine image is generated for each frequency of a set of frequencies selected between 0.8 MHz and 1.3 MHz for example. Other embodiments can use different sets of frequencies, which may overlap for the three grids mentioned here. Note the ‘coarse’ grid refers to voxels with generally larger dimensions and/or anisotropy.

Accordingly, multiple images are generated for the preliminary image—each based on a different frequency signal/band. For each image, large-scale optimization can be performed (“running inversions”). In particular, as will be described in more detail herein, acoustic wave propagation through an object is simulated and the properties of the object are optimized to make the simulated propagation fit the observed propagation from the transmission data. The optimization problem can be solved using a stochastic gradient algorithm and the optimization results are used to assign values to pixels and form images. The pixels are squares on a 2D grid. Each 2D grid corresponds to a cross-section, or “data level” of the object being imaged. In some cases, as described in more detail herein, an adaptable anisotropic pixel may be used as a parameter in the reconstruction algorithm.

The reconstruction manager 310 provides the stopping criteria (e.g., included in test(s) 382) which in various embodiments may involve various data (e.g., residuals) associated with the minimization procedure, and subsets chosen for each iteration by the stochastic gradient algorithm and these characteristics can be dynamic for different iterations. The number of iterations for optimization may generally increase with each image formed (e.g., from the TOF, to the 2D image, to the coarse 3D image, to the fine 3D image), leading to higher quality images at the end of the sequence.

As part of the test(s) 382, detection of local minima can be performed early in the optimization process, for example, detection of local minima during the 3D coarse image generation where the residual is above a threshold can trigger the operations to cease and restart using a different recipe. This test of residual behavior may also be referred to as a process detecting whether a salt and pepper image is being formed—such an image is a pseudo-random juxtaposition of high and low values in place of physiologically correct estimates of the speed of sound. In such a case, the test may communicate this failure to the reconstruction manager 310, which can then obtain a different preliminary configuration, and restart the transmission image formation process.

Other tests 382 can also evaluate the object being imaged. For example, there may be a test to detect the presence of a silicone (or other) implant if there is a high residual or a high number of voxels which have a large number of pixels above a certain threshold (e.g., using pixel counts and speed of sound estimation). The test may determine the number of voxels having this value above the certain threshold is too large (above a certain number, for example). Tests can be performed to identify whether high speed voxels occur near the chest wall in the case of breast tissue (or other anomalous values that may be different than expected at a particular location) and this number is above a certain threshold. Indeed, tests 382 can include pixel (voxel) counts and speed of sound estimation, evaluation of gradients and residual magnitude, ratios of magnitudes of gradients and their behavior such as rate of decrease, and the like. Accordingly, tests can be included based on identified objects and based on the image formation process, which provide information used by the reconstruction manager 310 to select an updated reconstruction configuration (used to restart the preliminary image reconstruction 332 and/or generate the reconstruction image 336).

Indeed, in addition to stopping criteria and certain tests performed during the preliminary reconstruction, evaluation 334 of the resulting preliminary image is conducted. For example, for an image of a breast, quantitative measures such as percentage of fibroglandular tissue (sometimes referred to as mammographic density) or fibroglandular tissue volume or ratio (FGV, FGR)—especially when skin volume is omitted) and the size of the breast can be obtained. These measures are then used to update the reconstruction configuration so as to best address the type of tissue and other object characteristics during the reconstruction algorithms. For example, the number of sections/data levels can be selected based on the size of the object/breast. Here, ‘sections’ may refer to the number of data levels used in the reconstruction—in one embodiment the top 24 mm (say 12 data levels at 2 mm separation) may be used for the reconstruction of the ‘top’ section, the bottom 32 mm may be used for the reconstruction of the bottom part of the image and intermediate consecutive data levels may be used for the reconstruction of the middle part of the image. In addition, the frequency ranges may be adjusted based on the size, mammographic density, and results of test(s) 382.

In the following discussion, examples are not meant to be restrictive to a particular body part. Adaptive imaging is applicable to a variety of imaging applications including, but not limited to, the pediatric, orthopedic, and whole body imaging contexts. Tissue types vary with different applications (pediatric, etc.) and need not refer to breast imaging only. The presence of bone and air is a known complication that can be addressed using the described techniques. For example, the percentage of bone and air in a particular imaged volume can determine different recipes similar to that described in more detail below.

The updated reconstruction configuration (e.g., Config B 390) is then used to generate the reconstructed image 336. Compared to the preliminary image, the transmission image is a relatively slower image, using more frequencies, for more iterations, and is high-resolution. The process for forming the transmission image is similar to the process for the preliminary image. That is, acoustic wave propagation through an object is simulated and the properties of the object are optimized to make the simulated propagation fit the observed propagation from the transmission data. The optimization problem can be solved using a stochastic gradient algorithm and the optimization results are used to assign values to pixels and form images.

In some cases, the updated reconstruction configuration includes a set of instructions involving capturing a portion of the volume of the 3-dimensional image space; capturing a portion of the volume of the 3-dimensional image space; capturing a portion of the volume of the 3-dimensional image space; and using an increased number of iterations to generate the reconstruction image. As an illustrative example, the recipe for the updated reconstruction configuration can include generating a 3D coarse image, generating a first 3D fine image, and generating a second 3D fine image, where the 3D coarse image is generated for each frequency of a set of frequencies selected between 0.4 MHz and 0.8 MHz, the first 3D fine image is generated for each frequency of a set of frequencies selected between 0.8 MHz and 1.2 MHz, and the second 3D fine image is generated for each frequency of a set of frequencies selected between 1.2 MHz and 1.3 MHz. In some cases, the 3D coarse image and/or 3D fine image generated during the preliminary image reconstruction 332 is used as a starting point for the 3D coarse image during the image reconstruction 336. As with the preliminary image, multiple images are generated based on a different frequency signal/band and the various adaptions and stopping criteria tests described with respect to the preliminary image reconstruction are applicable. More or fewer images and/or processes may be part of the updated reconstruction configuration (e.g., Config B 390) and tests 392. However, unlike for the preliminary image reconstruction, fewer than all data levels may be used. Here, the number of data levels/sections can be based on the size of the object.

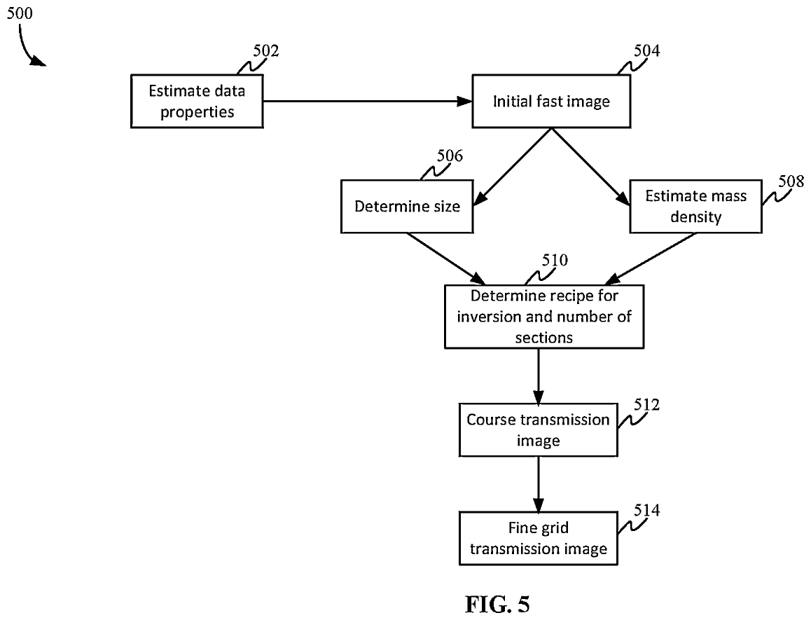

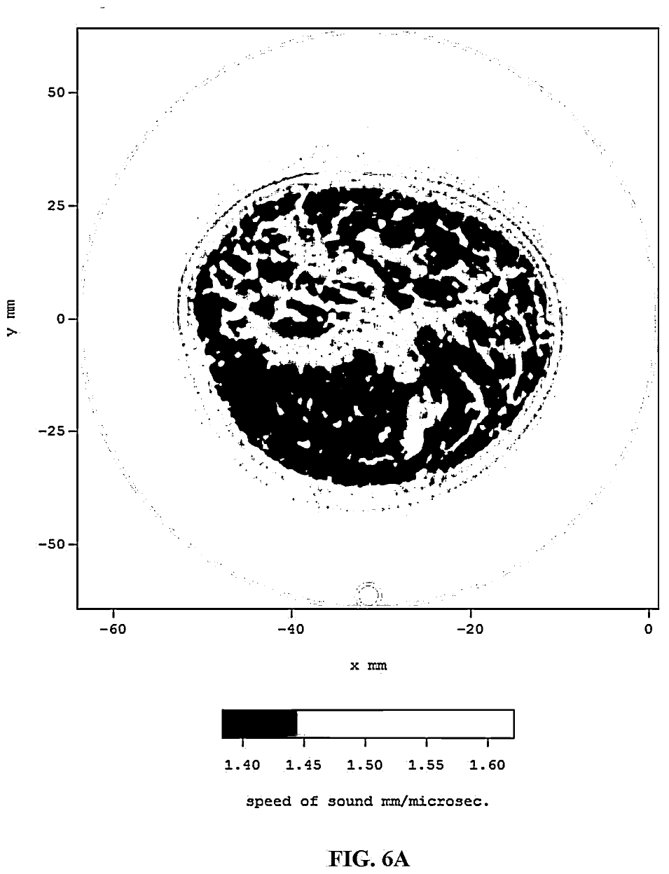

FIG. 5 illustrates an example adaptive imaging process for generating transmission/attenuation image(s). Referring to FIG. 5, an example adaptive imaging process 500, which may be implemented as code executed by a computing system such as computing system 5600 of FIG. 56, can begin with an estimate of data properties 502. The estimate of data properties 502 may be received via a user input or be provided with the captured data (e.g., associated with a patient). From the estimate of data properties 502, an initial number of data levels (e.g., based on breast size) is determined for use by the fast reconstruction algorithm. An initial fast image is generated (504), for example using a coarse grid, 2D reconstruction, and/or time of flight algorithm. In some cases, the initial image is a transmission image. In some cases, a reflection image and/or data can be used to supplement the transmission image.

The generation of the initial fast image can be carried out such as described with respect to preliminary image reconstruction 332 of FIG. 3 and preliminary image reconstruction 410 of FIG. 4A. From the initial fast image, size of the breast can be estimated (506) and mammographic density estimated (508). The breast size estimation (506) can involve determining the size category for the breast, for example, using categories of small, medium, and large. The estimation of the mammographic density (508) involves determining the percentage of fibro-glandular tissue and determining the density category for the breast, for example, using categories of fatty, scattered/heterogenous, and dense. Based on the breast size and mammographic density, the recipe for inversion and number of sections can be determined (510). For example, the adaptive imaging process 500 can use a lookup table (LUT) or matrix, which in the case of breast can be such as follows.

### Example LUT

| Density\size | Small | Medium | Large |
|---|---|---|---|
| Fatty | Config 1 | Config 2 | Config 3 |
| Scattered/heterogeneous | Config 4 | Config 5 | Config 6 |
| dense | Config 7 | Config 8 | Config 9 |

In some cases, each configuration/recipe for the different combinations of breast tissue characteristics is different. In some cases, a same or similar recipe may be used for a subset of different combinations of breast tissue characteristics. Percentages of bone and air may also be part of the characteristics used in whole body/pediatric/partial body imaging. Similarly, for other anatomical regions of the body, a similar LUT could be created. For instance, for human body extremities and musculoskeletal tissue, it could be based on the expected ratio of fat vs muscle vs bone tissue.

Using the matrix/table one or more reconstruction configuration/recipes can be selected for use on each section of the breast. In the illustrated example, a coarse transmission image 512 is generated and a fine grid transmission image 514 is generated. These images can be generated such as described with respect to image reconstruction 336 of FIG. 3 and image reconstruction 430 of FIG. 4A.

### Stopping Criteria and Tests

The reconstruction configurations can be selected and/or changed on the basis of various stopping criteria and tests (e.g., test(s) 382, 392). The criteria and tests can be based on image characteristics and can trigger the restart of an inversion process using a different set of parameters (e.g., frequencies, iterations, anisotropic pixels, or coordinate systems more suited to the data). The tests can include whether there is a silicone implant, whether there is a salt and pepper local minimum developing, and whether the image is going to be blurry.

Indeed, as mentioned above, as part of the test(s) 382, detection of local minima can be performed early in the optimization process, for example, detection of local minima during the 3D coarse image generation where the residual is above a threshold can trigger the operations to cease and restart using a different recipe. As another example in breast tissue, there is a test to detect the presence of a silicone implant if there is a high residual (e.g., using pixel counts and speed of sound estimation). Tests can be performed to identify whether abdominal fat is identified near the chest wall (or other anomalous values that may be different than expected at a particular location). Indeed, tests 382 can include pixel counts and speed of sound estimation, evaluation of gradients and residual magnitude, ratios of magnitudes of gradients and their behavior such as rate of decrease, and the like.

Thus, according to certain embodiments, an adaptive imaging method can thus include detecting one or more errors in the preliminary reconstruction image in real time while generating the preliminary reconstruction image; stopping generating the preliminary reconstruction image; selecting a new preliminary reconstruction configuration from the database of reconstruction configurations based on the detected one or more errors in the preliminary reconstruction image; and restarting the method beginning with generating the preliminary reconstruction image using the new preliminary reconstruction configuration.

In some cases, generating the preliminary reconstruction image comprises assigning values to a plurality of voxels, wherein detecting one or more errors in the preliminary reconstruction image in real time while generating the preliminary reconstruction image comprises: evaluating values of voxels located near each other; and detecting an error if the values of voxels located near each other are above or below a voxel value threshold. In an example implementation where the object being imaged is a patient's breast, detecting an error if the values of voxels located near each other are above or below a voxel value threshold can include detecting the presence of a silicone implant if the values of voxels located near each other are above a silicone implant voxel value threshold. Another test involving evaluating values of voxels located near each other includes evaluating values of voxels near a patient's chest wall, wherein detecting an error if the values of voxels located near each other are above or below a voxel value threshold comprises detecting the presence of fatty tissue or non-biological material near the patient's chest wall if the values of voxels located near the patient's chest wall are above a chest wall voxel value threshold.

Similarly, tests are applied during generating the updated reconstruction image. For example, the adaptive imaging method can include detecting one or more errors in the reconstruction image in real time while generating the reconstruction image; and stopping generating the reconstruction image. Similar to tests during the preliminary reconstruction, detecting one or more errors in the reconstruction image in real time while generating the reconstruction image can include evaluating values of a residual of the reconstruction image; and detecting a local minimum if the values of the residual are above a local minimum threshold value.

Once stopped, either a new preliminary reconstruction configuration can be selected (e.g., to start the process over at step 330) or a new updated reconstruction configuration can be selected (e.g., to restart process 350). For example, an adaptive imaging method can include selecting a new preliminary reconstruction configuration from the database of reconstruction configurations based on the detected one or more errors in the reconstruction image; and restarting the method beginning with generating the preliminary reconstruction image using the new preliminary reconstruction configuration. Of course, in other cases, the adaptive imaging method can include selecting a new reconstruction configuration from the database of reconstruction configurations based on the detected one or more errors in the reconstruction image; and restarting the method beginning with generating the reconstruction image using the new reconstruction configuration.

Although not described in detail, generation of reflection images can also include adaptive parameters and tests for the reflection image configurations can include, but are not limited to, whether there is a bright spot in the reflection image and whether there is a dark spot in the reflection image.

### Adaptive Sectioning and Frequencies

The breast is a 3D object but has different characteristics relevant to wave propagation near the chest wall and in the sub-areolar regions. Based on the acquisition system, it is possible to include data redundancy in the vertical direction.

This means it is possible to collect data every 2 or 4 mm (which also could vary with the particular part of the volume of breast that is being imaged.) This vertical redundancy supports image quality in the presence of compromised SNR and data quality. By simultaneously inverting (imaging) from data at several different close levels, it is possible to increase the redundancy over not incorporating the information at the same time in inversion. It is noted that trying to invert from many levels simultaneously has an over-regularizing effect that creates a ‘smoother’ image that may not be appropriate for the Reader or for diagnostic purposes. By separating the breast into sections, it is possible to optimize for the particular tissue/anatomy. In various embodiments, the breast can be divided into 2, 3, . . . N sections.

### Recipes

It is possible to determine a ‘recipe’ for imaging the breast. A recipe includes the sequence of operations, including frequencies selected for use in the simulations. For example, a subset of frequencies within a range of frequencies corresponding to the frequencies used in the data acquisition can be selected (e.g., 0.4, 0.5, 0.6, 0.7, 0.8, 0.9, 1.0, 1.1, 1.2, 1.3, . . . 1.35 MHz). The change between frequencies is determined empirically and is dynamic, it may change during the inversion process. In addition, the order in which frequencies are used is not required to be consecutive/monotonic That is, the frequency steps do not have to be monotonic increasing. In one embodiment the frequencies rise, then fall then continue to rise and this cycle can repeat. For example, a sequence can be 0.4, 0.425, 0.45, 0.475, 0.45, 0.425, 0.45, 0.475, 0.5, . . . .

In one embodiment a set of frequencies is chosen from 0.4, 0.425, . . . , 1.3 MHz, where the step/difference between frequencies is chosen here as 0.025 MHz. In another embodiment a subset of frequencies are imaged simultaneously. The spacing between the simultaneous frequencies being used can also be adaptive intra and inter-breast. That is, the spacing may change depending on the section. The code/program determines which set of frequencies and how many iterations to carry out at a given frequency to optimize image quality and also avoid ‘local minima’.

Accordingly, with reference to FIG. 3 and FIG. 4A, a preliminary reconstruction configuration can include a set of instructions, comprising forming a sequence of intermediate preliminary reconstruction images involving a sequence of intermediate preliminary reconstruction images, where each intermediate preliminary reconstruction image has an increased resolution relative to the previous intermediate preliminary reconstruction image. In some cases, the sequence of intermediate preliminary reconstruction images starts at a low frequency preliminary reconstruction image and proceeds in a stepwise fashion to higher frequency intermediate preliminary reconstruction images. In some cases, the sequence of intermediate preliminary reconstruction images is carried out at frequencies in a non-monotonic progression.

Similarly, an updated reconstruction configuration can include a set of instructions, comprising forming a sequence of intermediate reconstruction images involving a sequence of intermediate reconstruction images, where each intermediate reconstruction image has an increased resolution relative to the previous intermediate reconstruction image. In some cases, the sequence of intermediate reconstruction images starts at a low frequency reconstruction image and proceeds in a stepwise fashion to higher frequency intermediate reconstruction images. In some cases, the sequence of intermediate reconstruction images is carried out at frequencies in a non-monotonic progression. The sequence of frequencies for the updated reconstruction configuration may be the same or different than that used in the preliminary reconstruction configuration. In addition, for reconstruction configurations involving multiple levels of reconstruction (see e.g., examples provided for Config A 380 and Config B 390), where the intermediate images increase in resolution (e.g., from coarse to fine) the frequency sequence used in the reconstruction images may differ between the coarse reconstruction and the fine reconstruction.

### Frequency Recipes

How the frequencies are chosen can impact whether local minima are encountered (see also stopping criteria). fⱼ, j=1, . . . , Nₖ, where k=1, . . . , N_{freq}. In one embodiment the frequencies are equally spaced. In another embodiment the frequencies are chosen so that delta lambda (δλ) is constant since the change in wavelength will determine whether cycle skipping takes place. In such an embodiment, to maintain a constant δλ, the relationship between f (frequency) and δλ is not linear. That is, since

$$δ {f}_{j} = - \frac{{c}_{o}}{{λ}_{j}^{2}} δ {λ}_{j}$$

so that at a particular frequency j, the step size in frequency is

$$δ {f}_{j} = - \frac{{f}_{j}}{{λ}_{j}} δ {λ}_{j} or δ {f}_{j} = - \frac{{f}_{j}^{2}}{{c}_{o}} δ {λ}_{j} ,$$

that is the step size in frequency can in fact increase quadratically with frequency to keep the change in the wavelength the same. If the relative increase in wavelength is required to be constant than the formula

$$δ {f}_{j} = - ( \frac{δ {λ}_{j}}{{λ}_{j}} ) {f}_{j} ,$$

indicates the step size increase linearly with frequency. The optimal step size may be determined empirically.

### Adaptable Anisotropic Pixel

An anisotropic pixel selection can be carried out as part of an adaptive imaging process. That is, a size of steps used in an inverse scattering algorithm (e.g., propagation and backscattering) for generating a reconstruction image can be based on an anisotropic pixel selection, where the anisotropy is through the geometry (e.g., having different lengths in different directions). An anisotropic pixel selection includes a distance in one direction between surfaces of the steps used in the inverse scattering algorithm. The one direction may be in the direction of propagation, perpendicular to the direction of propagation or some other angle with respect to the direction of propagation. In some cases, at least two iterations of the inverse scattering algorithm use different anisotropic pixel selections. For example, the distance in one direction between surfaces of the steps used in the inverse scattering algorithm can change from one iteration to another iteration. As an illustrative example, a first distance may be used for five iterations and then a second distance is used for the next three iterations followed by a return to the first distance or a change to a third distance, which may be larger or smaller than the first and/or second distance.

The anisotropic pixel selection can vary between frequencies and iterations in any manner of combinations. In addition, to the distance in one direction that can vary, the direction in which that distance is adjusted may vary between frequencies and/or iterations.

### Coordinate System

As mentioned above, the adaptive imaging process can be adapted to different coordinate systems. Available coordinate systems include, but are not limited to, rectangular, circular-cylinder, elliptic-cylinder, parabolic-cylinder, spherical, prolate spheroidal, oblate spheroidal, parabolic, conical, ellipsoidal, and paraboloidal. Indeed, available coordinate systems can be a cartesian coordinate system, a curvilinear coordinate system, or even a hybrid/expanding coordinate system. As long as an exact solution exists, it is possible to perform an inverse scattering algorithm based on propagation a short distance through free space (or empty water) via a closed or semi-closed form solution, for example, and application of a phase mask. The reconstruction configurations for the different coordinate systems utilize an appropriate phase mask. For example, cartesian coordinates have the plane wave, cylindrical coordinates have Bessel functions and exponentials, spherical coordinates have spherical Bessel (half integer order) and spherical harmonics (associated Legendre functions and exponentials), and expanding coordinates have narrow waist Gaussian functions multiplied by solutions to a paraxial approximation equation.

The ability to adapt the processes for a particular coordinate system such as curvilinear supports the receipt of data collected using a cylindrical array of transmitters and/or receivers. Such data can be transformed into cylindrical coordinates during preprocessing (e.g., operation 320 of FIG. 3) and subsequently analyzed in cylindrical coordinates.

In some cases, an indication of a particular coordinate system from a selection of a cartesian coordinate system and a curvilinear coordinate system can be received; and available reconstruction configurations at the database of reconstruction configurations can be filtered according to the particular coordinate system before generating the preliminary reconstruction image using the preliminary reconstruction configuration. In some cases, the system is adapted to a curvilinear coordinate system, wherein available reconstruction configurations including for the preliminary reconstruction configuration and the updated reconstruction configuration are based on the curvilinear coordinate system.

An example for spherical expanding coordinates via Gaussian beam is as follows.

Note the system involves propagation of energy primarily in the z direction. Here, the paraxial approximation equation can be obtained by factoring out the predominant wave propagating in the z direction:

Acoustic energy field: u(r)=uₚ(r)e^{-iβz}.

### Given the Helmholtz Equation

$$\frac{{\partial }^{2} u}{\partial {x}^{2}} + \frac{{\partial }^{2} u}{\partial {y}^{2}} + \frac{{\partial }^{2} u}{\partial {z}^{2}} + {k}^{2} ( r ) u = 0$$

We can make the assumption that the energy moves predominantly in the z direction and factor that out, as above, i.e.: u(r)=uₚ(r)e^{-iβz}

This gives us

$$( \frac{{\partial }^{2} {u}_{p}}{\partial {x}^{2}} + \frac{{\partial }^{2} {u}_{p}}{\partial {y}^{2}} - 2 i β \frac{\partial {u}_{p}}{\partial z} - {β}^{2} {u}_{p} + \frac{{\partial }^{2} {u}_{p}}{\partial {z}^{2}} + {k}^{2} {u}_{p} ) {e}^{- i β z} = 0$$

Finally, the variation in the acoustic field in the z direction is considered small compared to the variation that is factored out:

$$\vert β \frac{\partial {u}_{p}}{\partial z} \vert >> \vert \frac{{\partial }^{2} {u}_{p}}{\partial {z}^{2}} \vert$$

This gives the paraxial approximation:

$$\frac{{\partial }^{2} {u}_{p}}{\partial {x}^{2}} + \frac{{\partial }^{2} {u}_{p}}{\partial {y}^{2}} - 2 i β \frac{\partial {u}_{p}}{\partial z} + ( {k}^{2} - {β}^{2} ) {u}_{p} = 0$$

Put β=k for the following: (the energy is propagating at the appropriate frequency)

### Gaussian Beam Factor:

Note the Helmholtz equation does have the exact solution: (Green's function in 3D)

$$u = \frac{{e}^{- ik \vert r - {r}^{′} \vert}}{4 π \vert r - {r}^{′} \vert} ,$$

Now using r′=(0,0, −ib)
gives, with the Fresnel approximation:

$$u = {u}_{o} \frac{i b {e}^{- i k \frac{{x}^{2} + {y}^{2}}{( z + i b )}}}{( z + i b )} ,$$

Now put

$$b = \frac{β {w}_{o}^{2}}{2} .$$

In this formulation this is a Gaussian beam with waist w₀

So we thus have the diverging solution:

$$u ( x ) = \frac{{e}^{- i k ( {x}^{2} + {y}^{2} ) / q ( z )}}{q ( z )}$$

Where

$$q ( z ) = z + i \frac{π {w}_{o}^{2}}{λ} ≡ z + i b , b = \frac{2 π {w}_{o}^{2}}{2 λ} = \frac{β {w}_{o}^{2}}{2}$$

Now factor out the Gaussian beam with w₀ waist size and use expanded coordinates for the remaining part of the wave, where for now α is an arbitrary complex value:

$${x}^{′} ≡ \frac{α x}{z + i b}$$

$${y}^{′} ≡ \frac{α y}{z + i b}$$

$${z}_{2}^{′} - {z}_{1}^{′} = \frac{{α}^{2} ( {z}_{2} - {z}_{1} )}{( {z}_{2} + i b ) ( {z}_{1} + i b )}$$

$$u ( x ) = \frac{v ( {x}^{′} )}{q ( z )} {e}^{- i k ( {x}^{2} + {y}^{2} ) / q ( z )}$$

The function variation v(x′) uses the ‘expanding coordinates’ for reasons seen below:

We take a particular form of the diverging beam:

Now we assume that the Gaussian waist is infinitesimal, e, so the wave is approximately spherical. Note the transformation:

$${x}^{′} ≡ \frac{α x}{z + i ε} , {y}^{′} ≡ \frac{α y}{z + i ε} , {z}_{2}^{′} - {z}_{1}^{′} = \frac{{α}^{2} ( {z}_{2} - {z}_{1} )}{( {z}_{2} + i ε ) ( {z}_{1} + i ε )}$$

In the limit as E goes to zero and a E R (ax real). a will be constrained by the calculations below.

$${x}^{′} = {lim}_{ε \to 0} \frac{α x}{z + i ε} = \frac{α x}{z} , {y}^{′} ≡ \frac{α y}{Z} , {z}_{2}^{′} - {z}_{1}^{′} = \frac{{α}^{2} ( {z}_{2} - {z}_{1} )}{{z}_{2} {z}_{1}}$$

For calculation purposes we use:

$${z}^{′} - {z}_{1}^{′} = \frac{{α}^{2} ( z - {z}_{1} )}{( z + i ε ) ( {z}_{1} + i ε )}$$

Use

$$\frac{\partial }{\partial z} = \frac{\partial {x}^{′}}{\partial z} \frac{\partial }{\partial {x}^{′}} + \frac{\partial {y}^{′}}{\partial z} \frac{\partial }{\partial {y}^{′}} + \frac{\partial {z}^{′}}{\partial z} \frac{\partial }{\partial {z}^{′}} = \frac{α x}{{z}^{2}} \frac{\partial }{\partial {x}^{′}} + \frac{α y}{{z}^{2}} \frac{\partial }{\partial {y}^{′}} + \frac{{α}^{2}}{{z}^{2}} \frac{\partial }{\partial {z}^{′}}$$

since

$$\frac{\partial {z}^{′}}{\partial z} = \frac{{α}^{2}}{{zz}_{1}} - \frac{{α}^{2}}{{z}^{2} {z}_{1}} ( z - {z}_{1} ) = \frac{{α}^{2}}{{z}^{2}}$$

$$and \frac{\partial }{\partial x} = \frac{\partial {x}^{′}}{\partial x} \frac{\partial }{\partial {x}^{′}} = \frac{α}{z} \frac{\partial }{\partial {x}^{′}}$$

$$\frac{\partial }{\partial y} = \frac{\partial {y}^{′}}{\partial y} \frac{\partial }{\partial {y}^{′}} = \frac{α}{z} \frac{\partial }{\partial {y}^{′}}$$

And so

$$\frac{{\partial }^{2}}{\partial {x}^{2}} = \frac{α}{z} \frac{\partial {x}^{′}}{\partial x} \frac{{\partial }^{2}}{\partial {x}^{′2}} = {( \frac{α}{z} )}^{2} \frac{{\partial }^{2}}{\partial {x}^{′2}} ,$$

with similar expression for y coordinate, and

$$\frac{{e}^{- i k ( {x}^{2} + {y}^{2} ) / q ( z )}}{q ( z )}$$

solves the paraxial equation.

Now factor out the expanding wave and transform to the expanded coordinate system.

$$u ( x ) = ν ( {x}^{′} ) w ( x ) = \frac{v ( {x}^{′} )}{q ( z )} {e}^{- i k ( {x}^{2} + {y}^{2} ) / q ( z )} ,$$

and we get:

Using,

$$\frac{{\partial }^{2} v ( {x}^{′} ) w ( x )}{\partial {x}^{2}} = \frac{{\partial }^{2} v ( {x}^{′} )}{\partial {x}^{2}} w ( x ) + 2 \frac{\partial v ( {x}^{′} )}{\partial x} \frac{\partial w ( x )}{\partial x} + v ( {x}^{′} ) \frac{{\partial }^{2} w ( x )}{\partial {x}^{2}} ,$$

and similar for y and chain rules above, so that:

$$\frac{{\partial }^{2} v}{\partial {{x}^{′}}^{2}} + \frac{{\partial }^{2} ν}{\partial {{y}^{′}}^{2}} - 2 i k \frac{\partial v}{\partial {z}^{′}} = 0$$

Propagation in the primed coordinates:

The total field is

$$u ( x ) = \frac{v ( {x}^{′} )}{z} {e}^{- i k ( {x}^{2} + {y}^{2} ) / z}$$

The first fact v satisfies the paraxial equation (by above)

$$\frac{{\partial }^{2} v}{\partial {{x}^{′}}^{2}} + \frac{{\partial }^{2} v}{\partial {{y}^{′}}^{2}} - 2 i k \frac{\partial v}{\partial {z}^{′}} = 0 ,$$

so use the ‘transverse’ Fourier transform, F_{T}:

$$\hat{v} ( {k}_{{x}^{′}} , {k}_{{y}^{′}} , {z}^{′} ) = \int \int v ( {x}^{′} , {y}^{′} , {z}^{′} ) {e}^{- {k}_{T} \cdot {x}^{′}} {dx}_{T}^{′} = {F}_{T} ( v )$$

Equation is now

$$( {k}_{x}^{′2} + {k}_{x}^{′2} ) \hat{v} - 2 ik \frac{\partial \hat{v}}{\partial {z}^{′}} = 0 and so \frac{\partial \hat{v}}{\partial {z}^{′}} = \frac{1}{2 i {k}_{o}} ( {k}_{x}^{′2} + {k}_{x}^{′2} ) \hat{v}$$

has the exact solution:

$$\hat{v} ( {k}_{{x}^{′}} , {k}_{{y}^{′}} , {z}^{′} ) = {e}^{\frac{1}{2 i {k}_{o}} ( {k}_{x}^{′2} + {k}_{x}^{′2} ) ( {z}^{′} - {z}_{o}^{′} )} A ,$$

constant A: that is:

$$\hat{v} ( {k}_{{x}^{′}} , {k}_{{y}^{′}} , {z}^{′} ) = {e}^{\frac{1}{2 i {k}_{o}} ( {k}_{x}^{′2} + {k}_{x}^{′2} ) ( {z}^{′} - {z}_{o}^{′} )} \hat{v} ( {k}_{{x}^{′}} , {k}_{{y}^{′}} , {z}_{o}^{′} )$$

Is the exact solution. Using

$$δ {z}^{′} = ( {z}^{′} - {z}_{o}^{′} )$$

$$ν ( {x}^{′} , {y}^{′} , {z}^{′} + δ {z}^{′} ) = {F}_{T}^{- 1} ( \hat{v} ( {k}_{{x}^{′}} , {k}_{{y}^{′}} , {z}^{′} + δ {z}^{′} ) ) ,$$

putting in all the operators:

$${F}_{T}^{- 1}$$

is the inverse transverse Fourier transform:

$$v ( {x}^{′} , {y}^{′} , {z}^{′} + δ {z}^{′} ) = {F}_{T}^{- 1} ( {e}^{( {k}_{x}^{′2} + {k}_{x}^{′2} ) δ {z}^{′}} \hat{v} ( {k}_{{x}^{′}} , {k}_{{y}^{′}} , {z}^{′} ) ) = {F}_{T}^{- 1} ( {e}^{\frac{1}{2 i {k}_{o}} ( {k}_{x}^{′2} + {k}_{x}^{′2} ) δ {z}^{′}} {F}_{T} ( v ( {x}^{′} , {y}^{′} , {z}^{′} ) ) )$$

In operator theoretic notation (read from right to left) this is just the transport in free space formula.

$$ν ( {x}^{′} , {y}^{′} , {z}^{′} + δ {z}^{′} ) = {F}_{T}^{- 1} [ {P}_{o} ] {F}_{T} v$$

with free space propagator:

$${P}_{o} = {e}^{\frac{1}{2 i {k}_{o}} ( {k}_{x}^{′2} + {k}_{x}^{′2} ) δ {z}^{′}}$$

The total solution is:

$$u ( x ) = \frac{v ( {x}^{′} )}{q ( z )} {e}^{- i k ( {x}^{2} + {y}^{2} ) / q ( z )}$$

$$q ( z ) = z + i \frac{π {w}_{o}^{2}}{λ} = z + i b , b = \frac{2 π {w}_{o}^{2}}{2 λ} = \frac{β {w}_{o}^{2}}{2}$$

Letting ε go to zero to get the used coordinate system:

$${lim}_{ε \to 0} \frac{α x}{z + i ε} = \frac{α x}{z} , {y}^{′} = \frac{α y}{z} , {z}_{2}^{′} - {z}_{1}^{′} = \frac{{α}^{2} ( {z}_{2} - {z}_{1} )}{{z}_{2} {z}_{1}}$$

The coordinate transformation valid for zⱼ₋₁<z<zⱼ

Gives for the original coordinates:

$$y ≡ \frac{z}{α} {y}^{′} , x ≡ \frac{z}{α} {x}^{′} , α = {z}_{i} ; {R}_{i} = z / {z}_{i}$$

$${x}^{′} = {lim}_{ε \to 0} \frac{α x}{z + i ε} \to {x}^{′} = \frac{x}{{R}_{i}}$$

z and x, y coordinates valid for zⱼ₋₁<z<zⱼ
use α=zⱼ; Rᵢ=z/zⱼ;
so now

$${z}_{j}^{′} - {z}_{j - 1}^{′} = \frac{{α}^{2} ( {z}_{j} - {z}_{j - 1} )}{{z}_{j} {z}_{j - 1}} = \frac{( {z}_{j} - {z}_{j - 1} )}{{R}_{j - 1}}$$

$$δ {z}_{j}^{′} = \frac{δ {z}_{j}}{{R}_{j - 1}} ; {y}^{′} = \frac{y}{{R}_{i - 1}} ; {x}^{′} = \frac{x}{{R}_{i - 1}}$$

$$δ {z}_{j}^{′} = \frac{δ {z}_{j}}{{R}_{j - 1}} = \frac{( {z}_{j} - {z}_{j - 1} )}{{R}_{j - 1}}$$

Propagate from zⱼ₋₁→zⱼ in primed coordinates using the propagator derived above, then evaluate Gaussian part at

$${y}_{j + 1} ≡ \frac{z}{α} {y}_{j + 1}^{′} = {R}_{i} {y}_{j + 1}^{′} ; x = \frac{z}{α} {x}^{′} ; x = {R}_{i} {x}^{′} ;$$

and z=zⱼ+δzⱼ=zⱼ₊₁ (recall that

$${z}_{j - 1}^{′} ( z = {z}_{j - 1} ) = 0$$

by construction so,

$$\frac{{e}^{- i k ( {x}_{j + 1}^{2} + {y}_{j + 1}^{2} ) / 2 {z}_{j + 1}}}{{z}_{j + 1}} ,$$

then use

$$u ( x ) = v ( {x}^{′} ( {x}_{j + 1} ) ) \frac{{e}^{- i k ( {x}_{j + 1}^{2} + {y}_{j + 1}^{2} ) / 2 {z}_{j + 1}}}{{z}_{j + 1}}$$

Then apply the phase mask for zⱼ₋₁→zⱼ which is

$${e}^{{\int }_{{z}_{j - 1}}^{{z}_{j}} {k}_{o} γ dz} ,$$

dz is the infinitesimal along the coordinate x, y path in x, y, z space:

$$x = \frac{z}{α} {x}^{′} ; x ; x = {R}_{i} {x}^{′} and y = \frac{z}{α} {y}^{′} = {R}_{i} {y}^{′}$$

$$u ( x ) \to u ( x ) {e}^{{\int }_{{z}_{j - 1}}^{{z}_{j}} {k}_{o} γ dz}$$

In this way we march along for z₀, . . . , zⱼ₋₁, zⱼ Notice the x, y coordinates do expand due to the Rᵢ>>1 and increasing as z increases.

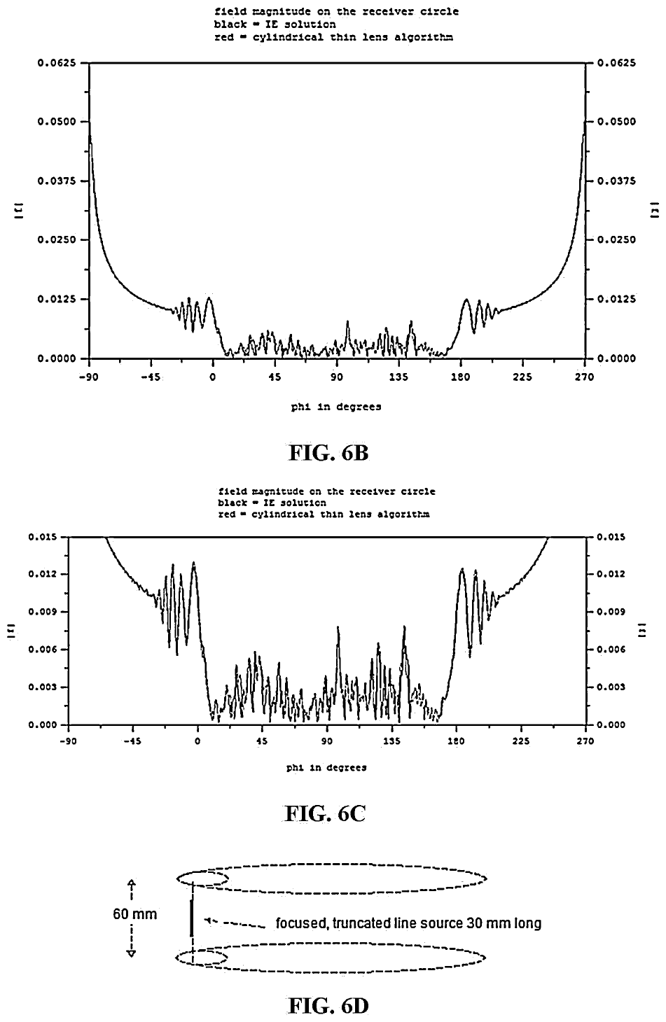

FIGS. 6A-6D illustrate results of a cylindrical coordinate system-based algorithm.

Referring to FIG. 6A, a simulation model was created from a breast image and is shown along with the position of the cylindrical algorithm circular array and the circle defining the point source boundary condition.

The solution was first computed by solution of the integral equation. The simulation model was 1024 by 1024, ⅛ wavelength pixels at 1.5 MHz (128 by 128 mm). The point source solution was computed with the Bi-Stab, FFT algorithm with a k-average preconditioner which converged in 40 steps. The total field on the receiver circle was then computed from the rectangular grid values using bi-quadratic interpolation.

The total field on the receiver circle was then computed via a cylindrical FFT-propagator, phase-mask algorithm.

FIG. 6B shows a comparison of a total field magnitude on the receiver circle; and FIG. 6C shows a magnified solution match. It can be seen that there is agreement of the cylindrical propagation algorithm with the simulation solution (referred to as IE solution) for all scattering angles.

FIG. 6D illustrates a cylindrical problem space with a radius of 100 mm and a height of 60 mm. Referring to FIG. 6D, the source is shown as a truncated, focused line source 30 mm high. The line source is also apodized with a Hamming window. The line source focal range=100 mm. The small, offset circle containing the source has a radius, a₁=5 mm. The line source position in the small circle is x₁=−2.5 mm, y₁=1.5 mm. This offset was done to ensure proper propagation of an asymmetric field.

The goal is now to evaluate the field on the 5 mm radius cylinder by Green's theorem and then to propagate the field onto the 100 mm radius cylinder using the cylindrical propagator algorithm. The Green's theorem calculation from the focused line source to the 5 mm cylinder is given by:

$${f}_{1} ( {ϕ}_{1} , z ) = {\int }_{- \frac{{h}_{L}}{2}}^{\frac{{h}_{L}}{2}} \frac{w ( {z}^{′} ) {e}^{- {ik}_{0} \sqrt{{( {a}_{1} cos ( {ϕ}_{1} ) - {x}_{L} )}^{2} + {( {a}_{1} sin ( {ϕ}_{1} ) - {y}_{L} )}^{2} + {( z - {z}^{′} )}^{2}} - \sqrt{{z}^{′ 2} + {f}_{L}^{2}}}}{4 π \sqrt{{( {a}_{1} cos ( {ϕ}_{1} ) - {x}_{L} )}^{2} + {( {a}_{1} sin ( {ϕ}_{1} ) - {y}_{L} )}^{2} + {( z - {z}^{′} )}^{2}}} {dz}^{′}$$

where w(z′) is a Hamming window, a₁=5 mm, h_{L}=30 mm, f_{L}=100 mm, X_{L}=−2.5 mm, y_{L}=1.5 mm,

$${k}_{0} = \frac{2 π f}{{c}_{0}} ,$$

f=1.5 MHz, c₀=1.5 mm/microsecond.

Returning again to FIG. 3, after generating the transmission image, reconstruction manager 310 directs generation of reflection image(s) 340 from the reflection data 314. During the preprocessing 320, the intensity of acoustic reflection based on the time it takes the waves to travel back to the source can be obtained from the reflection data 314 (e.g., a Fourier transform may be applied such as described with respect to the transmission data 312). In addition, other preprocessing operations may be carried out such as coordinate system adjustments, etc. Any suitable reconstruction algorithm may be used to generate the reflection image. In some cases, the reflection image is corrected for refraction based on the transmission image. For example, more detail is added to interfaces within the object being imaged through use of the attenuation image (from the transmission data). The interface data can also be added after image formation. As an example implementation, an attenuation image can be obtained of the reconstruction image (e.g., generated in process 336), a reflection image can be generated (e.g., via suitable reconstruction algorithm), a morphological operation can be performed with respect to the attenuation image to generate a processed attenuation image, and the processed attenuation image can be fused with the reflection image to generate a final reflection image.

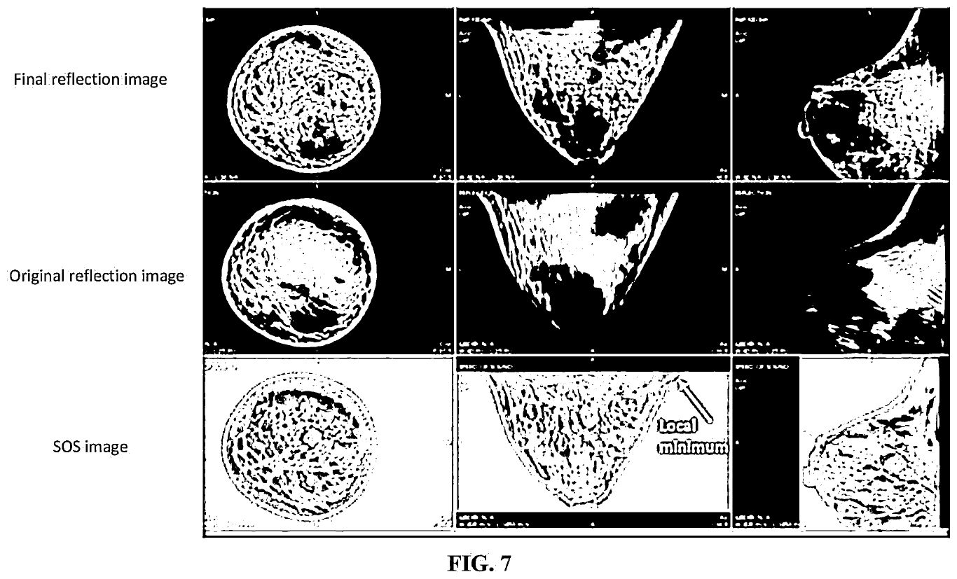

FIG. 7 shows reflection images of a breast, including the fusing of a processed attenuation image and reflection image. As shown in FIG. 7, the original reflection images correspond to the reflection image generated by any suitable reconstruction algorithm. Attenuation images are obtained and processed using a morphological operation. Examples of morphological operations that may be carried out on the attenuation images include, but are not limited to, erosion, dilation, opening, and closing. The processed attenuation images are then fused with the original reflection images to generate the final reflection images shown. As a comparison, the speed of sound (SOS) image is shown in the figure to illustrate that the information now visible in the final reflection image are not artifacts being added to the image. Advantageously, when the attenuation images highlight interface information, that interface information can improve the reflection image.

With the transmission image(s) and the reflection image(s) generated by processes 330 and 340, appropriate image evaluation processes 350 with respect to the generated images can be applied. The quantitative data obtained from the generated images can be similar to that obtained with respect to the preliminary image, but with more resolution/accuracy. For example, the quantitative data obtained from the generated images can be used for diagnostic and other purposes. Post processing 360 can be applied to the images to remove noise, perform decluttering, as well as apply desired metadata to make the images into a useful package for viewing and/or as training data for machine learning. As an example, the final reflection image(s) and transmission image(s) can be converted to DICOM format.

In some implementations, data for transmission data 312 and/or reflection data 314 can be acquired using a system such as described with respect to FIG. 1 and/or FIG. 2. Indeed, data captured by an imaging procedure using a multifrequency data collection method such as by transmitting a pulse containing multiple frequencies can be received.

In some cases, adaptive imaging architecture 300 includes or is in communication with a data resource of data collected using vertically limited diffraction-less beams. For such implementations, image reconstruction is carried out on the data collected using vertically limited diffraction-less beams. For example, in some of such implementations, the preliminary reconstruction image and/or the reconstruction image is generated by applying a 2D reconstruction algorithm. Through the adjusting of the reconstruction configuration as described herein, it is possible to reduce artifacts and speed up reconstruction.

In other of such implementations, the preliminary reconstruction image and/or the reconstruction image is generated by applying a 3D reconstruction algorithm. In some cases, the thickness of the diffraction-less beams are varied (and the 3D reconstruction can reflect such variance). In some cases, the diffraction-less beams are rotationally limited.

Creating diffraction-less beams to sonicate a breast (or other tissue) has the advantage that only second order scattering with be out of plane. The acoustic energy must scatter out of the plane, then scatter back into the plane of the receiver array.

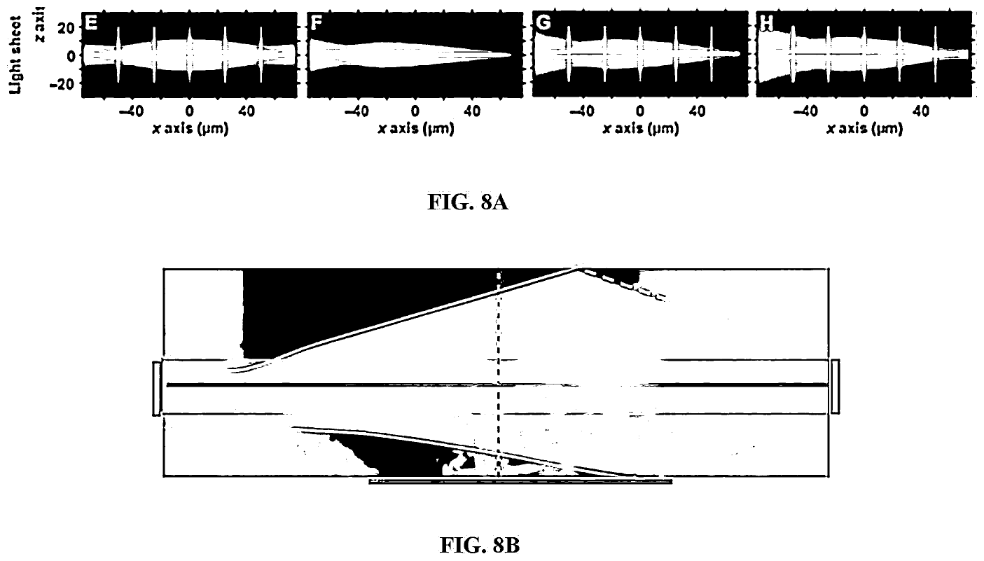

FIGS. 8A and 8B illustrate light wave propagation for diffraction-less beams. FIG. 8A illustrates a cross-section of non-diverging diffraction-less beams and FIG. 8B illustrates a cross-section of a present acoustic total field. A system for creating diffraction-less beams can form beams similar to optical light sheets used in lightsheet microscopy. As illustrated in FIG. 8A, it can be seen that the beam remains narrow and can hold form for approximately 171λ, which would be 252 mm at 1 MHz (acoustic). These beams can be concatenated to create the horizontal ‘sheets’.

### Formulation:

The well known expression generates a Gaussian profile Bessel beam

$$ψ ( ρ , z ) ≡ - \frac{ikA}{2 z Q ( z )} {e}^{ik ( z + \frac{{ρ}^{2}}{2 z} )} {J}_{0} ( \frac{{ikk}_{ρ} ρ}{2 z Q ( z )} ) {e}^{- \frac{1}{4 Q ( z )} ( {k}_{ρ}^{2} + \frac{{k}^{2} {ρ}^{2}}{{Z}^{2}} )}$$

where Q(z)≡(q−ik/2z), k_{ρ} is transverse (x-y) wavenumber,

It is also possible to use superposition as these represent solutions to the wave equation. They are reasonable approximations when considering finite apertures.

$${ψ}_{j} ( ρ , z ) ≡ - \frac{{ikA}_{j}}{2 {zQ}_{j} ( z )} {e}^{i k ( z + \frac{{ρ}^{2}}{2 z} )} {J}_{0} ( \frac{{ikk}_{ρ} ρ}{2 {zQ}_{j} ( z )} ) {e}^{- \frac{1}{4 Q ( z )} ( {k}_{ρ}^{2} + \frac{{k}^{2} {ρ}^{2}}{{z}^{2}} )}$$

with Qⱼ(z)≡(q−ik/2z)

It is known these beams do not ‘diffract’ or spread out as normal propagating wavefields do. This spreading requires the 3D algorithms to account for first order scattering from planes outside of the central plane. Only second order scattering effects occur with the ‘confined’ beam. Using these Gaussian-Bessel beams (and superpositions of them) means that only second order scattering will occur—scattering out of plane followed by scattering back into plane.

Such a superposition is:

$$ψ ( ρ , z ) ≡ {\sum }_{j} {ψ}_{j} ( ρ , z )$$

This formulation can create a pseudo-plane wave with limited diffraction in the vertical direction (a ‘sheet’ of acoustic energy). This is similar to ‘light sheet microscopy’ fields.

It is also possible to use a time domain plane wave (pseudo plane wave).

Advantageously, the confinement of the acoustic signal means that out of plane scattering is second order and so a 2D algorithm of any kind will produce a much better image than with standard unconstrained ultrasound incident fields. These beams can be produced in the time domain as well, as constrained pulses and 2D plane wave pulses.

### Evaluation Processes

As noted above with respect to operations for evaluating a preliminary reconstruction image 334 (including obtaining (422) preliminary information from the preliminary reconstruction image) and image evaluation 350, mammographic density can be measured (see also operation 508 of FIG. 5). In some cases, estimating mammographic density includes separating exterior voxels from breast voxels of the preliminary reconstruction image; segmenting high speed value breast voxels from other breast voxels of the preliminary reconstruction image to generate a first segmented image, wherein high speed value breast voxels are breast voxels having a speed value above a threshold; removing, from the first segmented image, high speed value breast voxels corresponding to skin tissue of the patient's breast to generate a second segmented image; and calculating mammographic density by determining a percentage of the high speed value breast voxels in the second segmented image.

Other information can also be obtained from the preliminary reconstruction image and/or final/updated reconstruction image. As also noted above, multifrequency images are collected. While generating a final multifrequency image, intermediate multifrequency images can be kept and used to obtain information from the preliminary reconstruction image and/or final/updated reconstruction image. As such, certain characteristics of the imaged object that vary with frequency can be used to extract useful information. While attenuation and speed of sound images are described in detail, the principle applies to images based on the backscatter (reflection) data, including with respect to attenuation coefficient slope estimate (ACS) and envelope statistics and derived parameters such as the effective scatterer diameter (ESD), effective acoustic concentration (EAC), and the μ parameter (effective scatterer density) from the HK distribution. Some of the estimates can be determined directly from the backscattered signal. As provided in detail herein certain parameters can be estimated based on tomographic reconstructed images using the transmitted signal.

### Tissue Type Identification

It has been verified in the literature that certain mammalian tissue types behave in a particular manner with frequency. It is known that attenuation varies with frequency via the power law dependency. Advantageously, by using the Kramers-Kronig relationship that relates the frequency dependence of the attenuation to the speed of sound frequency dependence, it is possible to yield a corresponding power law for the speed of sound (and use the described reconstruction images to obtain useful information to identify tissue/lesions). From the power law relationship, it can be seen that the rate of change frequency for the SOS is less than for attenuation. In fact, the frequency dependence for speed can be shown to be

$$\frac{1}{c ( x , ω )} - \frac{1}{c ( x , {ω}_{o} )} = {α}_{o} ( x ) tan ( \frac{a π}{2} ) ( {ω}^{a - 1} - {ω}_{o}^{a - 1} )$$

when α(ω)=α₀ω^{a} (see below).

This is reflected in measured values as well, where the dependence of speed is hard to measure, whereas the dependence of attenuation is measured relatively easily. There is strong evidence from the literature that attenuation varies with frequency in a way that may help to characterize biological tissue.

In particular, the standard tissue model used for attenuation is

$$α ( ω ) = {α}_{o} {ω}^{a} , ω = 2 π f$$

which leads to the linear relationship:

$$log α ( x , ω ) = log {α}_{o} ( x ) = a log ω .$$

This is acknowledged in the literature as being an approximation since the true model involves a summation of terms which represent ‘relaxation processes’ at various frequencies. The phenomenological results of investigations indicate the power law to be generally satisfied.

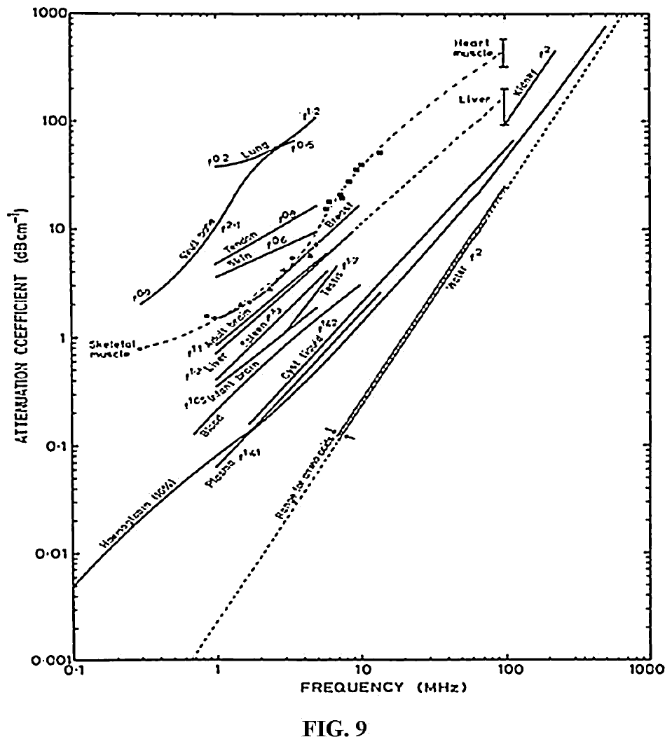

FIG. 9 shows a log plot of attenuation coefficient vs frequency from Bamber, 1986, Attenuation and absorption. Physical Principles of Medical Ultrasonics, ed. C R Hill. The plot of FIG. 9 shows the power law behavior for most tissue types is a power law variance with frequency.

The tissue type (bone, skin, ducts, glands, fat, fibroglandular tissue, cancer, etc.) may affect the attenuation coefficient as well as the ‘power law’. Speed varies with frequency by virtue of the Kramers-Kronig relations. Note that these relations are non-local in their exact form. They involve integration over the entire real line in fact. There are, however, semi-local forms that can be used in an approximate manner. Note also that an argument based on operator series shows that for the special case of a power law variation of attenuation with frequency, i.e., α(ω)=α₀ω^{a} the speed of sound must also have variation with frequency given by the formula: (proved below)

$$\frac{1}{c ( x , ω )} - \frac{1}{c ( x , {ω}_{o} )} = {α}_{o} ( x ) tan ( \frac{a π}{2} ) ( {ω}^{a - 1} - {ω}_{o}^{a - 1} ) .$$

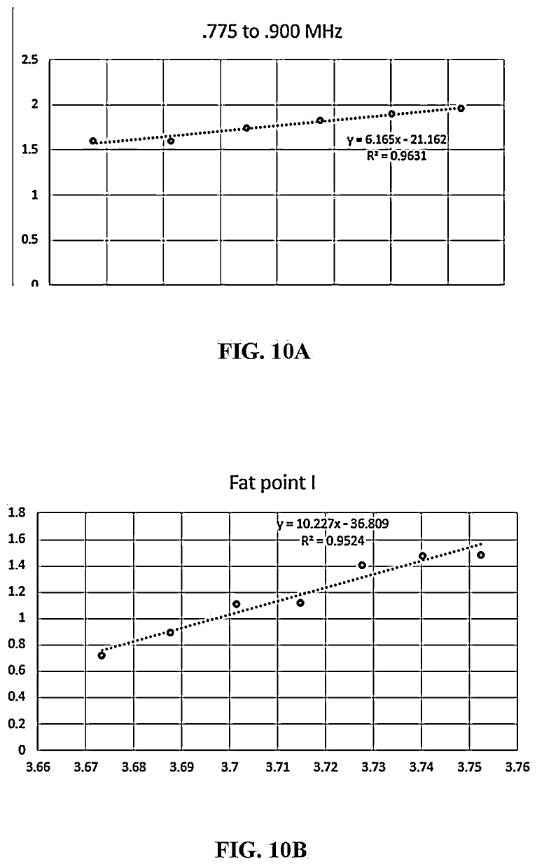

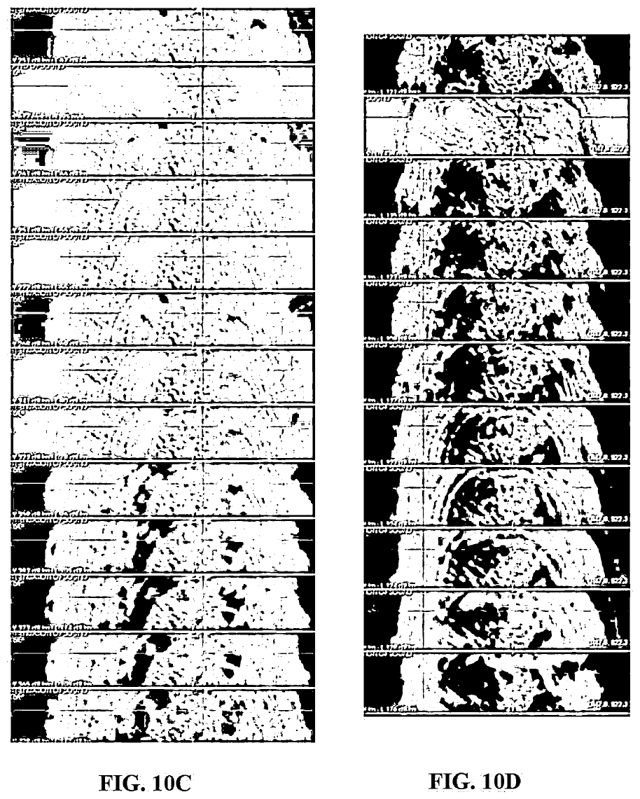

These formula show that the variation of speed with frequency will not necessarily be large since a≈1.3. This is borne out by experimental evidence as shown in FIGS. 10A-10D.

FIGS. 10A-10D illustrate identification of tissue type through the power law variation of attenuation with frequency. FIG. 10A shows a log-log plot of attenuation vs frequency (0.775, 0.8, 0.825, 0.85, 0.875, 0.9 MHz) for an image with a known cancer lesion. The region of inspection (ROI) is a 2 mm diameter centered in the known cancer lesion as determined by the speed of sound map. FIG. 10B shows a log-log plot of attenuation vs frequency for an image with fat. FIG. 10C shows panels of intermediate multifrequency images of a point in cancer and FIG. 10D shows panels of intermediate multifrequency images of a point in fat. Each image of the multifrequency images correspond to an image at a particular frequency (e.g., starting from the top with attenuation/SOS at 1.3 MHz and subsequent panels shown the attenuation images at sequentially lower frequencies to 0.400 MHz).

The calculation of these parameters over multiple frequencies can be carried out over a volume or region of interest to increase signal-to-noise (SNR) and stability. As can be seen, there is a different relationship between attenuation and frequency for a fat ROI as compared to a cancer ROI.

Using formula: log α(x,ω)=log α₀(x)=a log ω

The coefficient here is a=10.2.

Power law result: Note these are empirical results subject to noise and are for illustrative purposes only. More accurate estimates can be obtained using a volume of interest in place of a single voxel.

| | Power law |
|---|---|
| Fat | 10.2 |
| cancer | 6.16 |

Advantageously, this strong separation through the power aw parameters can be used to assign a cancer risk.

Accordingly, it is possible to obtain reconstruction information from a reconstruction image by identifying frequency-dependent characteristics using values across frequencies of the intermediate multifrequency images, including attenuation and speed of sound. For example, it is possible to calculate a frequency-independent wavenumber by removing a frequency dependent part of a wavenumber from a Helmholtz equation based model using information across frequencies of the multifrequency image. By quantitatively determining the spectral behavior of the speed of sound and attenuation images, it is possible to determine any correlation to the linear coefficients to the tissue type and/or abnormality. It is also possible to estimate mass density and porosity using such spectral behavior (e.g., the frequency-independent wavenumber). Indeed, as described herein, it is possible to estimate porosity based on identified frequency-dependent characteristics.

As an illustrative example of estimating mass density, once the attenuation is determined spatially (see e.g., examples in the imaging algorithms), mass density can be determined by first isolating the frequency independent wavenumber, solving a simple forward problem to estimate density distribution, and dividing out the density to yield the bulk modulus.

It should be noted that in the linear approximation (Born approximation) of the forward problem a simple calculation indicates that different parts of the data originate from either the monopole scattering from the bulk modulus or the dipole scattering from the density variations.

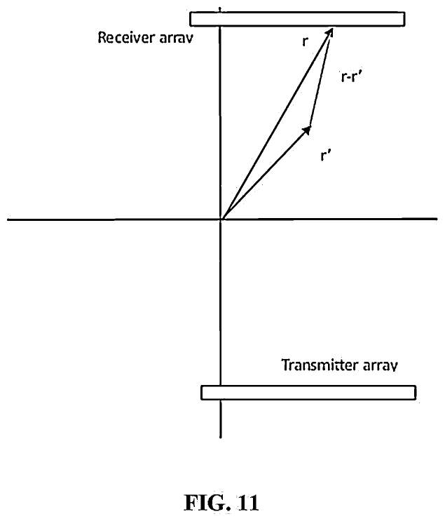

As illustrated, the data comes from different scattering potentials depending on where it is located: Note this is not an inversion scheme, rather it shows that there is some separation in the scattered data that may be exploited by a suitable scheme that has suitable regularization (see below for regularization schemes). Given a configuration such as shown in FIG. 11, which illustrates propagation between a transmitter and receiver. Note the Lippmann-Schwinger (LS) equation reads:

$${f}^{i} ( r ) = f ( r ) - {k}_{o}^{2} {\int }_{{R}^{3}} {γ}_{κ} ( {r}^{′} ) f ( {r}^{′} ) {g}_{{k}_{o}} ( R ) {dr}^{′} + {\int }_{{R}^{3}} {∇}^{′} \cdot ( {γ}_{ρ} ( {r}^{′} ) \{ {∇}^{′} f ( {r}^{′} ) \} ) {g}_{{k}_{o}} ( R ) {dr}^{′}$$

Directly from the differential equation. Integrate by parts and use Gauss theorem to remove total divergence:

$${f}^{i} ( r ) = f ( r ) - {k}_{o}^{2} \int {γ}_{κ} ( {r}^{′} ) f ( {r}^{′} ) {g}_{{k}_{o}} ( R ) {dr}^{′} + \int {γ}_{ρ} ( {r}^{′} ) \{ {∇}^{′} f ( {r}^{′} ) \} \cdot {∇}^{′} {g}_{{k}_{o}} ( R ) {dr}^{′}$$

Using R=r−r′ and f=fⁱ(r)=e^{ik}^{ᵢ}^{·r} (plane wave incident) and the standard approximation:

$${g}_{{k}_{o}} ( R ) == \frac{{e}^{- i k R}}{4 π R} = \frac{{e}^{- ik \vert r - {r}^{′} \vert}}{4 π \vert r - {r}^{′} \vert} \approx \frac{{e}^{- i k ( r - {u}_{r} \cdot {r}^{′} )}}{4 π \vert r \vert} = \frac{{e}^{- i k r}}{4 π r} {e}^{- i k {u}_{r} \cdot {r}^{′}}$$

∇′gₖ_{ₒ}(R)=−ikuᵣe^{-iku}^{ᵣ}^{·r′} for Green's function with approximation.

Define: kᵣ=kuᵣ vector pointing toward the receiver array element.

Substitute plane wave and Green's function approximation in the IE to get

$${f}^{s} ( r ) \approx \frac{{e}^{- \dot{t} k R}}{4 π R} ( {k}^{2} \int {γ}_{κ} ( {r}^{′} ) {e}^{- i ( {k}_{r} - {k}_{i} ) \cdot {r}^{′}} {dr}^{′} + {k}_{i} \cdot {k}_{r} \int {γ}_{ρ} ( {r}^{′} ) \{ {e}^{- i ( {k}_{r} - {k}_{i} ) \cdot {r}^{′}} \} {dr}^{′} )$$

Inner product is cos of angle between them times k² so:

$${f}^{s} ( r ) \approx \frac{{e}^{- i k r}}{4 π r} {k}^{2} ( \int {γ}_{κ} ( {r}^{′} ) {e}^{- i ( {k}_{r} - {k}_{i} ) \cdot {r}^{′}} {dr}^{′} + cos θ \int {γ}_{ρ} ( {r}^{′} ) \{ {e}^{- i ( {k}_{r} - {k}_{i} ) \cdot {r}^{′}} \} {dr}^{′} )$$

These are Fourier transforms and give expression for scattered field.

$${f}^{s} ( r ) \approx \frac{{e}^{- i k r}}{4 π r} {k}^{2} ( {Γ}_{κ} ( {k}_{r} - {k}_{i} ) + cos θ {Γ}_{ρ} ( {k}_{r} - {k}_{i} ) )$$

where θ is the angle between kᵣ, kᵢ.

This gives the relation in the linear approximation between scattered data and the object functions for density and bulk modulus. To complete the separation, the following correlations between mass density and speed can be utilized, particularly for the speed values of interest for mammalian tissue.

$$K = ( 1706.507 {c}^{2} - 1611.7 \; 37 ) MPa$$

$$K = 89 \; 3 {c}^{3} - 349 {c}^{2} = {c}^{2} ( 893 c - 3 \; 49 ) MPa$$

The above correlations are particularly useful as a regularizing term in density reconstruction and can be used during the preliminary image reconstruction. For example, it is possible to estimate the bulk modulus and shear modulus for breast and mammalian tissue from these correlations. Note:
K=c²ρ∝c³ which indicates that the cube of the speed image can be considered a stiffness image.

Bulk modulus and shear wave estimation is possible from the speed of sound images (e.g., as reconstruction information from the reconstruction images). For example, the shear modulus turns out to be more sensitive in some sense to the presence of a hard lesion, since a cancer can have a shear speed 30 times greater than normal tissue.

$$Accepting ( based on {c}_{s} = \sqrt{\frac{μ}{ρ}} )$$

$$\frac{c}{{c}_{s}} = \sqrt{\frac{K}{μ}} gives ( see below )$$

$$μ \approx \frac{K}{9000} so that μ ∝ \frac{{c}^{3}}{9000}$$

Accordingly, it can be seen that a shear moduli estimation is possible.

In particular, the estimate of μ based on the shear wave speed of tissue being approximately 5 m/sec is

$$μ = \frac{K}{{( c / {c}_{s} )}^{2}} \approx \frac{K}{{( 300 )}^{2}} = \frac{K}{9 * 1 {0}^{4}}$$

The Bulk modulus formula:

K=893c³=c²(893c−349), c in mm/μ sec, K in MPa.

Using this formula, it is possible to show ‘stiffness’ directly in the images.

Here ‘stiffness’ is bulk modulus. Thus it is possible to show a quantitative representation of stiffness that others are showing qualitatively. Note that it is possible to determine the Young's modulus as ˜3μ, the shear modulus presumably using the perturbation formula from (E is Young's modulus):

$$E = μ \frac{3 λ + 2 μ}{λ + μ} \approx μ 3 ( 1 + 2 μ / 3 λ ) ( 1 - \frac{μ}{λ} + … )$$

The perturbation expansion to first order in p being E≈3μ.

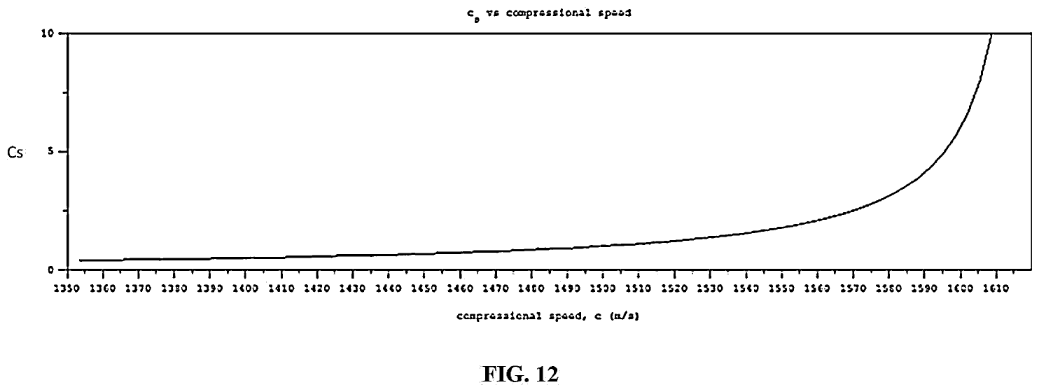

Note also, as illustrated by the plot of FIG. 12, the shear wave speed greatly magnifies the differences in speed between compressional wave speed 1560 to 1610. This is the range for differentiating between fibroadenomas and cancer.

Using the formula:

$$μ = \frac{K}{{( c / {c}_{s} )}^{2}} , where K = 893 {c}^{3} - 349 {c}^{2} = {c}^{2} ( 893 c - 3 \; 49 ) MPa$$

### Mass Density Estimation

Accordingly, it is possible to utilize the multifrequency images in attenuation and remove the frequency dependent part of the ‘equivalent wavenumber’ that results from the square root transformation utilized to justify neglect of density. Once the frequency independent of the ‘equivalent wavenumber’ is determined, there remains a nonlinear second order differential equation that can be transformed in two stages into a linear second order equation (essentially a Helmholtz equation) with known wavenumber and boundary conditions known (it is an elliptic equation).

The problem can be solved once (since it is not an inverse problem), and the spatial distribution of the density can be retrieved.

Once the density is known it is possible to estimate the bulk modulus by virtue of the formula:

$$c = \sqrt{\frac{K}{ρ}}$$

The density dependent wave equation is known to be

$${∇}^{2} p + \frac{ω}{{c}_{o}^{2}} p = ∇ \cdot ( {γ}_{ρ} ∇ p ) - \frac{ω}{{c}_{o}^{2}} {γ}_{κ} p$$

where γ_{κ}≡(κ−κₒ)/κₒ, γ_{ρ}≡(ρ−ρₒ)/ρ are the gamma object functions for compressibility and density respectively. Pressure is p, frequency is ω=2πf and background speed of sound is cₒ. Wavenumber

$${k}_{o}^{2} ≡ {ω}^{2} / {c}_{o}^{2} .$$

### Using

$${p}^{′} = \frac{p}{\sqrt{ρ}}$$

the equation becomes ∇²p′(x)+k′²(x)p′(x)=0 with pseudo-wavenumber given by:

$${k}^{′ 2} ( x ) = {k}^{2} ( x ) - \frac{3}{4 {ρ}^{2}} {( ∇ ρ )}^{2} + \frac{1}{2 ρ} {∇}^{2} ρ .$$

Note the true wavenumber

$$k ( x ) ≡ \frac{ω}{c ( x )} + i α ( x ) .$$

As noted the attenuation generally follows a power law with exponent of about 1.2 or so for mammalian tissue. Accordingly, it is possible to remove the frequency dependent part. Kramer's Kronig relations can be exactly integrated in this case and the power law for speed of sound ˜0.2 k_{ind}(x) is the frequency independent part that remains when the frequency dependent part is subtracted out.

Because multiple frequency images are generated, there is access to this information. We use the identity

$$\frac{1}{ρ} {∇}^{2} ρ = ∇ \cdot ( \frac{1}{ρ} ∇ ρ ) + \frac{1}{{ρ}^{2}} ∇ ρ ∇ ρ :$$

(proved by direct calculation).

The equation now reads:

$${k}^{′ 2} ( x ) - {k}^{2} ( x ) ≡ {k}_{ind}^{2} = - \frac{1}{4} ∇ ln ρ \cdot ∇ ln ρ + \frac{1}{2} ∇ .$$

(∇ ln ρ), To solve this equation we use the transformation: To solve this equation, we first transform to a new y variable:

$$\frac{1}{2} {\partial }_{i} ln ρ = - {\partial }_{i} ln y .$$

The equation now reads

$$- \frac{{∇}^{2} y}{y} = {k}_{ind}^{2} ( x )$$

which can be rewritten as

$${∇}^{2} y ( x ) + {k}_{ind}^{2} ( x ) y ( x ) = 0 .$$

The method can include generating images at the multiple frequencies; estimating the attenuation at multiple frequencies, removing the frequency dependent part of the wavenumber, leaving k_{ind}(x), solving

$${∇}^{2} y ( x ) + {k}_{ind}^{2} ( x ) y ( x ) = 0 ,$$

the forward Helmholtz problem for y (suitable BCs), and estimating density as

$$ρ = \frac{{e}^{c}}{{y}^{2}} = \frac{K}{{y}^{2}} ,$$

for suitably determined constant, K determined by the known density of water at the boundary and in the water bath.

### Biot—Porous Media

It is noted that the integral equation formulation for the Biot Theory in the Acoustic approximation is provided in the imaging algorithm section (see the vector Lippmann-Schwinger equation—Fredholm of Second Kind) as well as the paraxial approximation to the Helmholtz.

Expanding upon the imaging algorithm provided below, a conversion to the paraxial form is provided. An explicit method for determination of the porosity parameters based on the spectral determination of speed of sound and attenuation is included.

The full elastic Biot model is shown as follows:

$$( \begin{matrix}{\hat{ρ}}_{11} & {\hat{ρ}}_{12} \\ {\hat{ρ}}_{21} & {\hat{ρ}}_{22}\end{matrix} ) ( \begin{matrix}{\partial }_{t}^{2} u \\ {\partial }_{t}^{2} U\end{matrix} ) = ( \begin{matrix}P & Q \\ Q & R\end{matrix} ) ( \begin{matrix}∇ ( ∇ . u ) \\ ∇ ( ∇ . U )\end{matrix} ) - ( \begin{matrix}N ( ∇ \times ∇ \times u ) \\ 0\end{matrix} )$$

This is reduced via homogenization theory to effective parameters (see below for definition of the parameters):

$$μ Δ {U}_{s} + ( λ + μ ) grad div ( {U}_{s} ) = - {ω}^{2} ( {ρ}_{11} {U}_{s} + {ρ}_{12} {U}_{l} ) + i ω η ( {U}_{s} - {U}_{l} )$$

$$n α / β grad div [ ( α / n - 1 ) {U}_{s} - {U}_{l} ] = - {ω}^{2} ( {ρ}_{21} {U}_{s} + {ρ}_{22} {U}_{l} ) - i ω η ( {U}_{s} - {U}_{l} ) ,$$

$$\begin{matrix}strain & inertial & viscous \\ energy & terms & dissipation\end{matrix}$$

where

$$H = {H}_{1} + {iH}_{2} = 1 / K ( ω )$$

with

$${ρ}_{11} = ( 1 - n ) {ρ}_{s} + α ( {nH}_{2} / ω - {ρ}_{l} )$$

$${ρ}_{12} = n ( {ρ}_{l} - α {H}_{2} / ω ) η = α n {H}_{1}$$

$${ρ}_{21} = α ( {ρ}_{l} - n {H}_{2} / ω )$$

$${ρ}_{22} = α n {H}_{2} / ω .$$

This is a full elastic representation and the details are in Boutin et al. (C. Boutin, G. Bonnet, and P. Y. Bard, “Green functions and associated sources in infinite and stratified poroelastic media,” Geophysical Journal International, vol. 90, pp. 521-550, 1987).

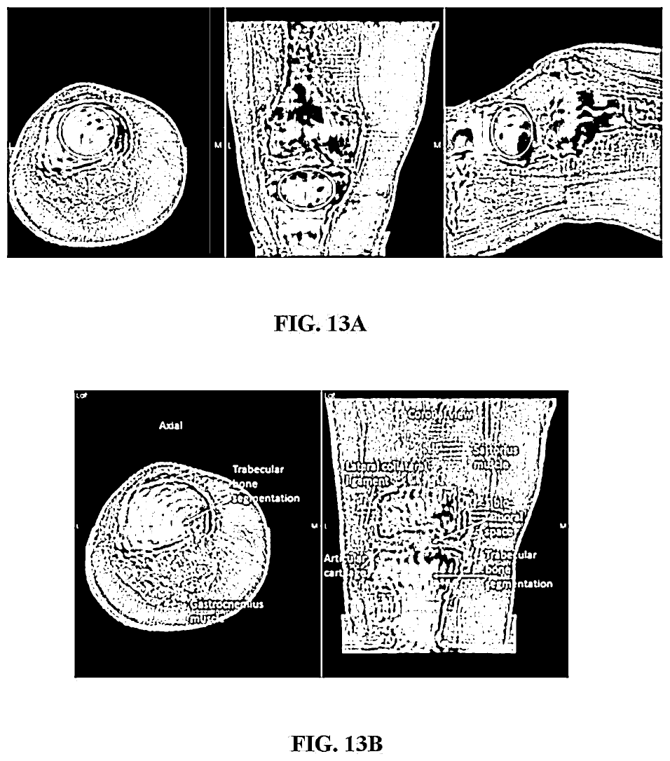

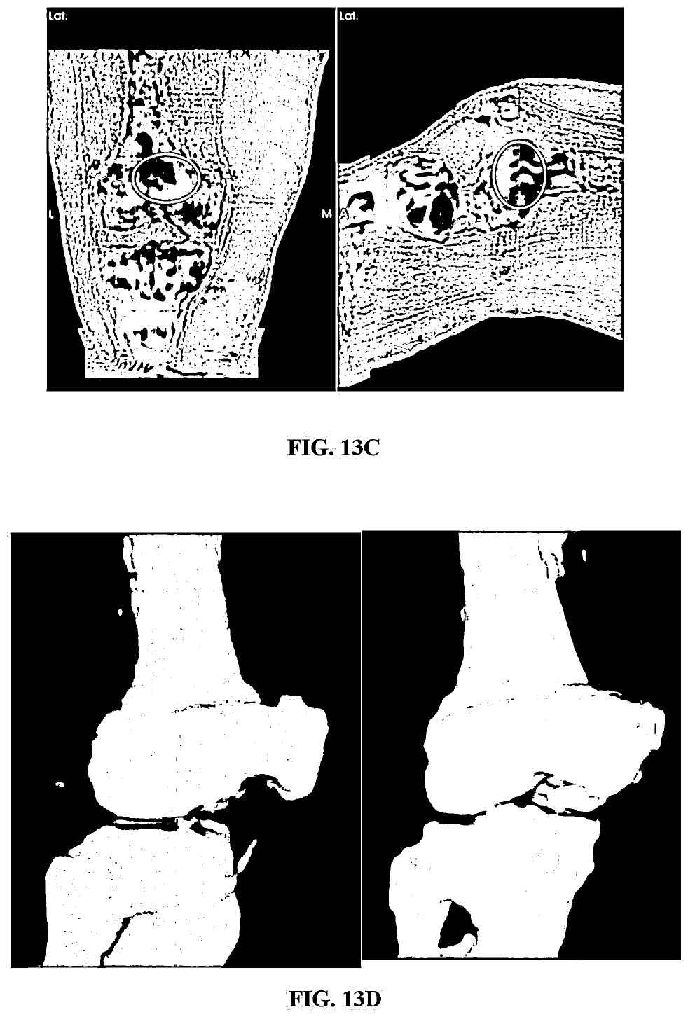

FIGS. 13A-13D provide images showing application of porosity estimation using Biot as described herein.

As shown in FIG. 13A, segmentation of marrow in tibia based on the SOS and constrained by ellipsoid gives average value 1402.2 m/s which agrees with literature values. FIG. 13B shows bone segmentation (trabecular) in tibia. FIG. 13C shows segmentation of trabecular bone in coronal and sagittal views in femur.

The Table 1 below gives the quantitative accuracy of the bone marrow and speed of sound of the Biot slow wave.

TABLE 1
comparison of segmented vs literature
values for marrow and Biot slow wave
human bone marrow
Biot slow wave SOS

| Literature values | QTUS measured values |
|---|---|
| ~1410 m/s | 1402, 1389 |
| ~1470 m/s [2, 3] | 1472, 1466 m/s |

### Homogenization in Biot Context:

Note that often in mathematical analysis of wave or diffusion phenomena in disordered or periodically structured media, homogenization is used. When the characteristic size of the periodicity for example is size ε<<1 compared with the O(1) size of the macrostructure, analytic expressions relating coefficients at the macroscopic scale based on the microstructure and the concomitant coefficients can be formulated. Furthermore, as the ratio ε→0, characteristic homogenization parameters are created. These macroscopic parameters are useful for studying wave and diffusion parameters in microstructured media. In particular in porous media:

Using the notation of Boutin et al., the following model for wave propagation in porous media results:

$$( λ + 2 μ ) {∇}^{2} {P}_{s} + {ω}^{2} ρ {P}_{s} - {α}_{0} {∇}^{2} P = - ∇ \cdot F ( x )$$

$$θ {∇}^{2} P - β P - {α}_{0} {P}_{s} = - V ( x )$$

Where Pₛ≡∇Uₛ is the divergence of particle motion in the solid matrix, and
α₀, θ, ρ are parameter's defined in the following way:

$$ρ = ( 1 - n ) {ρ}_{s} + {ρ}_{l} [ n + {ρ}_{l} {ω}^{2} K ( ω ) / ιω ]$$

$$θ = K ( ω ) / ιω$$

$${α}_{o} = α + {ρ}_{l} {ω}^{2} K ( ω ) / ιω = α + {ρ}_{l} {ω}^{2} θ$$

Where, α=1−K_{b}/Kₛ, with K_{b}=3λ+2μ/3, the bulk modulus, and l, m are the Lame' parameters in standard linear elastic theory. Also β=α−n/Kₛ+n/K_{f}, and:
ω is the frequency of the interrogating wave
ρₗ is the density of the liquid phase
ρₛ is the density of the solid phase
n is a saturation parameter
K(ω) the generalized, explicitly frequency-dependent Darcy coefficient introduced via homogenization theory

In the above, an approximate value based on homogenization theory for straight ducts can be used:

$$K ( ω ) = ( ik ) / ( ν {ω}^{*} ) {J}_{2} ( √ - 8 i {ω}^{*} ) / {J}_{0} ( √ - 8 i {ω}^{*} ) ; k = n {a}^{2} / 8 ; {ω}^{*} = ω k / nv$$

with J₀, J₂ being the Bessel functions, a the radius of the ducts, and ν the kinematic viscosity of the fluid

This is an acoustic approximation to the full elastic equations but presents an opportunity for an exact solution to the Green's function equation:

BG_{Biot}+I_{2×2}δ(x)=0, where I_{2×2} is the 2D identity and

G_{Biot}:R^{2,3}→R²

Is a matrix Biot Green's operator. B is the operator matrix

$$B = [ \begin{matrix}θ {∇}^{2} - β & - {α}_{0} \\ - α {∇}^{2} & λ {∇}^{2} + {ω}^{2} ρ\end{matrix} ]$$

Using the adjoint of this matrix (i.e., the signed matrix of cofactors, not the linear adjoint), the following solution is obtained for the Biot Green's operator.

$${G}_{Biot} = \frac{1}{4 π θ λ} \frac{{α}_{0}}{( {δ}_{2}^{2} - {δ}_{1}^{2} )} \{ ( w \cdot d ) [ \begin{matrix}λ & 0 \\ {α}_{0} & θ\end{matrix} ] + ( w \cdot v ) [ \begin{matrix}\overline{ρ} {ω}^{2} & {α}_{0} \\ 0 & - \overline{β}\end{matrix} ] \}$$

This is used in the Generalized acoustic Biot Lippmann-Schwinger equation:

$${r}_{0} ( x ) = r ( x ) - [ \begin{matrix}- {\overline{b}}_{1} & 0 \\ 0 & {\overline{b}}_{2}\end{matrix} ] \int \int \int {G}_{2 \times 2} ( x - ξ ) {D}^{′} ( ξ ) r ( ξ ) {d}^{3} ξ$$

$${D}^{′} ( x ) = [ \begin{matrix}{γ}_{1} & 0 \\ 0 & {γ}_{2}\end{matrix} ]$$

Contains the object functions that contain the tissue characteristics. Also where r: R^{2,3}→R², and s: R^{2,3}→R² are vector-valued functions on R³ or R² given by

$$r ( x ) = [ \begin{matrix}P ( x ) \\ {P}_{s} ( x )\end{matrix} ] , s = [ \begin{matrix}V ( x ) \\ ∇ \cdot F ( x )\end{matrix} ]$$

where w: R³→R² is the 2D vector defined by:

$$w ≡ \begin{matrix}\frac{{e}^{- i {δ}_{1} \vert x \vert}}{\vert x \vert} \\ \frac{{e}^{- i {δ}_{2} \vert x \vert}}{\vert x \vert}\end{matrix}$$

Where it is seen that the dᵢ are effective wavenumbers in porous media and the vectors

$$d = ( \begin{matrix}- {δ}_{1}^{2} \\ {δ}_{2}^{2}\end{matrix} ) , v = ( \begin{matrix}1 \\ - 1\end{matrix} )$$

Are so defined.

The δᵢ are the effective wavenumbers.

Using this Green's function the forward problem is well defined.

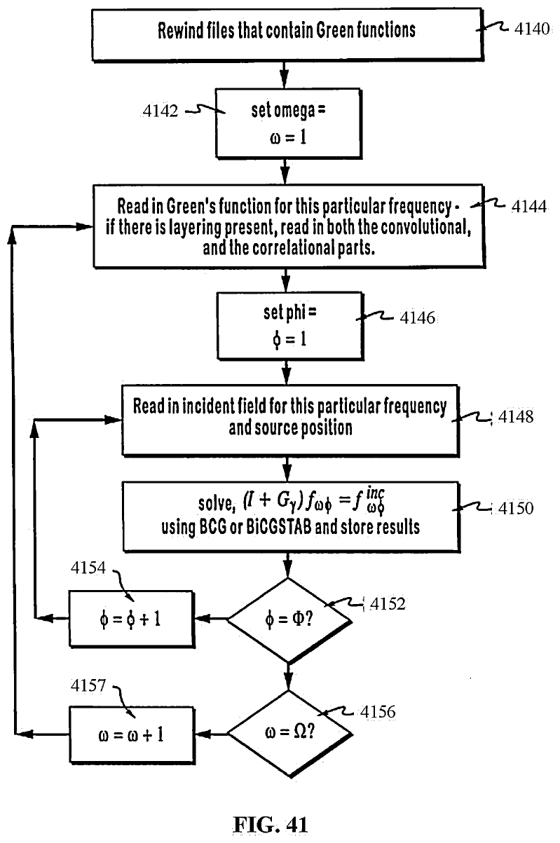

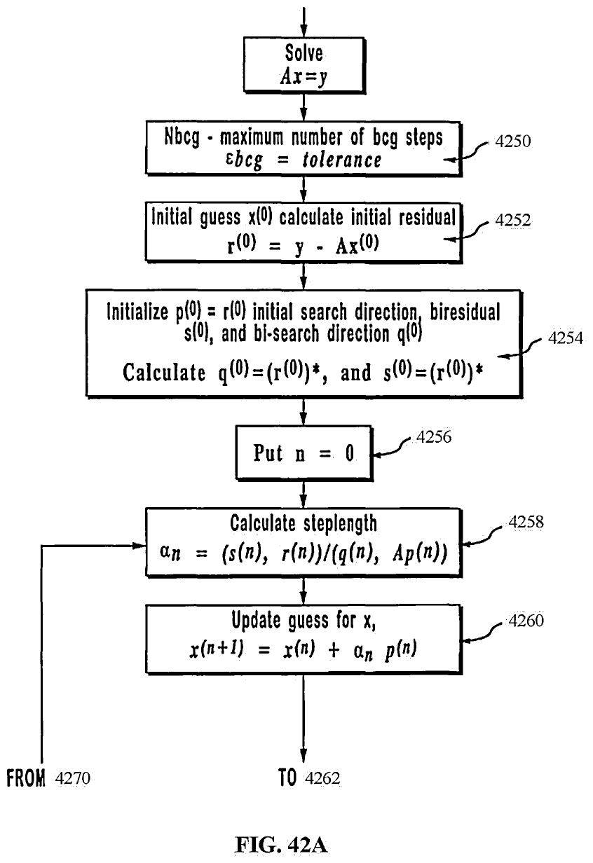

Furthermore, the Jacobian calculation and adjoint of the Jacobian calculation proceeds as described herein (see e.g., step 4150 of FIG. 41 and SCIENTIFIC BACKGROUND ON SQUARING AN OVERDETERMINED SYSTEM TO APPLY BiSTAB)

These operations respectively provide a step length and the gradient direction.

This allows for the full implementation of the inversion algorithm.

In one embodiment the imaged parameters are: Darcy coefficient and Porosity. FIG. 13D shows an attenuation image of human (ex vivo) cadaver knee. The patella, tibia, fibula and femur are present, the tibiofemoral space is clear. This anomalous attenuation is related to the Biot generalized D'Arcy coefficient.

Other embodiments can include more or fewer parameters in addition to the standard wave speed and attenuation. The other parameters that are not ‘imaged’ via this disclosure can be determined from literature values.

These calculations can be carried out on CUDA or similar AMD cards/processors for speed.

### Acoustic Theory Appropriate for Multiple Organs

Note that although orthopedic images are shown here, the porosity and density parameters apply to lungs, kidneys, liver, and other organs or abdominal imaging.

### Paraxial Approximation for Biot Wave Theory:

Recall that pressure in the solid is: Pₛ≡∇□Uₛ Pₛ≡∇Uₛ The inhomogeneous Biot equation from above is:

$$( \begin{matrix}[ ( λ + 2 μ {∇}^{2} + {ω}^{2} ρ ] & - {α}_{o} {∇}^{2} \\ - {α}_{o} & θ {∇}^{2} - β\end{matrix} ) ( \begin{matrix}{P}_{s} \\ P\end{matrix} ) = ( \begin{matrix}- ∇ F \\ - V\end{matrix} )$$

Rewriting this as:

$$( \begin{matrix}( λ + 2 μ ) & - {α}_{o} \\ 0 & θ\end{matrix} ) {∇}^{2} + ( \begin{matrix}{ω}^{2} ρ & 0 \\ - {α}_{o} & - β\end{matrix} ) ( \begin{matrix}{P}_{s} \\ P\end{matrix} ) = ( \begin{matrix}- ∇ F \\ - V\end{matrix} )$$

And using the matrix definitions:

$$A ≡ ( \begin{matrix}( λ + 2 μ ) & - {α}_{o} \\ 0 & θ\end{matrix} ) , B ≡ ( \begin{matrix}{ω}^{2} ρ & 0 \\ - {α}_{o} & - β\end{matrix} )$$

Gives the following form for the Biot acoustic approximation.

$$( A {∇}^{2} + B ) ( \begin{matrix}{P}_{s} \\ P\end{matrix} ) = 0$$

This can be solved using the Biot Acoustic Green's function developed above. This leads to a generalized Lippmann-Schwinger equation that is rigorous and leads to an inversion algorithm by itself and the concomitant step length and gradient direction calculations.

As can be seen from the images, it is possible to detect the slow Biot nonstandard compression wave (P wave) and have segmented the trabecular bone region interior to the bone and obtained a value commensurate with the predicted speed of sound for this slow Biot wave, yielding clinically valuable effective parameters.

### Paraxial Approximation:

This vector Helmholtz equation can be approximately factored into

$$( \begin{matrix}( λ + 2 μ ) & - {α}_{o} \\ 0 & θ\end{matrix} ) {∇}^{2} + ( \begin{matrix}{ω}^{2} ρ & 0 \\ - {α}_{o} & - β\end{matrix} ) \approx ( \sqrt{A} ∇ + i \sqrt{B} ) ( \sqrt{A} ∇ - i \sqrt{B} )$$

Where
β=(α−n)/Kₛ+n/K_{f} is a volume averaged ‘averaged’ Bulk modulus

And a is a relative difference modulus, and aₒ is a frequency dependent relative difference modulus with the effective Darcy coefficient K:

$$α = 1 - {K}_{b} / {K}_{s} , {α}_{o} ≡ α + {ρ}_{l} {ω}^{2} K ( ω ) / i ω$$

The matrix square root can be found in the standard way for A and B:

This leads to the parabolic PDE (paraxial approximation);

$$( \sqrt{A} ∇ - i \sqrt{B} ) P \approx 0$$

By direct calculation then:

$$\sqrt{B} = ( \begin{matrix}ω \sqrt{ρ} & 0 \\ \frac{{α}_{o} ( i \sqrt{β} - ω \sqrt{ρ} )}{{ω}^{2} ρ + β} & i \sqrt{β}\end{matrix} ) , and$$

$$\sqrt{A} = ( \begin{matrix}\sqrt{λ + 2 μ} & \frac{{α}_{o} ( \sqrt{θ} - \sqrt{λ + 2 μ} )}{( λ + 2 μ ) - θ} \\ 0 & \sqrt{θ}\end{matrix} )$$

We are working in the acoustic approximation, so μ≈0 and

$$\sqrt{A} = ( \begin{matrix}\sqrt{λ + 2 μ} & \frac{{α}_{o} ( \sqrt{θ} - \sqrt{λ + 2 μ} )}{( λ + 2 μ ) - θ} \\ 0 & \sqrt{θ}\end{matrix} ) \approx ( \begin{matrix}\sqrt{λ} & \frac{{α}_{o} ( \sqrt{θ} - \sqrt{λ} )}{λ - θ} \\ 0 & \sqrt{θ}\end{matrix} )$$

So that finally the acoustic approximation (μ≈0) Biot theory reads:

$$( ( \begin{matrix}\sqrt{λ} & \frac{{α}_{o} ( \sqrt{θ} - \sqrt{λ} )}{λ - θ} \\ 0 & \sqrt{θ}\end{matrix} ) ∇ - i ( \begin{matrix}ω \sqrt{ρ} & 0 \\ \frac{{α}_{o} ( i \sqrt{β} - ω \sqrt{ρ} )}{{ω}^{2} ρ + β} & i \sqrt{β}\end{matrix} ) ) ( \begin{matrix}{P}_{s} \\ P\end{matrix} ) \approx 0$$

### Paraxial Approximation in Suitable Form

The wave equation for the Biot acoustic model in the paraxial approximation now reads in a form that allows for determination of the effective speed of sound and other parameters:

$$( {I}_{2 \times 2} ∇ - i {\sqrt{A}}^{- 1} \sqrt{B} ) ( \begin{matrix}{P}_{s} \\ P\end{matrix} ) \approx 0$$

Now the inverse of sqrt(A) can be calculated and the appropriate calculation carried out explicitly to yield:

$${\sqrt{A}}^{- 1} \sqrt{B} = {( \begin{matrix}\sqrt{λ} & \frac{{α}_{o} ( \sqrt{θ} - \sqrt{λ} )}{λ - θ} \\ 0 & \sqrt{θ}\end{matrix} )}^{- 1} ( \begin{matrix}ω \sqrt{ρ} & 0 \\ \frac{{α}_{o} ( i \sqrt{β} - ω \sqrt{ρ} )}{{ω}^{2} ρ + β} & i \sqrt{β}\end{matrix} )$$

So that now the wave (factored) equation is using:

$${\sqrt{A}}^{- 1} \sqrt{B} = \frac{1}{\sqrt{λ} \sqrt{θ}} ( \begin{matrix}\sqrt{θ} ω \sqrt{ρ} - \frac{{α}_{o} ( \sqrt{θ} - \sqrt{λ} )}{λ - θ} \frac{{α}_{o} ( i \sqrt{β} - ω \sqrt{ρ} )}{{ω}^{2} ρ + β} & - i \frac{{α}_{o} ( \sqrt{θ} - \sqrt{λ} )}{λ - θ} \sqrt{β} \\ \sqrt{λ} \frac{{α}_{o} ( i \sqrt{β} - ω \sqrt{ρ} )}{{ω}^{2} ρ + β} & i \sqrt{β} \sqrt{λ}\end{matrix} )$$

And the acoustic approximation. μ≈0 as below:

That is, explicitly:

$$( {I}_{2 \times 2} ∇ - i \frac{1}{\sqrt{λ} \sqrt{β}} ( \begin{matrix}\sqrt{θ} ω \sqrt{ρ} - \frac{{α}_{o} ( \sqrt{θ} - \sqrt{λ} )}{λ - θ} \frac{{α}_{o} ( i \sqrt{β} - ω \sqrt{ρ} )}{{ω}^{2} ρ + β} & - i \frac{{α}_{o} ( \sqrt{θ} - \sqrt{λ} )}{λ - θ} \sqrt{β} \\ \sqrt{λ} \frac{{α}_{o} ( i \sqrt{β} - ω \sqrt{ρ} )}{{ω}^{2} ρ + β} & i \sqrt{β} \sqrt{λ}\end{matrix} ) ) ( \begin{matrix}{P}_{s} \\ P\end{matrix} ) = 0$$

If we neglect terms that have w², w in the denominator, we get:

$$( {I}_{2 x 2} ∇ - i \frac{1}{\sqrt{λ} \sqrt{θ}} ( \begin{matrix}\sqrt{θ} ω \sqrt{ρ} & - i \frac{{α}_{o} ( \sqrt{θ} - \sqrt{λ} )}{λ - θ} \sqrt{β} \\ 0 & i \sqrt{β} \sqrt{λ}\end{matrix} ) ) ( \begin{matrix}{P}_{s} \\ P\end{matrix} ) = 0$$

Note also the simplification:

$$- \frac{{α}_{o} ( \sqrt{θ} - \sqrt{λ} )}{λ - θ} = \frac{{α}_{o}}{\sqrt{θ} + \sqrt{λ}}$$

So that

$$( {I}_{2 x 2} ∇ - i ( \begin{matrix}ω \frac{1}{\sqrt{λ}} \sqrt{ρ} & i \frac{{α}_{o}}{\sqrt{λ} \sqrt{θ} ( \sqrt{θ} + \sqrt{λ} )} \sqrt{β} \\ 0 & i \frac{1}{\sqrt{θ}} \sqrt{β}\end{matrix} ) ) ( \begin{matrix}{P}_{s} \\ P\end{matrix} ) = 0$$

It is possible to separate out parameters

$$( {I}_{2 x 2} ∇ - i ( \begin{matrix}\frac{1}{\sqrt{λ}} & 0 \\ 0 & 1\end{matrix} ) ( \begin{matrix}ω & i \frac{{α}_{o}}{( \sqrt{θ} + \sqrt{λ} )} \\ 0 & i\end{matrix} ) ( \begin{matrix}\sqrt{ρ} & 0 \\ 0 & \frac{\sqrt{β}}{\sqrt{θ}}\end{matrix} ) ) ( \begin{matrix}{P}_{s} \\ P\end{matrix} ) = 0$$

Note this form makes clear that there is only one component that truly corresponds to a propagating wave. (although the complex values of the effective parameters indicates a mixture of modes).

Note that as expected the relevant parameters, q, b, l all occur in square root form and the frequency occurs linearly in this approximation.

The bottom equation is diffusion since

β=(α−n)/Kₛ+n/K_{f} is a volume averaged ‘averaged’ Bulk modulus and l is real.

Also a is a relative difference modulus, and aₒ is a frequency dependent relative difference modulus with the effective Darcy coefficient K:

$$α = 1 - {K}_{b} / {K}_{s} , {α}_{o} ≡ α + {ρ}_{l} {ω}^{2} K ( ω ) / i ω$$

The top equation reads:

$$∇ {P}_{s} - i \frac{ω \sqrt{ρ}}{\sqrt{λ}} {P}_{s} + \frac{{α}_{o} \sqrt{β}}{\sqrt{λ} \sqrt{θ} ( \sqrt{θ} + \sqrt{λ} )} P = 0$$

The last term is diffusion like (although sqrt(q) is complex so there is a propagation component) and corresponds to evanescent waves that won't propagate to the receiver arrays.

Recall that β=(α−n)/Kₛ+n/K_{f}, and
that α=1−K_{b}/Kₛ, αₒ≡α+ρₗω²K(ω)/iω

Also recall
αₒ, θ, ρ are parameters defined in the following way:

$$ρ = ( 1 - n ) {ρ}_{s} + {ρ}_{l} [ n + {ρ}_{l} {ω}^{2} K ( ω ) / ι ω ]$$

$$θ = K ( ω ) / ιω$$

$${α}_{o} = α + {ρ}_{l} {ω}^{2} K ( ω ) / ιω = α + {ρ}_{l} {ω}^{2} θ$$

The equation approximately yields: (ignoring the evanescent waves)

$$∇ {P}_{s} - i \frac{ω \sqrt{ρ}}{\sqrt{λ}} {P}_{s} = 0 ,$$

This has form of a paraxial wave propagation with an effective wavenumber

$${k}_{eff} = \frac{ω}{{c}_{eff}} = \frac{ω \sqrt{ρ}}{\sqrt{λ}}$$

Where

$${c}_{eff} = \frac{\sqrt{λ}}{\sqrt{ρ}} ,$$

Define the effective wavenumber and speed of sound. Note the numerator is interpreted as the known Lame' coefficient (first). The denominator is complex valued and has effective components involving porosity and the effective Darcy coefficient K. This K also encapsulates information about the size of the ‘ducts’ in the porous medium and its tortuosity.

Under certain assumptions, it is possible to measure the effective parameters which contain porosity (n), and the effective Darcy coefficient.

These parameters may thus be effectively estimated in parts of the body and give phenomenological basis for the measurements related to and affected by osteoporotic conditions.

This may allow monitoring and/or early detection of osteoporosis.

To consider the frequency dependence of q or r, the following is provided.

$${k}_{R} = \frac{ω}{{c}_{meas}} = Re ( {k}_{eff} )$$

$${k}_{eff} = \frac{ω}{{c}_{eff}} = \frac{ω}{\sqrt{λ}} \sqrt{{ρ}_{R} + i {ρ}_{l}} = {k}_{R} - i α$$

$${c}_{eff} = \frac{\sqrt{λ}}{\sqrt{{ρ}_{R} + i {ρ}_{l}}} = \frac{\sqrt{λ} \sqrt{{ρ}_{R} - i {ρ}_{l}}}{\vert ρ \vert} = \frac{\sqrt{λ} ( A - iB )}{\vert ρ \vert}$$

$${c}_{eff} = \frac{\sqrt{λ} ( \sqrt{\vert ρ \vert + {ρ}_{R}} - i \sqrt{\vert ρ \vert - {ρ}_{R}} )}{\sqrt{2} \vert ρ \vert}$$

From which the real and imaginary parts of the c_{eff} can be read off.

Also note that

$${ρ}_{R} = ( 1 - n ) {ρ}_{s} + {ρ}_{l} n + {ρ}_{l}^{2} ω {K}_{l} ( ω )$$

$${ρ}_{I} = - {ρ}_{l}^{2} ω {K}_{R} ( ω )$$

$$Re ρ = ( 1 - n ) {ρ}_{s} + {ρ}_{l} n + {ρ}_{l}^{2} ω {K}_{I} ( ω ) = \frac{λ}{{ω}^{2}} ( \frac{{ω}^{2}}{{c}_{meas}^{2}} - {α}^{2} )$$

$$Im ρ = - {ρ}_{l}^{2} ω {K}_{R} ( ω ) = - 2 α \frac{λ}{ω {c}_{meas}}$$

Therefore, the following overdetermined system can be set up to solve for the porosity and the effective complex D'Arcy coefficients. (K, real and imaginary parts).

$$( \begin{matrix}{ρ}_{l} - {ρ}_{s} & 0 & {ρ}_{l}^{2} {ω}_{1} \\ 0 & - {ρ}_{l}^{2} {ω}_{1} & 0 \\ ⋮ & ⋮ & ⋮ \\ {ρ}_{l} - {ρ}_{s} & 0 & {ρ}_{l}^{2} {ω}_{N} \\ 0 & - {ρ}_{l}^{2} {ω}_{N} & 0\end{matrix} ) ( \begin{matrix}n \\ {K}_{R} \\ {K}_{I}\end{matrix} ) = ( \begin{matrix}- {ρ}_{s} + \frac{λ}{{ω}_{1}^{2}} ( \frac{{ω}_{1}^{2}}{{c}_{meas}^{2}} - {α}^{2} ) \\ - 2 α \frac{λ}{{ω}_{1} {c}_{meas}} \\ ⋮ \\ - {ρ}_{s} + \frac{λ}{{ω}_{N}^{2}} ( \frac{{ω}_{N}^{2}}{{c}_{meas}^{2}} - {α}^{2} ) \\ - 2 α \frac{λ}{{ω}_{N} {c}_{meas}}\end{matrix} )$$

This can be solved in multiple ways including pseudo-inverse in the sense of Penrose:

$$\hat{x} = {argmin}_{x} \frac{1}{2} {‖ Ax - b ‖}^{2}$$

$$\hat{x} = {( {A}^{T} A )}^{- 1} {A}^{T} b$$

$$\hat{x} = ( \begin{matrix}n \\ {K}_{R} \\ {K}_{I}\end{matrix} )$$

Note that a parametric dependence of K on frequency yields the relationships:

For frequencies:

$${ω}_{j} , j = 1 , … , N$$

$$2 N \times ( 1 + 2 ( {N}_{K} + 1 ) ) = 2 N \times ( 3 + 2 {N}_{K} )$$

Where the constraints on the parametric form of K is given by:

$${K}_{R} ( ω ) = {κ}_{o} + {κ}_{1} {ω}^{1} ; {K}_{R} ( ω ) = {κ}_{o} + {κ}_{j} {ω}^{j} ; j = 1 , … {N}_{K}$$

$${K}_{R} ( ω ) = {κ}_{j} {ω}^{j} ; j = 0 , … {N}_{K}$$

$${N}_{K} \leq \frac{2 N - 3}{2} = N - \frac{3}{2} ; {N}_{K} < N - 2$$

This places constraints on the number of parameters but N_{K}˜2 is likely and N is ˜20 or more so in fact N_{K}<<N−2 and the system is greatly overdetermined which allows for noisy data to be compensated for.

Accordingly, it is possible to detect clinically relevant parameters by measuring the speed of sound in trabecular bone as outlined above by segmentation or similar means, estimating porosity, and estimating Effective Darcy coefficient K In one embodiment the relationship:

ρ≡(1−n)ρₛ+ρₗ(n−iρₗωK(ω)) is used:
(1)

$${c}_{eff} = \frac{\sqrt{λ}}{\sqrt{ρ}}$$

is measured by segmentation based on obtained images.
(2) r is determined from

$${c}_{eff} = \frac{\sqrt{λ}}{\sqrt{ρ}}$$

where l is the first Lame' coefficient for bone and is assumed known, since the bone matrix itself is assumed to change in a known way with osteoporosis. For example, the bone itself loses volume content, but the chemical nature of the small amount of bone remaining is the same.
(3) The relation:

$${c}_{eff} = \frac{\sqrt{λ}}{\sqrt{{ρ}_{R} + i {ρ}_{I}}} = \frac{\sqrt{λ}}{\vert ρ \vert} \sqrt{{ρ}_{R} - i {ρ}_{I}}$$

shows that c_{eff} has a complex part. See above and \sqrt{ρ_{R}−iρₗ}=\sqrt{1/2}(\sqrt{|ρ|+ρ_{R}}−i\sqrt{|ρ|−ρ_{R}}).
(4) The relationship

$$ρ = \frac{λ}{{c}_{eff}^{2}}$$

is used to determine real and imaginary parts of r.
(5) Re ρ=(1−n)ρₛ+ρₗn is used to estimate porosity.
(6) Im

$$ρ = - {ρ}_{l}^{2} ω K ( ω )$$

is used to estimate the Darcy coefficient since other parameters are known.

The imaginary part of c_{eff} is known to contain attenuation. This can be removed based on the known power law for attenuation of bone.

The remaining anomalous attenuation is used in the above calculations.

This anomalous attenuation has been observed in our images and it is related to the porosity parameters as well. (see above).

The generalized Darcy coefficient (based on homogenization) is treated as a phenomenological parameter that is calculated for various stages of osteoporosis and interpreted after the fact in clinical situations after numerous data have been obtained.

In one particular embodiment, only the real part is used to estimate porosity.

Over time clinical results would be tabulated so that the relative effective values could be used for diagnosis or as an aid.

Another embodiment involves using l+2m in place of l as an effective first Lame' coefficient in the above derivation.

Another embodiment: The Darcy coefficient (based on homogenization) is treated as a phenomenological parameter that is calculated for various stages of osteoporosis and interpreted after the fact in clinical situations after numerous data have been obtained.

Of course, instead of using the paraxial approximation, the full acoustic Biot approximation may be used. In this case the more complete formula for the effective wavenumber from the integral equation developed above may be used. In this case the simplified wavenumber developed above for the paraxial approximation may be used.

Furthermore, as a body of images and effective values are developed it is possible to train a U-net, or encoder-decoder or other relevant NN to determine porosity.

Transfer learning could be used to utilize ResNet or a multitude of other “Universal” models (LLM) that are trained on the described data.

In another embodiment the image used for segmentation is created with machine learning (ML), including, but not limited to, deep neural nets (DNN), encoder-decoder architecture, GAN (including e.g. cycleGAN), transfer learning, etc.

The acoustic Biot approximation equations can also be solved in the standard way with finite difference frequency domain methods (FDFD), or with finite element methods.

This disclosure shows the ability to estimate porosity and other medically relevant parameters using the acoustic Biot model.

In an embodiment, a method of imaging an object having variable density can include determining images of the object at multiple frequencies; estimating the attenuation of the object at multiple frequencies from the images of the object at multiple frequencies; estimating the speed of sound of the object at multiple frequencies from the images of the object at multiple frequencies; expressing the attenuation of the object at multiple frequencies and the speed of sound of the object at multiple frequencies as an elastic Biot model; performing a paraxial approximation of the elastic Biot model; and obtaining an image of the object based on the paraxial approximation of the elastic Biot model, wherein the image shows the variable density of the object.

### Topological Complexity

As a result of imaging using any of the imaging techniques described herein, it is possible to obtain various information from the images. For example, through various techniques described herein it is possible to perform selective segmentation of ductal tissue, identifying the ductal tissue independent of glandular, fat, connective tissue and skin tissue in the breast. Once the ductal tissue has been segmented, the structure of the human breast ducts/tissues can be analyzed. A quantitative measure of topological complexity can be obtained from the analysis. Similar analyses can be carried out on segmented images of glandular tissue (independent of ductal tissue) and images containing both glandular tissue and ductal tissue. As used herein, “ductal tissue” refers to the ducts, or tubes, that transport milk to the nipple, while “glandular tissue” is used to refer to the glands in the breast including lobes and lactiferous lobules (that make milk).

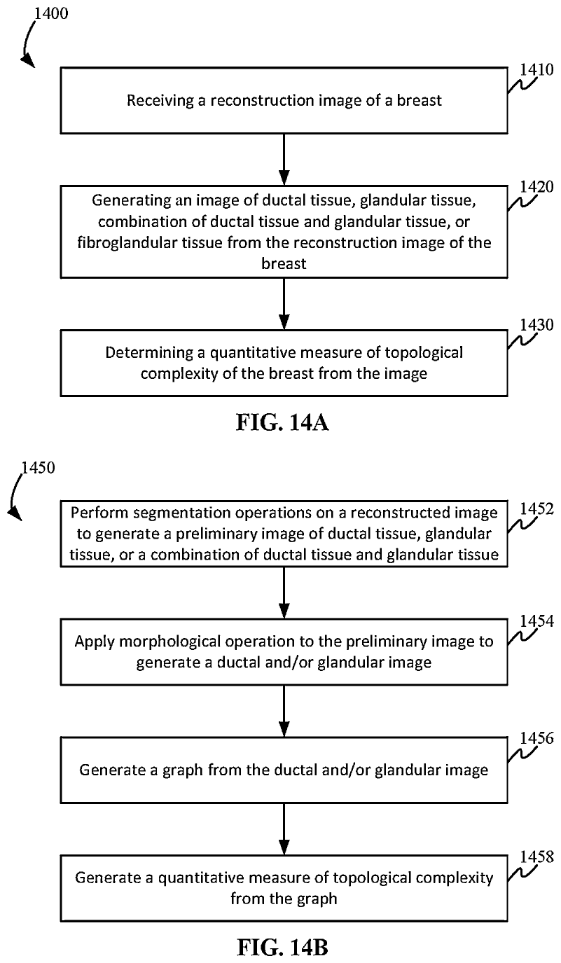

FIG. 14A shows a method of generating a quantitative measure of topological complexity. For example, referring to FIG. 14A, a method 1400 can be performed including receiving 1410, at a computing system, a reconstruction image of a breast, the reconstruction image comprising image data corresponding to a plurality of transmission frequencies used by a transmitter of an imaging system; generating 1420, by the computing system, an image of ductal tissue, glandular tissue, combination of ductal tissue and glandular tissue, or fibroglandular tissue from the reconstruction image of the breast; and determining 1430, by the computing system, a quantitative measure of topological complexity of the breast from the image. The computing system can be implemented as described with respect to computing system 5600 of FIG. 56 with appropriate instructions stored thereon for the any of the methods described herein for generating and/or using a quantitative measure of topological complexity.

The reconstruction image of the breast that includes image data corresponding to a plurality of transmission frequencies can be a high resolution image. A high resolution reconstruction image can be generated as described herein, for example, using predicate images (in the reconstruction algorithm) of multiple frequencies (at increasing frequencies or some other pattern such as described above with respect to recipes and frequency recipes) to generate a high resolution image (which may be considered a multi-frequency image since it contains information from predicate images of different frequencies in its reconstruction). While details have been provided herein of various reconstruction algorithms that may be used, it should be noted that the quantitative measure of topological complexity of a breast may be carried out on images obtained from not only quantitative transmission ultrasound but also MRI.

Generating (1420) the image can include performing, by the computing system, segmentation operations on the reconstruction image to generate a preliminary image of ductal tissue, glandular tissue, a combination of ductal tissue and glandular tissue, or fibroglandular tissue.

Morphological operations, such as erosion, can be applied to the preliminary image to generate the glandular image.

For determining (1430) a quantitative measure of topological complexity, a graph can be generated from the image and a quantitative measure of topological complexity can be generated from the graph. The graph can be a 2D graph or a 3D graph. The 3D graph may be nonplanar or planar.

The ductal tissue can have a tree-like structure. By applying erosion to an image comprising ductal tissue, a 3D non-planar graph can be created. The graph generated from the ductal image can be analyzed for cycles, etc.

Alternatively, layers can be analyzed independently as a series of planar graphs. In such cases, each level graph may be independently analyzed.

The size and order of relevant graphs and sub-graphs can be determined. The graph(s) can be evaluated using adjacency, incidence, distance, Laplacian matrices, or a combination thereof. The spectral properties of the adjacency matrix and derived matrices can be considered characteristics of the breast ductal and/or glandular system.

In some cases, certain quadrants or sub-volumes of the breast are separately evaluated to generate an associated quantitative measure of topological complexity. These different quadrants or sub-volumes can be separately tracked over time and/or used in tracking and/or diagnosing disease.

In various implementations, a software program, plug-in, or module may be provided with instructions to generate a quantitative measure of topological complexity using the graph-based approach for any suitable reconstructed image (e.g., of sufficient resolution). FIG. 14B shows an example process of a graph-based approach for determining topological complexity. Referring to FIG. 14B, process 1450 includes instructions to perform (1452) segmentation operations on a reconstructed image to generate a preliminary image of ductal tissue, glandular tissue, or a combination of ductal tissue and glandular tissue; apply (1454) morphological operations to the preliminary image to generate a ductal and/or glandular image; generate (1456) a graph from the ductal and/or glandular image; and generate (1458) a quantitative measure of topological complexity from the graph. In some cases, an image of fibroglandular tissue may be generated and converted to a graph for generating a quantitative measure.

Returning to FIG. 14A, instead of the graph-based approach, for determining (1430) a quantitative measure of topological complexity, the preliminary image can be analyzed as a 2D (or even 1D) manifold representation. Here, persistent homology is applied. As such, determining (1430) the quantitative measure of topological complexity of the breast from the image can include evaluating the preliminary image based on algebraic topology, which can include applying a Morse function to the image, obtaining Betti numbers from the image, determining homology groups, cohomology ring, homotopy, or a combination thereof. Cohomological data are determined by treating the homological data as vector spaces. The cohomology refers to the space of continuous linear mappings from these homology vector spaces to R (real numbers) (also referred to as the adjoint or dual of the vector space).

As with the graph approach, in some cases of the algebraic topology approach, certain quadrants or sub-volumes of the breast are separately evaluated to generate an associated quantitative measure of topological complexity. These different quadrants or sub-volumes can be separately tracked over time and/or used in tracking and/or diagnosing disease.

As an illustrative example, connected components (0^{th} homology group), first homology group, and second homology group can be obtained. The zeroth (0^{th}) homology group can be used to count connected components. These may be further counted as different connected components within each quadrant of the breast or each quadrant of the breast at each level. The zeroth homology group will determine the number of connected components in the breast ductal or glandular structures. The first homology group is a measure of the number of 1D ‘loops’ that are the boundary of some surface in the 2D manifold. In particular they do not disconnect the manifold upon their removal. For example, for a 2-torus the H₀(T²)=Z and β₂=1.

The first homology group of a manifold Hⱼ(M), j=1, is a measure of the number of independent 1-cycles that are boundaries of a 2-simplex. For example a 2-torus has H₁ (T²)=Z⊕Z, the direct sum of the two copies of the integers, and corresponding Betti number β₁=2.

The 2nd (second) Homology group is related to the number of 3D holes in the 2D manifold. This can all be made precise in the context of Homology theory (e.g. Vick). Since the 2D manifold is isometrically embedded into 3D space many of the intricacies of the fully developed theory are not required. In particular, torsion does not occur since the manifold is oriented and embedded in 3-space so the coefficient group can be taken as the real numbers for simplicity. For example, for a 2-torus the H₂(T²)=Z and β₂=1.

There is no torsion since the 2D manifold is embedded in 3D space and orientable. Thus, the Betti numbers can reflect topological complexity. The homology groups and cohomology rings measure the 2D and 3D holes in the structure and provide a unique index for each patient related to the Betti numbers for the ductal and/or glandular tissue structures treated as a 2D manifold.

In addition to the graph techniques and homology/cohomology based techniques, geometric estimation may be used for estimating topological complexity. For example, topological complexity can be determined by calculating a surface area and a volume of breast fibroglandular tissue. Calculating the surface area and the volume of the breast fibroglandular tissue can include evaluating fibroglandular tissue, glandular tissue, ductal tissue, or a combination of glandular and ductal tissue from a segmented image of the reconstruction image. A complexity value can be generated by calculating a ratio of the surface area to the volume of the breast fibroglandular tissue, calculating Betti numbers, calculating surface area and/or volume of fibroglandular tissue or glandular or ductal tissue separately or in combination, calculating a Morse Index, or a combination thereof.

The above described indices can be considered characteristics of a patient and not only monitored over time, but also have application to risk assessments as the indices may be correlated to various disease states.

For example, the interface between the fibroglandular tissue and fat is where it is hypothesized most cancers develop and so the complexity of the fibroglandular (ductal and glandular together or separately) tissue may be a risk factor for cancer. Surface area vs volume, as well as the graph theory-based approach and persistent homology evaluations can produce quantitative markers that correlate with disease states of the breast. Furthermore, estimating fibroglandular tissue is an important risk factor for cancer, in particular the ratio of fibroglandular volume to total breast volume (minus the skin) is an important measure known as mammographic breast density (also referred to as QBD).

Accordingly, in some implementations, the quantitative measure of topological complexity (from any of the graph, homology, and geometric techniques) can be provided as an input to a risk assessment model. For example, the described quantitative measures may be used for a Tyrer-Cuzick risk assessment model. As another example, the quantitative measure of topological complexity can be an input to a machine learning model for breast cancer risk assessment.

It is possible to extract radiomic features, including aspects of the quantitative measures of topological complexity as described herein, from the images of ductal tissue, glandular tissue, combination of ductal tissue and glandular tissue, or fibroglandular tissue. These radiomic features can be used in prognostic, predictive, and therapeutic response assessment. To this end, a system can be provided for prognostic, predictive, and response assessments based on quantitative imaging maps (e.g., from the described reconstructed images). Such a system can include an imaging module configured to generate quantitative imaging maps (which in some cases can involve the adaptable imaging methods described herein), a feature extraction module that can be configured to derive radiomic features from the quantitative imaging maps, and a prediction module that uses the derived radiomic features to forecast clinical outcomes. The feature extraction module can extract features including the various quantitative measures of topological complexity and other characteristics that can be observed in the imaging maps. In some cases, the prediction module can include a machine learning model trained on historical patient data to predict individual patient outcomes based on the radiomic features (e.g., and associated patterns). In some cases, the feature extraction module is configured to quantify feature changes over the course of treatment, providing real-time feedback for therapeutic adjustments. In some cases, the prediction module uses an algorithm that is optimized to distinguish between various prognostic groups, therapeutic response profiles, or adverse event risks based on specific patterns within the radiomic features.

It is possible to develop a large data set with quantitative measures of topological complexity so as to train any number of different machine learning models for cancer risk assessment utilizing topological complexity as one of the features. Not only can single measures be used, but the change of the quantitative measure over time may be used.

Indeed, it is possible to generate and store quantitative measures of topological complexity of a patient's breast over a period of time, tracking the quantitative measure of topological complexity of the breast over a period of time from reconstruction images of the breast of a patient captured at different times over the period of time. Indeed, it is possible to follow glandular tissue (by itself) or ductal tissue (by itself) over time, including over one or more menstrual cycles. These topological and time dependent characteristics can be tracked for sub-volumes of the breast. For example, it is possible to track quadrants at different levels of the breast (and over time) or at sub-areolar sub-volumes or near axillary sub-volumes of interest.

A method can include determining a presence of or likelihood of developing a disease using a model that includes a correlation of the quantitative measure of topological complexity of the breast over the period of time to the presence of or the likelihood of developing the disease. In this manner, it is possible to determine a patient's prognosis by correlating specific radiomic feature values (including the described quantitative measures) with survival rates, disease recurrence probabilities, or disease progression risk levels. In addition, the radiomic features (including the described quantitative measures) can be used to predict patient response to a designated therapy by assessing feature changes that correlate with responsiveness, resistance, or adverse event likelihood. These features can be repeatedly assessed over time to monitor and quantify a patient's response to therapy, facilitating adaptive modifications in the therapeutic regimen based on detected changes in these features.

It is possible to integrate clinical and demographic data with the extracted radiomic features to enhance prognostic, predictive, and response assessment accuracy, wherein the combination of clinical, demographic, and radiomic data provides a composite score for patient stratification.

The quantitative measure of topological complexity can be used in conjunction with other features including tissue type identification (which may optionally be included in available radiomic features that can be extracted from images generated as described herein). For example, the power law parameters extracted from the speed of sound/attenuation images such as described above can be used to improve the accuracy of the quantitative measure of topological complexity by further differentiating tissue types and assisting in identifying regions of the breast that are more likely to contain cancerous tissue. In some cases, a method can include quantitatively determining a spectral behavior of speed of sound and attenuation images of a reconstruction image to identify tissue types based on a power law correlation of linear coefficients associated with attenuation, speed of sound or other tissue characteristic (see e.g., power law section above) to a tissue type and/or abnormality; and using the tissue type and/or abnormality information with the quantitative measure of topological complexity (e.g., for the breast as a whole, a segment of the breast, or a specific region based on the abnormality information) for further assessments.

The various images may be displayed in a graphical user interface and visual representations related to the quantitative measures of topological complexity can be provided. For example, the quantitative measure of topological complexity can be determined for a right breast and left breast of a patient. A visual indicator for a difference between the topological complexity of the right breast and the left breast of the patient can be provided for display. For example, it is possible to highlight differences in the presence of cancer in one breast compared to the other.

In some cases, a visual representation of the quantitative measure of topological complexity of breasts for a plurality of patients having one or more similar characteristics can be generated. The one or more similar characteristics comprise one or more demographic characteristics, genetic characteristics, or a combination thereof. Demographic characteristics can include, but are not limited to, age. Genetic characteristics can include, but are not limited to, epidermal growth factor receptor—HER1-2 and gene expressions (e.g., BRCA1 or BRCA2). For example, a visual representation of the quantitative measure of topological complexity of breasts for a plurality of patients having similar gene expressions can be generated.

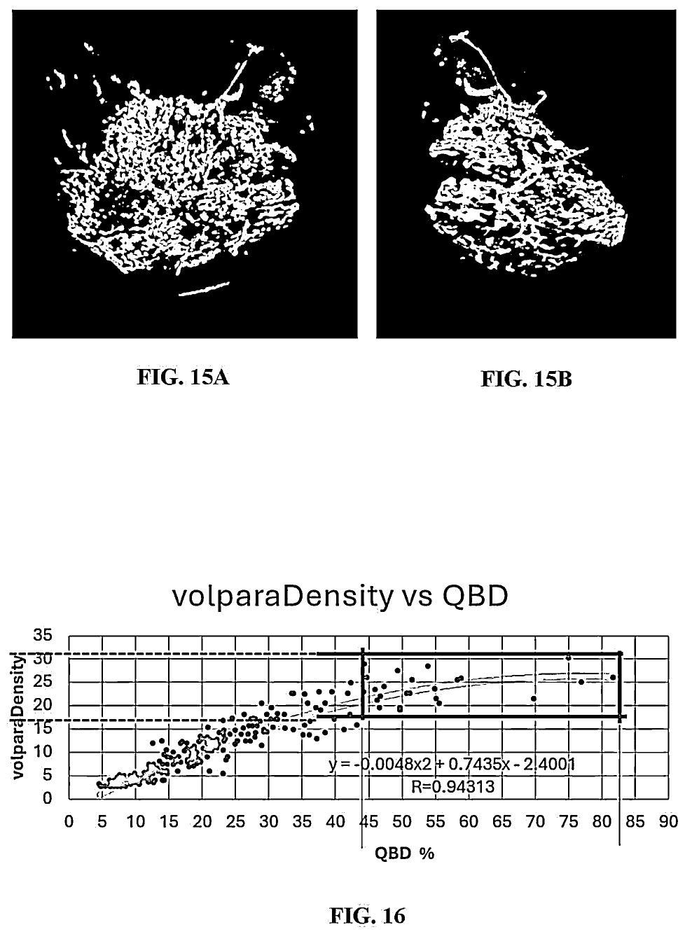

FIGS. 15A and 15B show example images of ductal structures. The complexity of the breast ductal tissue is apparent from the images.

FIG. 16 shows a plot of VolparaDensity to mammographic density estimated by techniques described herein. Referring to FIG. 16, the relationship between the projection based method (Volpara™) applied to digital breast tomosynthesis (DBT) and the mammographic density methods such as described herein shows the inability of the Volpara™ method to resolve high density values correctly. Related to this is the complexity of the breast. The more complex the topological nature of the fibroglandular tissue is, the more likely the estimate is to be incorrect. Therefore, a quantitative determination of the complexity of the breast is important. The homological index provides, through persistent homology, Morse functions, and graph theory quantitative measures of topological complexity that are computable with modern compute power.

The quantitative measure of topological complexity can be used to provide a correction factor to the Volpara estimate or other surrogate estimates of breast density (e.g., other volumetric breast density estimate provided by mammography based software). The correction factor can be based on size, density, and complexity of the fibroglandular tissue. For example, the complexity in the form of a complexity value calculated from geometric estimations or quantitative measures from graph theory or homology/cohomology based techniques can be used.

### Imaging Algorithms

There will be several types of algorithms discussed herein:
(1) sine basis, rectangular coordinate, convolutional algorithms
(2) cylindrical and rectangular coordinate recursion
(3) Parabolic (Spectral and Finite difference) marching methods
(4) Refraction corrected reflectivity and brightness functional gradient method adaptive focus.
(5) Calibration algorithms employed in the cases (1), (2), and (3), which can be used to optimize resolution and quantitative accuracy (e.g., enhancement of inversion capability).

These methods can be used for generating images from wave field energy applied to an unknown scattering object, which is then measured at some finite distance away from said scattering object. The incorporation of these algorithms support the production of accurate reconstructions of distributions of parameters which characterize the scattering object. These parameters may be “reflectivity” in the case methods such as (4) above, or they may be speed of sound, attenuation, compressibility, electromagnetic dielectric constants, conductivity, or Lame' parameters in the case of the more advanced and time intensive algorithms such as described with respect to (1-3) above. Furthermore, the reconstructions may or may not be quantitatively accurate depending upon the computational complexity of a given algorithm, and the amount of computational effort expended to obtain the reconstruction image.

Certain algorithms described herein use the scattering potential (γ) as the sole independent variable in the nonlinear minimization problem related to inverse scattering. (see example 1 below, for details).

It is defined herein a functional F≡∥R∥² where the residual R is defined as the difference between two values at the detectors: these two values being (1) the scattered field value at the detectors predicted by the forward problem on the basis of a postulated scattering potential distribution and (2) the measured value of the scattered field, i.e. R=f^{calc}−f^{meas}.

The essence of the described method (apart from the appropriate techniques to substantially reduce the computational cost of the algorithm) is the iterative construction of γ⁽ⁿ⁾, for n=1, 2, . . . , such that γ⁽ⁿ⁾≈γ^{true}, until finally ∇F≈0. Given a guess γ⁽ⁿ⁾, one calculates the derivative of F with respect to γ, in order to calculate the next guess, γ⁽ⁿ⁺¹⁾. The actual calculation of this derivative is detailed below. The functional to minimize is interpreted as depending on the scattering potential alone and not both the internal fields and the scattering potential. Symbolically, if one calls the functional that is minimized F, one can write: F≡F(γ, f) to indicate the dependence upon γ, the scattering potential (see glossary), and f, the total field inside of the object. Here, the internal fields f are considered as intermediate variables dependent upon γ, i.e. f=f(γ) so that the functional F≡F(γ, f(γ))≡F(γ).

This employment of one variable instead of two involves much more than merely neglecting (or holding constant) one of the variables. Rather, as indicated above, the effect of changing variable γ, has a nontrivial effect upon the other variables f_{ωφ}(γ), the field due to the incident wave from position φ and at frequency ω, for each possible φ and ω. The net result is a functional that is highly nonlinear in the remaining variable γ, and therefore much more difficult to solve numerically.

As used herein “real-time” is defined as the performance which allows for practical, clinical implementation of breast scanners, or of geophysical imaging apparatus, or of Non-Destructive Imaging of composite material. The data is collected and processed on-site, not sent off-site to be processed.

It can be seen that the construction and utilization of suitable Green's functions (e.g., the layered Green's functions) enables improved applicability of the inverse scattering algorithm. The layered Green's function takes the place of the free space Green's function in the presence of multiple layering in the environment surrounding the space to be imaged. This layered Green's function allows the quantitative imaging of objects located within an arbitrary distribution of layers of constant speed. The presence of these layers within the Green's function obviates the need to encompass them within the computation grid.

The described convolutional structure is incorporated into not only-the free space Green's function, but also into the layered Green's, the acoustic Biot Green's function, the elastic (including shear wave motion) Green's function, and into all combinations of these Green's functions. Furthermore the direct application of this convolutional structure to the inverse scattering algorithm is used in conjunction with operations including the use of biconjugate gradients and BiConjugate Gradients Stabilized [BiSTAB].

For the inverse scattering problem, iterative methods are used for the overall nonlinear system and the linear systems that arise during its solution. Three different nonlinear iterations are used: the Gauss-Newton (GN) iteration, the Fletcher-Reeves (FR) nonlinear conjugate gradient iteration, and a modification of the Fletcher-Reeves algorithm—the Ribiere-Polak (RP) iteration. All three nonlinear iterations are described in [Fletcher, R. D., 1980, Practical Methods of Optimization, Vol. I, Unconstrained Optimization, John Wiley and Sons, New York] herein incorporated by reference.

The GN iteration is the fastest in CPU time per step but is not guaranteed to be globally convergent unless CPU time intensive exact line searches are used. Empirically it was found that the GN method sometimes fails or requires more steps in the presence of high contrast in the scattering parameters. The more CPU intensive FR and RP iterations have been found to succeed for many of these high contrast/large size problems. The optimum strategy is often to use a combination: start with FR or RP when far from the solution and then switch to the faster GN iteration as the solution is neared. All three methods require utilization of the Jacobian of the scattering equations.

As provided herein, the Jacobian can be implemented entirely with shift invariant operations (FFT computable), thus avoiding explicit storage and time consuming direct matrix calculations. This is opposed to other techniques which require explicit storage of the Jacobian (requiring a very large amount of memory) and which implement its effect on a given vector by a direct matrix product (requiring enormous CPU time).

During the computation of a nonlinear step with the above methods, linear systems are encountered. The use of the minimum residual conjugate gradient (MRCG) method and the biconjugate gradient (BCG) (and the stabilized BCG or “BiSTAB”) method, for the efficient solution of these linear systems are among the other ideas that can be present to create a workable and practical method of imaging in real time. Biconjugate gradients (or BiSTAB) is used to solve the forward problems. These problems are amenable to the BCG method because they have the same number of unknowns as they have equations to solve (they are “square” systems). The use of BCG results in approximately the square root of the number of iterations required by traditional conjugate gradients (CG) used in our previous patent. This BCG implementation of the forward problem also plays a critical role in the shift invariant operator implementation of the Jacobian.

The MRCG algorithm can be utilized to solve the overdetermined (non-square) Jacobian equation that is encountered when computing a GN linearization. This obviates the need for a computationally intensive matrix inversion and avoids the introduction of a “regularizing” parameter since the MRCG algorithm is self-regularizing. This also results in a substantial savings in time. The FR and RP iterations are themselves nonlinear versions of the MRCG algorithm.

The examples given herein assume that the different frequencies, ω, and source positions, φ, are all computed in serial fashion. It is important to note, however, that the different frequencies and different views are independent computations (in both the forward problem and Jacobian calculations), and therefore can be computed in parallel. The implementation of this parallelization is explained in detail below.

A Born-like approximation can be made within the Jacobian. This is not the standard Born approximation common in optics and acoustical imaging (diffraction imaging). This approximation has a much greater radius of convergence than the standard Born approximation. [Borup, 1992]. Its purpose is to give a much faster way of computing the Jacobian in the presence of high contrast objects than would be possible if the exact Jacobian (see glossary for definition of terms) were used in the Gauss-Newton algorithm (or FR and RP algorithms).

Since operators are provided based on Green's theorem that allow the scattering at the border of the convolution range to be extrapolated to receivers at any position external to the border, it is possible for receivers with complicated shapes (non-point receivers) to be used in data collection. Furthermore, empirical measurements of the radiation patterns of the receiver elements (and their mutual coupling and cross talk) can be built into these projection operators.

The described algorithms can handle geometries where the source or receiver locations do not completely circumscribe the object or where the solid angles defined by the source or receivers with respect to the body are small. This applies to not only the acoustic case, but also to the elastic and electromagnetic scenarios. The presence of layering is now incorporated into the Green's function so that it actually helps to increase the resolution in the incomplete view problem.

In addition to exploiting the Cartesian convolution structure of the scattering equations, it is possible to utilize cylindrical coordinates since, when expressed in cylindrical coordinates, the scattering equations become separably symmetric in the radial coordinate while retaining convolutional form in the angular coordinate. A general 1-D operator requires order N² arithmetic operations. One dimensional convolutions can be implemented by FFT in order N log₂(N) arithmetic operations while separably symmetric kernels can be implemented recursively with order N arithmetic. Thus, cylindrical coordinates offer considerable improvement in speed. An even greater savings can be realized by going a step further by radially recurring multiple view scattering operators. This allows the calculation of the solution of the forward problems for all views (source positions) in order N³ log₂(N) arithmetic as compared to order N_{bcg}N³ log₂(N) arithmetic for the 2-D FFT, BCG approach (N_{bcg} is the number of BCG iterations required for adequate convergence). This approach to forward problems scattering also has the advantage that it is non-iterative—it requires a fixed amount of computation. This is particularly fortuitous since there are cases where the BCG iteration fails to converge.

In addition to cylindrical coordinate recursion, a scattering matrix recursion in rectangular coordinates is possible. This new approach can be shown to require only order N³ operations for computing all views of a 2-D scattering problem.

The following explanation of notation is given to facilitate obtaining an understanding of certain algorithms described herein. The scattering potential γ changes from point to point within an object or body as well as changing with frequency of the incident field. Thus γ_{ωj}≡γ_{ω}(xⱼ), xⱼ∈R³ or R³ used to signify the scattering potential at pixel j or point j, for the incident field at frequency ω, γ_{ω} can be considered to be the vector composed of all values of j, i.e. for all pixels: γ_{ω}≡(γ_{ω1}γ_{ω2} . . . γ_{ωN})^{T}, where T indicates “transpose”. For purposes of exposition and simplicity, the following considers the case where γ is independent of frequency, although this is not necessary.

### Notation: Vector Field Notation

The following notation denotes a vector field describing elastic waves, or electromagnetic waves:

$$f ( r ) ≡ ( \begin{matrix}{f}_{x} ( x , y , z ) \\ {f}_{y} ( x , y , z ) \\ {f}_{z} ( x , y , z )\end{matrix} ) ≡ {( {f}_{x} {f}_{y} {f}_{z} )}^{T} \\ {f}^{i} ( r ) ≡ ( \begin{matrix}{f}_{x}^{i} ( x , y , z ) \\ {f}_{y}^{i} ( x , y , z ) \\ {f}_{z}^{i} ( x , y , z )\end{matrix} ) ≡ {( {f}_{x}^{i} {f}_{y}^{i} {f}_{z}^{i} )}^{T}$$

T denotes “transpose” to represent the total field. The incident field is the field that would be present if there was no object present to image. In the case of layering in the ambient medium, it is the field that results from the unblemished, piecewise constant layering.

The scattered field is the difference between the total field and the incident field, it represents that part of the total field that is due to the presence of the inhomogeneity, e.g., the “mine” for the undersea ordnance locater, the hazardous waste cannister for the hazardous waste locator, the school of fish for the echo-fish locator/counter, and the malignant tumor for the breast scanner: f^{s}(r)≡f(r)−fⁱ(r)

$${f}^{s} ( r ) ≡ ( \begin{matrix}{f}_{x}^{s} ( x , y , z ) \\ {f}_{y}^{s} ( x , y , z ) \\ {f}_{z}^{s} ( x , y , z )\end{matrix} ) ≡ {( {f}_{x}^{s} {f}_{y}^{s} {f}_{z}^{s} )}^{T}$$

T—denotes “transpose”

f_{ωϕ}^{inc}(r) denotes the scalar incident field coming from direction (source position) φ at frequency ω. The r could represent either a 3 dimensional, or a 2 dimensional vector of position.

### Scalar Field Notation

A scalar field is indicated by a nonbold f(r), r∈R³.

### Example 1

### Acoustic Scattering—Scalar Model Equations

This first example is designed to give the basic structure of the algorithm and to point out why it is so fast in this particular implementation compared to present state of the art. The background medium is assumed to be homogeneous medium (no layering). This example will highlight the exploitation of convolutional form via the FFT, the use of the Frechet derivative in the Gauss-Newton and FR-RP algorithms, the use of the biconjugate gradient algorithm (BCG) for the forward problems, the independence of the different view, and frequency, forward problems. It will also set up some examples which will elucidate the patent terminology. The field can be represented by a scalar quantity f. The object is assumed to have finite extent The speed of sound within the object is c=c(x). The speed of sound in the background medium is the constant cₒ. The mass density is assumed to be constant. The attenuation is modeled as the imaginary part of the wavespeed. These are simplifying assumptions, which we make so as to focus on the important aspects of the imaging algorithm. By no means is the imaging algorithm restricted to the scalar case, either in theory, or, more importantly, in practical implementation.

We consider the two dimensional case first of all (equivalently the object and incident fields are assumed to be constant in one direction). Given an acoustic scatterer with constant mass density and constant cross-section in z, illuminated by a time harmonic incident field, f_{ωφ}^{inc}, with e^{iωt} time dependence and source position (incident angle), φ, the total field satisfies the following integral equation.

$${f}_{ω ϕ}^{i n c} ( \overline{ρ} ) = {f}_{ω ϕ} ( \overline{ρ} ) - \int \int γ ( \overline{{ρ}^{′}} ) {f}_{ω ϕ} ( \overline{{ρ}^{′}} ) {g}_{ω} ( \vert \overline{ρ} - \overline{{ρ}^{′}} \vert ) {dx}^{′} {dy}^{′} where γ = \frac{{c}_{0}^{2}}{{c}^{2}} - 1 \qquad ( 1 )$$

ρ̅=(x, y) and where f≡f_{ωφ} is the field internal to the object at frequency ω and resulting from the incident field from source position φ: f_{ωφ}^{inc}. The 2-D Helmholtz Green's function is given by:

$${g}_{ω} ( \vert \overline{ρ} - {\overline{ρ}}^{{}^{′}} \vert ) = \frac{{k}_{ω}^{2}}{4 i} {H}_{0}^{( 2 )} ( {k}_{ω} \vert \overline{ρ} - {\overline{ρ}}^{{}^{′}} \vert ) , {k}_{ω} = \frac{ω}{{c}_{0}}$$

where

$${H}_{0}^{( 2 )}$$

is the Hankel function of the second kind, and zeroth order.

Now it is required to compare the field measured at the detectors, with the field as predicted by a given guess, γ⁽ⁿ⁾, for γ. To accomplish this, first define the scattered field at the detectors as the total field minus the incident field.

$${f}_{ω ϕ}^{sc} ( \overline{ρ} ) ≡ {f}_{ω ϕ} ( \overline{ρ} ) {f}_{ω ϕ}^{inc} ( \overline{ρ} )$$

This represents the field due entirely to the scattering potential. Using this relation to rewrite (1), gives

$${f}_{ω φ}^{sc} ( \overline{ρ} ) = \int \int γ ( \overline{{ρ}^{′}} ) {f}_{ω φ} ( \overline{{ρ}^{′}} ) {g}_{ω} ( \vert \overline{ρ} - \overline{{ρ}^{′}} \vert ) {dx}^{′} {dy}^{′} \qquad ( 2 )$$

These two equations, (1) and (2) are the basis of the imaging algorithm. They must be discretized, then exported to the computer in order to solve practical imaging problems. The purpose of the apparatus and method herein described, is to solve the discretized form of these equations without making any linearizing assumptions (such as used in conventional diffraction tomography), and in real time, with presently available computers.

### Discretization of the Acoustic Free-Space Lippmann Schwinger Integral Equation and Green'S Function—2D Free Space Case

Let us for a moment drop the frequency and source position subscripts. The scalar field “γf” is given by

$$γ f ( \begin{matrix}{x}^{′} \\ {y}^{′}\end{matrix} ) = γ ( \begin{matrix}{x}^{′} \\ {y}^{′}\end{matrix} ) f ( \begin{matrix}{x}^{′} \\ {y}^{′}\end{matrix} )$$

Discretization of the integral equation is achieved by first decomposing this function into a linear combination of certain (displaced) basis functions

$$γ f ( \begin{matrix}{x}^{′} \\ {y}^{′}\end{matrix} ) \approx {\sum }_{{n}^{′} = 1}^{{N}_{x}} {\sum }_{{m}^{′} = 1}^{{N}_{y}} {a}_{{n}^{′} {m}^{′}} S ( \begin{matrix}{x}^{′} - {n}^{′} δ \\ {y}^{′} - {m}^{′} δ\end{matrix} ) \qquad ( 3 )$$

where it has been assumed that the scatterer γ lies within the support [0, Nₓδ]×[0, N_{y}δ]—a rectangular subregion.

The basis functions S can be arbitrary except that we should have cardinality at the grid nodes:

$$s ( \begin{matrix}0 \\ 0\end{matrix} ) = 1$$

whereas for all other choices of n′, m′,

$$S ( \begin{matrix}{n}^{′} δ \\ {m}^{′} δ\end{matrix} ) = 0 , {n}^{′} , {m}^{′} \neq 0$$

An example of such a function is the 2-D “hat” function. The algorithm uses the “sinc” function as its basic building block—the Whittaker sine function which is defined as:

$$sin c ( x ) = \frac{sin ( π x )}{π x}$$

The two dimensional basis functions are defined by the tensor product:

$$S ( \begin{matrix}{x}^{′} - {n}^{′} δ \\ {y}^{′} - {m}^{′} δ\end{matrix} ) = sinc ( ( {x}^{′} - {n}^{′} δ ) / δ ) \cdot sinc ( ( {y}^{′} - {m}^{′} δ ) / δ ) \qquad ( 4 )$$

If the equality in equation (3) is presumed to hold at the grid points x=nδ, y=mδ: the coefficients αₙₘ can be determined to be precisely the value of the field γf:

$$γ f ( \begin{matrix}{x}^{′} \\ {y}^{′}\end{matrix} ) = {\sum }_{{n}^{′} = 1}^{{N}_{x}} {\sum }_{{m}^{′} = 1}^{{N}_{y}} γ ( \begin{matrix}{n}^{′} δ \\ {m}^{′} δ\end{matrix} ) f ( \begin{matrix}{n}^{′} δ \\ {m}^{′} δ\end{matrix} ) S ( \begin{matrix}{x}^{′} - {n}^{′} δ \\ {y}^{′} - {m}^{′} δ\end{matrix} ) \qquad ( 5 )$$

Now this expression for the product γf can be substituted into (1):

$${f}^{i} ( \begin{matrix}x \\ y\end{matrix} ) = f ( \begin{matrix}x \\ y\end{matrix} ) - \frac{{k}_{ω}^{2}}{4 i} {\sum }_{{n}^{′}} {\sum }_{{m}^{′}} {γ}_{{n}^{′} {m}^{′}} {f}_{{n}^{′} {m}^{′}} \int {H}_{0}^{( 2 )} ( {k}_{ω} \vert \overline{ρ} - {\overline{ρ}}^{{}^{′}} \vert ) S ( \begin{matrix}{x}^{′} - {n}^{′} δ \\ {y}^{′} - {m}^{′} δ\end{matrix} ) {d}^{2} {ρ}^{′} \qquad ( 6 )$$

In particular this equation holds at

$$( \begin{matrix}x \\ y\end{matrix} ) = ( \begin{matrix}n δ \\ m δ\end{matrix} )$$

for which we get:

$${f}_{n m}^{i} = {f}_{n m} - {\sum }_{{n}^{′} = 1}^{{N}_{x}} {\sum }_{{m}^{′} = 1}^{{N}_{y}} {γ}_{{n}^{′} {m}^{′}} {f}_{{n}^{′} {m}^{′}} {g}_{n - {n}^{′} , m - {m}^{′}} , \begin{matrix}n = 1 , … , {N}_{x} \\ m = 1 , … , {N}_{y}\end{matrix} \qquad ( 7 )$$

where the 2-D discrete Green's function is defined as:

$$g ( n - {n}^{′} , m - {m}^{′} ) ≡ \frac{{k}^{2}}{4 i} \int {H}_{0}^{( 2 )} ( k \vert ( \begin{matrix}n δ - {x}^{′} \\ m δ - {y}^{′}\end{matrix} ) \vert ) S ( \begin{matrix}{x}^{′} - {n}^{′} δ \\ {γ}^{′} - {m}^{′} δ\end{matrix} ) {d}^{2} {ρ}^{′} \qquad ( 8 )$$

Although it is not clear that the dependence of g on m and n is as so stated, in is indeed the case, since we have by the substitution of the transformation

$${x}^{′} \to {x}^{′} + n δ \\ {y}^{′} \to {y}^{′} + {m}^{′} δ$$

into this last equation shows explicitly that the discretized Green's function “g”, does in fact depend only on the differences n−n′, and M−m″. Thus the discrete equation (7) has inherited the convolutional form of the continuous equation (1). It is this fact which allows the use of the 2-D FFT to compute the summation in (8) in only order NₓN_{y} log₂(NₓN_{y}) arithmetic operations and is therefore critical to the real-time speed of the algorithm.
   Extension of the Method to Remote Receivers with Arbitrary Characteristics

There is a case that the receivers do not lie within the range of the convolution and/or they are not simple point receivers. It is well known that, given source distributions within an enclosed boundary, the scattered field everywhere external to the boundary can be computed from the values of the field on the boundary by an application of Green's theorem with a suitably chosen Green's function, i.e.:

$${f}^{s} ( \overline{ρ} ) = {∮}_{B} {f}^{s} ( {\overline{ρ}}^{{}^{′}} ) P ( \overline{ρ} , {\overline{ρ}}^{{}^{′}} ) {dl}^{′} , \overline{ρ} outside boundary B \qquad ( 12 )$$

where P is the Green's function, the integral is about the boundary, and dl′ is the differential arclength (in the 3-D case, the integral is on an enclosing surface). Equation (12) allows for the construction of a matrix operator which maps the boundary values of the rectangular support of the convolution (2Nₓ+2N_{y}−4 values in the discrete case) to values of the scattered field external to the rectangle. Furthermore, this “propagator matrix” can be generalized to incorporate more complex receiver geometries. For example, suppose that the receiver can be modeled as an integration of the scattered field over some support function, i.e.;

$${v}_{n} = signal from receiver n = \int \int {f}^{s} ( \overline{ρ} ) {S}_{n} ( \overline{ρ} ) d s \qquad ( 13 )$$

where Sₙ is the support of receiver n. Then from (12):

$${ν}_{n} = {∮}_{B} {f}^{s} ( {\overline{ρ}}^{{}^{′}} ) \{ \int \int {S}_{n} ( \overline{ρ} ) P ( \overline{ρ} , {\overline{ρ}}^{{}^{′}} ) ds \} d {l}^{′} , \overline{ρ} outside boundary B = {∮}_{B} {f}^{s} ( {\overline{ρ}}^{′} ) {P}^{′} ( n , {\overline{ρ}}^{′} ) {dl}^{′} , {P}^{′} ( n , {\overline{ρ}}^{′} ) = \int \int {S}_{n} ( \overline{ρ} ) P ( \overline{ρ} , {\overline{ρ}}^{′} ) ds \qquad ( 14 )$$

Discretizing the integral gives the matrix equation:

$${v}_{n} = {\sum }_{l = 1}^{{N}_{b}} {{P}_{nl}^{′}}^{} {f}_{l}^{s} , n = 1 , … , {N}_{d} \qquad ( 15 )$$

where N_{b}=2Nₓ+2N_{y}−4 is the number of boundary (border) pixels and N_{d} is the number of receivers. Equation (15) defines the matrix that we shall henceforth refer to as P or the “propagator matrix” for a given distribution of external receivers. The equation:

$${v}_{ω ϕ}^{m e a s} = {P}_{ω} ( {G}_{ω} [ γ ] {f}_{ω ϕ} ) \qquad ( 16 )$$

Note that P is a function of frequency, but is not a function of source position.

$${v}_{ω ϕ}^{m e a s}$$

is a vector of dimension N_{d}.

The added flexibility of this propagator matrix formulation is particularly advantageous when interfacing our algorithms with real laboratory or field data. Often times the precise radiation patterns of the transducers used will not be known a priori. In this event, the transducers must be characterized by measurements. The results of these measurements can be easily incorporated into the construction of the propagator matrix P allowing the empirically determined transducer model to be accurately incorporated into the inversion.

Equations (16) then provides in compact notation the equations which we wish to solve for the unknown scattering potential, γ. First, we consider the forward problem, i.e. the determination of the field f_{ωφ} for a known object function γ and known incident field, f_{ωφ}ⁱ. Then we establish how this forward problem is then incorporated into the solution of the inverse problem, i.e., the determination of γ when the incident fields, and the received signals from a set of receivers are known. Note that the internal field to the object is also—along with the object function γ, an unknown in the inverse problem.

Since (7) is linear in f_{ωφ} it can be solved by direct inversion of the linear system:

$$f = {( I - G [ γ ] )}^{- 1} {f}^{i} \qquad ( 17 )$$

In discrete form, this is a matrix equation (after linearly ordering the 2-D supports of the domain and range into vectors). Since the dimension of the range and domain of the linear system is NₓN_{y}, the dimension of the linear system is the same giving a total of (NₓN_{y})² elements.

The arithmetic required to solve the system by standard direct means is thus order (NₓN_{y})³. In other words, a doubling of the edge dimension of the problem space will increase the CPU time required by a factor 26=64 times! The arithmetic work required will quickly become intolerable as the size of the problem increases. It is precisely this large growth rate which has convinced many researchers not to pursue inverse scattering approaches based on integral equations. One could, of course, go to an iterative method. A single iteration of an iterative linear system solver, such as bi-conjugate gradients requires order (NₓN_{y})² operations (essentially a matrix-vector product). It can be shown that the growth rate in the number of required iterations for sufficient convergence for the BCG algorithm is order N for this equation. Thus, the overall computational complexity is order N⁵—only one order of N has been saved over direct inversion. Since inverse problems in 2-D generally require order N views, the iteration must be done N times and we are back to order N⁶ computation for BCG.

The key to overcoming this objection is the convolutional form of (7). If this is exploited by use of the FFT algorithm, the computation needed to perform a matrix-vector product is only order NₓN_{y} log₂(NₓN_{y}). This allows the BCG algorithm to be applied to a single view with order N³ log₂(N) operations and to all views with order N⁴ log₂(N) operations. Due to the slow growth rate of log₂(N), this is essentially a reduction of two orders of N over nonconvolutional methods. Also, this convolutional approach avoids the necessity of storing the order N⁴ computer words needed to contain the linear system in full matrix form. Only order N² words are needed due to the shift invariance of the discrete Green's kernel in (7). It is these two savings, more than any other advance that we have made, that allows us to perform inverse scattering in reasonable time for meaningfully sized problems. The use of BCG over the original CG algorithm also represents a major advance since it converges in essentially the square root of the number of iterations needed by CG. This combination of FFT and CG algorithm was originally developed in [Borup, 1989, Ph. D. dissertation, Univ. of Utah, Salt Lake City], herein incorporated by reference.

### The Imaging or Inverse Problem

In order to solve the imaging problem, we need a set of equations relating the unknown scattering potential, γ, and total fields, f_{ωφ}, with measurements of the scattered field on a set of external detectors. These detector equations are given in (16). Equation (16), and the internal field equations are the equations which are solved simultaneously to determine γ and the f_{ωφ}. There are NₓN_{y} unknowns corresponding to the γ values at each of the grid points, and Nₓ×N_{y}×Ω×Φ, unknowns corresponding to the unknown fields, Ω is the number of frequencies, and Φ is the number of source positions (angles). We have improved upon the state of the art by considering the internal field equations to define f_{ωφ}. for a given γ. Thus, the total number of unknowns is reduced to NₓN_{y}.

The total number of measurement equations is N_{d}×Ω×Φ where N_{d} is the number of detectors. In the general case, where, the sources and detectors do not completely surround the object, the problem of determining γ is “ill-posed”, in a precise mathematical sense, therefore, in order to guarantee a solution, the number of equations N_{d}×Ω×Φ>NₓN_{y}, is chosen to be larger than the number of pixel values for over determination. Then the system is solved in the least squares sense. More specifically the solution of (9,16) for γ and the set of fields, f_{ωφ}, in the least squares sense is obtained by minimizing the real valued, nonlinear functional:

$${min}_{γ} {\sum }_{ωϕ} {‖ {r}_{ω ϕ} ‖}^{2} ≡ {min}_{γ} {\sum }_{ω , ϕ} {‖ {v}_{ω ϕ}^{m e a s} - {P}_{ω} {G}_{ω} [ {f}_{ω ϕ} ] γ ‖}^{2} \qquad ( 18 )$$

subject to the satisfaction of the total field equations, (9), as constraints. The vector r_{ωϕ} of dimension N_{d} is referred to as the “residual” for frequency, ω, and angle, ϕ.

The methods used to solve the nonlinear optimization problem in our inverse scattering algorithms are, thus far, all “gradient methods” in that second derivative information is not used (as it is in the full Newton and quasi-Newton methods). The principal computation involves the calculation of the gradient vector of (18). A straight forward calculation gives the gradient formula: ∇f(x)=−J^{H}(x)r(x) where the superscript H denotes the Hermitian transpose (complex conjugate transpose of the matrix) and J is the Jacobian of the nonlinear system:

$$J ( x ) = [ \begin{matrix}\frac{\partial }{\partial {x}_{1}} {a}_{1} & … & \frac{\partial }{\partial {x}_{N}} {a}_{1} \\ ⋮ & ⋱ & ⋮ \\ \frac{\partial }{\partial {x}_{1}} {a}_{M} & … & \frac{\partial }{\partial {x}_{N}} {a}_{M}\end{matrix} ] \qquad ( 19 )$$

The simplest gradient algorithm is the Gauss-Newton (GN) iteration. The GN iteration for finding a solution to ∇f=0 is given by:

$${x}^{{}_{( n )}} = y - a ( {x}^{{}_{( n )}} ) \qquad ( 20.1 )$$

$$δ {x}^{{}_{( n )}} = {( {J}_{n}^{H} {J}_{n} )}^{- 1} {J}_{n}^{H} {r}^{{}_{( n )}} \qquad ( 20.2 )$$

$${x}^{{}_{( n + 1 )}} = {x}^{{}_{( n )}} + δ {x}^{{}_{( n )}} \qquad ( 20.3 )$$

where a is the vector of nonlinear equations. This iteration is well defined assuming that the columns of Jₙ remain linearly independent. Since (20.2) is equivalent to the quadratic minimization problem:

$${min}_{δ x ( n )} {‖ {J}_{n} δ {x}^{( n )} - {r}^{( n )} ‖}_{M}^{2} \qquad ( 21 )$$

it can be solved by the minimum residual conjugate gradient method (MRCG). This approach also ensures that small singular values in Jₙ will not amplify noise if care is taken not to overconverge the iteration.

Here, the fields f_{ωφ} are considered to be dependent variables, with dependence upon γ given implicitly by

$${f}_{ω φ}^{( n )} = {( I - {G}_{ω} [ {γ}^{( n )} ] )}^{- 1} {f}_{ω φ}^{inc} .$$

In order to find the Jacobian expression we must then differentiate the residual vector defined in (18) with respect to γ. The details of this calculation are given in [Borup, 1992.]. The result is:

$${J}_{ω ϕ} = \frac{δ {r}_{ωϕ}}{δ γ} = {{P}_{ω} ( I - [ γ ] {G}_{ω} )}^{- 1} [ {f}_{ωϕ} ] \qquad ( 22 )$$

The final Gauss-Newton algorithm for minimizing (18) subject to equality constraints is:
1. GN Select an initial guess, γ⁽⁰⁾.
2. GN Set n=0
3. GN Solve the forward problems using (biconjugate gradients)

$$BCG : {f}_{ω ϕ}^{( n )} = {( I - {G}_{ω} [ {γ}^{( n )} ] )}^{- 1} {f}_{ωϕ}^{inc} , \begin{matrix}ω = 1 , … , Ω \\ ϕ = 1 , … , Φ .\end{matrix}$$

4. GN Compute the detector residuals:

$${r}_{ω ϕ}^{( n )} = {v}_{ω ϕ}^{m e a s} - {P}_{ω} {G}_{ω} [ {f}_{ω ϕ}^{( n )} ] {γ}^{( n )} , \begin{matrix}ω = 1 , … , Ω \\ ϕ = 1 , … , Φ .\end{matrix}$$

5. GN If

$${\sum }_{ωϕ} {‖ {r}_{( ωϕ )}^{( n )} ‖}^{2} < ε$$

6. GN Use MRCG to find the least squares solution to the quadratic minimization problem

$${min}_{{δγ}^{( n )}} {\sum }_{ω ϕ} {‖ {r}_{( ω ϕ )}^{( n )} + {P}_{ω} {G}_{ω} {( I - [ {γ}^{( n )} ] {G}_{ω} )}^{- 1} [ {f}_{ω ϕ}^{( n )} ] {δγ}^{( n )} ‖}^{2}$$

for δγ⁽ⁿ⁾
7 GN Update γ: γ⁽ⁿ⁺¹⁾=γ⁽ⁿ⁾+δγ⁽ⁿ⁾
8 GN Set n=n+1, go to GN 3

The crux of the GN iteration is GN 6 where the overdetermined quadratic minimization problem is solved for the scattering potential correction. This correction is approximated by applying a set of M iterations of the MRCG algorithm. The details of GN 6 are:
6.1 GN Initialize the zeroth iterate of MRCG: δy⁰=0
6.2 GN Initialize the MRCG residuals equal to the GN outer loop residuals:

$${r}_{ω , ϕ}^{0} = {r}_{ω ϕ}^{( n )}$$

Note that iterates pertaining to the MRCG iteration are indexed without the ( ) in order to distinguish them from outer GN loop iterates.
6.3 GN Compute the gradient of the MRCG quadratic functional:

$${g}^{0} = {\sum }_{ω ϕ} [ {f}_{ω ϕ}^{* ( n )} ] {( I - {G}_{ω}^{*} [ {γ}^{* ( n )} ] )}^{- 1} {G}_{ω}^{*} {P}_{ω}^{H} {r}_{ω ϕ}^{0}$$

6.4 GN Set the initial search direction equal to minus the gradient:

$${p}^{0} = - {g}^{0}$$

6.5 GN Set m=0
6.6 GN Compute

$${t}_{ω ϕ}^{m} = {P}_{ω} {{G}_{ω} ( I - [ {γ}^{( n )} ] {G}_{ω} )}^{- 1} [ {f}_{ω ϕ}^{( n )} ] {p}^{m} , \begin{matrix}ω = 1 , … , Ω \\ ϕ = 1 , … , Φ .\end{matrix}$$

6.7 GN Compute the quadratic step length:

$${a}^{m} = {\sum }_{ω ϕ} {‖ {t}_{ω ϕ}^{m} ‖}^{2} / {‖ {g}^{m} ‖}^{2}$$

6.8 GN Update the solution of the quadratic minimization:

$$δ {γ}^{m + 1} = δ {γ}^{m} + {a}^{m} {p}^{m}$$

6.9 GN Update the MRCG residuals:

$${r}_{ω ϕ}^{m + 1} = {r}_{ω ϕ}^{m} + {a}^{m} {t}_{ω ϕ}^{m}$$

6.10 GN Compute the gradient of the MRCG quadratic functional:

$${g}^{m + 1} = {\sum }_{ω ϕ} [ {f}_{ω ϕ}^{* ( n )} ] {( I - {G}_{ω}^{*} [ {γ}^{* ( n )} ] )}^{- 1} {G}_{ω}^{*} {P}_{ω}^{H} {r}_{ω ϕ}^{m + 1}$$

6.11 GN Compute:

$${β}^{m} = {‖ {g}^{m + 1} ‖}^{2} / {‖ {g}^{m} ‖}^{2}$$

6.12 GN Update the MRCG search direction:

$${p}^{m + 1} = - {g}^{m + 1} + {β}^{m} {p}^{m}$$

6.13 GN If m=M, go to GN 6.15
6.14 GN m=m+1, go to GN 6.8
6.15 GN Equate the solution of the quadratic minimization problem with the last iterate MRCG:

$$δ {γ}^{( n )} = δ {γ}^{M}$$

6.16 GN Return to the GN algorithm at GN 7.

A problem with the algorithm above is the presence of the inverse of the transposed total field equation (I−[γ⁽ⁿ⁾]G_{ω})⁻¹ in the computation of the Jacobian and its adjoint in 6.3 and 6.10 Since we do not invert (or even store) these large linear systems, the occurrence of these inverse operators must be dealt with. This problem can however be overcome by computing the action of this operator on a given vector, as needed during the MRCG iteration, by a few iterations of BCG. When performed in this way, the shift invariance of the Green's function is exploited in a maximal way. No large matrices need to be inverted or stored. This is because the Jacobian implementation now consists exclusively of shift invariant kernels (diagonal kernels such as pointwise multiplication by γ or f and the shift invariant kernel composed of convolution with the Green's function) Such shift invariant kernels can be implemented efficiently with the FFT as previously described.

An even greater increase in numerical efficiency can be obtained in cases for which the approximation:

$${( I - [ {γ}^{( n )} ] {G}_{ω} )}^{- 1} \approx ( I + [ {γ}^{( n )} ] {G}_{ω} ) \qquad ( 23 )$$

(which is similar to the Born approximation of the forward problem) can be used in the Jacobian. This has been found to be the case for many acoustic scattering problems for biological tissue, and for EM problems for which the contrast in dielectric contrast is small.

The other two gradient algorithms that are used to solve the inverse scattering equations are the Fletcher-Reeves and Ribiere-Polak algorithms. For the inverse scattering equations, they are given by the iteration:
1 RP Select an initial guess,
2 RP Solve the forward problems using (biconjugate gradients) BCG:

$${f}_{ω ϕ}^{i n c} = ( I - {G}_{ω} [ {γ}^{( 0 )} ] ) {f}_{ω ϕ}^{( 0 )} , \begin{matrix}ω = 1 , … , Ω \\ ϕ = 1 , … , Φ .\end{matrix}$$

for the internal fields,

$${f}_{ω ϕ}^{( 0 )}$$

3 RP Compute the detector residuals:

$${r}_{ω ϕ}^{( 0 )} = {P}_{ω} {G}_{ω} [ {f}_{ω ϕ}^{( 0 )} ] {γ}^{( 0 )} - {v}_{ω ϕ}^{meas} , \begin{matrix}ω = 1 , … , Ω \\ ϕ = 1 , … , Φ .\end{matrix}$$

4 RP Compute the gradient:

$${r}_{ω ϕ}^{( 0 )} = {P}_{ω} {G}_{ω} [ {f}_{ω ϕ}^{( 0 )} ] {γ}^{( 0 )} - {v}_{ω ϕ}^{meas} , \begin{matrix}ω = 1 , … , Ω \\ ϕ = 1 , … , Φ .\end{matrix}$$

5 RP Compute the search direction: p⁽⁰⁾=−g⁽⁰⁾
6 RP n=0
7 RP Compute the Jacobian operating on the search direction

$${t}_{ω ϕ}^{( 0 )} = {P}_{ω} {{G}_{ω} ( I - [ {γ}^{( 0 )} ] {G}_{ω} )}^{- 1} [ {f}_{ω ϕ}^{( 0 )} ] {p}^{( 0 )} , \begin{matrix}ω = 1 , … , Ω \\ ϕ = 1 , … , Φ .\end{matrix}$$

8 RP Compute the step length (quadratic approximation):

$${α}_{n} = - Re {\sum }_{ω ϕ} ( {r}_{ω ϕ}^{( n )} , {t}_{ω ϕ}^{( n )} ) / {\sum }_{ω ϕ} {‖ {t}_{ω ϕ}^{( n )} ‖}^{2}$$

9 RP Update the solution: γ^{(n+1}}=γ⁽ⁿ⁾+aₙp⁽ⁿ⁾
10 RP Solve the forward problems using (biconjugate gradients) BCG:

$${f}_{ω ϕ}^{i ω c} = ( 1 - {G}_{ω} [ {γ}^{( n + 1 )} ] ) {f}_{ω ϕ}^{( n + 1 )} , \begin{matrix}ω = 1 , … , Ω \\ ϕ = 1 , … , Φ .\end{matrix}$$

11 RP Compute the detector residuals:

$${r}_{ω ϕ}^{( n + 1 )} = {P}_{ω} {G}_{ω} [ {f}_{ω ϕ}^{( n + 1 )} ] {γ}^{( n + 1 )} - {v}_{ω ϕ}^{m e a s} , \begin{matrix}ω = 1 , … , Ω \\ ϕ = 1 , … , Φ .\end{matrix}$$

12 RP If

$${\sum }_{ωϕ} {‖ {r}_{ωϕ}^{( n )} ‖}^{2} < ε ,$$

stop.
13 RP Compute the gradient:

$${g}^{( n + 1 )} {\sum }_{ωϕ} [ {f}_{ω ϕ}^{* ( n + 1 )} ] {( I - {G}_{ω}^{*} [ {γ}^{* ( n + 1 )} ] )}^{- 1} {G}_{ω}^{*} {P}_{ω}^{H} {r}_{ω ϕ}^{( n + 1 )}$$

14 RP Compute.

$${β}_{n} = \{ \begin{matrix}{‖ {g}^{( n + 1 )} ‖}^{2} / {‖ {g}^{( n )} ‖}^{2} , Flechers - Reeves \\ ( {g}^{( n + 1 )} , {g}^{( n + 1 )} - {g}^{( n )} ) / {‖ {g}^{( n )} ‖}^{2} , Ribere - Pollack\end{matrix}$$

15 RP Update the search direction: p⁽ⁿ⁺¹⁾=−g⁽ⁿ⁺¹⁾+βₙp⁽ⁿ⁾
16 RP n=n+1, go to RP 7

The distinction between FR and RP lies in the calculation of β in RP 14. It is generally believed that the RP calculation is more rapidly convergent. Hence, the RP iteration may be used in most cases rather than FR.

Comparison of the linear MRCG iteration and the nonlinear RP iteration reveals that they are very similar. In fact, RP is precisely a nonlinear version of MRCG. Note that the only difference between them lies in the fact that the RP residuals, computed in steps RP.10 and RP.11 involve recomputation of the forward problems while the MRCG residuals are simply recurred in 6.9 (additional, trivial, differences exist in the computation of the α's and β's). In other words, the RP iteration updates the actual nonlinear system at each iteration, while the MRCG simply iterates on the quadratic functional (GN linearization). The overall GN-MRCG algorithm contains two loops—the outer linearization loop, and the inner MRCG loop, while the RP algorithm contains only one loop. Since the RP algorithm updates the forward solutions at each step, it tends to converge faster than GN with respect to total iteration count (number of GN outer iterations times the number, M, of inner loop MRCG iterates). The GN method is, however, generally faster since an MRCG step is faster than an RP step due to the need for forward recomputation in the RP step. The overall codes for the GN-MRCG algorithm and the RP algorithm are so similar that a GN-MRCG code can be converted to an RP code with about 10 lines of modification.

Before leaving this example, it is important to note that the examples given assume that the different frequencies, ω, and views, φ, are all computed in serial fashion. It is important to note however, that the different frequencies and different views are independent in both the forward problem and Jacobian calculations, and therefore can be computed independently and in parallel. This should be clear by examining, for example, the computations in GN.3 and GN.6.3. The forward problem calculations, GN.3 are completely independent and in the gradient calculation, GN.6.3, the computations are independent in frequency and view up to the point at which these contributions are summed to give the gradient. The GN and RP algorithms could thus be executed on a multinode machine in parallel with node intercommunication required only 2 to 3 times per step in order to collect sums over frequency and view number and to distribute frequency/view independent variables, such as scattering potential iterates, gradient iterates, etc., to the nodes.

### Example 2

Image Reconstruction in Layered Ambient Media Using Optimization of a Bilinear or Quadratic Objective Function Containing all Detector Measurement Equations, and with Internal Fields Represented as a Function of the Scattering Potential

Example 1 has a characteristic that the object function γ is considered to be the sole independent variable, and the internal field resulting from the incident field and γ interaction is considered to be a function of this γy. Thus the scattered field can be written as

$${f}_{ω ϕ}^{s c} = {f}_{θ ω}^{s c} ( γ ) ∀ \begin{matrix}θ = 1 , … , Θ \\ ω = 1 , … , Ω\end{matrix} \qquad ( 24 )$$

The difference between Example 2, and Example 1 is that the background medium, in which the object is assumed to be buried, is assumed to be homogeneous in the previous case, whereas it is here assumed that the background is layered. The residual is defined in the same way as the previous example, with de, used to represent the N_{d}-length vector of measured scattered field at the detector positions. That is, the residual vectors for all ω, θ are defined in the following way.

$${r}_{θ ω} ≡ {f}_{θ ω}^{s c} ( γ ) - {d}_{θ ω} 0 ∀ \begin{matrix}θ = 1 , … , θ \\ ω = 1 , … , Ω\end{matrix} \qquad ( 25 )$$

The functional F(γ), dependent upon γ is defined in the same way as example 1:

$$F ( γ ) = {\sum }_{θ ω} {‖ {r}_{θ ω} ‖}_{2}^{2} \qquad ( 26 )$$

This is the functional, dependent upon the object function γ in a highly nonlinear manner, which must be minimized in order to determine the γ values at each gridpoint in the object space.

It is again necessary to compute the Jacobian:

$$( \frac{\partial {f}_{θ ω}^{s c}}{\partial γ} ) = T ∘ ( {( I - {G}_{ω} \cdot [ γ ] )}^{- 1} ( {G}_{ω} \cdot [ {f}_{θ ω} ] ) ) ∀ \begin{matrix}θ = 1 , … , Θ \\ ω = 1 , … , Ω\end{matrix} \qquad ( 27 )$$

where θ again refers to the multiple views and the ω to the multiple frequencies available to us. That is, we again assume that the noise level in the system is zero. We apply the Gauss Newton algorithm to the associated least squares problem and get the same result. This leads to the overdetermined system described above (the notation is identical to the previous example, the difference lies in the actual form of the layered Green's function).

$$[ T ∘ ( {[ I - {G}_{k} \cdot [ γ ] ]}^{- 1} {G}_{k} ) ⊗ {I}_{Θ \times Θ} ] [ \begin{matrix}[ {f}_{1 k} ] \\ ⋮ \\ [ {f}_{Θ k} ]\end{matrix} ] [ {δγ}^{( n )} = - [ \begin{matrix}[ {r}_{1 k}^{( n )} ] \\ ⋮ \\ [ {r}_{Θ k}^{( n )} ]\end{matrix} ] ∀ k : \qquad ( 28 )$$

which must be solved for the γ-update δγ. The left hand side of the above formula is given by (29)

$$T ∘ ( {[ I - {G}_{k} \cdot [ γ ] ]}^{- 1} {G}_{k} ) ⊗ {I}_{Θ \times Θ} = \{ [ \begin{matrix}{{TG}_{k} ( I - [ {γ}^{( n )} ] {G}_{k} )}^{- 1} & 0 .. & 0 \\ 0 & .. & . \\ . & … & . \\ . & .. & 0 \\ 0 & . 0 & {TG}_{k} {( I - [ {γ}^{( n )} ] {G}_{k} )}^{- 1}\end{matrix} ] \}$$

Again, it is possible to use the complex analytic version of the Hestenes overdetermined conjugate gradient algorithm, adapted for least squares problems to iteratively solve this system. This is equivalent to finding the minimum norm solution.

The formula for the Jacobian in the layered medium situation in the presence of multiple frequencies, is,

$$( \frac{\partial {f}_{θ ω}^{s c}}{\partial γ} ) = T ∘ ( {( I - {G}_{ω} \cdot [ γ ] )}^{- 1} ( {G}_{ω} \cdot [ {f}_{θ ω} ] ) ) ∀ \begin{matrix}θ = 1 , … , Θ \\ ω = 1 , … , Ω\end{matrix} \qquad ( 30 )$$

where G_{ω} is the Layered Green's function for the frequency ω. Therefore, in effect, to determine the γ-update, δγ, we merely solve the multiple view problem for each particular frequency, that is, we solve the overdetermined system:

$$[ T ∘ ( {[ I - {G}_{k} \cdot [ γ ] ]}^{- 1} {G}_{k} ) ⊗ {I}_{Θ \times Θ} ] [ \begin{matrix}[ {f}_{1 k} ] \\ ⋮ \\ [ {f}_{Θ k} ]\end{matrix} ] [ {δγ}^{( n )} = - [ \begin{matrix}[ {r}_{1 k}^{( n )} ] \\ ⋮ \\ [ {r}_{Θ k}^{( n )} ]\end{matrix} ] \qquad ( 31 )$$

$$∀ k = 1 , … , Ω$$

which in component form is:

$$[ T ∘ ( {[ I - {G}_{k} \cdot [ γ ] ]}^{- 1} {G}_{k} ) ] [ {f}_{jk} ] δ {γ}^{( n )} = - [ {r}_{jk}^{( n )} ] ∀ \begin{matrix}k = 1 , … , Ω \\ j = 1 , … , Θ\end{matrix} \qquad ( 32 )$$

For multiple view and multiple frequency inversion then, the inversion scheme in layered media reads:
1. STEP Choose an initial guess for the scattering potential, γ⁽⁰⁾
2. STEP Set n=0
3. STEP solve the forward problems

$$( I - G [ γ ] ) {f}_{θ ω} = {f}_{θω}^{i n c} , ∀ \begin{matrix}θ = 1 , … , Θ \\ ω = 1 , … , Ω ,\end{matrix}$$

using biconjugate gradients, and use the result to compute the ϕ-forward maps

$${ϕ}_{θ ω} ( {γ}^{( n )} ) and {r}_{θ ω}^{( n )} = {ϕ}_{θ ω} ( {γ}^{( n )} ) - {d}_{θ ω} = 0 ∀ \begin{matrix}θ = 1 , … , Θ \\ ω = 1 , … , Ω ,\end{matrix}$$

4. STEP Determine the L₂ norm of the total residual vector:

$${tr}^{( n )} ≡ [ \begin{matrix}[ \begin{matrix}[ {r}_{11}^{( n )} ] \\ ⋮ \\ [ {r}_{Θ1}^{( n )} ]\end{matrix} ] \\ ⋱ \\ [ \begin{matrix}[ {r}_{1 Ω}^{( n )} ] \\ ⋮ \\ [ {r}_{Θ Ω}^{( n )} ]\end{matrix} ]\end{matrix} ]$$

5. STEP If

$${‖ {tr}^{( n )} ‖}^{2} ≡ {\sum }_{ω = 1}^{Ω} {\sum }_{θ = 1}^{Θ} {‖ {r}_{θ ω}^{( n )} ‖}^{2} < ε$$

then stop.
6. STEP Solve the N_{d}ΘΩ by N overdetermined system:

$$T ◦ [ \begin{matrix}( {[ I - {G}_{1} \cdot [ g ] ]}^{- 1} {G}_{1} ) ⊗ {I}_{Θ \times Θ} & … & 0 \\ ⋮ & ⋱ & ⋮ \\ 0 & … & ( {[ I - {G}_{Ω} \cdot [ g ] ]}^{- 1} {G}_{Ω} ) ⊗ {I}_{Θ \times Θ}\end{matrix} ]$$

$$[ \begin{matrix}[ \begin{matrix}[ {f}_{11} ] \\ ⋮ \\ [ {f}_{Θ1} ]\end{matrix} ] \\ ⋱ \\ [ \begin{matrix}[ {f}_{1 Ω} ] \\ ⋮ \\ [ {f}_{Θ Ω} ]\end{matrix} ]\end{matrix} ] δ {g}^{( n )} = - [ \begin{matrix}[ \begin{matrix}[ {r}_{11}^{( n )} ] \\ ⋮ \\ [ {r}_{Θ1}^{( n )} ]\end{matrix} ] \\ ⋱ \\ [ \begin{matrix}[ {r}_{1 Ω}^{( n )} ] \\ ⋮ \\ [ {r}_{Θ Ω}^{( n )} ]\end{matrix} ]\end{matrix} ]$$

in the least squares sense for δγ⁽ⁿ⁾, using the conjugate gradient algorithm adapted for least squares problems
7. STEP Update γ via the formula

$${γ}^{( n + l )} = {γ}^{( n )} + δ {γ}^{( n )}$$

8. STEP Set n=n+1, go to begin

The actual values of the angles of incidence θ are chosen to diminish as much as possible the ill conditioning of the problem. Equally spaced values 0≤θ≤2π are ideal, but experimental constraints may prohibit such values, in which case the multiple frequencies are critical.

For biological contrasts (γ<0.15 say) the following approximation, which is essentially identical to the assumption in the previous example, is valid:

$${[ I - {G}_{ω} [ γ ] ]}^{- 1} \approx [ I + {G}_{ω} [ γ ] ] \qquad ( 33 )$$

This is the same Born-like approximation restricted to the Jacobian calculation only, that is assumed in example 1. This approximation has a much larger radius of convergence than the standard born approximation, but substantially reduces the computational complexity of the problem.

### How to Implement the Convolution and Correlation in Layered Medium Imaging

All of the above calculations have been carried out without using the fact that the operation of matrix multiplication with G is in fact the sum of a convolution and a correlation, which is transformed into a pair of convolutions. Symbolically G^{L}=G_{R}+G_{V}, where G_{R} is the correlation and G_{V} is a convolution operator. The actual numerical implementation of the above formulas, therefore, is somewhat different than would naively appear. The implementation of the correlation with convolution is a little more challenging than the convolution in the homogeneous case, in that a change of variables must be used to convert the correlation to a convolution before the Fast Fourier Transform (FFT)s can be applied. A “reflection” operator must be applied to γ before the convolution is applied, this operator is denoted by Z, and is defined by:

$$( Zf ) ( x , y , z ) = f ( x , y , h - z ) \qquad ( 34 )$$

The use of this operator is incorporated into the action of the Lippmann-Schwinger equation on the field f. That is, the equation for the internal fields, which in example 1 read: (I−Gγ)f=f^{inc}, Now becomes (I−G_{V}γ−G_{R} Zγ)f=f^{inc}.

This change has non-trivial ramifications for the construction of the algorithms discussed above. The FFT implementation of this is given below. To prevent the notation from getting out of hand we use [ ] to indicate that a diagonal matrix is constructed out of an n vector, so that [γ⁽ⁿ⁾]f_{Φ}⁽ⁿ⁾ denotes pointwise multiplication of the vectors γ and f. Also F is used to denote the Fourier transform, and * is used to indicate “convolution”. In this notation, the Lippmann-Schwinger equation becomes, in the symbolic form adopted above,

$$( I - {G}_{V} γ - {G}_{R} Z γ ) f = ( f - {G}_{V} * ( [ γ ] f ) - {G}_{R} * Z ( [ γ ] f ) ) = ( f - {ℱ}^{- 1} \{ ( ℱ ( {G}_{V} ) \cdot ℱ ( [ γ ] f ) ) - ( ℱ ( {G}_{R} ) \cdot ℱ \{ Z ( [ γ ] f ) \} ) \} ) \qquad ( 35 )$$

Substantial savings is achieved through the observation that the Fourier Transforms of G_{V} and G_{R} need only be done once, then stored, and finally, that the reflection operator, Z, will commute with Fourier transformation in the x and y (horizontal) directions, since it only involves reflection in the z (vertical) direction.

There are changes also, for example in the computation of f. The biconjugate gradient algorithm requires the use of the adjoint in the solution of the above equation. This adjoint differs from the homogeneous ambient medium case with the introduction of the correlation operator. Also, there are substantial changes in the implementation of the overdetermined conjugate gradient algorithm used to solve (in the least squares sense) the overdetermined system of equations. In this section, as before, we incorporate the k_{sc}² factor into the G_{V} and G_{R} terms. Of course, rather than carry out the inversion of a matrix, we use biconjugate gradients and Fast Fourier Transforms (FFT's), which require the Lippmann Schwinger operator, which we denote by LS, and its adjoint, which are defined by the following formulas:

$$( LS ) f ≡ ( I - {F}^{- 1} [ ( {\tilde{G}}_{V} \cdot F ) + ( {\tilde{G}}_{R} \cdot F \cdot Z ) ] ) ( [ γ ] f )$$

where \tilde{G}_{V}≡F(G_{V}) and \tilde{G}_{R} ≡F(G_{R}) are the Fourier Transforms of G_{V} and G_{R}, and need only be calculated once, then stored. The unitarity of F is used below to determine the adjoint (LS)^{H}.

$${( LS )}^{H} = {( ( I - {ℱ}^{- 1} [ ( {\tilde{G}}_{V} \cdot ℱ ) + ( {\tilde{G}}_{R} \cdot ℱ ◦ Z ) ] ) [ γ ] )}^{H} = [ \text{} \overline{γ} ] ( I - [ ( {ℱ}^{- 1} {\tilde{G}}_{V}^{H} ) + ( {Z}^{H} {ℱ}^{- 1} {\tilde{G}}_{R}^{H} ) ] {( {ℱ}^{- 1} )}^{H} ) = [ \overline{γ} ] ( I - [ ( {ℱ}^{- 1} \overline{{\tilde{G}}_{V}} ) + ( Z {ℱ}^{- 1} \overline{{\tilde{G}}_{R}} ) ] ℱ )$$

where we have used the unitarity of Fourier Transformation F:FH=F⁻¹, and the fact that point wise multiplication is used, and the fact that Z^{H}=Z. For practical problems it is best to obtain a reasonable first guess using low frequency data, then to build on this using the higher frequency information.

### The Actual Construction of the Layered Greens Function

Up to this point, it has been assumed there exists the layered Green's function. For simplicity in describing the construction of this function, we deal with the scalar case, although the inclusion of shear wave phenomena is conceptually no more difficult than this case. The details are given in [Wiskin, 1991]. The general idea is based upon standard ideas in the literature concerning reflection coefficients in layered media. See for example [Muller, G., 1985, Journal of Geophysics, vol. 58] which is herein incorporated by reference. Here, the combination of the reflection coefficients into a bona fide Green's function, and the utilization of this in a forward problem, then more importantly, in the inverse problem solution is described. The procedure involves decomposing the point response in free space into a continuum of plane waves. These plane waves are multiply reflected in the various layers, accounting for all reverberations via the proper plane wave reflection/transmission coefficients. The resulting plane waves are then re-summed (via a Weyl-Sommerfeld type integral) into the proper point response, which in essence, is the desired Green's function in the layered medium. The final result is:

$${G}_{V} ( x - {x}^{′} ; \vert Δ z - Δ {z}^{′} \vert ) = {\int }_{- ∞}^{∞} {e}^{i ω u ( x - {x}^{′} )} C ( u ) [ {e}^{- i ω {b}_{s c} \vert Δ z - Δ {z}^{′} \vert} + {R}^{+} {R}^{-} {e}^{i ω {b}_{s c} \vert Δ z - Δ {z}^{′} \vert} ] du , \qquad ( 38 )$$

$$and$$

$${G}_{R} ( x - {x}^{′} ; \vert Δ z - Δ {z}^{′} \vert ) = {\int }_{- ∞}^{∞} {e}^{i ω u ( x - {x}^{′} )} C ( u ) [ {R}^{+} {e}^{- i ω {b}_{s c} ( Δ z + Δ {z}^{′} )} ] [ {R}^{-} {e}^{- i ω {b}_{s c} ( Δ z - Δ {z}^{′} )} ] du , \qquad ( 39 )$$

$$where$$

$$C ( u ) = {( l - {R}^{+} {R}^{-} )}^{- 1} \frac{i ω u}{{b}_{s c}} ,$$

R⁻ and R⁺ are the recursively defined reflectivity coefficients described in Muller's paper,
u is the horizontal slowness,

$$u = \frac{sin θ}{c}$$

Recall that Snell's law guarantees that u will remain constant as a given incident plane wave passes through several layers.
ω is the frequency
b_{sc} is the vertical slowness for the particular layer hosting the scattering potential

This Green's function for imaging inhomogeneities residing within Ambient Layered Media must be quantized by convolving with the sine (really Jinc) basis functions described below. This is done analytically in [Wiskin, 1991], and the result is given below.

### Discretization of Layered Medium Lippmann-Schwinger Equation

Unfortunately the basis functions that were used in the free space case (Example 1) cannot be used to give the discretization in the layered medium case because of the arbitrary distribution of the layers above and below the layer which contains the scattering potential. The sine functions may continue to be used in the horizontal direction, but the vertical direction requires the use of a basis function with compact support (i.e., should be nonzero only over a finite region). The sampled (i.e., discrete) Green's operator in the layered case is defined by

$${G}^{L} ( j , k , m ) = {G}^{L} ( j δ , k δ , m δ ) \qquad ( 40 )$$

where the three dimensional basis functions are of the form:

$$B ( x ) = B ( x , y , z ) = {S}_{( j , δ )} ( x ) {S}_{( k , δ )} ( y ) {Λ}_{r n} ( z ) \qquad ( 41 )$$

(I is the set of integers) and where:

$$x = ( \begin{matrix}x \\ y \\ z\end{matrix} ) ∈ {ℛ}^{3}$$

$${x}_{jkm} = ( \begin{matrix}j δ \\ k δ \\ m δ\end{matrix} ) j , k , m ∈ I$$

The basis functions in the horizontal (x-y) plane S_{(j,δ)} are based upon the Whittaker sine function:

$$sin c ( x ) = \frac{sin ( π x )}{π x}$$

The basis functions in the vertical (z) direction, on the other hand, are given by

$${Λ}_{m} ( z ) = Λ ( z - m δ ) ,$$

where Λ(z) is the “tent” function:

$${Λ}_{( z )} = \{ \begin{matrix}\frac{1}{δ} ( z + δ ) & z ∈ [ - δ , 0 ] \\ - \frac{1}{δ} ( z - δ ) & z ∈ [ 0 , δ ]\end{matrix}$$

The form of the discretized Lippmann Schwinger equation is

$${f}^{inc} ( \begin{matrix}n δ \\ m δ \\ l δ\end{matrix} ) = {f}_{nml}^{inc} = f ( \begin{matrix}n δ \\ m δ \\ l δ\end{matrix} ) - {k}_{sc}^{2} {\sum }_{{n}^{′} , {m}^{′} , {l}^{′}} {G}_{π / δ}^{V} ( \begin{matrix}n - {n}^{′} \\ m - {m}^{′} \\ l - {l}^{′}\end{matrix} ) {γ}^{L} f ( \begin{matrix}{n}^{′} δ \\ {m}^{′} δ \\ {l}^{′} δ\end{matrix} ) {k}_{sc}^{2} - {\sum }_{{n}^{′} , {m}^{′} , {l}^{′}} {G}_{π / δ}^{V} ( \begin{matrix}n - {n}^{′} \\ m - {m}^{′} \\ l - {l}^{′}\end{matrix} ) {γ}^{L} f ( \begin{matrix}{n}^{′} δ \\ {m}^{′} δ \\ {l}^{′} δ\end{matrix} ) , \qquad ( 42 )$$

with the vectors

$${G}_{π / δ}^{R} and {G}_{π / δ}^{y}$$

are the result of first convolving the Green's function with the basis functions, and then sampling at the gridpoints. The superscript R refers to the correlation part, whereas the superscript V refers to the convolution part of the Green's function. Both are computed via Fast Fourier Transforms, and are very fast. In the free space case the correlation part is zero. That is, we make the definition for L=V or R—(i.e., convolution or correlation):

$${G}_{π / δ}^{L} ( n , m , l ) ≡ \int \int {\int }_{{R}^{3}} S ( {x}^{′} ) S ( {y}^{′} ) Λ ( {z}^{′} ) {G}^{L} ( \begin{matrix}n δ - {x}^{′} \\ m δ - {y}^{′} \\ lδ - {z}^{′}\end{matrix} ) {dx}^{′} {dy}^{′} {dz}^{′} \qquad ( 43 )$$

substitution into equation (17) yields the form (42).

In matrix notation, this equation gives:

$$f = {( I - {k}_{sc}^{2} {G}_{V}^{*} {γ}_{L} - {k}_{sc}^{2} {\hat{G}}_{R}^{*} Z {γ}_{L} )}^{- 1} {f}^{inc} \qquad ( 44 )$$

where now * represents discrete convolution in 3D. Now that we have constructed the Integral equations in discrete form, we must derive the closed form expression for the layered Green's operators. The construction of the closed form expression of the sampled and convolved acoustic layered Green's function

$${G}_{π / δ}^{L} ,$$

from the layered Green's function given in equations (38 and 39) above is given by performing the integration in (43) analytically. This process is carried out in [Wiskin, 1991]. The final result is, for the convolutional part,

$${G}_{v} ( m , n ) = \{ \begin{matrix}A {\int }_{u = 0}^{u δ} {J}_{0} C ( u ) \frac{2}{i ω {b}_{sc}} \{ {R}^{-} {R}^{+} \{ - 1 + \frac{( {e}^{i ω {b}_{sc} δ} - 1 )}{i ω {b}_{sc} δ} \} + \{ 1 - {e}^{- i ω {b}_{sc} δ} \frac{( {e}^{i ω {b}_{sc} δ} - 1 )}{i ω {b}_{sc} δ} \} \} du & n = 0 \\ A {\int }_{u = 0}^{u δ} {J}_{0} C ( u ) \frac{l}{{ω}^{2} {b}_{sc}^{2} δ} \{ 2 - {e}^{i ω {b}_{sc} δ} - {e}^{- i ω {b}_{sc} δ} \} {e}^{- i ω {b}_{sc} \vert n δ \vert} + {R}^{-} {R}^{+} {e}^{i ω {b}_{sc} \vert n δ \vert} \} du & \vert n \vert \geq 1\end{matrix} \qquad ( 45 )$$

$$∀ m ∈ [ 0 , M - 1 ] , ∀ n ∈ [ - N + 1 , N - 1 ]$$

$$and$$

$${G}_{v} ( m , n ) = A {\int }_{u = 0}^{u δ} {J}_{0} C ( u ) \frac{\{ 2 - {e}^{i ω {b}_{m} δ} - {e}^{- i ω {b}_{m} δ} \}}{{ω}^{2} {b}_{sc}^{2} δ} \{ {R}^{+} ( {e}^{- i ω {b}_{sc} ( h + 2 d )} {e}^{- i ω {b}_{sc} n δ} ) + {R}^{-} ( {e}^{i ω {b}_{sc} ( h + 2 d )} {e}^{i ω {b}_{sc} n δ} ) \} \qquad ( 46 )$$

$$∀ m ∈ [ 0 , M - 1 ] , ∨ n ∈ [ - N + 1 , N - 1 ]$$

In the above formulas J₀≡J₀(uω|m|δ) is the zero^{th} order Bessel Function, and the upper limit u_{δ} is defined as:

$${u}_{δ} = 1 / {c}_{δ} = {k}_{δ} \frac{1}{ω} = \frac{π / δ}{ω}$$

for wavenumber k_{δ}=π/δ. Also, C(u)≡(1−R⁻R⁺)⁻¹S^{c} where

$${S}^{c} = \frac{i ω u}{{b}_{sc}}$$

with b_{sc} defined as the vertical slowness in the layer containing the scattering point. When this layer is assumed to have acoustic wave velocity β_{sc}, it is given explicitly by:

$${b}_{sc} = \sqrt{\frac{l}{{β}_{sc}^{2}} - {u}^{2}}$$

These expressions give the discretized 3D Acoustic layered Green's function, which is used in the layered media discretized Lippmann-Schwinger equation to obtain the solution field within the inhomogeneity.

As can be seen, the correlation part of the green's function is included. This correlation part is zero for the free space case. This correlational part is directly applicable as it occurs above to the fish echo-locator/counter, and to the mine detection device in the acoustic approximation. (There are specific scenarios, where the acoustic approximation will be adequate, even though shear waves are clearly supported to some degree in all sediments). The inclusion of shear waves into the layered media imaging algorithm (the technical title of the algorithm at the heart of the hazardous waste detection device, the fish echo-locator, and the buried mine identifier) is accomplished in Example 6 below. A generalized Lippmann-Schwinger equation is proposed. This general equation is a vector counterpart to the acoustic Layered Green's function, and is discretized before it can be implemented. The process of discretization is virtually identical to the method revealed above. Furthermore, the BCG method for solving the forward problem, the use of the sine basis functions, and the use of the Fast Fourier Transform (FFT) are all carried out identically as they in the acoustic (scalar) case.

### Example 3

Imaging with Electromagnetic Wave Energy

The use of electromagnetic energy does not greatly affect the basic idea behind the imaging algorithm, as this example will demonstrate. Furthermore, we will see that it is possible to combine the layered Green's function with the electromagnetic free space Green's function to image materials within layered dielectrics. In fact this process of combining the layered Green's function with Green's functions derived for other structures and/or modalities than the free space acoustic case can be extended almost indefinitely. Another example is given below, where the combination of the Acoustic Biot Green's function with the layered Green's function is carried out. Further extensions that are not detailed here, are: (1) Combining Elastic (including shear wave energy) Wave equations with the layered Green's function, (3) Combining the Elastic Biot equations with the layered Green's function, (4) Combining the elastic and electromagnetic wave equations to model transducers. For simplicity, we consider a homogeneous, isotropic, non-magnetic background material. The time dependence will be e^{iωt}, the magnetic properties of free space are summarized in \hat{Z}ₒ≡iωμₒ, and the electric properties are summarized in ŷₒ ≡iωεₒ. Within the object being imaged the magnetic properties are \hat{Z}≡\hat{Z}ₒ ≡iωμₒ (the equivalence with the free space value is the nonmagnetic media assumption). The electric properties of the object being imaged are summarized in ŷ≡σ+ŷ₀≡σ+iωεᵣε₀, which is the complex admittivity of the object. The larger G is, the less able the medium is able to support electromagnetic wave transport. These properties are combined in

$${k}_{o}^{2} = - {\hat{y}}_{o} {\hat{z}}_{o} = {ω}^{2} {μ}_{o} {ε}_{o} = \frac{{ω}^{2}}{{c}_{o}^{2}} \qquad ( 47 )$$

which is a measure of the number of wave cycles present per unit distance for a given frequency ω, when the wave speed of propagation is cₒ. The object's electrical properties (recall it is assumed non-magnetic for simplicity) are summarized in the “object function”, γ. (the normalized difference from the surrounding medium):

$$γ ( r ) ≡ \frac{\hat{y} - {\hat{y}}_{o}}{{\hat{y}}_{o}} = \frac{σ + i {ωε}_{r} {ε}_{o}}{i {ωε}_{o}} - 1 = - i \frac{σ}{{ωε}_{o}} + {ε}_{r} - 1 \qquad ( 48 )$$

The total electric field equations are:

$${E}^{i} ( r ) = E ( r ) - ( {k}_{o}^{2} + ∇ ∇ \cdot ) \int γ ( {r}^{′} ) E ( {r}^{′} ) \frac{{e}^{- {ik}_{o} \vert r - {r}^{′} \vert}}{4 π \vert r - {r}^{′} \vert} {d}^{3} {r}^{′} \qquad ( 49 )$$

where Eⁱ(r) is the 3-D incident field, E(r) is the 3-D total field.

The construction of the sine basis discretization and the GN and RP algorithms for this 3-D EM case is essentially equivalent to the 2-D scalar acoustic case described above. See [Borup, 1989] for the details of the discretization and solution of the forward problem by FFT-BCG. The vector—tensor nature of the fields—Green's function is the only new feature and this is easily dealt with.

For simplicity, we now look at the situation where there is no z dependence in either the object function, i.e γ(x, y, z)=γ(x, y), neither in the incident field. The vector ρ=(x, y) is used for the position vector in (x,y)-space.

$${E}^{i} ( ρ ) = E ( ρ ) - ( {k}_{o}^{2} + ∇ ∇ \cdot ) \int γ ( {ρ}^{′} ) E ( {ρ}^{′} ) \frac{1}{4 i} {H}_{0}^{( 2 )} ( {k}_{o} \vert ρ - {ρ}^{′} \vert {d}^{2} {ρ}^{′} \qquad ( 50 )$$

in matrix form the equation (50) looks like:

$${E}^{i} ( ρ ) = E ( ρ ) - [ \begin{matrix}{k}_{o}^{2} + \frac{{\partial }^{2}}{\partial {x}^{2}} & \frac{{\partial }^{2}}{\partial x \partial y} & \\ \frac{{\partial }^{2}}{\partial y \partial x} & {k}_{o}^{2} + \frac{{\partial }^{2}}{\partial {y}^{2}} & \\ & & {k}_{o}^{2}\end{matrix} ] \int γ ( {ρ}^{′} ) E ( {ρ}^{′} ) \frac{1}{4 i} {H}_{0}^{( 2 )} ( {k}_{o} \vert ρ - {ρ}^{′} \vert {d}^{2} {ρ}^{′} \qquad ( 51 )$$

From this form of the equation, it is clear that the electric field in the z-direction is uncoupled with the electric field in the x-y plane. Thus, in this situation the field consists of two parts. The so-called transverse electric (TE) mode, in which the electric field is transverse to the z direction, and the transverse magnetic (TM) mode, in which the electric field has a nonzero component only in the z direction. The TM mode is governed by the scalar equation:

$${E}_{z}^{i} ( ρ ) = {E}_{z} ( ρ ) - \frac{{k}_{o}^{2}}{4 i} \int γ ( {ρ}^{′} ) {E}_{z} ( {ρ}^{′} ) {H}_{0}^{( 2 )} ( {k}_{o} \vert ρ - {ρ}^{′} \vert {d}^{2} {ρ}^{′} \qquad ( 52 )$$

$$where$$

$${E}^{i} ( ρ ) = ( \begin{matrix}{E}_{x}^{i} \\ {E}_{y}^{i} \\ {E}_{z}^{i}\end{matrix} ) and E ( ρ ) = ( \begin{matrix}{E}_{x} \\ {E}_{y} \\ {E}_{z}\end{matrix} ) ( x , y )$$

which is identical to the 2-D acoustic equation discussed previously. Thus, the 2-D TM electromagnetic imaging algorithm is identical to the 2-D acoustic case discussed in detail above.

The TE mode is given by the following equation:

$$( \begin{matrix}{E}_{x}^{i} ( ρ ) \\ {E}_{y}^{i} ( ρ )\end{matrix} ) = ( \begin{matrix}{E}_{x} ( ρ ) \\ {E}_{y} ( ρ )\end{matrix} ) - \frac{1}{4 i} [ \begin{matrix}{k}_{o}^{2} + \frac{{\partial }^{2}}{\partial {x}^{2}} & \frac{{\partial }^{2}}{\partial x \partial y} \\ \frac{{\partial }^{2}}{\partial y \partial x} & {k}_{o}^{2} + \frac{{\partial }^{2}}{\partial {y}^{2}}\end{matrix} ] \int γ ( {ρ}^{′} ) ( \begin{matrix}{E}_{x} ( {ρ}^{′} ) \\ {E}_{y} ( {ρ}^{′} )\end{matrix} ) {H}_{0}^{( 2 )} ( {k}_{o} \vert ρ - {ρ}^{′} \vert ) {d}^{2} {ρ}^{′} \qquad ( 53 )$$

The field is a two component vector and the Green's function is a 2×2 tensor Green': function:

$$G = [ \begin{matrix}{g}_{xx} & {g}_{xy} \\ {g}_{xy} & {g}_{yy}\end{matrix} ] = \frac{1}{4 i} [ \begin{matrix}{k}_{o}^{2} + \frac{{\partial }^{2}}{\partial {x}^{2}} & \frac{{\partial }^{2}}{\partial x \partial y} \\ \frac{{\partial }^{2}}{\partial y \partial x} & {k}_{o}^{2} + \frac{{\partial }^{2}}{\partial {y}^{2}}\end{matrix} ] {H}_{0}^{( 2 )} ( {k}_{o} \vert ρ - {ρ}^{′} \vert ) \qquad ( 54 )$$

In compact notation:

$${E}^{i} = ( I - G Γ ) E$$

where:

$${E}^{i} = [ \begin{matrix}{E}_{x}^{i} \\ {E}_{y}^{i}\end{matrix} ] , E = [ \begin{matrix}{E}_{x} \\ {E}_{y}\end{matrix} ] , I = [ \begin{matrix}I & 0 \\ 0 & I\end{matrix} ] \qquad ( 55 )$$

$$and$$

$$Γ = [ \begin{matrix}γ & 0 \\ 0 & γ\end{matrix} ]$$

This equation also has a convolution form and can thus be solved by the FFT-BCG algorithm as described in [Borup,1989] The construction of the GN-MRCG and RP imaging algorithms for this case is identical to the 2-D acoustic case described above with the exception that the fields are two component vectors and the Green's operator is a 2×2 component Green's function with convolutional components.

Finally, note that the presence of layering parallel to the z direction can also be incorporated into these 2-D algorithms in essentially the same manner as above. Special care must be taken, of course, to insure that the proper reflection coefficient is in fact, used. The reflection coefficient for the TM case is different from the TE case.

### Example 5

### Extension to Biot Theory (Acoustic Approximation)

Note: an extensive discussion of Biot Theory in the acoustic approximation and a resulting imaging method with corresponding implementations are described above in the section entitled Biot—porous media. In the current example provided below, an initial imaging theory is discussed.

In the article [Boutin, 1987, Geophys. J. R. astr. Soc., vol. 90], herein incorporated by reference, a Green's function for the determination of a point response to a scattering point located in an isotropic, homogeneous, porous medium that supports elastic waves is developed. The implementation of this Greens' function into an imaging algorithm has never been carried out before. In this section, we have adapted their approach to derive an acoustic approximation to the fully elastic Biot theory that enables us to present a simplified practical tool for the imaging of porosity-like parameters in a geophysical context. The implementation of the full elastic Biot imaging algorithm using the Green's function of [Boutin, 1987] in place of the one derived in [Wiskin, 1992] is no different from the discretization employed here. The use of the sine basis functions, the FFT's, the biconjugate gradients, and so on, is identical.

We have developed a model that incorporates the parameters of fluid content, porosity, permeability, etc., but instead of the 3+1 degrees of freedom of the two phase elastic model, has only 1+1, corresponding to the two independent variables, ρₛ, and ρₗ, the pressure field within the solid (modelled as a liquid in this approximation) and truly liquid phase respectively. As with the previous example. The use of the Biot theory Green's function can be combined profitably with layering, to image material in a layered background.

Define (see [Boutin, 1987], for more complete discussion of the parameters):
ω as the frequency of the interrogating wave
ρₗ as the density of the liquid phase
ρₛ as the density of the solid phase
n as a saturation parameter
K(ω) as the generalized, explicitly frequency-dependent Darcy coefficient introduced via homogenization theory.

αₒ, θ, ρ are parameter's defined in the following way:

$$ρ = ( 1 - n ) {ρ}_{s} + {ρ}_{1} [ n + {ρ}_{1} {ω}^{2} K ( ω ) / i ω ] θ = K ( ω ) / i ω )$$

$${α}_{o} = α + {ρ}_{1} {ω}^{2} K ( ω ) / i ω = α + {ρ}_{1} {ω}^{2} θ$$

The “acoustic two phase Green's function” is the Green's function obtained by solving the above system with a distributional right hand side, where δ(x) is the Dirac delta distribution. That is we seek the solution to the following matrix operator equation: BG_{Biot}+I_{2×2}δ(x)=0, where G_{Biot}:R^{2,3}→R² is a function on R³ or R² and I_{2×2} is the two dimensional identity matrix. B is given by:

$$B = [ \begin{matrix}θ {∇}^{2} - β & - {α}_{0} \\ - {α}_{0} {∇}^{2} & λ {∇}^{2} + {ω}^{2} ρ\end{matrix} ]$$

This author obtained

$${G}_{Biot} = \frac{1}{4 π θ λ} \frac{{α}_{0}}{( {δ}_{2}^{2} - {δ}_{1}^{2} )} \{ ( w \cdot d ) [ \begin{matrix}λ & 0 \\ {α}_{0} & θ\end{matrix} ] + ( w \cdot v ) [ \begin{matrix}\overline{p} {ω}^{2} & {α}_{0} \\ 0 & - \overline{β}\end{matrix} ] \}$$

where w:R³→R² is the 2D vector defined by:

$$w = ( \begin{matrix}\frac{{e}^{- i {δ}_{1} \vert x \vert}}{\vert x \vert} \\ \frac{{e}^{- i {δ}_{2} \vert x \vert}}{\vert x \vert}\end{matrix} )$$

and x∈R³ for the acoustic two-phase 3D model, and where d and v are 2 component vectors (representing the two phases) given by

$$d = ( \begin{matrix}- {δ}_{1}^{2} \\ {δ}_{2}^{2}\end{matrix} ) , v = ( \begin{matrix}1 \\ - 1\end{matrix} )$$

A very similar analysis gives an equation and corresponding Green's Operator for the acoustic two-phase 2D (two spatial independent variables) case, in which w: R²→R² and x∈R² The operator is very similar except that the w vector contains Hankel functions of the zeroth order:

$$w = ( \begin{matrix}\frac{i}{4} {H}_{0}^{( 2 )} ( {δ}_{1} \vert x \vert ) \\ \frac{i}{4} {H}_{0}^{( 2 )} ( {δ}_{2} \vert x \vert )\end{matrix} )$$

Notice the existence of the two wavenumbers δ₁ and δ₂ in this acoustic approximation to Biot theory guarantee the existence of a fast and slow compressional wave—a distinguishing characteristic of the classical elastic Biot theory.

More importantly this algorithm provides us with a computationally feasible means of inverting for phenomenological parameters derived from the classical elastic two phase Biot Theory.

Finally, it is important to point out that convolving the above functions with the sine basis function analytically is done in exactly the same manner with G_{Biot}=G_{2×2} above as it was done above because x only occurs in the form of the scalar acoustic Green's operator. This is why it is possible to create very efficient algorithms based upon the sinc-Galerkin method and FFT's as described in [Borup, 1992], using the Green's Operator described above.

It is now possible to obtain an algorithm for the inversion of the 2D Biot porous medium by analogy with the algorithm based upon the Lippmann-Schwinger equation used earlier. This new algorithm is based upon a “two phase” Lippmann-Schwinger type equation derived in a future publication.

$${r}_{0} ( x ) = r ( x ) - [ \begin{matrix}- {\overline{b}}_{1} & 0 \\ 0 & {\overline{b}}_{2}\end{matrix} ] \int \int \int {G}_{2 \times 2} ( x - ξ ) {D}^{′} ( ξ ) r ( ξ ) {d}^{3} ξ$$

$$where$$

$${D}^{′} ( x ) = [ \begin{matrix}{γ}_{1} & 0 \\ 0 & {γ}_{2}\end{matrix} ]$$

and the object functions γᵢ are given by

$${γ}_{j} ( x ) = {( - 1 )}^{j} ( \frac{{b}_{j} ( x )}{{\overline{b}}_{j}} - 1 ) j = 1 , 2$$

This equation is discretized in the same manner as above, and the convolution character is preserved. With the explicit form of the discretized Biot Lippmann-Schwinger Integral Equation the imaging problem is solved by applying the analogous conjugate gradient iterative solution method to the Gauss-Newton Equations in a manner exactly analogous to the discussion above. See above for the paraxial approximation to this imaging theory (e.g., in the section entitled “Biot—porous media” and the section entitled “paraxial approximation”).

Finally, as mentioned above, it is certainly possible to construct, using the above principles, a layered Green's function for the Biot theory in exact analogy with the construction for the acoustic layered Green's Operator. This operator will be a matrix operator (as is the acoustic two phase Green's Operator constructed above), and will consist of a convolutional and correlational part (as does the acoustic single phase Green's Operator constructed earlier). Because of these convolutional and correlational parts, the FFT methods discussed above are directly applicable, making the algorithm feasible from a numerical standpoint.

### Example 6

Non-Perturbative Inversion of Elastic Inhomogeneous Structures Buried within a Layered Half Space

In elastic media (by convention in this patent, media that supports shear wave activity) the relevant parameters are γ, μ, (Lame' parameters), ρ (density) and absorption. The inversion for these elastic parameters (i.e. the first and second Lame' parameters, λ and μ, follows virtually the same prescription as was outlined above for the acoustic scalar case. In a manner similar to the Electromagnetic Inversion problem discussed above, it is possible to break up the arbitrary 3 dimensional vector u(x,y,z), representing the displacement into components that propagate independently. The exact description of this procedure is given in [Wiskin, 1991], and [Muller, 1985]. This example will not deal with this decomposition since the idea behind this is more easily seen by looking at the electromagnetic example given earlier.

The idea here is to incorporate the above solution to the layered inversion problem directly into the Green's operator. As before, in the electromagnetic and the acoustic case, this Green's function is used in the integral equation formulation of the inversion problem of imaging an inhomogeneous body extending over an arbitrary number of layers.

The following is the general elastic partial differential equation which governs the displacement vector field u in the general case where λ and μ both depend upon x∈R³. When X and μ are independent of x, we have the homogeneous elastic case.

$$- {ω}^{2} ρ ( x ) {u}^{i} ( x ) - \frac{\partial }{\partial {x}^{j}} [ λ ( x ) \frac{\partial }{\partial {x}^{k}} {u}^{k} ( x ) ] + \frac{\partial }{\partial {x}^{i}} [ μ ( x ) \{ \frac{\partial }{\partial {x}^{i}} {u}^{k} ( x ) + \frac{\partial }{\partial {x}^{k}} {u}^{j} ( x ) \} ] = {f}^{j} ( x )$$

fⁱ(x) represents the applied body force, and μ(x) and λ(x) are the Lame' parameters, their dependence upon x∈R³ is the result of both the inhomogeneity to be imaged and the ambient layered medium. In particular uᵢ(\vec{y})\vec{y}∈R³ is the i^{th} component (i=1,2,3) of the displacement field at point \vec{y}∈R³

$${u}_{i}^{0} ( \vec{y} )$$

is the i^{th} component of the incident field.

ρ₁(\vec{x})+ρ₀(z)=ρ(\vec{x}) is the total density variation, it consists of the 3-D variation in ρ₁ and the vertical 1-D variation in ρ₀.

λ₁(\vec{x})+λ₀(z)=λ(\vec{x}) is the total variation in λ, the first Lame' parameter, λ₁ has 3-D variation, and λₒ has 1-D vertical variation.

μ₁(\vec{x})+μ₀(z)=μ(\vec{x}) is the total variation in the second Lame' parameter μ. The μ₁ has variation in 3-D, the μ₂ has variation in 1-D (vertical).

ρ(\vec{x})=density=ρ₁(\vec{x})+ρ₀(z), where ρ₀(\vec{x}) represents the layered medium without the object, and ρ₁(\vec{x}) represents the object to be imaged.

λ₀(z) and μ₀(z) are the z-dependent Lame' parameters representing the layered medium

λ₁(\vec{x}) and μ₁(\vec{x}) are the Lame' parameters representing the object to be imaged

The above differential equation is converted to the following elastic “Generalized Lippmann-Schwinger equation”

$${u}_{i} ( \vec{y} ) = {u}_{i}^{0} ( \vec{y} ) - \int \int {\int }_{Vol} \{ {G}_{i m}^{( LAY )} ( \vec{y} , \vec{x} ) \} {u}_{m} ( \vec{x} ) d \vec{x} , i = 1 , 2 , 3$$

by means of the kernel

$${G}_{i m}^{( L A Y )} ( \vec{y} , \vec{x} )$$

which is constructed below. Where the volume integral is performed over the finite volume that contains the image space (and the object being imaged, for example, an ore body, submarine mine, oil reservoir, etc.). This is the basic integral equation which forms the theoretical foundation for the Layered Green's Operator approach to Inverse Scattering in a layered medium.

This kernel is a 3 by 3 matrix of functions which is constructed by a series of steps:

### Step 1:

Beginning with the acoustic (scalar or compressional) Green's function given by

$$G ( {k}_{o} \vert r - {r}^{′} \vert ) = \frac{{e}^{- i {k}_{o} \vert r - {r}^{′} \vert}}{4 π \vert r - {r}^{′} \vert}$$

### Step 2:

The free space elastic Green's matrix is a 3 by 3 matrix of functions, built up from the free space Green's function. It's component functions are given as

$${G}_{i m} ( {k}_{T} ; {k}_{L} \vert r - {r}^{′} \vert ) = μ G ( {k}_{T} \vert r - {r}^{′} \vert ) {δ}_{i m} + \frac{μ}{{k}_{T}^{2}} \frac{{\partial }^{2}}{\partial {x}^{i} \partial {x}^{m}} [ G ( {k}_{T} \vert r - {r}^{′} \vert ) - G ( {k}_{L} \vert r - {r}^{′} \vert ) ]$$

### Step 3:

Next the layered Green's function, Gᵢₘ^{L}(\vec{y}, \vec{x}),f or a layered elastic medium is defined. It is defined in terms of Gᵢₘ, the components i,m=1, . . . , 3, of the elastic Green's function in homogeneous elastic media given above. The components of the layered Green's matrix are integrals over Bessel functions and reflection coefficients in essentially the same manner as the acoustic layered Green's function consisted of integrals over wavenumber, of the acoustic reflection coefficients. This dyadic is patterned after [Muller, 1985], in the manner discussed in [Wiskin, 1992].

### Step 4:

Finally the layered Green's kernel

$${G}_{i m}^{( L A Y )} ( \vec{y} , \vec{x} ) ,$$

is constructed
x∈R³

$${G}_{i m}^{( LAY )} ( \vec{y} , \vec{x} ) = - {ω}^{2} {G}_{im}^{L} ( \vec{y} , \vec{x} ) {ρ}_{1} ( \vec{x} ) - \frac{\partial }{\partial {x}^{m}} \{ \frac{\partial }{\partial {x}^{j}} \{ ( \frac{\partial }{\partial {x}^{j}} {G}_{ij}^{L} ( \vec{y} , \vec{x} ) ) {λ}_{l} ( \vec{x} ) \} + \frac{\partial }{\partial {x}^{j}} \{ ( \frac{\partial }{\partial {x}^{m}} {G}_{ij}^{L} ( \vec{y} , \vec{x} ) + \frac{\partial }{\partial {x}^{j}} {G}_{im}^{L} ( \vec{y} , \vec{x} ) {μ}_{1} ( \vec{x} ) \}$$

where

$${G}_{i m}^{L} ( \vec{y} , \vec{x} )$$

is the layered Green's function for the elastic layered medium. The progressive constructions can be represented in the following way, beginning with the acoustic free space Green's function:

$$\begin{matrix}G ( {k}_{o} \vert r - {r}^{′} \vert ) = \frac{{e}^{- {ik}_{o} \vert r - {r}^{′} \vert}}{4 π \vert r - {r}^{′} \vert}\end{matrix} ⇒ ⇒ \begin{matrix}{G}_{im} ( \vec{y} , \vec{x} )\end{matrix} ⇒ ⇒ \begin{matrix}{G}_{im}^{L} ( \vec{y} , \vec{x} )\end{matrix} ⇒ ⇒ \begin{matrix}{G}_{i m}^{( LAY )} ( \vec{y} , \vec{x} )\end{matrix}$$

Alternatively, in words:

| Acoustic | elastic | | |
|---|---|---|---|
| free | free | elastic | kernel |
| space | space | layered | for elastic |
| Green's | Greens | Green's | layered Lippmann- |
| function | function | function | Schwinger equation |

Using the last “kernel” in this series in the generalized elastic layered Lippmann-Schwinger Equation gives the vector displacement.

$${u}_{i} ( \vec{y} ) = {u}_{i}^{0} ( \vec{y} ) - \int \int {\int }_{Vol} \{ {G}_{i m}^{( L A Y )} ( \vec{y} , \vec{x} ) \} {u}_{m} ( \vec{x} ) d \vec{x}$$

which is then discretized, using the sine basis in exactly the same way as for the previous examples, and, the FFT, and biconjugate gradient, and conjugate gradient algorithms are then applied to this vector equation, in the same way as done above. Thus the elastic modality (including shear waves) is accounted for.

The construction of the layered Green's function in the full elastic case (with shear wave energy included is slightly more complicated than the purely scalar case. For this reason, we look at the construction in more detail, see also [Wiskin, 1993]

For discretization of the resulting equations see the electromagnetic case discussed in example 3.

The Construction of the Continuous Form of the Layered Green's Operator G^{L}-3D and with full elastic mode conversion Case

Now we proceed with the construction of the continuous variable form of the Layered Green's operator. The closed form expression for the discretized form of the bandlimited approximation to this continuous variable Green's operator is constructed in a manner exactly analogous to the vector electromagnetic case discussed in example 3.

By analogy with the acoustic (scalar) situation, the construction of the layered Green's operator can be viewed as a series of steps beginning with the 3D free space Green's operator.

Step a) Decompose a unit strength point source into a plane wave representation (Weyl-Sommerfeld Integral). For the elastodynamic (vector) case we must consider the three perpendicular directions of particle motion, which we take to be the horizontal direction, the direction of propagation, and the direction perpendicular to the previous two. Muller has given the representation for a point source in elastic media in [Muller, 1985].

Step b) Propagate and reflect the plane waves through the upper and lower layers to determine the total field at the response position. The total field will consist of two parts: u^{up(r)}, the upward propagating particle velocity, and u^{d}(r), the downward propagating particle velocity at position r. This propagation and reflection is carried out analytically by means of the reflection coefficients, which in the elastic case, are the matrices R⁻ and R⁺ (R⁻ and R⁺ are 2×2 matrices), and the scalars, r⁻, and r⁺. The matrices correspond to the P-SV (compressional and shear vertical polarized waves) for the case of horizontal stratification. (x is the horizontal, and z is the vertical coordinate, z is positive downward). The scalar coefficients correspond to the SH (horizontally polarized) shear waves, which propagate without mode conversion to the other types for the case of horizontally layered media.

R⁻ and r⁻, represent the wave field reflected from the layers below the source position, and R⁺ and r⁺ represent the cumulative reflection coefficient from the layers above the source position, for the matrix and scalar cases respectively.

These total reflection coefficients are formed recursively from the standard reflection coefficients found in the literature (e.g., Muller [1985], and Aki and Richards, Quantitative Seismology, Freeman and Co., 1980, herein included as a reference].

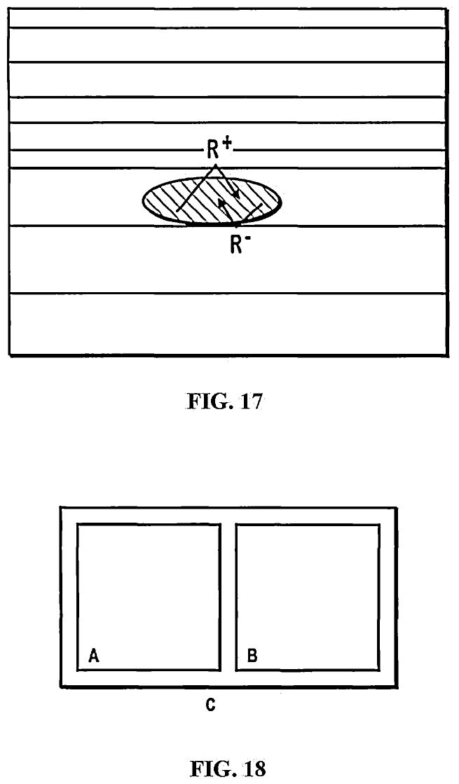

FIG. 17 is schematic showing the Reflection coefficients relevant to the layered medium Green's function approach. With reference to FIG. 14, as in the scalar case, in order to have a single reference position, R⁺ and R⁻ are both given with respect to the top of layer that contains the scattering point, which is denoted by “sc”. That is, R⁺ represents the reflection coefficient for an upgoing wave at the interface between layer sc and sc−1.

In our case the expressions S^{u} and S^{d}, S^{d} and S^{u}, and are those derived from the Sommerfeld integral representation of a point source of unit strength, given in Muller. [see also Aki and Richards, 1980]. For example:

$${S}^{d} = {e}^{i ω {b}_{s c} ( {z}^{′} - {z}_{sc} )} = {e}^{i ω {b}_{s c} Δ {z}^{′}}$$

$${S}^{u} = {e}^{- i ω {b}_{s c} ( {z}^{′} - {z}_{s c} )} = e - i ω {b}^{s c} Δ {z}^{′}$$

for the scalar case.

As shown in detail, by Muller, the total contribution of the upward travelling wave is given by

$${S}^{u} + {R}^{-} {R}^{+} {S}^{u} + {R}^{-} {R}^{+} {R}^{-} R + {S}^{u} + {R}^{-} {R}^{+} {R}^{-} {R}^{+} {S}^{u} + … = [ \text{} 1 + {R}^{-} {R}^{+} + {R}^{-} {R}^{+} {R}^{-} {R}^{+} + {R}^{-} {R}^{+} {R}^{-} {R}^{+} {R}^{-} {R}^{+} + … ] {S}^{u} = {[ 1 - {R}^{-} {R}^{+} ]}^{- 1} {S}^{u}$$

for the transverse shear wave (SH or horizontal shear wave), and

$${S}^{u} + {R}^{-} {R}^{+} {S}^{u} + {R}^{-} {R}^{+} {R}^{-} R + {S}^{u} + {R}^{-} {R}^{+} {R}^{-} {R}^{+} {S}^{u} … = [ \text{} 1 + {R}^{-} {R}^{+} + {R}^{-} {R}^{+} {R}^{-} {R}^{+} + {R}^{-} {R}^{+} {R}^{-} {R}^{+} {R}^{-} {R}^{+} + … ] {S}^{u} = {[ 1 - {R}^{-} {R}^{+} ]}^{- 1} {S}^{u}$$

for the P-SV matrix case. Note that some care must be exercised in the convergence of the matrix series, however, for practical situations we can omit this detail.

Step c) The process of forming the total field at the response point must be broken up into two cases:
Case 1: the response point is above the scatter point, that is Δz−Δz′<0 (Δz is the distance from the interface above the scattering layer, to the response point, the z axis is positive downward), and
Case 2, where the response point is below the scattering point Δz−Δz′<0.

Furthermore each case consists of an upward travelling, and a travelling wave:

### Upward Travelling Wavefield:

First, in case 1:

[1−R⁻R⁺]⁻¹ S^{u} and [1−R⁻R⁺]⁻¹ S^{u} represent the contribution to u^{up} from the upward travelling part of the source. A similar expression can be formed for the contribution from the downward travelling part of the source, it is

$$[ 1 - {R}^{-} {R}^{+ 1 - 1} {R}^{-} {S}^{d} , {and [ 1 - {R}^{-} {R}^{+} ]}^{- 1} {R}^{-} {S}^{d}$$

Thus, the total upward component of the wave field at the response point is formed from:

$${u}^{up} ≡ ( \begin{matrix}{( I - {R}^{-} {R}^{+} )}^{- 1} ( {S}^{u} + {R}^{-} {S}^{d} ) \\ {( I - {r}^{-} {r}^{+} )}^{- 1} ( {S}^{u} + {r}^{-} {S}^{d} )\end{matrix} )$$

Case 2: the response point is below the scatter point, that is Δz−Δz′>0 The result here is similar, the change occurring in the coefficient of the S^{u}, and S^{u}.

$${u}^{up} = ( \begin{matrix}{I - {R}^{-} {R}^{+} )}^{- 1} ( {R}^{-} {R}^{+} {S}^{u} + {R}^{-} {S}^{d} ) \\ {( I - {r}^{-} {r}^{+} )}^{- 1} ( {S}^{d} + {r}^{-} {S}^{d} )\end{matrix} )$$

### Downward Component of Wavefield

Case 1: A similar expression gives the downward component of the total wave field at the response point r, for case 1:

$${u}^{down} ≡ {u}^{d} ≡ ( \begin{matrix}{( I - {R}^{+} {R}^{-} )}^{- 1} {R}^{+} {R}^{-} {S}^{d} \\ {( I - {r}^{+} {r}^{-} )}^{- 1} {r}^{+} {r}^{-} {S}^{d}\end{matrix} ) + ( \begin{matrix}{( I - {R}^{+} {R}^{-} )}^{- 1} {R}^{+} {S}^{u} \\ {( I - {r}^{+} {r}^{-} )}^{- 1} {r}^{+} {S}^{u}\end{matrix} )$$

$$or$$

$${u}^{down} ≡ {u}^{d} ≡ ( \begin{matrix}{( I - {R}^{+} {R}^{-} )}^{- 1} ( {R}^{+} {R}^{-} {S}^{d} + {R}^{+} {S}^{u} ) \\ {( I - {r}^{+} {r}^{-} )}^{- 1} ( {r}^{+} {r}^{-} {S}^{d} + {r}^{+} {S}^{u} )\end{matrix} )$$

Case 2: For case 2, the result is similar, here the response point resides below the scatter point, that is Δz−Δz′>0

$${u}^{down} ≡ {u}^{a} ≡ ( \begin{matrix}{( I - {R}^{+} {R}^{-} )}^{- 1} ( {S}^{d} + {R}^{+} {S}^{u} ) \\ {( I - {r}^{+} {r}^{-} )}^{- 1} ( {r}^{+} {r}^{-} {S}^{d} + {r}^{+} {S}^{u} )\end{matrix} )$$

Step d) The final step in the process is the recombination of the plane waves to obtain the total wavefield (upgoing and downgoing waves) at the response position:

For the scalar case 1 Δz−Δz′<0, and:

$${G}^{L} ( x - {x}^{′} , y - {y}^{′} , z \vert {z}^{′} ) = {f}^{u} + {f}^{d} ,$$

with

$${u}^{up} = \int {{J}_{0} ( u ω / r - {r}^{′} \vert ) [ 1 - {R}^{-} {R}^{+} ]}^{- 1} [ {S}^{u} + {R}^{-} {S}^{d} ] {exp}^{- i ω bsc ( z - z s c )} du$$

$${u}^{d} = \int {{J}_{0} ( u ω / r - {r}^{′} \vert ) [ 1 - {R}^{-} {R}^{+} ]}^{- 1} [ {R}^{+} {S}^{u} + {R}^{+} {R}^{-} {S}^{d} ] {exp}^{- i ω bsd ( z - z s c )} du$$

so that

$${G}^{L} ( x - {x}^{′} , y - {y}^{′} , z / {z}^{′} ) = \int {{J}_{0} ( u ω / r - {r}^{′} \vert ) [ 1 - {R}^{-} {R}^{+} ]}^{- 1} [ {S}^{u} + {R}^{-} {S}^{d} ] [ {e}^{- i ω {b}_{s c} ( z - {z}_{s c} )} + {R}^{+} {e}^{- i ω {b}_{s c} ( z - {z}_{s c} )} ] du ,$$

While for case 2, Δz−Δz′>0, similar algebra gives:

$${G}^{L} ( x - {x}^{′} , y - {y}^{′} , z / {z}^{′} ) = \int {{J}_{0} ( u ω / r - {r}^{′} \vert ) [ 1 - {R}^{-} {R}^{+} ]}^{- 1} [ {R}^{+} {S}^{u} + {S}^{d} ] [ {e}^{- i ω {b}_{s c} ( z - {z}_{s c} )} + {R}^{-} {e}^{- i ω {b}_{s c} ( z - {z}_{s c} )} ] d u .$$

For case 1 this can be rewritten as:

$${G}^{L} ( r - {r}^{′} , z \vert {z}^{′} ) = A {\int }_{u = 0}^{∞} C ( u : r - {r}^{′} ) \{ {S}^{u} {e}^{i ω {b}_{s c} Δ z} + {R}^{-} {S}^{d} {e}^{i ω {b}_{s c} Δ z} + {R}^{+} {S}^{u} {e}^{- i ω {b}_{s c} Δ z} + {R}^{+} {R}^{-} {S}^{d} {e}^{- i ω {b}_{s c} Δ z} \} d u$$

where the coefficient function C(u: r−r′) is given by:

$$C ( u : r - {r}^{′} ) = {J}_{0} ( u ω \vert r - r ' \vert ) {( 1 - {R}^{-} {R}^{+} )}^{- 1} \frac{i ω u}{{b}_{s c}}$$

For case 2, G^{L} can be written as:

$${G}^{L} ( r - {r}^{′} , z \vert {z}^{′} ) = A {\int }_{u = 0}^{∞} C ( u : r - {r}^{′} ) \{ {R}^{+} {R}^{-} {S}^{u} {e}^{i ω {b}_{s c} Δ z} + {R}^{-} {S}^{d} {e}^{i ω {b}_{s c} Δ z} + {R}^{+} {S}^{u} {e}^{- i ω {b}_{s c} Δ z} + {S}^{d} {e}^{- i ω {b}_{s c} Δ z} \} du$$

Finally, using the definitions for S^{u} and S^{d} and recognizing that products such as

$${S}^{{}_{d}} {e}^{{}_{i ω {b}_{s c} Δ z}}$$

can be rewritten as

$${S}^{{}_{d}} {e}^{{}_{i ω {b}_{s c} Δ z}} = {e}^{i ω {b}_{s c} Δ {z}^{′}} {e}^{i ω {b}_{s c} Δ z} = {e}^{i ω {b}_{s c} ( Δ z + Δ {z}^{′} )}$$

the operator G^{L} turns out (after rearrangement of the above equations) to be the sum of a convolution and a correlation kernel:

$${G}^{L} ( r - {r}^{′} , z \vert {z}^{′} ) = {G}_{R} ( r - {r}^{′} , z + {z}^{′} ) + {G}_{v} ( r - {r}^{′} , z - {z}^{′} )$$

Case I has (Δz−Δz′)<0, where Δz=z−Z_{sc} and Δz′=z′−z_{sc} with z_{sc} being the z co-ordinate of the top part of the layer that contains the scatterer. In fact G_{R} and G_{V} turn out to be given by the following formulae (they appear to differ from the integrals above because of the rearrangement that leads to the decomposition into convolutional and correlational parts):

$${G}^{L} ( r - {r}^{′} , Δ z \vert Δ {z}^{′} ) = A {\int }_{u = 0}^{∞} {J}_{0} ( u ω \vert r - {r}^{′} \vert ) C ( u ) \{ {R}^{+} {e}^{- \dot{k} ω {b}_{s c} ( Δ z + Δ {z}^{′} )} + {R}^{-} {e}^{i ω {b}_{s c} ( Δ z + Δ {z}^{′} )} \} du \\ {G}_{V} ( r - {r}^{′} , Δ z \vert Δ {z}^{′} ) = A {\int }_{u = 0}^{∞} {J}_{0} ( u ω \vert r - {r}^{′} \vert ) C ( u ) \{ {R}^{+} {R}^{-} {e}^{- i ω {b}_{s c} ( Δ z + Δ {z}^{′} )} + {e}^{i ω {b}_{s c} ( Δ z + Δ {z}^{′} )} \} d u ,$$

where, now:

$$C ( u ) = {( 1 - {R}^{+} {R}^{-} )}^{- 1} \frac{i ω u}{{b}_{s c}} ,$$

R⁻ and R⁺ are the recursively defined reflectivity coefficients described in Muller's paper,
u is the horizontal slowness,

$$u = \frac{sin θ}{c}$$

Recall that Snell's law guarantees that u will remain constant as a given incident plane wave passes through several layers.
ω is the frequency
b_{sc} is the vertical slowness for the particular layer hosting the scattering potential

Case II consists of the case where (Δz−Δz′)≥0. The G_{R} and G_{V} are now given by the following:

$${G}_{V} ( r - {r}^{′} , Δ z \vert Δ {z}^{′} ) = {\int }_{u = 0}^{∞} {J}_{0} ( u ω \vert r - {r}^{′} \vert ) C ( u ) \{ {R}^{+} {R}^{-} {e}^{i ω {b}_{s c} ( Δ z + Δ {z}^{′} )} + {e}^{- i ω {b}_{s c} ( Δ z + Δ {z}^{′} )} \} du \\ and \\ {G}_{R} ( r - {r}^{′} , Δ z \vert Δ {z}^{′} ) = A {\int }_{u = 0}^{∞} {J}_{0} ( u ω \vert r - {r}^{′} \vert ) C ( u ) \{ {R}^{-} {e}^{i ω {b}_{s c} ( Δ z + Δ {z}^{′} )} + {R}^{+} {e}^{- i ω {b}_{s c} ( Δz + Δ {z}^{′} )} \} d u ,$$

These expressions can be combined into one equation by the judicious use of absolute values. The correlational part of the Green's operator can be transformed into a convolution by a suitable change of variables. The resulting Green's function for imaging inhomogeneities residing within Ambient Layered Media must be quantized by convolving with the sine (really Jinc) basis functions described below. This is done analytically and the result is given below.

The same process is carried out as detailed above, in order to determine the P-SV total wavefield (the 2 by 2 matrix case), and is not repeated.

The matrix case is handled in exactly the same manner, with allowance for the differing algebra associated with matrices, the preservation of the convolution is preserved for the same reason as shown above in the scalar situation.

On avoiding convergence problems due to the presence of derivatives in the elastic Green's function (i.e. with shear wave motion)

One difficulty with this formulation is the presence of four derivatives acting upon the acoustic free space Green's function in the construction of the dyadic G^{(LAY)}. This problem we have overcome (see related code in Appendix E) by the following method:

Given an inhomogeneous distribution of isotropic density ρ and Lame parameters λ and μ, imbedded in a homogeneous medium with density ρ₀ and Lame parameters λ₀ and μ₀, the total infinitesimal displacement vector u satisfies the partial differential equation

$${ω}^{2} ρ {u}_{i} + {( λ {u}_{j , j} )}_{, j} + {[ μ ( {u}_{i , j} + {u}_{j , i} ) ]}_{, j} = 0 \qquad ( 1 )$$

while the incident displacement field u satisfies

$${ω}^{2} ρ {u}_{i}^{i} + {( {λ}_{0} {u}_{j , j}^{j} )}_{, i} + {[ {u}_{0} ( {u}_{i , j}^{i} + {u}_{j , i}^{i} ) ]}_{, j} = 0 \qquad ( 2 )$$

where ρ₀, λ₀, and μ₀ are the homogeneous parameters of the imbedding medium. Subtracting (2) from (1) and rearranging results in

$${u}_{i}^{s} + ( \frac{1}{{k}_{p}^{2}} - \frac{2}{{k}_{s}^{2}} ) {( {u}_{j , j}^{s} )}_{, i} + {\frac{1}{{k}_{s}^{2}} [ ( {u}_{i , j}^{s} + {u}_{j , i}^{s} ) ]}_{, j} = - {f}_{i} \qquad ( 3 )$$

for the scattered displacement field u^{s}=u−uⁱ. The inhomogeneous term f is given by

$${f}_{i} = ( \frac{ρ}{{ρ}_{0}} - 1 ) {u}_{i} - ( \frac{1}{{k}_{p}^{2}} - \frac{2}{{k}_{s}^{2}} ) {\{ ( \frac{λ}{{λ}_{0}} - 1 ) {u}_{j , j} \}}_{, i} + \frac{1}{{k}_{s}^{2}} {\{ ( \frac{μ}{{μ}_{0}} - 1 ) ( {u}_{i , j} + {u}_{j , j} ) \}}_{, j} \qquad ( 4 )$$

where the respective shear-wave and compression-wave velocities cₛ and cₚ and corresponding wave numbers ks and kp are given by

$${c}_{s}^{2} = \frac{{μ}_{0}}{{ρ}_{0}} ; {k}_{s}^{2} = \frac{{ω}^{2}}{{c}_{s}^{2}} ; {c}_{p}^{2} = \frac{{λ}_{0} + 2 {μ}_{0}}{{ρ}_{0}} ; {k}_{p}^{2} = \frac{{ω}^{2}}{{c}_{p}^{2}} \qquad ( 5 )$$

for the imbedding medium. Introducing the scattering potentials

$${γ}_{ρ} = \frac{ρ}{{ρ}_{0}} - 1 ; {γ}_{λ} = ( \frac{1}{{k}_{p}^{2}} - \frac{2}{{k}_{s}^{2}} ) ( \frac{λ}{{λ}_{0}} - 1 ) ; γ μ = \frac{1}{{k}_{s}^{2}} ( \frac{μ}{{μ}_{0}} - 1 ) \qquad ( 6 )$$

gives

$${f}_{i} = {γ}_{ρ} {u}_{i} + {\{ {γ}_{λ} {u}_{j , j} \}}_{, i} + {\{ {γ}_{μ} ( {u}_{i , j} + {u}_{j , i} ) \}}_{, j} \qquad ( 7 )$$

Solution of (3) by the introduction of the free-space Green's function results in

$${u}_{i}^{s} = {G}_{ij} * {f}_{j} = {\int }_{v} {f}_{j} ( {r}^{′} ) {G}_{ij} ( F - {r}^{′} ) {dv}^{′} \qquad ( 8 )$$

where * denotes 3-D convolution, v is the support of the inhomogeneity, and the Green's function is given by [Aki and Richards, Quantitative Seismology, Freeman and Co., 1980, herein included as a reference]:

$${G}_{ij} ( r - {r}^{′} ) = {k}_{s}^{2} g ( {k}_{s} \vert r - {r}^{′} \vert ) {δ}_{ij} + \frac{{\partial }^{2}}{\partial {x}_{i} \partial {x}_{j}} \{ g ( {k}_{s} \vert r - {r}^{′} \vert - g ( {k}_{p} \vert r - {r}^{′} \vert ) \} \qquad ( 9 )$$

where g(kR) is the scalar Helmholtz Green's function

$$g ( kR ) = \frac{{e}^{ikR}}{4 π R} . \qquad ( 10 )$$

where e^{iωt} time dependence has been assumed. Inserting (7) into (8) and integrating by parts yields the following integral wave equation:

$${u}_{i}^{s} = {G}_{ij}^{*} \{ {γ}_{p} {u}_{j} \} + {G}_{ij , j}^{*} \{ {γ}_{λ} {u}_{k , k} \} + {G}_{ij , k}^{*} \{ {γ}_{μ} ( {u}_{j , k} + {u}_{k , j} ) \} . \qquad ( 11 )$$

For now, consider the case where γ_{μ}=0 and note that:

$${G}_{ij , j} = {k}_{s}^{2} \frac{\partial }{\partial {x}_{i}} g ( {k}_{s} R ) + \frac{\partial }{\partial {x}_{i}} ( {∇}^{2} g ( {k}_{s} R ) - {∇}^{2} g ( {k}_{p} R ) ) . \qquad ( 12 )$$

using

$$( {∇}^{2} + {k}^{2} ) g ( kR ) = - δ ( \vert r - {r}^{′} \vert ) \qquad ( 13 )$$

reduced (12) to:

$${G}_{ij , j} = {k}_{p}^{2} \frac{\partial }{\partial {x}_{i}} g ( {k}_{p} R ) \qquad ( 14 )$$

which will henceforth be denoted Cᵢ. We have now arrived at the integral equation:

$$[ \begin{matrix}{u}_{x}^{i} \\ {u}_{y}^{i} \\ {u}_{z}^{i}\end{matrix} ] = [ \begin{matrix}{u}_{x} \\ {u}_{y} \\ {u}_{z}\end{matrix} ] - [ \begin{matrix}{G}_{xx} & {G}_{x y} & {G}_{x z} & {C}_{x} \\ {G}_{y x} & {G}_{y y} & {G}_{y z} & {C}_{y} \\ {G}_{z x} & {G}_{z y} & {G}_{zz} & {C}_{z}\end{matrix} ] * [ \begin{matrix}{γ}_{ρ} & 0 & 0 & 0 \\ 0 & {γ}_{ρ} & 0 & 0 \\ 0 & 0 & {γ}_{ρ} & 0 \\ 0 & 0 & 0 & {γ}_{λ}\end{matrix} ] [ \begin{matrix}{u}_{x} \\ {u}_{y} \\ {u}_{z} \\ ∇ \cdot u\end{matrix} ] \qquad ( 15 )$$

since u_{k,k}=∇·u

The integral equation (15) can be solved by application of the 3-D FFT to compute the indicated convolutions, coupled with the biconjugate gradient iteration, or similar conjugate gradient method. [Jacobs, 1981]. One problem with (15) is, however, the need to compute ∇·u at each iteration. Various options for this include taking finite differences or the differentiation of the basis functions (sinc functions) by FFT. Our experience with the acoustic integral equation in the presence of density inhomogeneity indicates that it is best to avoid numerical differentiation. Instead, the system is augmented as:

$$[ \begin{matrix}{u}_{x}^{i} \\ {u}_{y}^{i} \\ {u}_{z}^{i} \\ ∇ \cdot {u}^{i}\end{matrix} ] = [ \begin{matrix}{u}_{x} \\ {u}_{y} \\ {u}_{z} \\ ∇ \cdot u\end{matrix} ] - [ \begin{matrix}{G}_{xx} & {G}_{xy} & {G}_{xz} & {C}_{x} \\ {G}_{yx} & {G}_{yy} & {G}_{yz} & {C}_{y} \\ {G}_{zx} & {G}_{zy} & {G}_{zz} & {C}_{z} \\ {C}_{x} & {C}_{y} & {C}_{z} & D\end{matrix} ] * [ \begin{matrix}{γ}_{ρ} & 0 & 0 & 0 \\ 0 & {γ}_{ρ} & 0 & 0 \\ 0 & 0 & {γ}_{ρ} & 0 \\ 0 & 0 & 0 & {γ}_{λ}\end{matrix} ] [ \begin{matrix}{u}_{x} \\ {u}_{y} \\ {u}_{z} \\ ∇ \cdot u\end{matrix} ] \qquad ( 16 )$$

$$where$$

$$D = {C}_{x , x} + {C}_{y , y} + {C}_{z , z} = {k}_{ρ}^{2} {∇}^{2} g ( {k}_{ρ} R ) = - {k}_{ρ}^{2} \{ {k}_{ρ}^{2} g ( {k}_{ρ} R ) + δ ( R ) \} . \qquad ( 17 )$$

Iterative solution of (16) for the four unknown fields uₓ, u_{y}, u_{z}, and ∇·u now involves no numerical differentiation of u, since all derivative operators have now been applied analytically to the Green's function. The incident component ∇·uⁱ is assumed known. Equation (16) can be written symbolically as

$${U}^{i} = ( I - {G [ γ ]}_{diag} ) U \qquad ( 18 )$$

where Uⁱ and U are the augmented 4-vectors in Eq. (16), [γ]_{diag} is the diagonal operator composed of γ_{ρ} and γ_{λ}, and is the 4×4 dyadic Green's function in Eq. (16).

Inclusion of γ_{μ} Scattering to Give the General Elastic-wave Integral Equations.

We now give a method for solving integral equations in the general case for the inhomogeneous material properties ρ, λ, and μ.

Because the inclusion of the more complicated term in γ_{μ} causes the matrix notation used above to require breaking the equations into parts on several lines, we elect for efficiency of space to use the more compact tensor notation. This should cause no difficulty because the translation is clear on comparing the special case of γ_{μ}=0 in the general tensor equations which follow with the previous matrix notation for this same case.

First we give again (see Eq (11)) the integral equations (which in practice have been discretized by sine basis functions) for inhomogeneous ρ, λ, and μ. Here * means convolution (the integration operation):

$${u}_{i}^{s} = {G}_{ij}^{*} [ {γ}_{p} {u}_{j} ] + {G}_{ij , j}^{*} [ {γ}_{λ} {u}_{k , k} ] + {G}_{ij , k}^{*} [ {γ}_{μ} ( {u}_{j , k} + {u}_{k , j} ) ] \qquad ( 19 )$$

We note that the displacement field uⱼ and its derivatives also u_{j,k} appear. We choose not to compute derivatives of the fields numerically and avoid the associated numerical instability. Instead, we augment the above equation with additional equations to compute the derivatives in a more stable way by solving for them directly. Thus, solving for (u_{i,l}+u_{l,i}) directly is more efficient and replaces computation of nine fields with only six. Forming the six unique symmetric-derivative pairs, we obtain an integral equation for these pairs.

$${u}_{i , 1}^{s} + {u}_{1 , i}^{s} = ( {G}_{ij , 1} + {G}_{1 j , i} ) * [ {γ}_{p} {u}_{j} ] + ( {G}_{ij , k 1} + {G}_{1 j , ki} ) * [ {γ}_{μ} ( {u}_{jk} + {u}_{kj} ) ] + ( {G}_{ijj 1} + {G}_{1 jji} ) * [ {γ}_{λ} {u}_{k , k} ] \qquad ( 20 )$$

Note that

$${G}_{ij , j} = {k}_{p}^{2} \frac{{\partial }^{2}}{\partial {x}_{i} \partial {x}_{j}} g ( {k}_{p} R ) \qquad ( 21 )$$

We now augment the system Eq. (19) for the three components of uᵢ^{s} with the system Eq. (20) which has six pairs of unique component derivatives.

These nine equations can also be placed in a matrix form similar to that of the augmented system given in Eq (16). The augmented system of nine components could be solved for γ_{ρ}, γ_{λ}, and γ_{μ}, assuming knowledge of the fields uⁱ and derivatives

$${u}_{i , k}^{i} .$$

Since the fields and derivatives are also unknown, we must solve for them simultaneously with γ_{ρ}, γ_{λ}, and γ_{μ}. This is done by adding these constraint equations. These nine equations are composed of the three equations for uᵢ

$${u}_{i}^{i} = {u}_{i} - {G}_{ij}^{*} ( {γ}_{p} {u}_{j} ) - {G}_{ij , j} * [ {γ}_{λ} {u}_{k , k} ] - {G}_{ij , k} * [ {γ}_{μ} ( {u}_{j , k} + {u}_{k , j} ) ] \qquad ( 22 )$$

and the six equations for U_{j,k}+u_{k,j}

$$( {u}_{i , l}^{i} + {u}_{l , i}^{j} ) = ( {u}_{i , l} - {u}_{l , i} ) - ( {G}_{ij , l} + {G}_{lj , j} ) * [ {γ}_{p} {u}_{j} ] - ( {G}_{ij , kl} + {G}_{lj , kl} ) * [ {γ}_{μ} ( {u}_{jk} + {u}_{kj} ) ] - ( {G}_{ij , ji} + {G}_{ij , ji} ) * [ {γ}_{λ} {u}_{k , k} ] \qquad ( 23 )$$

These 9 equations can also be placed in a matrix form similar to that of the augmented system given in Eq (16).

Solving these twelve equations (Eq. (19, 22, 23)) for (γ_{ρ}, γ_{λ}, γ_{μ}), the fields, and their derivatives is accomplished using the Frechet derivative methods described next. As in Eq. (16) for the μ=μ₀ case, Eq. (23) can be written symbolically as

$${U}^{i} = ( I - {G [ γ ]}_{diag} ) U \qquad ( 24 )$$

where the operators [symbol] and [γ]_{diag} are now 9×9, and the vectors U and Uⁱ consist of the three components of u and its six symmetric derivative sum-pairs.

### Example 7

### Cylindrical Coordinate Methods for Numerical Solution of the Sie

The above examples all use the FFT-BCG algorithm for imaging. This method is one of three methods discussed in this patent. The other two methods are
(1) The cylindrical coordinate recursion method (Cylindrical Recursion)
(2) Rectangular recursion of scattering matrix method (Rectangular Recursion for short reference)

We now discuss the cylindrical recursion method:

### 7.1 Cylindrical Coordinate Sie Formulation and Discretization

We begin with the acoustic scattering integral equation (SIE) for the constant density case:

$${f}^{i} ( ρ ) = f ( ρ ) - \frac{{k}_{0}^{2}}{4 i} \int \int γ ( {ρ}^{′} ) f ( {ρ}^{′} ) {H}_{0}^{( 2 )} ( {k}_{0} \vert ρ - {ρ}^{′} \vert ) {d}^{2} ρ \qquad ( 1.1 )$$

Let each field be expanded in a Fourier series in the angular variable:

$$f ( ρ ) = {\sum }_{n} {f}_{n} ( ρ ) {e}^{in ϕ} .$$

Then using Graf's addition theorem:

$${H}_{0}^{( 2 )} ( {k}_{0} \vert ρ - {ρ}^{′} \vert ) = \{ \begin{matrix}{\sum }_{n} {H}_{n}^{( 2 )} ( {k}_{0} ρ ) {J}_{n} ( {k}_{0} {ρ}^{′} ) {e}^{in ( ϕ - {ϕ}^{′} )} & ρ > {ρ}^{′} \\ {\sum }_{n} {H}_{n}^{( 2 )} ( {k}_{0} {ρ}^{′} ) {J}_{n} ( {k}_{0} ρ ) {e}^{in ( ϕ - {ϕ}^{′} )} & ρ < {ρ}^{′}\end{matrix} \qquad ( 1.2 )$$

results in the cylindrical coordinate form of (1.1):

$${f}_{n}^{i} ( ρ ) = {f}_{n} ( ρ ) - \frac{π {k}_{0}^{2}}{2 i} {\int }_{0}^{a} \{ {\sum }_{m} {γ}_{n - m} ( {ρ}^{′} ) {f}_{m} ( {ρ}^{′} ) {B}_{m} ( {k}_{0} ρ ) {C}_{m} ( {k}_{0} {ρ}^{′} ) {ρ}^{′} d {ρ}^{′} \qquad ( 1.3 )$$

where the erne is separably symmetric:

$${B}_{m} ( {k}_{0} ρ ) {C}_{m} ( {k}_{0} {ρ}^{′} ) = \{ \begin{matrix}{H}_{n}^{( 2 )} ( {k}_{0} ρ ) {J}_{n} ( {k}_{0} {ρ}^{′} ) & ρ > {ρ}^{′} \\ {H}_{n}^{( 2 )} ( {k}_{0} {ρ}^{′} ) {J}_{n} ( {k}_{0} ρ ) & ρ < {ρ}^{′}\end{matrix} \qquad ( 1.4 )$$

Henceforth assume that k₀=1. Discretizing the radial integral by trapezoidal rule using an increment of A results in:

$${f}_{l , n}^{i} = {f}_{nl , n} - \{ {h}_{l , n} {\sum }_{{l}^{′} = 1}^{i} {( γ f )}_{{l}^{′} , η} {j}_{{l}^{′} , n} + {j}_{{l}^{′} , n} {\sum }_{{l}^{′} = l + 1}^{L} {( γ f )}_{{l}^{′} , n} {h}_{{l}^{′} , n} \} \qquad ( 1.5 )$$

$$where$$

$${f}_{l , n} = {f}_{n} ( l Δ ) , {h}_{l , n} = Δ \sqrt{\frac{π}{2 i}} {H}_{n}^{( 2 )} ( l Δ ) , {j}_{l , n} = Δ \sqrt{\frac{π}{2 i}} {J}_{n} ( l Δ ) \qquad ( 1.6 )$$

$$and$$

$${( γ f )}_{{l}^{′} , n} = {l}^{′} {\sum }_{m} {γ}_{{l}^{′} , n - m} {f}_{{l}^{′} , m} \qquad ( 1.7 )$$

Notice that the extra l′ resulting from the ρ′ in the integral in (1.3) has been placed in the definition of (γf)l_{l,n}. Equation (1.5) is the final discrete linear system for the solution of the scattering integral equation in cylindrical coordinates. It can be rewritten as:

$${f}_{l}^{i} = {f}_{l} - \{ [ {h}_{l} ] {\sum }_{{l}^{′} = 1}^{l} [ {j}_{{l}^{′}} ] [ {γ}_{{l}^{′}}^{*} ] {f}_{{l}^{′}} + [ {j}_{l} ] {\sum }_{{l}^{′} = l + 1}^{L} [ {h}_{{l}^{′}} ] [ {γ}_{{l}^{′}}^{*} ] {f}_{{l}^{′}} \} \qquad ( 1.8 )$$

where the vector components are the Fourier coefficients:

$${f}_{l} = [ \begin{matrix}{f}_{l , - N} \\ ⋮ \\ {f}_{l , N}\end{matrix} ] ,$$

or, equivalently,

$${\{ {f}_{l} \}}_{n} = {f}_{l , n} , n = - N , … , N \qquad ( 1.9 )$$

where N is the range of the truncated Fourier series (The vectors are length 2N+1). The notation [x] denotes a diagonal matrix formed from the vector elements:

$$[ x ] = [ \begin{matrix}{x}_{- N} & 0 & ⋯ & 0 \\ 0 & ⋱ & & \\ ⋮ & & ⋱ & \\ 0 & & & {x}_{N}\end{matrix} ] \qquad ( 1.1 )$$

and the notation [γ_{l′}*] denotes a convolution (including the l′ factor):

$${\{ [ {γ}_{{l}^{′}}^{*} ] {f}_{{l}^{′}} \}}_{n} = {l}^{′} {\sum }_{m = - N}^{N} {γ}_{{l}^{′} , n - m} {f}_{{l}^{′} , m} , n = - N , … , N . \qquad ( 1.11 )$$

Writing (1.8) out in full gives the matrix equation:

$$[ \begin{matrix}{f}_{1}^{i} \\ ⋮ \\ {f}_{L}^{i}\end{matrix} ] = A [ \begin{matrix}{f}_{1} \\ ⋮ \\ {f}_{L}\end{matrix} ] \qquad ( 1.12 )$$

$$where$$

$$A = [ \begin{matrix}I & 0 & ⋯ & ⋯ & 0 \\ 0 & ⋱ & & & ⋮ \\ ⋮ & & ⋱ & & ⋮ \\ ⋮ & & & ⋱ & 0 \\ 0 & ⋯ & ⋯ & 0 & I\end{matrix} ] - [ \begin{matrix}[ {j}_{1} ] [ {h}_{1} ] & [ {j}_{1} ] [ {h}_{2} ] & ⋯ & [ {j}_{1} ] [ {h}_{L} ] \\ [ {h}_{2} ] [ {j}_{1} ] & & & ⋮ \\ ⋮ & & & ⋮ \\ [ {h}_{L} ] [ {j}_{1} ] & [ {h}_{L} ] [ {j}_{2} ] & ⋯ & [ {j}_{L} ] [ {h}_{L} ]\end{matrix} ] \cdot [ \begin{matrix}[ {γ}_{1} ] & 0 & ⋯ & 0 \\ 0 & & & ⋮ \\ ⋮ & & & 0 \\ 0 & ⋯ & 0 & [ {γ}_{L} ]\end{matrix} ] \qquad ( 1.13 )$$

Notice that the kernel is composed of a L×L block matrix with 2N+1×2N+1 diagonal matrix components and that the L×L block matrix is symmetric-separable, i.e.:

$${L}_{mn} = \{ \begin{matrix}[ {J}_{n} ] [ {H}_{m} ] , & n \leq m \\ [ {h}_{n} ] [ {j}_{m} ] , & n > m\end{matrix} \qquad ( 1.14 )$$

where Lₙₘ is one of the L×L component matrices.

For reference in the next section, we make the definitions:

$${s}_{l} = {\sum }_{{l}^{′} = l}^{L} [ {h}_{{l}^{′}} ] [ {γ}_{{l}^{′}}^{*} ] {f}_{{l}^{′}} \qquad ( 1.15 )$$

$${p}_{l} ≡ {\sum }_{{l}^{′} = 1}^{l - 1} [ {h}_{{l}^{′}} ] [ {γ}_{{l}^{′}}^{*} ] {f}_{{l}^{′}} \qquad ( 1.16 )$$

### 7.2 Recursive Solution of the Cylindrical Coordinate Sie

The recursion for the 2-D cylindrical equation is:

### Initialize:

$${P}_{L} = 0 \qquad ( 2.1 )$$

$${\{ {s}_{L} \}}_{n} = {f}_{L , n}^{s} / {h}_{L , n}$$

$$For 1 = L , … , 1$$

$${S}_{l - 1} = {S}_{l} [ {j}_{l} ] [ {γ}_{l} * ] \{ [ {h}_{l} ] {s}_{l} + [ {j}_{l} ] {p}_{l} \}$$

$${p}_{l - 1} = {p}_{l} + [ {h}_{l} ] [ {γ}_{l}^{*} ] \{ [ {h}_{l} ] {s}_{l} + [ {j}_{l} ] {p}_{l} \}$$

Next 1.

Thus far, we have assumed that the number of angular coefficients is the same for each radius l. In fact, the number of angular coefficients should decrease as l decreases. We find that for Δ=λ/4, an accurate interpolation is achieved with N₁=21−1, n=−Nₗ, . . . , Nₗ Fourier coefficients. To introduce this modification, we must slightly redefine the operators as:

$${\{ [ {j}_{i} ] x \}}_{n} = \{ \begin{matrix}{j}_{l , {n}^{X} n} , & \vert n \vert \leq {N}_{l} \\ 0 , & \vert n \vert > {N}_{l}\end{matrix} \qquad ( 2.2 )$$

$${\{ [ {γ}_{l}^{*} ] x \}}_{n} = \{ \begin{matrix}l {\sum }_{m = - {N}_{l}}^{{N}_{l}} {γ}_{i , n - m} {x}_{m} , & \vert n \vert \leq {N}_{l} \\ 0 , & \vert n \vert > {N}_{l}\end{matrix} \qquad ( 2.3 )$$

where the vector operated on is always length N where N is now the maximum number of Four er coefficients, N=N_{L}. The total field at each layer, l, is given by:

$${f}_{l , n} = \{ \begin{matrix}{h}_{l , n} {s}_{l , n} + {j}_{l , n} {p}_{l , n} + {f}_{l , n}^{i} , & \vert n \vert \leq {N}_{l} \\ 0 , & \vert n \vert > {N}_{l}\end{matrix} \qquad ( 2.4 )$$

In order to eliminate the need to know the starting values, SL, we note that at each iteration, sₗ and pₗ are linear functions of SL:

$${s}_{L} = {A}_{l} {s}_{L} + {b}_{l} , {p}_{l} = {C}_{1} {s}_{L} + {d}_{l} \qquad ( 2.5 )$$

where the matrices Aₗ, Cₗ (dimension 2N+1×2N+1) and the vectors bₗ, dₗ (dimension 2N+1) have the initial values:

$${A}_{L} = l , {C}_{L} = 0 {b}_{l} = 0 , {d}_{l} = 0 \qquad ( 2.6 )$$

Using (2.5) and (2.6) in (1.1) and equating common terms leads a matrix and a vector recursion:

### Initialize:

$${A}_{L} = l , {C}_{L} = 0 {b}_{L} = 0 , {d}_{L} = 0 \qquad ( 2.7 )$$

For l=L, . . . , 1

$${A}_{l - 1} = {A}_{l} - [ {j}_{l} ] [ {γ}_{l}^{*} ] \{ [ {h}_{l} ] {A}_{l} + [ {j}_{l} ] {C}_{J} \}$$

$${b}_{l - 1} = {b}_{l} - [ {j}_{l} ] [ {γ}_{l}^{*} ] \{ [ {h}_{l} ] {b}_{l} + [ {j}_{l} ] {d}_{l} + {f}_{l}^{i} \}$$

$${C}_{l - 1} = {C}_{l} + [ {h}_{l} ] [ {γ}_{l}^{*} ] \{ [ {h}_{l} ] {A}_{l} + [ {j}_{l} ] {C}_{l} \}$$

$${d}_{l - 1} = {d}_{l} + [ {h}_{l} ] [ {γ}_{l}^{*} ] \{ [ {h}_{l} ] {b}_{l} + [ {j}_{l} ] {d}_{l} + {f}_{l}^{j} \}$$

Then using the fact that S_{L}=0 leads to the solution:

$${A}_{0} {s}_{L} + {b}_{0} = 0 , {s}_{L} = - {A}_{0}^{- 1} {b}_{0} , {f}_{L , n}^{s} = {s}_{L , n} / {h}_{L , n} \qquad ( 2.8 )$$

for the scattered field at the outer boundary. Iteration (1.1) with (1.4) can then be used to evaluate the total field internal to the object.

Notice that the LHS matrix recursion in (2.7) is independent of the incident field. Once it is used to compute A₀, the RHS vector recursion can be done for any number of incident fields. Concurrent solution for any number of incident fields can be obtained by replacing the RHS vector recursion with the matrix recursion:

$${B}_{L} = 0 , {D}_{L} = 0 \qquad ( 2.9 )$$

$${B}_{l - 1} = {B}_{l} - [ {j}_{l} ] [ {Y}_{l}^{*} ] \{ [ {h}_{l} ] {B}_{l} + [ {j}_{l} ] {D}_{l} + {F}_{l}^{i} \}$$

$${D}_{l - 1} = {D}_{l} + [ {h}_{l} ] [ {Y}_{l}^{*} ] \{ [ {h}_{l} ] {B}_{l} + [ {j}_{l} ] {D}_{l} + {F}_{l}^{i} \}$$

where the matrices Bₗ, Dₗ are 2N+1×Nᵥ where Nᵥ is the number of views and the matrix of incident fields, Fₗⁱ is given by:

$${F}_{l}^{\dot{r}} = [ \begin{matrix}{f}_{h , - N}^{{i}_{,} 1} & ⋯ & {f}_{h , - N}^{i , {N}_{v}} \\ ⋮ & & ⋮ \\ {f}_{l , N}^{i , 1} & ⋯ & {f}_{h , N}^{i , {N}_{v}}\end{matrix} ] \qquad ( 2.1 )$$

where the superscript after i is the view number (note that although the incident matrix is written as 2N+1×Nᵥ, the entries are zero for the row index Nₗ<|n|≤N). A more compact recursion can be obtained by concatenating the two recursions to give:

$${G}_{L} = [ {A}_{L} , {B}_{L} ] = [ I , 0 ] , {H}_{L} = [ {C}_{L} , {D}_{L} ] = [ 0 , 0 ] \qquad ( 2.11 )$$

For l=L, . . . , 1

$${G}_{l - 1} = {G}_{l} - [ {j}_{l} ] [ {Y}_{l}^{*} ] \{ [ {h}_{l} ] {A}_{l} + [ {j}_{l} ] {C}_{l} + [ 0 , {F}_{l}^{i} ] \}$$

$${H}_{l - 1} = {H}_{l} + [ {h}_{l} ] [ {γ}_{l}^{*} ] \{ [ {h}_{l} ] {A}_{l} + [ {j}_{l} ] {C}_{l} + [ 0 , {F}_{l}^{i} ] \}$$

where the matrices Gₗ, Hₗ are 2N+1×2N+1+Nᵥ and in the notation [A,B] the first matrix is 2N+1×2N+1 and the second is 2N+1×Nᵥ. Then Gₒ=[A₀, B₀] and the solution for all views is given by:

$${F}_{L}^{S} = [ \begin{matrix}{f}_{L , - N}^{s , 1} & ⋯ & {f}_{l , - N}^{s , {N}_{v}} \\ ⋮ & & ⋮ \\ {f}_{L , N}^{s , 1} & ⋯ & {f}_{L , N}^{s , {N}_{v}}\end{matrix} ] = [ {h}_{L} ] {A}_{0}^{- 1} {B}_{0} \qquad ( 2.12 )$$

A slightly modified recursion can be shown to yield the scattering matrix of the object. Recall that the total field is given by:

$${f}_{r} = [ {h}_{l} ] {s}_{l} + [ {j}_{l} ] {p}_{l} + {f}_{i}^{l} \qquad ( 2.13 )$$

Any externally generated incident field can be expanded in a bessel series which implies that there exists a sequence, g_{-N}ⁱ, . . . , g_{N}ⁱ such that:

$${f}^{i} ( ρ , ϕ ) = Δ \sqrt{\frac{π}{2 i}} {\sum }_{n} {g}_{j}^{i} {J}_{n} ( ρ ) {e}^{in ϕ} \qquad ( 2.14 )$$

(recall that we are assuming that k₀=1) which means that

$${f}_{l}^{i} = [ {j}_{l} ] {g}^{i} \qquad ( 2.15 )$$

Redefining pₗ→pₗ+gⁱ then gives fₗ=[hₗ]sₗ+[jₗ]pₗ and leads easily to the iteration:

$${G}_{L} = [ I , 0 ] , {H}_{L} = [ 0 , I ] \qquad ( 2.16 )$$

$$For 1 = L , … , 1$$

$${G}_{l - 1} = {G}_{l} - [ {j}_{l} ] [ {Y}_{l}^{*} ] \{ [ {h}_{l} {A}_{l} + [ {j}_{l} {C}_{l} \}$$

$${H}_{l - 1} = {H}_{l} + [ {h}_{l} ] [ {γ}_{l}^{*} ] \{ [ {h}_{l} ] {A}_{l} + [ {j}_{l} ] {C}_{l} \}$$

where the matrices are now 2N+1×2(2N+1). The last iterate G₀=[A₀, B₀] yields the scattering matrix:

$$S ( γ ) = {A}_{0}^{- 1} {B}_{0} \qquad ( 2.17 )$$

for the body y which relates the incident field coefficients to the scattering coefficients:

$${g}^{s} = S {g}^{i} , where {f}^{s} ( ρ , ϕ ) = Δ \sqrt{\frac{π}{2 i}} {\sum }_{n} {g}_{n}^{s} {H}_{0}^{( 2 )} ( ρ ) {e}^{in ϕ} \qquad ( 2.18 )$$

for all incident field coefficient vectors, g. Notice that in the previous notation:

$${g}_{n}^{i} = {f}_{L , n}^{i} {j}_{L , n} , {g}_{n}^{s} = {f}_{L , n}^{s} / {h}_{L , n} \qquad ( 2.19 )$$

### 7.3 Computational Formulas for the Jacobian and its Adjoint

In order to apply the Gauss-Newton iteration to the solution of the imaging problem we must first derive recursive formulas for the application of the Jacobian and its adjoint. The Jacobian of the scattering coefficient vector, SL, operating on a perturbation in γ is given by:

$$J δγ = {\sum }_{{l}^{′} = 1}^{L} {\sum }_{n = - {N}_{{l}^{′}}}^{{N}_{{l}^{′}}} , δ {γ}_{{l}^{′} , {n}^{′}} \frac{\partial }{\partial {γ}_{{l}^{′} , {n}^{′}}} {s}_{L} \qquad ( 3.1 )$$

From (2.8) we get:

$${s}_{L}^{′} = - {A}_{0}^{- 1} ( {b}_{0}^{′} + {A}_{0}^{′} {s}_{L} ) \qquad ( 3.2 )$$

where the prime denotes differentiation followed by summation over the elements of δγ.

$${s}_{L}^{′} = J δγ = {\sum }_{{l}^{′} = 1}^{L} {\sum }_{{n}^{′} = - {N}_{{l}^{′}}}^{{N}_{{l}^{′}}} {δγ}_{{l}^{′} , {n}^{′}} \frac{\partial }{\partial {γ}_{{l}^{′} , {n}^{′}}} {s}_{L} \qquad ( 3.3 a )$$

$${b}_{0}^{′} = {\sum }_{{l}^{′} = 1}^{L} {\sum }_{{n}^{′} = - {N}_{{l}^{′}}}^{{N}_{{l}^{′}}} {δγ}_{{l}^{′} , {n}^{′}} \frac{\partial }{\partial {γ}_{{l}^{′} , {n}^{′}}} {b}_{0} \qquad ( 3.3 b )$$

$${A}_{0}^{′} = {\sum }_{{l}^{′} = 1}^{L} {\sum }_{{n}^{′} = - {N}_{{l}^{′}}}^{{N}_{{l}^{′}}} {δγ}_{{l}^{′} , {n}^{′}} \frac{\partial }{\partial {γ}_{{l}^{′} , {n}^{′}}} {A}_{0} \qquad ( 3.3 c )$$

Equation (3.2) provides the formula for computing Jδγ is recursive formulas for

$${A}_{0}^{′}$$

and b′ can be found. Define the notation:

$${A}_{l}^{′} = {\sum }_{{l}^{′} = l + 1}^{L} {\sum }_{{n}^{′} = - {N}_{{l}^{′}}}^{{N}_{{l}^{′}}} {δγ}_{{l}^{′} , {n}^{′}} \frac{\partial }{\partial {γ}_{{l}^{′} , {n}^{′}}} {A}_{l} \qquad ( 3.4 a )$$

$${C}_{l}^{′} = {\sum }_{{l}^{′} = l + 1}^{L} {\sum }_{{n}^{′} = - {N}_{{l}^{′}}}^{{N}_{{l}^{′}}} {δγ}_{{l}^{′} , {n}^{′}} \frac{\partial }{\partial {γ}_{{l}^{′} , {n}^{′}}} {C}_{l} \qquad ( 3.5 b )$$

(note the lower limit of the l′ summation). Then using the LHS recursion in (2.7), it is simple to show that:

$${A}_{l - 1}^{′} = {A}_{l}^{′} [ {j}_{l} ] \{ [ {γ}_{l}^{*} ] \{ [ {h}_{l} {A}_{l}^{′} + [ {j}_{l} ] {C}_{l}^{′} \} + [ δ {γ}_{l}^{*} ] \{ [ {h}_{l} {A}_{l} + [ {j}_{l} ] {C}_{l} \} \} \qquad ( 3.5 a )$$

$${C}_{l - 1}^{′} = {C}_{l}^{′} + [ {h}_{l} ] \{ [ {γ}_{l}^{*} ] \{ [ {h}_{l} ] {A}_{l}^{′} + [ {j}_{l} ] {C}_{l}^{′} \} + [ δ {γ}_{l}^{*} ] \{ [ {h}_{l} ] {A}_{l} + [ {j}_{l} ] {C}_{l} \} \} \qquad ( 3.5 b )$$

A further reduction in computational requirements can be achieved by noting from (3.2) that we do not need

$${A}_{0}^{′}$$

but rather

$${A}_{0}^{′} {S}_{L}$$

where S_{L} is the matrix whose columns are the S_{L}'s for each view. The matrix A₀ is 2N+1×2N+1 while the matrix

$${A}_{0}^{′} {S}_{L}$$

is 2N+1×Nᵥ. Thus define:

$${A}_{l}^{′ s} = {A}^{′} {S}_{L} , {C}_{l}^{′ s} = {C}_{l}^{′} {S}_{L} , {A}_{l}^{s} = {A}_{l} {S}_{L} , {C}_{l}^{S} = {C}_{l} {S}_{L} \qquad ( 3.6 )$$

Then we get the recursion:

$${A}_{L}^{s} = {A}_{L} {S}_{L} = {IS}_{L} = {S}_{L} , {C}_{L}^{s} = 0 , {A}_{L}^{s} = 0 , {C}_{L}^{′ s} = 0 \qquad ( 3.7 a )$$

$$For 1 = L , … , 1$$

$${A}_{l - 1}^{′ s} = {A}_{l}^{′ s} - [ {j}_{l} ] \{ [ {γ}_{l}^{*} ] \{ [ {h}_{l} ] {A}_{l}^{′ s} + [ {j}_{l} ] {C}_{l}^{′ s} \} + [ δ {γ}_{l}^{*} ] \{ [ {h}_{l} ] {A}_{l}^{s} + [ {j}_{l} ] {C}_{l}^{s} \} \} \qquad ( 3.7 b )$$

$${C}_{l - 1}^{′ s} = {C}_{l}^{′ s} + [ {h}_{l} ] \{ [ {γ}_{l}^{*} ] \{ [ {h}_{l} {A}_{l}^{′ s} + {j}_{l} ] {C}_{l}^{′ s} \} + [ δ {γ}_{l}^{*} ] \{ [ {h}_{l} ] {A}_{l}^{s} + [ {j}_{l} ] {C}_{l}^{s} \} \} {(}_{{l}^{s}} \} \qquad ( 3.7 c )$$

$${A}_{l - 1}^{s} = {A}_{l}^{s} - [ {j}_{l} ] [ {γ}_{l}^{*} ] \{ [ {h}_{l} {A}_{l}^{s} + [ {j}_{l} ] {C}_{l}^{s} \} \qquad ( 3.7 d )$$

$${C}_{l - 1}^{s} = {C}_{l}^{s} + [ {h}_{l} ] [ {γ}_{l}^{*} ] \{ [ {h}_{l} ] {A}_{l}^{s} + [ {j}_{l} ] {C}_{l}^{s} \} \qquad ( 3.7 e )$$

Similarly, for the b's and d's we get the recursion:

$${B}_{L} = 0 , {D}_{L} = 0 , {B}_{L}^{′} = 0 , {D}_{L}^{′} = 0 \qquad J ( 3.8 a )$$

$$For 1 = L , … , 1$$

$${B}_{l - 1}^{′} = {B}_{l}^{′} - [ {j}_{l} ] \{ [ {γ}_{l}^{*} ] \{ [ {h}_{l} ] {B}_{l}^{′} + [ {j}_{l} ] {D}_{l}^{′} \} + [ δ {γ}_{l}^{*} ] \{ [ {h}_{l} ] {B}_{l} + [ {j}_{l} ] {D}_{l} + {F}_{l}^{i} \} \} \qquad ( 3.8 b )$$

$${D}_{l - 1}^{′} = {D}_{l}^{′} + [ {h}_{l} ] \{ [ {γ}_{l}^{*} ] \{ [ {h}_{l} ] {B}_{l}^{′} + [ {j}_{l} ] {D}_{l}^{′} \} [ {γ}_{l}^{*} ] \{ [ {h}_{l} ] {B}_{l} + [ {j}_{l} ] {D}_{l} + {F}_{l}^{i} \} \} \qquad ( 3.8 c )$$

$${B}_{l - 1} = {B}_{r} [ {j}_{l} ] [ {γ}_{l}^{*} ] \{ [ {h}_{l} ] {B}_{l} + [ {j}_{l} ] {D}_{l} + {F}_{l}^{i} \} \qquad ( 3.8 d )$$

$${D}_{l - 1} = {D}_{l} + [ {h}_{l} ] [ {γ}_{l}^{*} ] \{ [ {h}_{l} ] {B}_{l} + [ {j}_{l} ] {D}_{l} + {F}_{l}^{i} \} \qquad ( 3.8 e )$$

where the matrices are all 2N+1×Nᵥ. The final Jacobian is then:

$${S}_{L}^{′} = - {A}_{0}^{- 1} ( {B}_{0}^{′} + {A}_{0}^{′ s} ) \qquad ( 3.9 )$$

where the columns of S′_{L} are the vectors Jₙδγ for each view n=1, . . . , Nᵥ.

We can of course concatenate these two recursions to give:

$${G}_{L} = [ {S}_{L} , 0 ] , {H}_{L} = [ 0 , 0 ] , {G}_{L}^{′ s} = [ 0 , 0 ] , {H}_{L}^{′} = [ 0 , 0 ] \qquad ( 3.1 a )$$

$$For 1 = L , … , 1$$

$${G}_{l - 1}^{′} = {G}_{l}^{′} - [ {j}_{l} ] \{ [ {γ}_{l}^{*} ] \{ [ {h}_{l} ] {G}_{l}^{′} + [ {j}_{l} ] {H}_{l}^{′} \} + [ {δγ}_{l}^{*} ] \{ [ {h}_{l} ] {G}_{l} + [ {j}_{l} ] {H}_{l} + [ 0 , {F}_{l}^{i} ] \} \} \qquad ( 3.1 b )$$

$${H}_{l - 1}^{′} = {H}_{l}^{′} + [ {h}_{l} ] \{ [ {γ}_{l}^{*} ] \{ [ {h}_{l} ] {G}_{l}^{′} + [ {j}_{l} ] {H}_{l}^{′} \} + [ {δγ}_{l}^{*} ] \{ [ {h}_{l} ] {G}_{l} + [ {j}_{l} ] {H}_{l} + [ 0 , {F}_{l}^{i} \} \} \qquad ( 3.1 c )$$

$${G}_{l - 1} = {G}_{t} - [ {j}_{t} ] [ {Y}_{l}^{*} ] \{ [ {h}_{t} ] {G}_{l} + [ {j}_{l} ] {H}_{l} + [ 0 , {F}_{l}^{i} ] \} \qquad ( 3.1 d )$$

$${H}_{l - 1} = {H}_{l} + [ {h}_{l} ] [ {γ}_{l}^{*} ] \{ [ {h}_{l} ] {G}_{l} + [ {j}_{l} + {H}_{l} + [ 0 , {F}_{l}^{i} ] \} \qquad ( 3.1 e )$$

where, at the last iterate,

$${G}_{0} ′ = [ {A}_{0}^{′ S} , {B}_{0}^{′} ] .$$

The matrices (G's and H's) in this recursion are all 2N+1×2Nᵥ. This is the form of the Jacobian recursion used in the imaging programs.

### Example 8

### Rectangular Scattering Matrix Recursion

This section describes a new recursive algorithm the used scattering matrices for rectangular subregions. The idea for this approach is an extension and generalization of the cylindrical coordinate recursion method discussed in the previous section. The computational complexity is even further reduced over CCR. The CCR algorithm derives from the addition theorem for the Green's function expressed in cylindrical coordinates. The new approach generalizes this by using Green's theorem to construct propagation operators (a kind of addition theorem analogue) for arbitrarily shaped, closed regions. In the following, it is applied to the special case of rectangular subregions, although any disjoint set of subregions could be used.

FIG. 18 illustrates a rectangular scattering matrix according to the present invention. Consider two rectangular subregions A and B of R² as shown in FIG. 18. Although the regions are drawn as disjoint, assume that they touch at the center of the figure. Let C denote the external boundary of the union of A and B. Define the scattering operator, SA, of region A as the operator that gives the outward moving scattered field on the boundary A, given the inward moving field, due to external sources, evaluated on the boundary A. Similarly, define the scattering matrix for boundary B. The goal is to find the scattering matrix for boundary C, given S_{A} and S_{B}—the scattering matrices for A and B.

Let the incident field due to sources external to boundary C, evaluated on boundary A be denoted

$${f}_{A}^{i}$$

and similarly define

$${f}_{B}^{i} .$$

For the total problem (A and B both containing scatterers) there exists a net, inward moving field at boundary A. Denote this field

$${f}_{A}^{in}$$

and similarly define

$${f}_{B}^{in}$$

The total field leaving boundary A is then

$${f}_{A}^{out} = {S}_{A} {f}_{A}^{in} .$$

Knowledge of the radiated field on a closed boundary due to internal sources allows the field external to the boundary to be computed at any point. Let the operator that maps f_{A}^{ou} onto the boundary B be denoted T_{BA} (rectangular translation operator from A to B). Similarly denote the operator mapping

$${f}_{B}^{out}$$

to boundary A be denoted T_{AB}.

The total, inward moving field at boundary A has two parts—that due to the incident field external to C and that due to sources internal to boundary B. From the forgoing definitions, it should be obvious that the inward moving fields satisfy:

$${f}_{A}^{in} = {f}_{A}^{i} + {T}_{AB} {S}_{B} {f}_{B}^{in} \qquad ( 1 a )$$

$${f}_{B}^{in} = {f}_{B}^{i} + {T}_{BA} {S}_{A} {f}_{A}^{in} \qquad ( 1 b )$$

Solving for the total inward moving fields gives:

$$[ \begin{matrix}{f}_{A}^{in} \\ {f}_{B}^{in}\end{matrix} ] = {[ \begin{matrix}I & - {T}_{AB} {S}_{B} \\ - {T}_{BA} {S}_{A} & I\end{matrix} ]}^{- 1} [ \begin{matrix}{f}_{A}^{i} \\ {f}_{B}^{i}\end{matrix} ] \qquad ( 2 )$$

The total scattered field at boundary A has two components—one from inside A and one from inside B. It should be obvious that the total scattered fields at boundaries, A and B are given by:

$${f}_{A}^{S} = {S}_{A} {f}_{A} {A}_{A}^{in} + {T}_{AB} {S}_{B} {f}_{B}^{in} \qquad ( 3 a )$$

$${f}_{B}^{S} = {T}_{BA} {S}_{A} {f}_{A}^{in} + {S}_{B} {f}_{B}^{in} \qquad ( 3 b )$$

$$or$$

$$[ \begin{matrix}{f}_{A}^{S} \\ {f}_{B}^{S}\end{matrix} ] = [ \begin{matrix}{S}_{A} & {T}_{AB} {S}_{B} \\ {T}_{BA} {S}_{A} & {S}_{B}\end{matrix} ] [ \begin{matrix}{f}_{A}^{in} \\ {f}_{B}^{in}\end{matrix} ] \qquad ( 4 )$$

Combining (2) and (4) gives:

$$[ \begin{matrix}{f}_{A}^{S} \\ {f}_{B}^{S}\end{matrix} ] = {[ \begin{matrix}{S}_{A} & {T}_{AB} {S}_{B} \\ {T}_{BA} {S}_{A} & {S}_{B}\end{matrix} ] [ \begin{matrix}I & - {T}_{AB} {S}_{B} \\ - {T}_{BA} {S}_{A} & I\end{matrix} ]}^{- 1} [ \begin{matrix}{f}_{A}^{i} \\ {f}_{B}^{i}\end{matrix} ] \qquad ( 5 )$$

Assuming that the scattering operators are invertible, then we have the equivalent form:

$$[ \begin{matrix}{f}_{A}^{S} \\ {f}_{B}^{S}\end{matrix} ] = {[ \begin{matrix}I & {T}_{AB} \\ {T}_{BA} & I\end{matrix} ] [ \begin{matrix}{S}_{A}^{- 1} & - {T}_{AB} \\ - {T}_{BA} & {S}_{B}^{- 1}\end{matrix} ]}^{- 1} [ \begin{matrix}{f}_{A}^{i} \\ {f}_{B}^{i}\end{matrix} ] \qquad ( 6 )$$

The scattered field on the boundary C can be obtained from

$${f}_{A}^{s} and {f}_{B}^{s}$$

by simple truncation (and possible re-ordering depending on how the boundaries are parameterized). Let the operator that does this be denoted:

$${f}_{C}^{s} = [ {C}_{A} {C}_{B} ] [ \begin{matrix}{f}_{A}^{S} \\ {f}_{B}^{S}\end{matrix} ] \qquad ( 7 )$$

Let the incident field on boundary C due to external sources be denoted

$${f}_{C}^{i} .$$

There exist operators D_{A} and D_{B} that operate on

$${f}_{C}^{i}$$

to give

$${f}_{A}^{i} and {f}_{B}^{i}$$

(similar to the external translation operators T_{AB} and T_{BA}). Thus:

$${f}_{C}^{s} = {[ {C}_{A} {C}_{B} ] [ \begin{matrix}I & {T}_{AB} \\ {T}_{BA} & I\end{matrix} ] [ \begin{matrix}{S}_{A}^{- 1} & - {T}_{AB} \\ - {T}_{BA} & {S}_{B}^{- 1}\end{matrix} ]}^{- 1} [ \begin{matrix}{D}_{A} \\ {D}_{B}\end{matrix} ] {f}_{C}^{i} \qquad ( 8 )$$

from which we see that the scattering matrix for boundary C is given by:

$${S}_{C} = {[ {C}_{A} {C}_{B} ] [ \begin{matrix}I & {T}_{AB} \\ {T}_{BA} & I\end{matrix} ] [ \begin{matrix}{S}_{A}^{- 1} & - {T}_{AB} \\ - {T}_{BA} & {S}_{B}^{- 1}\end{matrix} ]}^{- 1} [ \begin{matrix}{D}_{A} \\ {D}_{B}\end{matrix} ] \qquad ( 9 )$$

Equation (9) then gives the core computation for our rectangular scattering matrix recursion algorithm. Technical details concerning the existence and discrete construction of the translation and other needed operators has been omitted in this write-up. We have, however written working first-cut programs that perform (9).

### An O(N³) Algorithm for Computing all Scattering Views Based on Rectangular Scattering Matrix Recursion

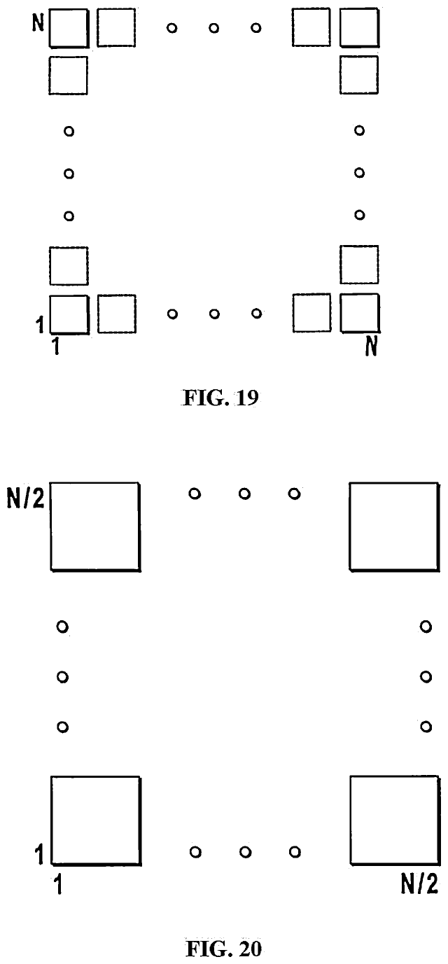

Consider a region containing scattering material covered by an N×N array of rectangular sub regions as shown in FIG. 19.

Again, although drawn as disjoint, assume that the subregions touch. Assume that the scattering matrix for each subregion is known (for example, if each subregion is only λ/2 on edge, the calculation of S is trivial given the material enclosed). Now coalesce 2×2 sets of these scattering matrices into larger scattering matrices (this coalesce of 4 subregions into one is similar to the algorithm defined above for coalescing two subregions). There are N/2×N/2 such 2×2 blocks to coalesce. When done, we have scattering matrices for the set of larger subregions shown in FIG. 20.

This process is continued until, at the final stage, the scattering matrix for the total region is computed. Note that the physical parameters of the scattering media (speed of sound, absorption, etc.) are used only in the first stage (computation of the N×N array of scattering matrices).

Assuming that N is a power of two, the algorithm will terminate after log₂(N) such stages with the scattering matrix for the whole region. A careful accounting of the computation required reveals that the total computation is O(N³). The end resulting scattering matrix then allows fast calculation of the scattered field anywhere external to the total region for any incident field (angle of view).

Although derived assuming that N is a power of 2, the algorithm can be generalized by including 3×3 (or any other sized) coalescing at a stage, allowing thereby algorithms for any N (preferably N should have only small, say 2,3,5, prime factors). Also, there is no reason that the starting array of subregions cannot be N×M (by using more general n×m coalescing at some stages).

In addition, the existence of layering can be included. If layering occurs above and below the total region so that the total inhomogeneity resides in a single layer, then the algorithm proceeds as before. Once the total region scattering matrix has been obtained, its interaction with the external layering can be computed. If inhomogeneous layer boundaries lie along horizontal borders between row of subscatterers, then the translation matrices can be modified when coalescing across such boundaries, properly including the layer effects. This is an advantage over our present layered Green's function algorithms which require that the inhomogeneity lie entirely within a single layer (This can be fixed in our present algorithms but at the expense of increased computation).

The O(N³) computation of this approach is superior to the O(N⁴ log₂(N)) computation of our original FFT-BCG approach and our present recursion based on cylindrical coordinates which is O(N³ log₂(N)).

### Example 9

### Modeling System Transfer Function Including Driving Voltage, Transmitting Transducers Receiving Transducers, Preamplifiers, Analog to Digital Converter Etc

Let the transfer function of the transmitting waveform generator, power amplifier, transmitting multiplexer, transmitting transducer, ocean/sediment, receiving transducers, preamplifiers, differential amplifier, differential waveform generator, and analog to digital converter be, respectively, T_{TWG}, T_{PA}, T_{TM}, T_{TT}, T_{O/S}, T_{RT}, T_{RM}, T_{DA}, T_{DWG}, and T_{DAC}. These separate transfer functions can be identified. Then the total transfer function is:

$${T}_{total} = {T}_{DAC} ( {T}_{DA 1} {T}_{RM} {T}_{RT} {T}_{O / S} {T}_{TT} {T}_{TM} {T}_{PA} {T}_{TWG} - {T}_{DA 2} {T}_{DWG} )$$

Note that the term T_{DA2} T_{DWG} is subtracted in order to remove direct path energy and to remove reverberations in the transducers and the platform; this subtraction effectively increases the analog to digital converter dynamic range. The signal in the differential waveform generator is programmed to produce a net zero signal output from the analog to digital converter for the case of no sediment present.

Recall that the equation for the scattered field f^{(sc)} (from the sediment) at a transducer (not the output voltage) at a given temporal frequency is given in terms of the transducer-to-sediment Green's function D, the sediment's acoustic properties γ and the internal field in the sediment f by

$${f}^{( sc )} = D γ f$$

The field (at a given temporal frequency) within the sediment itself f is given in terms of the incident field f^{(inc)}, the sediment's acoustic properties γ, and the sediment-to-sediment Green's function C by

$${f}^{( inc )} = ( I - C γ ) f$$

On combining these two equations we eliminate the internal field and find the scattered field in terms of the incident field and the sediment properties

$${f}^{( sc )} = D {γ ( I - C γ )}^{- 1} {f}^{( inc )}$$

These equations involving C and D are true for both the free space Green's function [“Nonperturbative Diffraction Tomography Via Gauss-Newton Iteration Applied to the Scattering Integral Equation, Borup, D. T. et al., Ultrasonic Imaging] and our newly developed layered Green's functions Johnson, S. A., D. T. Borup, M J. Berggren, Wiskin, J. W., and R. S. Eidens, 1992, “Modelling of inverse scattering and other tomographic algorithms in conjunction with wide bandwidth acoustic transducer arrays for towed or autonomous sub-bottom imaging systems,” Proc. of Mastering the Oceans through Technology (Oceans 92), 1992, pp 294-299.]. We now combine the scattering equations with the transfer functions. First, identify f^{(sc)} with T_{O/S}T_{TT}T_{TM}T_{PA}T_{TWG} and f^{(inc)} with T_{TT}T_{TM}T_{PA}T_{TWG}. Next we note that measuring f^{(inc)} is a direct way of finding the product T_{TT}T_{TM}T_{PA}T_{TWG}. We also note that Tₜₒₜₐₗ can be written as

$${T}_{total} ( γ , {T}_{TWG} ) = {T}_{DAC} ( {T}_{DA 1} {T}_{RM} {T}_{RT} D {γ ( I - C γ )}^{- 1} {T}_{TI} {T}_{TM} {T}_{PA} {T}_{TWG} - {T}_{DA 2} {T}_{DWG} ) .$$

Then Tₜₒₜₐₗ(γ, T_{TWG}) is a nonlinear operator that transforms (γ, T_{TWG}) into recorded signals T_{total-measured}. Thus, for a given set of T_{total-measured} measurements and for a given T_{TWG}, we may in principal find γ by a nonlinear inverse operator

$$γ = {T}_{total}^{- 1} ( {T}_{total - measured} ( γ , {T}_{TWG} ) ) .$$

Since the exact form of Tₜₒₜₐₗ⁻¹ is not known, we find γ by a Gauss-Newton iteration method.

This requires that the Jacobian of Tₜₒₜₐₗ(γ, T_{TWG}) be computed. The Jacobian is readily computed in closed form [Borup, 1992] and is given by

$$J ( γ ) = - \partial {T}_{total} ( γ {γ}^{( n )} , {T}_{TWG} ) / {\partial }_{γ} .$$

Then the Gauss-Newton iteration for computing y is given by: (1) set n=1, estimate a value for γ⁽ⁿ⁾; (2) compute J(γ⁽ⁿ⁾); (3) solve J^{T}(γ⁽ⁿ⁾)J(γ⁽ⁿ⁾)δγ⁽ⁿ⁾=−J^{T}(γ⁽ⁿ⁾)(γ⁽ⁿ⁾)[T_{total-measured}−Tₜₒₜₐₗ(γ⁽ⁿ⁾,T_{TWG})] for δγ⁽ⁿ⁾; (4) update γ⁽ⁿ⁾ by the formula γ⁽ⁿ⁺¹⁾=γ(n)+δγ⁽ⁿ⁾; (5) if T_{total-measured}−Tₜₒₜₐₗ(γ⁽ⁿ⁾,T_{TWG})<ϵ then set γ=γ⁽ⁿ⁺¹⁾ and quite, else go to step 2.

The extra dynamic range provided by the differential waveform generator/analog to digital converter circuit raises questions as to the optimal setup procedure (e.g., how many bits to span the noise present with no signal). We have modeled the signal to noise ratio that can be achieved by a beamformer which delays and sums multiple channel signals, each channel being such a circuit. We find as a rule of thumb, that the lowest order one or two bits should span either the noise or the signal, depending which ever is smaller (i.e., for signal to noise ratios greater than unity the noise should be spanned, but for signal to noise ratios less than unity the signal should be spanned). Upon using commercial 16 or 18 bit analog to digital converters this method may well extend their range to 20 bits or more.

### 2. Model Electrical Crosstalk, Acoustic Crosstalk

Electrical cross talk can be removed by use of knowledge of the cross coupling matrix M. Let Vₙ^{(true)} be true electrical signal at transducer n and let Vₘ^{(meas)} be the measured signal at transducer m. Then Vₙ^{(meas)}=MₙₘVₘ^{(true)}. We observe for small cross talk that matrix M has the form M=D₂(I+ϵ)D₁, where I is the identity matrix, D₁ and D₂ are diagonal matrices and E is the differential cross talk matrix whose elements are small in value. We seek Vₙ^{(true)}=M⁻¹Vₙ^{(meas)}. By the binomial theorem (I+ϵ)⁻¹≈(I−ϵ). Thus, M⁻¹≈D₁⁻¹(I−ϵ)D₂⁻¹. Once D₁, D₂, and M are measured the problem of removing cross talk is quite inexpensive numerically (even if ε is not small the exact inverse M⁻¹ can be computed once and stored). If the matrix M turns out to be noninvertible (as can be the case for large magnitude coupling) then we can alternatively concatenate M onto the inverse scattering equation to give:

$${ν}_{ω ψ}^{( meas )} = {M}_{ω} {P}_{ω} {G}_{ω} [ {f}_{ω ψ} ] γ$$

to which the inverse scattering algorithm can be directly applied.

We believe that cross talk can be removed by good fabrication techniques including careful shielding. Nevertheless, the above numerical method can be used if necessary.

Acoustic cross talk can be removed by several methods: (1) acoustic baffling of each transducers; (2) calibration of individual (isolated) transducers, computing the acoustic coupling in the array from wave equations methods, then inverting the model by a cross talk matrix as above; (3) direct measurement of the coupling in a finished array to find the cross talk matrix and then inverting as shown above. A more difficult type of cross talk to remove is the direct mechanical coupling between transducers. This problem will be attacked by using vibration damping techniques in mounting each transducer on the frame. We believe that such damping methods will eliminate direct mechanical coupling. As a backup we note that modeling of the coupled system are theoretically possible and has been successfully accomplished by the university's AIM Lab for circular mounting geometries (by derivation of a new “total system Green's function” for the imaging system that that includes cross coupling between elements).

### Example 10

### Inclusion of Transducer Coupling

In the event that significant coupling exists between the object to be imaged and the transducers (and/or coupling between transducers is not negligible), a computationally efficient means of incorporating this coupling into the inverse scattering algorithm is needed. Equivalently, the transducers must be included as part of the scattering problem This section details a computational algorithm for achieving this incorporation.

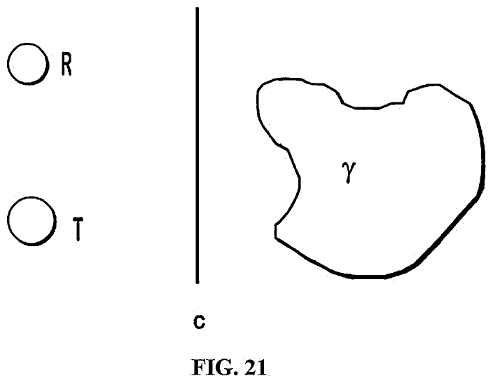

FIG. 21 shows a transducer coupling according to an embodiment of the present invention. Consider an object to be imaged, γ, illuminated by a transmitter, T, with the scattering received by a receiver, R, as shown in FIG. 21 Let C denote a closed surface separating y from the transceivers.

Let S be the scattering matrix of γ which, given the incident field generated from sources outside of C, gives the outward moving scattered field evaluated on C. This operator can be computed by solving a sufficient number of forward scattering problems for the object γ.

Let P_{R} denote the operator that computes the field impinging on R due to sources inside of C from the scattered field evaluated on C. This is a simple propagation operator computable by an angular spectrum technique.

Let P_{T} denote the operator that computes the field impinging on T due to sources inside of C from the scattered field evaluated on C. This is a simple propagation operator computable by an angular spectrum technique.

Let A_{RT} denote the operator that computes the field impinging on R due to scattering from T (it operates on the net total field incident on T). This operator is computed by a moment method analysis of the transmitter structure.

Let A_{TR} denote the operator that computes the field impinging on T due to scattering from R (it operates on the net total field incident on R). This operator is computed by a moment method analysis of the receiver structure.

Let B_{T} denote the operator that computes the field on C due to scattering from T (it operates on the net total field incident on T). This operator can be computed by a moment method analysis of the transmitter structure.

Let B_{R} denote the operator that computes the field on C due to scattering from R (it operates on the net total field incident on R). This operator can be computed by a moment method analysis of the receiver structure.

Assume that the transmitter T also produces a field, fⁱ, due to eternally applied excitation (electrical). Denote by

$${f}_{c}^{i}$$

the values of this field on C. Denote by

$${f}_{R}^{i}$$

the values of this field on the receiver surface. We assume that these field values are known (i.e., we know the free-space radiation characteristics of the transmitter).

Given these definitions, the total field incident from outside of C evaluated on C is given by:

$${f}_{c}^{tot} = {f}_{c}^{i} + {B}_{T} {f}_{T}^{tot} + {B}_{R} {f}_{R}^{tot} \qquad ( 1 )$$

where the superscript tot indicates the field incident on the particular element due to all other sources. For T and R we have:

$${f}_{T}^{lof} = {P}_{T} S {f}_{c}^{tot} + {A}_{TR} {f}_{R}^{lof} \qquad ( 2 )$$

$${f}_{R}^{tot} = {f}_{R}^{i} + {P}_{T} {Sf}_{c}^{tot} + {A}_{RT} {f}_{T}^{f o f} \qquad ( 3 )$$

Note that the formula for

$${f}_{T}^{tot}$$

has no superscript i term since the incident field emanates from its interior. Solving 1-3 for the tot fields gives:

$$[ \begin{matrix}{f}_{C}^{tot} \\ {f}_{T}^{tot} \\ {f}_{R}^{tot}\end{matrix} ] = [ \begin{matrix}I & - {B}_{T} & - {B}_{R} \\ {P}_{T} S & I & - {A}_{TR} \\ {P}_{R} S & - {A}_{RT} & I\end{matrix} ] [ \begin{matrix}{f}_{C}^{i} \\ 0 \\ {f}_{R}^{i}\end{matrix} ] = [ \begin{matrix}{f}_{C}^{tot} \\ {f}_{T}^{tot} \\ {f}_{R}^{tot}\end{matrix} ] \qquad ( 4 )$$

The size of this matrix operator is O(N×N) and so computation of its inverse does not require much CPU time (mere seconds). In order to compute the signal received by the receiver transducer, we take

$${f}_{R}^{tot}$$

computed in 4 and compute the surface currents (EM case) or surface velocities (acoustic case) from which the signal from the receiver can be computed.

This procedure for analyzing a scatterer in the presence of coupling between the T/R pair includes all orders of multiple interaction allowing transducers with complex geometries to be incorporated into our inverse scattering algorithms.

### Example 11

### Frequency Dependent Scattering Parameters

Throughout the previous sections it has been assumed that γ is independent of frequency. Suppose now that γ is a function of frequency. In the event that only a single frequency is needed (transmission mode with encircling transducers and only one complex parameter to be imaged) this is not a problem—the algorithm will simply image the 2 or 3-D distribution of γ evaluated at that frequency. However, in reflection mode or when imaging multiple parameters, multiple frequencies are needed. This increases the number of unknowns to Ω*Nₓ*N_{y} if we naively seek a separate image at each frequency. Since multiple frequencies were already needed to complete the data for a frequency independent unknown, we have no way of correspondingly increasing the number of equations by a factor of Ω. Instead, consider approximating the frequency variation with a set of parameters at each pixel:

$${γ}_{nm} ( ω ) \approx {γ}_{nm}^{( 0 )} {ψ}^{( 0 )} ( ω ) + {γ}_{nm}^{( 1 )} {ψ}^{( 1 )} ( ω ) + ⋯ + {γ}_{nm}^{( q - 1 )} {ψ}^{( q - 1 )}$$

where the basis functions ψ are selected based on the physics of the frequency dependence (we may, for example, simply use the monomial basis: ψ⁽ⁿ⁾(ω) ≡ωⁿ or, perhaps a rational function basis might be chosen since simple relaxation dispersion is a rational function). The formula for the GN update for this case is:

$${P}_{ω} {G}_{ω} ( {( I - [ {γ}_{ω} {G}_{ω} )}^{- 1} [ {f}_{ω ϕ} ] {M}_{ω , j} δ {γ}^{( j )} = - {r}_{ω ψ}$$

where summation over j is assumed and the matrix M is given by:

$$M = [ \begin{matrix}{ψ}^{( 1 )} ( {ω}_{1} ) & {ψ}^{( 2 )} ( {ω}_{1} ) & … & {ψ}^{( q - 1 )} ( {ω}_{1} ) \\ {ψ}^{( 1 )} ( {ω}_{2} ) & {ψ}^{( 2 )} ( {ω}_{2} ) & … & {ψ}^{( q - 1 )} ( {ω}_{2} ) \\ {ψ}^{( 1 )} ( {ω}_{Ω} ) & {ψ}^{( 2 )} ( {ω}_{Ω} ) & … & {ψ}^{( q - 1 )} ( {ω}_{Ω} )\end{matrix} ]$$

Solution for the q×Nₓ×N_{y} unknowns, assuming that q is sufficiently smaller than Q can then carried out via the GN-MRCG or RP algorithms. We have already verified the success of this approach using quadratic polynomial models of the frequency variation (q=3, ψ⁽ⁿ⁾(ω)≡ωⁿ).

### Example 12—Parabolic Marching Methods

Having seen in the previous pages how the full nonlinear inversion yields substantial increases in resolution and utility of image, we are now prepared to discuss the advanced marching technique employed in both the forward problem and the Jacobian calculations (as well as the closely related Hermitian conjugate of the Jacobian calculation).

### Scientific Background and Detailed Description to Advanced Parabolic Marching Method

The parabolic equation method is a very efficient method for modelling acoustic wave propagation through low contrast acoustic materials (such as breast tissue). The original or classical method requires for its applicability that energy propagate within approximately ±20° from the incident field direction. Later versions allow propagation at angles up to ±90° from the incident field direction. Further modifications provide accurate backscattering information, and thus are applicable to the higher contrasts encountered in nondestructive imaging, EM and seismic applications. [M. D. Collins, A two-way parabolic equation for acoustic backscattering in the ocean, Journ. Acoustical Society of America, 1992, 91, 1357-1368, F. Natterer and F. Wubbeling, “A Finite Difference Method for the Inverse Scattering Problem at Fixed Frequency,” Lecture Notes in Physics, 1993, vol. 422:157-166, herein included as reference]. The source/receiver geometry we use in this device, and the relatively low contrast of breast tissue, allow us to utilize this efficient approximation. The resulting speed up relative to the Gauss Newton, conjugate gradient method [Borup et al., 1992] described in the previous patent, is dependent upon the contrast but is estimated to be 100-400 times. Furthermore the coarse grain parallelization employed in the integral equation method is equally applicable to the parabolic algorithm. The basic structure of the parabolic inversion algorithm is also essentially unchanged. The main difference is in the use of the Parabolic equation approximation, and more exactly, the “split step Fourier Method” [R. H. Hardin and F. D. Tappert “Applications of the split-step fourier method to the solution of nonlinear and variable coefficient wave equations,” SIAM Rev. 15, 423 (1973). and M. D. Collins, “A Split-step Pade’ solution for the parabolic equation method,” J. Acoust. Soc. Am. 93, 1736-1742 (1993), herein included as reference] in the construction of both the Jacobian of the residual function, and the solution to the forward problems for each view. The source/receiver geometry requirements and the relatively low contrast of breast tissue, allow us to utilize this efficient approximation, for breast cancer scanner applications, for example. In particular because we may elect to use only transmission data in the breast problem, we are able to use this “parabolic approximation” in a straight-forward, simple manner [see Hardin and Tappert].

To derive and elucidate the parabolic equation method, we begin with the 2-D Helmholtz wave equation governing wave propagation in inhomogeneous media:

$$\{ \frac{{\partial }^{2}}{\partial {x}^{2}} + ( {k}^{2} ( x , y ) + \frac{{\partial }^{2}}{\partial {y}^{2}} ) \} f ( x , y ) = 0 \qquad 1$$

Now Fourier transforming y to λ results in:

$$[ \frac{{\partial }^{2}}{\partial {x}^{2}} + ( \frac{1}{2 π} ( λ ) - {λ}^{2} ) ] \tilde{f} ( x , λ ) = 0 \qquad 2$$

where:

$$( λ ) = ( x , λ ) = {\int }_{- ∞}^{∞} {k}^{2} ( x , y ) {e}^{- i λ y} dy \qquad 3$$

and the * notation denotes convolution:

$$f ( λ ) * g ( λ ) = {\int }_{- ∞}^{∞} f ( λ - {λ}^{′} ) g ( {λ}^{′} ) d {λ}^{′} \qquad 4$$

Eqn (2) can be factored in the sense of pseudo-differential operators [M. E. Taylor, “Pseudo-differential Operators,” Princeton University Press, Princeton, 1981, herein included as reference]

$$( \frac{\partial }{\partial x} + i \sqrt{\frac{1}{2 π} {( λ )}^{2} - {λ}^{2}} ) ( \frac{\partial }{\partial x} - i \sqrt{\frac{1}{2 π} {( λ )}^{2} - {λ}^{2}} ) \tilde{f} ( x , λ ) = 0 \qquad 5$$

An intuitive feel for the manner in which the Parabolic equation arises can be seen by looking at particularly simple case: when k is a constant, then we have, upon Fourier Transforming (1):

$$( \frac{\partial }{\partial x} + i \sqrt{{k}^{2} - {λ}^{2}} ) ( \frac{\partial }{\partial x} - i \sqrt{{k}^{2} - {λ}^{2}} ) \tilde{f} ( x , λ ) = 0 \qquad 6$$

where the square root is now simply the square root of a scalar. From 6, it is clear that in this special case, two solutions exist:

$$( \frac{\partial }{\partial x} + i \sqrt{{k}^{2} - {λ}^{2}} ) \tilde{f} ( x , λ ) = 0 , \tilde{f} ( x , λ ) = g ( λ ) {e}^{- ix \sqrt{{k}^{2} - {λ}^{2}}} \qquad 7$$

for an arbitrary function g, which represents a rightmoving wave for e^{iwt} time dependence and:

$$( \frac{\partial }{\partial x} - i \sqrt{{k}^{2} - {λ}^{2}} ) \tilde{f} ( x , λ ) = 0 , \tilde{f} ( x , λ ) = g ( λ ) {e}^{ix \sqrt{{k}^{2} - {λ}^{2}}} \qquad 8$$

representing a wave which “moves” to the left. Suppose that we know that the field is due to sources entirely on the LHS of the x-y plane. Then, for x>0 we must have:

$$\tilde{f} ( x , λ ) = g ( λ ) {e}^{- ix \sqrt{{k}^{2} - {λ}^{2}}} \qquad 9$$

Note that knowledge of f on the line x=0 (boundary condition) completes the solution since:

$$f ( 0 , λ ) = g ( λ ) \qquad 10$$

and so, on inverse Fourier Transforming:

$$f ( x , λ ) = \frac{1}{2 π} {\int }_{- ∞}^{∞} \tilde{f} ( 0 , λ ) {e}^{- ix \sqrt{{k}^{2} - {λ}^{2}}} {e}^{i λ y} d λ , x > 0 \qquad 11$$

In fact, for any x₀>0:

$$f ( x , y ) = \frac{1}{2 π} {\int }_{- ∞}^{∞} \tilde{f} ( {x}_{0} , λ ) {e}^{- i ( x - {x}_{0} ) \sqrt{{k}^{2} - {x}^{2}}} {e}^{i λ y} d λ , x > {x}_{0} > 0 \qquad 12$$

The idea behind the parabolic equation method is to try to factor a general inhomogeneous (i.e. k=k(x,y)) problem into forward and backward moving (in x) factors and then solve only the forward (+x) moving part (assuming that the field source is to the left (on the x-axis) of the scatterer).

Let xₙ=nΔ, n=0, . . . then 12 is:

$${f}_{n + 1} ( y ) = \frac{1}{2 π} {\int }_{- ∞}^{∞} \overline{{f}_{n}} ( λ ) {e}^{- i Δ \sqrt{{k}^{2} - {λ}^{2}}} {e}^{i λ y} d λ \qquad 13$$

Since 13 is local x=(n+1/2)Δ we consider the discretization/approximation:

$${f}_{n + 1} ( y ) = \frac{1}{2 π} {\int }_{- ∞}^{∞} {\tilde{f}}_{n} ( λ ) {e}^{- i Δ \sqrt{{k}^{2} ( {x}_{n + 1 / 2}} , y ) - {λ}^{2}} {e}^{i λ y} d λ \qquad 14$$

for propagating forward through k that is inhomogeneous in x and y i.e., k=k(x, y), and fₙ(γ)=f(xn, y). Computationally (14) would be an inverse Fourier transform except for the y-dependence of k. Defining

$${k}_{n}^{2} = {k}^{2} ( {x}_{n + 1 / 2} )$$

gives

$${f}_{n + 1} ( y ) = \frac{1}{2 π} {\int }_{- ∞}^{∞} {\tilde{f}}_{n} ( λ ) {e}^{- i Δ \sqrt{{k}^{2} ( {x}_{n + 1 / 2} , y ) - {λ}^{2}}} {e}^{i λ y} d λ$$

### Advanced Parabolic Marching Method

Now we desire to take the y-dependence out from under the square root in order that it may then be factored out from under the integral, which will result in the integral being an inverse Fourier Transform, and yield a substantial increase in efficiency. This goal can be achieved approximately in the following manner:

$${e}^{- i Δ {k}_{n , m} \sqrt{1 - α / {k}_{n , m} {r}^{2}}} \approx {e}^{i Δ ( {k}_{n , m} - {k}_{n} )} {e}^{- i Δ {k}_{n} \sqrt{1 - {( λ / {k}_{n} )}^{2}}}$$

so that, defining k_{n,m}≡k(x_{n+1/2}, yₘ), and yₘ=mΔ, then yields:

$${f}_{n + 1} ( {y}_{m} ) \approx \frac{1}{2 π} {\int }_{- ∞}^{∞} {\tilde{f}}_{n} ( λ ) {e}^{- i Δ ( {k}_{n , {m}^{-}} {k}_{n} )} {e}^{- i Δ {k}_{n} \sqrt{{1 - λ / {k}_{n} )}^{2}}} {e}^{i λ {y}_{m}} d λ or \qquad ( 15 )$$

$${f}_{n + 1} ( {y}_{m} ) \approx \frac{{e}^{- i Δ ( {k}_{n , m} - {k}_{n} )}}{2 π} {\int }_{- ∞}^{∞} {\tilde{f}}_{n} ( λ ) {e}^{- i Δ {k}_{n} \sqrt{1 - {( λ / {k}_{n} )}^{2}}} {e}^{i λ {y}_{m}} d λ$$

We have thus approximately maintained the γ variation in λ, while simultaneously giving a Fourier Transform formulation for the field fₙ+1.

$${f}_{n + 1} ( y ) \approx \frac{{e}^{- i Δ ( {k}_{n , m} - {k}_{n} )}}{2 π} {\int }_{- ∞}^{∞} {\tilde{f}}_{n} ( λ ) {e}^{- i Δ {k}_{n} \sqrt{1 - {( λ / {k}_{n} )}^{2}}} {e}^{i λ y} d λ$$

and in fact using the notation:

$$\hat{f} ( y ) = \frac{1}{2 π} {\int }_{- ∞}^{∞} f ( λ ) {e}^{- i λ y} d λ$$

to indicate the Inverse Fourier Transform, gives:

$${f}_{n + 1} ( y ) = \hat{\overline{{\tilde{f}}_{n} ( λ ) {P}_{n}}}$$

where Pₙ is the range dependent propagator defined as:

$${P}_{n} ( y ) = {e}^{- i Δ \sqrt{{k}^{2} ( {x}_{n + 1 / 2} ) - {λ}^{2}}}$$

This is one basic equation for the parabolic split step method. Various alternative forms can be used in similar algorithms. A common form for the parabolic equation is derived by using the binomial approximation:

$$\sqrt{1 - {( λ / {k}_{n} )}^{2}} \approx \frac{1}{2} {( \frac{λ}{{k}_{n}} )}^{2}$$

in the above integral. This yields a “standard” form of the split-step parabolic equation method:

$${f}_{n + 1} ( {y}_{m} ) = \frac{{e}^{- i Δ {k}_{n , m}}}{2 π} {\int }_{- ∞}^{∞} \tilde{f} ( λ ) {e}^{i Δ \frac{{λ}^{2}}{2 {k}_{0}}} {e}^{i λ {y}_{m}} d λ \qquad 16$$

Numerical experiments have indicated the superiority of 15 over 16. In Fourier notation, 16 is:

$${f}_{n + 1} ( {y}_{m} ) = {e}^{- i Δ {k}_{n , m}} {F}^{- 1} \{ {e}^{i Δ \frac{{λ}^{2}}{2 {k}_{0}}} F \{ {f}_{n} \} ( λ ) \} ( {y}_{m} ) \qquad 17$$

This is the usual form of the split-step PE method. Our more general equation is (in Fourier notation):

$${f}_{n + 1} ( {y}_{m} ) = {e}^{- i Δ ( {k}_{n , {m}^{-}} {k}_{n} )} {F}^{- 1} \{ {e}^{- i Δ {k}_{n} \sqrt{1 - {( λ / {k}_{n} )}^{2}}} F \{ {f}_{n} \} ( λ ) \} ( {y}_{m} )$$

An interesting interpretation of the split step method is evident from this: The algorithm step consists of an exact propagation a distance of Δ in kₒ (which includes diffraction) followed by a phase shift correction, i.e, multiplication by

$${e}^{- i Δ ( {k}_{n , m} - {k}_{0} )} \qquad 18$$

which corrects the phase of a plane wave travelling forward.

### Relation to the Generalized Born Approximation

The above split step Fourier method can be seen to be a generalization of the “Generalized Born” method as developed at TechniScan scientific research division in the following manner. Suppose that we assume that the field variation in y is very slow, i.e.,

$$F \{ {f}_{n} \} ( λ ) \neq 0 for λ \times 0$$

so that we may replace

$${e}^{- i Δ {k}_{n} \sqrt{1 - {( λ / {k}_{n} )}^{2}}} \approx {e}^{- i Δ {k}_{n}}$$

in the above integral which

$$\frac{1}{2 π} {\int }_{- ∞}^{∞} {\tilde{f}}_{n} ( λ ) {e}^{- i Δ \sqrt{{k}^{2} - {λ}^{2}}} {e}^{i λ y} d λ \approx {f}_{n} ( y ) {e}^{- i {k}_{0} Δ} \qquad 19$$

Then 28 is:

$${f}_{n + 1} ( {y}_{m} ) = {e}^{- i Δ {k}_{n , m}} {f}_{n} ( {y}_{m} ) , {f}_{n + 1} ( {y}_{m} ) = {e}^{- i Δ {\sum }_{n = 0}^{n} {k}_{n , m}} ( since {f}_{0} ( y ) = 1 ) \qquad 20$$

which is:

$$f ( x , y ) = {e}^{- i} {\int }_{0}^{x} k ( {x}^{′} , y ) {dx}^{′} \qquad 21$$

This is the generalized Born approximation to the field. Thus, the PE formula 17 reduces to the GB formula if straight line propagation and no diffraction are assumed.

The PE method should be significantly superior to GB, particularly if the PE total field is rescattered:

$${f}^{s} ( ρ ) = {k}_{0}^{2} \int \int γ ( {ρ}^{′} ) {f}^{P E} ( {ρ}^{′} ) \frac{{H}_{0}^{( 2 )} ( {k}_{0} \vert ρ - {ρ}^{′} \vert )}{4 i} {ds}^{′} \qquad 22$$

where f^{PE} is computed from 17 by the split step algorithm. This will also improve the calculation of side and backscatter. The PE formulation has no backscatter (or large angle scattering) in it. Equation 33 is a way to put it back in, in a manner that gives a good approximation for weak scattering.

It is important to note that the above formulation of the parabolic equation inversion method does not require the storage of any fields (unlike the integral equation method of the previous patent). Of course, this method (as in the previous method) does not require the storage of a large Jacobian, or its adjoint.

### Inverse Problem and Construction of Jacobian

The construction of the inversion algorithm requires the formula for the Jacobian:

$$\frac{\partial }{\partial {k}_{i , j}} {f}_{n + 1} ( {y}_{m} ) = {e}^{- i Δ ( {k}_{n , m} - {k}_{o} )} ( - i Δ {δ}_{[ n , m ]}^{[ i , j ]} ) {F}^{- 1} \{ {e}^{- i Δ {k}_{o} \sqrt{1 - {( λ / {k}_{o} )}^{2}}} F \{ {f}_{n} \} ( λ ) \} ( {y}_{m} ) + {e}^{- i {Δ ( {k}_{n , {m}^{-}} {k}_{o} )}^{2}} {F}^{- 1} \{ {e}^{- i Δ {k}_{o} \sqrt{1 - {( λ / {k}_{o} )}^{2}}} F \{ \frac{\partial }{\partial {k}_{i , j}} {f}_{n} \} ( λ ) \} ( {y}_{m} )$$

More exactly, we use the conjugate gradient algorithms in our inversion, and consequently require only the action of the Jacobian defined above on the perturbation δk_{i,j}, i.e. the total variation of f with respect to k:

$$δ {f}_{n + 1} ( {y}_{m} ) ≡ {\sum }_{i , j} \frac{\partial {f}_{n + 1}}{{k}_{i , j}} ( {y}_{m} ) δ {k}_{i , j}$$

The recursion formula for the action of the Jacobian on the perturbation in k is given by

$$δ {f}_{n + 1} ( {y}_{m} ) = {e}^{- i Δ ( {k}_{n , {m}^{-}} {k}_{o} )} ( - i Δ δ {k}_{n , m} ) {F}^{- 1} \{ {e}^{- i Δ {k}_{o} \sqrt{{1 - ( λ / {k}_{o} \}}^{2}}} F \{ {f}_{n} \} ( λ ) \} ( {y}_{m} ) + {e}^{- i Δ ( {k}_{n , m} - {k}_{o} )} {F}^{- 1} \{ {e}^{- i Δ {k}_{o} \sqrt{{1 - ( λ / {k}_{o} \}}^{2}}} F \{ δ {f}_{n} \} ( λ ) \} ( {y}_{m} )$$

It is advisable to rewrite the recursion expression for the field values fₙ₊₁(yₘ) as:

$${f}_{n + 1} ( {y}_{m} ) = {t}_{n , m} {\int }_{- ∞}^{∞} {\tilde{f}}_{n} ( λ ) {e}^{- i Δ \sqrt{{k}_{o}^{2} - {λ}^{2}}} {e}^{i λ {y}_{m}} d λ$$

Where either

$${t}_{n , m} = \frac{2}{1 + \sqrt{{γ}_{n , m} + 1}} {e}^{- i Δ ( {k}_{n , {m}^{-}} {k}_{o} )}$$

$$or$$

$${I}_{n , m} ≡ \frac{2}{1 + \sqrt{\frac{{γ}_{n - 1 , m} + 1}{{γ}_{n , m} + 1}}} {e}^{- i Δ ( {k}_{n , {m}^{-}} {k}_{n} )}$$

is the “transmission coefficient+phase mask” characterizing the medium, depending upon The form used depends upon whether the model used as the background medium includes a priori known layering or not. In this case the equation for the Jacobian itself reads:

$$\frac{\partial }{\partial {t}_{i , j}} {f}_{n + 1} ( {y}_{m} ) = {δ}_{[ n , m ]}^{[ i , j ]} {F}^{- 1} \{ {e}^{- i Δ \sqrt{{k}_{0}^{2} - {λ}^{2}}} F \{ {f}_{n} \} ( λ ) \} ( {y}_{m} ) + {t}_{n , m} {F}^{- 1} \{ {e}^{- i Δ \sqrt{{k}_{0}^{2} - {λ}^{2}}} F \{ \frac{\partial {f}_{n}}{\partial {t}_{i , j}} \} ( λ ) \} ( {y}_{m} )$$

and the total variation in terms of St is:

$$δ {f}_{n + 1} ( {y}_{m} ) ≡ {\sum }_{i , j} \frac{\partial {f}_{n + 1}}{\partial {l}_{i , j}} ( {y}_{m} ) δ {t}_{i , j}$$

The recursion formula for δf is therefore:

$$δ {f}_{n + 1} ( {y}_{m} ) = \text{} δ {t}_{n , m} {F}^{- 1} \{ {e}^{- i Δ \sqrt{{k}_{o}^{2} - {λ}^{2}}} F \{ {f}_{n} \} ( λ ) \} ( {y}_{m} ) + {t}_{n , m} {F}^{- 1} \{ {e}^{- i Δ \sqrt{{k}_{o}^{2} - {λ}^{2}}} F \{ δ {f}_{n} \} ( λ ) \} ( {y}_{m} )$$

The Formula for the Hermitian Conjugate of the Jacobian

The recursion for the total variation in the data δf_{N} can be written as:

$$δ {f}_{N} = δ {t}_{N} \hat{ψ} {ν}_{N} + {W}_{N} δ {f}_{N - 1}$$

where x denotes the Hadamard product, which is defined in the following manner

$$t ⊗ v ≡ [ \begin{matrix}{t}_{1} \\ ⋮ \\ {t}_{N - 1} \\ {t}_{N}\end{matrix} ] ⊗ [ \begin{matrix}{v}_{1} \\ ⋮ \\ {v}_{N - 1} \\ {v}_{N}\end{matrix} ] = [ \begin{matrix}{t}_{1} {v}_{1} \\ ⋮ \\ {t}_{N - 1} {v}_{N - 1} \\ {t}_{N} {v}_{N}\end{matrix} ]$$

Also, the matrix Wⱼ is defined as:

$${W}_{j} = [ {t}_{j} ] {A}_{j}$$

where [tⱼ] represents the diagonal matrix:

$$[ {I}_{j} ] ≡ [ \begin{matrix}{t}_{j , 1} & 0 & 0 & 0 \\ 0 & {t}_{j , 2} & 0 & 0 \\ 0 & 0 & ⋱ & 0 \\ 0 & 0 & 0 & {t}_{j , M}\end{matrix} ]$$

whose diagonal terms consist of the elements of the vector tⱼ, and Aⱼ is the matrix defined as (where juxtaposition always indicates matrix multiplication:

$${A}_{j} ≡ {F}^{- 1} {P}_{j} F$$

Also the vector vⱼ is defined as

$${v}_{j} = {A}_{j} {f}_{j - 1}$$

i.e.

$${v}_{j} = {F}^{- 1} {P}_{j} {Ff}_{j - 1}$$

Note that with the definitions:

$$A = [ \begin{matrix}{a}_{1} \\ ⋮ \\ {a}_{M}\end{matrix} ] t ≡ [ \begin{matrix}{t}_{1} & 0 & 0 & 0 \\ 0 & {t}_{2} & 0 & 0 \\ 0 & 0 & ⋱ & 0 \\ 0 & 0 & 0 & {t}_{M}\end{matrix} ]$$

for the M×M matrices A, and [t], it follows that the matrix product is given by:

$$[ t ] A = [ \begin{matrix}{I}_{1} {a}_{1} \\ ⋮ \\ {I}_{M} {a}_{M}\end{matrix} ]$$

That is, the i^{th} row of [t]A, is tᵢ multiplied by the i^{th} row of A.

With these notational assumptions, the recursion for the total variation becomes:

$${f}_{N} = [ {v}_{N} ] δ {t}_{N} + {W}_{N} [ {v}_{N - 1} ] δ {t}_{N - 1} + {W}_{N} {W}_{( N - 1 )} [ {V}_{N - 2} ] δ {t}_{N - 2} + ⋯ + ( {W}_{N} {W}_{( N - 1 )} … {W}_{1} ) [ {V}_{0} ] δ {t}_{0}$$

The vⱼ=F⁻¹Pⱼ Ffⱼ₋₁ are computed and stored as the forward fields are computed within the “subroutine jach” which computes the action of the Hermitian conjugate of the jacobian on (the complex conjugate of) the residual vector.

The updates for one view, then, are constructed in sequence using the formulae:

$$[ δ {t}_{N} ] = [ {v}_{N} ] \overline{[ δ {f}_{N} ]}$$

$$[ δ {t}_{N - 1} ] = [ {v}_{N - 1} ] {W}_{N}^{T} \overline{[ δ {f}_{N} ]} = [ {v}_{N - 1} ] {FP}_{N} {F}^{- 1} [ {t}_{j} ] \overline{[ δ {f}_{N} ]}$$

etc.

See FIGS. 19-24.

### Scientific Background for the Generalized Born Approximation:

The exact scattering 2D) integral equation is given by:

$${f}^{scat} ( ρ ) = {k}_{0}^{2} \int \int γ ( {ρ}^{′} ) f ( {ρ}^{′} ) \frac{{H}_{0}^{( 2 )} ( {k}_{0} \vert ρ - {ρ}^{′} \vert )}{4 i} {ds}^{′} \qquad 1$$

where f is the exact total field. The generalized Born approximation is obtained by approximating f in (1) with a straight-line phase integrated field approximation. For a plane wave traveling in the +x direction, this approximation is:

$$f ( {x}^{′} , {y}^{′} ) \approx {e}^{- {ik}_{0} {x}^{′}} {e}^{- i ω {\int }_{- ∞}^{{x}^{′}} ( 1 / c ( {x}^{′′} , {y}^{′} ) - 1 / {c}_{0} ) {dx}^{′′}} \qquad 2$$

which has the proper phase (time delay) assuming straight-line propagation. For a point source incident field, the approximation is:

$$f ( {x}^{′} , {y}^{′} ) \approx \frac{{H}_{0}^{( 2 )} ( ω {\int }_{{ρ}_{tran}}^{{ρ}^{′}} ( dl / c ( l ) ) )}{4 i} \qquad 3$$

where ρₜᵣₐₙ is the position of the point source. For a point receiver at ρ_{rec}, equation 1

$${f}^{scat} ( {ρ}_{rec} ) = {k}_{0}^{2} \int \int γ ( {ρ}^{′} ) \frac{{H}_{0}^{( 2 )} ( ω {\int }_{{ρ}_{tran}}^{{ρ}^{′}} ( dl / c ( l ) ) )}{4 i} \frac{{H}_{0}^{( 2 )} ( {k}_{0} \vert {ρ}_{rec} - {ρ}^{′} \vert )}{4 i} {ds}^{′} \qquad 4$$

Where the path of integration from ρₜᵣₐₙ to ρ′ is a straight line.

### Transformation to the Time Domain

Using the asymptotic approximation:

$${H}_{0}^{( 2 )} ( x ) {\to }_{x \to ∞} \sqrt{\frac{2 i}{π x}} {e}^{- ix} \qquad 5$$

gives the approximation:

$${f}^{sat} ( {ρ}_{rec} , {ρ}_{tran} , ω ) = - \frac{i ω}{8 π {c}_{0}} \int \int γ ( {ρ}^{′} ) \frac{( {e}^{- i ω {\int }_{C}^{( dl / c ( l ) )} ) ( {e}^{- i ω \vert {ρ}_{rec} - {ρ}^{′} \vert {c}_{0}} )}}{\sqrt{\vert {ρ}_{tran} - ρ \vert \vert {ρ}_{rec} - {ρ}^{′} \vert}} {ds}^{′} \qquad 6$$

where the path integral is given by:

$${\int }_{C} ( dI / c ( l ) ) = {\int }_{{ρ}_{tran}}^{{ρ}^{′}} ( dl / c ( l ) )$$

Transforming to the time domain gives:

$${f}^{scat} ( {ρ}_{rec} , {ρ}_{max} , t ) = - \frac{1}{8 π {c}_{0}} \frac{\partial }{\partial t} \int \int γ ( {ρ}^{′} ) \frac{δ ( t - {\int }_{{ρ}_{tran}}^{{ρ}^{′}} ( dl / c ( l ) ) - \vert {ρ}_{rec} - {ρ}^{′} \vert / {c}_{0} )}{\sqrt{\vert {ρ}_{tran} - {ρ}^{′} \vert \vert {ρ}_{rec} - {ρ}^{′} \vert}} {ds}^{′} \qquad 7$$

In reflection mode (ρ_{rec} on the same side of the body as ρₜᵣₐₙ), there is a problem with equation 7 of this section in that the scattering from point ρ′ arrives at the receiver at the wrong time. The transmitter pulse arrives at ρ′ at the properly delayed time,

$${\int }_{{ρ}_{tran}}^{{ρ}^{′}} ( dl / c ( l ) ) .$$

but then the response travels back to the receiver as if the body were absent (time back to receiver=|ρ_{rec}−ρ′/c₀). Thus, equation 7 in this section is acausal. To correct this, in reflection mode, we time delay the receiver path as well:

$${f}^{scat} ( {ρ}_{rec} , {ρ}_{max} , t ) = - \frac{1}{8 π {c}_{0}} \frac{\partial }{\partial t} \int \int γ ( {ρ}^{′} ) \frac{δ ( t - {\int }_{{ρ}_{tran}}^{{ρ}^{′}} ( dl / c ( l ) ) - {\int }_{{ρ}^{′}}^{{ρ}_{rec}} ( dl / c ( l ) ) )}{\sqrt{\vert {ρ}_{rman} - {ρ}^{′} \vert \vert {ρ}_{rec} - {ρ}^{′} \vert}} {ds}^{′} \qquad 8$$

Equation 8 has been found to be quite accurate in reflection mode. In transmission mode, we retain 7 because in transmission, the scattered field is, in fact, acausal (in the sense that part of the scattered field arrives as if no body were present).

In reality, transducers have a limited bandwidth. Let s(t) be the system response of the transducers. Then equation 8 becomes:

$${f}^{scal} ( {ρ}_{rec} , {ρ}_{max} , t ) = - \frac{1}{8 π {c}_{0}} {s}^{′} ( t ) \approx \int \int γ ( {ρ}^{′} ) \frac{δ ( t - {\int }_{{ρ}_{tran}}^{{ρ}^{′}} ( dl / c ( l ) ) - {\int }_{{ρ}^{′}}^{{ρ}_{rec}} ( dl / c ( l ) ) )}{\sqrt{\vert {ρ}_{ran} - {ρ}^{′} \vert \vert {ρ}_{rec} - {ρ}^{′} \vert}} {ds}^{′} \qquad 9$$

where the * denotes convolution in time. Equation 9 provides a very powerful algorithm for time-domain, reflection-mode scattering calculation. It's main advantage over, say, the parabolic algorithm, is that it is in the time domain. Reflection mode scattering using the parabolic method requires that each frequency be computed separately, while equ. 9 gives the full time waveform with one computation.

The addition of attenuation into equ. 9 is trivial:

$${f}^{scat} ( {ρ}_{rec} , {ρ}_{max} , t ) = - \frac{1}{8 π {c}_{0}} {s}^{′} ( t ) * \int \int γ ( {ρ}^{′} ) \frac{{e}^{{\int }_{{ρ}_{tran}}^{{ρ}^{′}} α ( l ) dl} {e}^{{\int }_{{ρ}^{′}}^{{ρ}_{rec}} α ( l ) dl} δ ( t - {\int }_{{ρ}_{tran}}^{{ρ}^{′}} ( dl / c ( l ) ) - {\int }_{{ρ}^{′}}^{{ρ}_{rec}} ( dl / c ( l ) ) )}{\sqrt{\vert {ρ}_{tran} - {ρ}^{′} \vert \vert {ρ}_{rec} - {ρ}^{′} \vert}} {ds}^{′}$$

where α is the inhomogeneous attenuation.

Imaging algorithms can be derived from 9-10 by using these equations as the nonlinear operator (in γ) for predicting the scattering data and applying our standard Fletcher-Reeves or Ribere-Polack approach.

### Basis for Fast Computational Algorithm Based Warping of Metric in Image Space

The 1-D Generalized Born formula in the frequency domain is:

$${f}^{s} ( ω , - d ) = \frac{ω {e}^{- i ω d / {c}_{0}}}{2 {ic}_{0}} {\int }_{0}^{a} γ ( x ) {e}^{- i ω ( \frac{2}{{c}_{0}} {\int }_{0}^{x} {S}_{r} ( {x}^{′} ) {dx}^{′} + {dlc}_{0} )} dx \qquad 1$$

Assuming fⁱ(ω, x)=e^{-iω(x+d)/c}^{₀}⁾ where:

Sᵣ(x)=relative slowness=\sqrt{γ(x)+1}

Inverse Fourier transformation of 1 to time gives:

$$- 2 {c}_{0} {F}^{- 1} \{ \frac{{f}^{s} ( ω , - d )}{i ω} \} ( t ) = {\int }_{0}^{a} γ ( x ) δ ( t - \frac{2}{{c}_{0}} {\int }_{0}^{x} {S}_{r} ( {x}^{′} ) {dx}^{′} - 2 d / {c}_{0} ) dx \qquad 2$$

$$or$$

$$- 2 {c}_{0} {\int }_{0}^{t} {f}^{s} ( {t}^{′} - 2 d / {c}_{0} , - d ) {dt}^{′} = {\int }_{0}^{a} γ ( x ) δ ( t - \frac{2}{{c}_{0}} {\int }_{0}^{x} {S}_{r} ( {x}^{′} ) {dx}^{′} ) dx \qquad 4$$

Change of variables:

$$z = {\int }_{0}^{x ( z )} {S}_{r} ( {x}^{′} ) {dx}^{′} , dx = \frac{dz}{{S}_{r} ( x ( z ) )} \qquad 5$$

to get:

$$- 2 {c}_{0} {\int }_{0}^{t} {f}^{s} ( t + 2 {dfc}_{0} - d ) {dt}^{′} = {\int }_{0}^{z ( a )} \frac{γ ( x ( z ) )}{{S}_{r} ( x ( {z}^{′} ) )} δ ( t - \frac{2}{{c}_{0}} {z}^{′} ) {dz}^{′} \qquad 6$$

which gives:

$$- 4 {\int }_{0}^{2 z / {c}_{0}} {f}^{s} ( {t}^{′} + 2 d / {c}_{0} , - d ) {dt}^{′} = \frac{γ ( x ( z ) )}{{S}_{r} ( x ( {z}^{′} ) )} \qquad 7$$

where x(z) is defined in 5 which is also:

$$x ( z ) = {\int }_{0}^{z} \frac{dz}{{S}_{r} ( x ( z ) )} \qquad 8$$

which provides a recursive formula for x(z) for the discretized case. For example, trapezoidal integration of 8 gives:

$${x}_{n} - \frac{Δ}{2} \frac{1}{{S}_{r} ( {x}_{n} )} = \frac{Δ}{2} \{ \frac{1}{{S}_{r} ( 0 )} + 2 {\sum }_{l = 1}^{n - 1} \frac{1}{{S}_{r} ( {x}_{l} )} \} , {x}_{n} = x ( {z}_{n} ) , {z}_{n} = Δ n \qquad 9$$

which is easy to solve for xₙ if, for example, Sr is piecewise constant.

This simple one dimensional example illustrates the technique for changing the metric to obtain a fast algorithm. The 2D case is exactly similar, with the direction of the incident plane wave being rotated to correspond to the x-axis in the above algorithm.

### Scientific Background for Propagation-Backpropagation Method and Propagation-Cg Generalization

This section is by nature technical.

The Helmholtz Equation (also referred to as the “reduced wave equation” is desired to be solved exactly (i.e., without any type of linearization or perturbation assumption) in order to reconstruct certain parameters in an object. The unknown object is illuminated with some type of wave energy (whether acoustic, or electromagnetic). The wave equation that is solved is of the form:

$${∇}^{2} f + {k}_{o}^{2} ( 1 - γ ) f = 0$$

where kₒ is the wavenumber in free space. Note that Natterer uses the notation ∇²≡Δ for the Laplacian, therefore in the following, this notation will be used. The γ is the object function:

$$\hat{γ} ( r ) = 1 - \frac{{c}_{o}^{2}}{{c}^{2} ( r )} = - ( \frac{{c}_{o}^{2}}{{c}^{2} ( r )} - 1 ) = - γ ( r )$$

Where \hat{γ} is the object function definition employed by Natterer. It is the negative of the standard definition of γ as defined and used in this patent. Furthermore, in the papers included as reference, written by Natterer, and Natterer and Wubbeling, the notation f is used to represent \hat{γ}.

For purposes of this discussion we will define v_{θ} in the following manner:

$$f ≡ {e}^{{ik}_{0} θ \cdot r} ( 1 + {v}_{θ} )$$

where θ is a unit vector in R².

In other words

$$f ≡ {e}^{i {k}_{0} θ \cdot r} + {e}^{i {k}_{0} θ \cdot r} {v}_{θ}$$

so that

$${e}^{i {k}_{0} θ \cdot r}$$

is the incident plane wave and v_{θ} is

$${e}^{- i {k}_{0} θ \cdot r} {f}^{s c}$$

where f^{sc} is the scattered field.

NOTE: The definition of \hat{γ}(r) here is designed to correspond to the object function used in “A Propagation-Backpropagation Method for Ultrasound Tomography”, Frank Natterer. This definition is the negative of the standard γ used in this patent, and previous patents.

When the definition for the field f is substituted into the Helmholtz equation, the result is the equation that v_{θ} must solve:

$$Δ {v}_{j} + 2 i {k}_{o} {θ}_{j} \cdot ∇ {v}_{j} - {k}_{o}^{2} ( 1 + {v}_{j} ) \hat{y} = 0$$

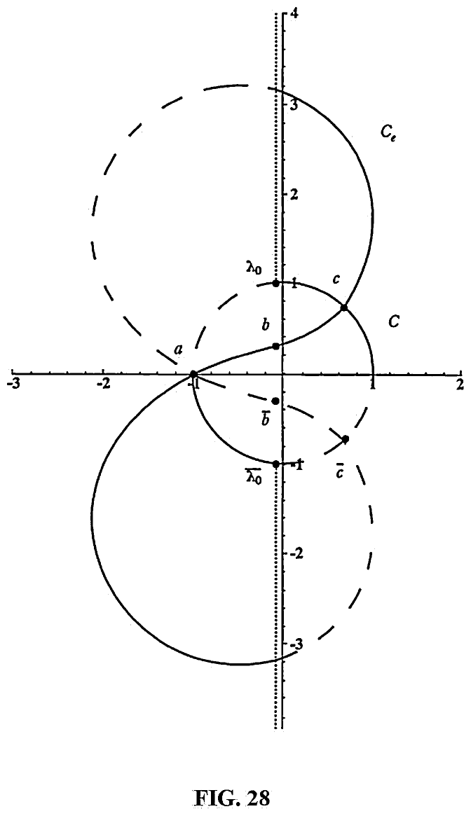

FIG. 28 shows the basic Geometry for the paper “A Propagation-Backpropagation Method for Ultrasound Tomography”, Frank Natterer, which is included herein as reference. This is referred to as FIG. 1 in this paper. The square Qⱼ is the square of sidelength 2ρ whose boundary is made up of Γⱼ (side-scatter directions),

$${Γ}_{j}^{-}$$

(Backscatter direction), and

$${Γ}_{j}^{+}$$

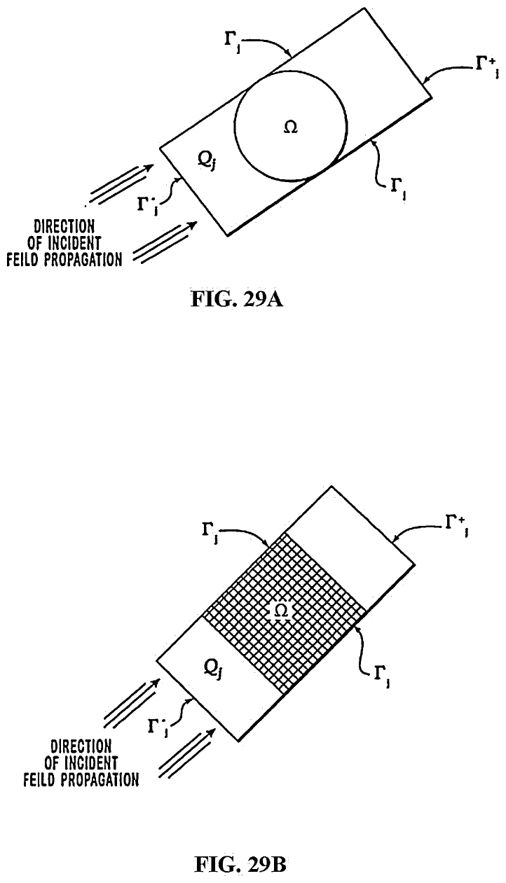

(forward scatter direction). It encompasses the region Ω, which contains the support of the object function γ. θⱼ is the direction of the incident wavefield propagation. FIGS. 29A and 29B show an elongated rectangle Qⱼ enclosing the region Ω, with boundaries Γⱼ (side-scatter directions),

$${Γ}_{j}^{-}$$

(Backscatter direction), and

$${Γ}_{j}^{+}$$

(forward scatter direction). θⱼ is the direction of the incident wavefield propagation (for FIG. 29A).The geometry of FIG. 28 shows the incident field direction θⱼ, the boundary in the backscattered direction,

$${Γ}_{j}^{-} ,$$

the boundary in the sidescattered direction, Γⱼ, as well as in the forward scattered direction

$${Γ}_{j}^{+}$$

The ultimate goal is to determine the distribution of appropriate scattering coefficients, γ, or γ, given the measured fields

$${g}_{{θ}_{j}} ≡ {g}_{j} on \partial {Q}_{j} ≡ {Γ}_{j}^{+} ⋃ {Γ}_{j} {⋃}_{j}^{-} ,$$

j=1, . . . , N_{view}, where N_{view} is the number of views. ∂Qⱼ is the boundary of Qⱼ.

This goal will be achieved by applying the Paige-Saunders Least Squares conjugate gradient algorithm to the functional which is the difference between the measured field on

$${Γ}_{j}^{+} ,$$

and the calculated scattered field on

$${Γ}_{j}^{+} .$$

The scattered field on

$${Γ}_{j}^{-} ,$$

and on the sides Γⱼ is also incorporated into the algorithm, since these values are used as boundary values in the numerical solution of the partial differential equations enumerated below.

As is well known, we are required to calculate the “derivative of vⱼ with respect to γ” in order to utilize the method of conjugate gradients. This derivative will be denoted by

$$\frac{\partial {v}_{j}}{\partial γ} ,$$

and is also referred to as the Frechet Derivative. It is the functional analysis equivalent of the Jacobian in the calculus of several variables. This “Frechet derivative” is a linear operator which acts upon object functions, δγ, and delivers up a calculated total field on Q: i.e.,

$$\frac{\partial {v}_{j}}{\partial γ} {δ}_{γ}$$

is a total calculated field. Now, in accordance with the papers by F. Natterer and F. Wubbeling included herein as references, we will also introduce the notation

$${R}_{j} ( γ ) ≡ {v}_{j} {\vert}_{{Γ}_{j}^{+}} .$$

That is, Rⱼ(γ)≡vⱼ restricted to

$${Γ}_{j}^{+} ,$$

the forward scattering part of the boundary of Q. By definition, the derivative

$$\frac{\partial {R}_{j} ( {γ}^{r} )}{\partial γ}$$

is the restriction of

$$\frac{\partial {v}_{j}}{\partial γ}$$

to the forward scattered direction:

$${( \frac{\partial {R}_{j} ( {γ}^{r} )}{\partial γ} = \frac{\partial {v}_{j}}{\partial γ} )}_{{Γ}_{j}^{+}}$$

We will calculate these operators explicitly below. To be exact, the conjugate gradient algorithm we employ requires the calculation of

$$\text{"} \frac{\partial {v}_{j}}{\partial γ}$$

acting on δγ, for specific, known δγ, ie the calculation of the function

$$\frac{\partial {v}_{j}}{\partial γ} {δ}_{γ} .$$

To make the equations easier to read, this function will be notated as ωⱼ, that is:

$${ω}_{j} ≡ \frac{\partial {v}_{j}}{\partial γ} δ γ$$

is a function representing a total field on region Q.

First, consider the forward problem in the direction j, which we have denoted (as in F. Natterer's papers) by Rⱼ: given some object function γ, which describes the distribution of parameters within the image grid Q, determine the solution vⱼ, to the following boundary value problem.

$$Δ {v}_{j} + 2 {ik}_{o} {θ}_{j} \cdot ∇ {v}_{j} - {k}_{o}^{2} ( 1 + {v}_{j} ) \hat{γ} = 0 for j = 1 , … {N}_{view}$$

subject to the conditions (from measured values gⱼ:

$${v}_{j} = {g}_{j} on {Γ}_{j} ⋃ {Γ}_{j}^{-} and \frac{\partial {v}_{j}}{\partial v} = \frac{\partial {g}_{j}}{\partial v} on {Γ}_{j}^{-}$$

Then Rⱼ(γ)≡vⱼ restricted to

$${Γ}_{j}^{+}$$

i.e.,

$${R}_{j} ( γ ) ≡ {v}_{j} {\vert}_{{Γ}_{j}^{+}} .$$

Where vⱼ is the solution to the above boundary value problem.

NOTE: The solution of this boundary value problem requires the knowledge of the total field on the sides and backscatter direction, and the normal derivative of the total field on the backscatter direction boundary

$${Γ}_{j}^{-} ,$$

by virtue of the finite difference marching method employed to solve the partial differential equation. Thus, from a physical point of view the aperture is 360°. The side scattered and back-scattered fields are both included in the solution.

The system that we require to solve for γ is a nonlinear system, of the form:

$${R}_{j} ( γ ) ≡ {g}_{j} {\vert}_{{Γ}_{j}^{+}} \qquad 1$$

Recall that gⱼ is the measured data in the direction θⱼ. Because it is a nonlinear system, it must be solved iteratively by means of the Newton-Raphson method. To this end, given a guess γ^{r}, as an approximation to the solution of (1) consider Rⱼ(γ^{r}+δγ^{r}). One can write:

$${R}_{j} ( {γ}^{r} + {δγ}^{r} ) \approx {R}_{r} ( {γ}^{r} ) + [ \frac{\partial {R}_{j}}{\partial γ} ] {δγ}^{r}$$

where

$$[ \frac{\partial {R}_{j}}{\partial γ} ]$$

is the “Jacobian” map which linearly approximates Rⱼ at yr. Note that this is a linear map:

$$[ \frac{\partial {R}_{j}}{\partial γ} ] : ( \begin{matrix}OBJECT \\ FUNCTIONS\end{matrix} ) ⇒ ( \begin{matrix}MEASURED \\ FIELDS\end{matrix} )$$

We can use this fact to obtain an explicit representation of the function

$$[ \frac{\partial {R}_{j}}{\partial γ} ] : ( \begin{matrix}OBJECT \\ FUNCTIONS\end{matrix} ) ⇒ ( \begin{matrix}MEASURED \\ FIELDS\end{matrix} )$$

Let (vⱼ+δvⱼ) be the total field resulting from applying the incident field in direction j to the object function (γ^{r}+δγ^{r}) That is, Δ(vⱼ+δvⱼ)+2ikₒθⱼ·∇(vⱼ+δvⱼ)−kₒ² (1+(vⱼ+δvⱼ)) (γ^{r}+δγ^{r})=0

Restricting (vⱼ+8vⱼ) to the forward scattering border gives the symbolic equation:

$${R}_{j} ( {γ}^{r} + {δγ}^{r} ) = ( {v}_{j} + δ {v}_{j} ) {\vert}_{{Γ}_{j}^{+}}$$

Using the fact that

$$Δ ( {v}_{j} ) + 2 {ik}_{o} {θ}_{j} \cdot ∇ ( {v}_{j} ) - {k}_{o}^{2} ( 1 + {v}_{j} ) ( {\hat{γ}}^{r} ) = 0$$

gives

$$Δ ( δ {v}_{j} ) + 2 {ik}_{o} {θ}_{j} \cdot ∇ ( δ {v}_{j} ) - {k}_{o}^{2} ( δ {v}_{j} ) ( {\hat{γ}}^{r} ) = {k}_{o}^{2} [ ( 1 + {v}_{j} ) δ {\hat{γ}}^{r} + δ {\hat{γ}}^{r} δ {v}_{j} ]$$

The last term on the right hand side contains the quadratic terms in δγ and so will be ignored, since we are interested in the linear variation of vⱼ with γ. Therefore, using the definition

$${ω}_{j} = \frac{\partial {v}_{j}}{\partial γ} δγ ,$$

which is the part of δvⱼ which is linear in δγ, it follows that ωⱼ is the solution to the following initial value problem with known, nonzero right hand side.

$$Δ ( {ω}_{j} ) + 2 {ik}_{o} {θ}_{j} ∇ ( {ω}_{j} ) - {k}_{o}^{2} {ω}_{j} {\hat{γ}}^{r} = {k}_{o}^{2} ( 1 + {v}_{j} ) \partial {\hat{γ}}^{r}$$

with the boundary values:

$${ω}_{j} = 0 on {Γ}_{j} ⋃ {Γ}_{j}^{-} ,$$

and initial value

$$\frac{\partial {ω}_{j}}{\partial v} = 0$$

on

$${Γ}_{j}^{-}$$

The boundary values follow from the following considerations: Since ωⱼ is the linear part of the total variation of vⱼ it follows that vⱼ+δvⱼ≡vⱼ+ωⱼ+higher order terms.

It follows that, at the boundaries: vⱼ+δvⱼ=gⱼ, but vⱼ=gⱼ at the boundaries, by definition of vⱼ, therefore δvⱼ=0 at the boundaries, as stated.

For purposes of the Paige-Saunders method, or for direct application as backpropagation, it is important to determine a similar explicit representation for the Hermitian adjoint or Hermitian transpose of the “Jacobian map”

$$[ \frac{\partial {R}_{j}}{\partial γ} ] :$$

Again, it is the action of the Hermitian transpose on a given function which is actually used by the conjugate gradient type algorithms.

$${[ \frac{\partial {v}_{j}}{\partial γ} ]}^{H} : ( \begin{matrix}TOTAL \\ FIELDS\end{matrix} ) ⇒ ( \begin{matrix}OBJECT \\ FUNCTIONS\end{matrix} )$$

Using the definition of Rⱼ as the restriction of the total field to

$${Γ}_{j}^{+} ,$$

it follows that the Hermitian conjugate of

$$[ \frac{\partial {R}_{j}}{\partial γ} ]$$

is a linear map:

$${[ \frac{\partial {R}_{j}}{\partial γ} ]}^{H} : ( \begin{matrix}MEASURED \\ FIELDS ON {Γ}_{j}^{+}\end{matrix} ) ⇒ ( \begin{matrix}OBJECT \\ FUNCTIONS\end{matrix} )$$

The calculation of the action of

$${[ \frac{\partial {R}_{j}}{\partial γ} ]}^{H}$$

on a given measured field on

$${Γ}_{j}^{+}$$

is a somewhat tedious process carried out in Natterer “A Propagation-Backpropagation Method for Ultrasound Tomography” [included in this patent as reference]. The final result is: Given gᵢ, a function on

$${Γ}_{j}^{+} ,$$

the action of

$${[ \frac{\partial {R}_{j}}{\partial γ} ]}^{H}$$

on gⱼ, is

$${[ \frac{\partial {R}_{j}}{\partial γ} ]}_{j}^{H} ( {g}_{j} ) ≡ {k}_{o}^{2} ( 1 + {\overline{v}}_{j} ) z$$

where z is the solution to the initial boundary value problem:

$$Δ z + 2 {ik}_{o} {θ}_{j} \cdot ∇ z - {k}_{o}^{2} \overline{γ} z = 0$$

(where γ̅(r) denotes the complex conjugate of \hat{γ}) with boundary values:
   z=0 on

$${Γ}_{j} ⋃ {Γ}_{j}^{+} ,$$

and initial value

$$\frac{\partial z}{\partial v} = {g}_{j}$$

on

$${Γ}_{j}^{+}$$

NOTE that gⱼ is used only on the forward scattering border

$${Γ}_{j}^{+} ,$$

which is as it should be since this is the only place that the function gⱼ is defined. Note that the gⱼ is “back-propagated” back across the region Q, in order to obtain the function z, which is then used to obtain the function

$${[ \frac{\partial {R}_{j}}{\partial γ} ]}^{H} {g}_{j}$$

which is defined on all of Q.

### Newton-Raphson Method Applied to Inversion

Now consider the system:

$${g}_{j} = {R}_{j} ( {γ}^{r} + δ {γ}^{r} ) \approx {R}_{j} ( {γ}^{r} ) + [ \frac{\partial {R}_{j}}{\partial γ} ] δ {γ}^{r}$$

Rewriting this gives the following linear system which must be solved in order to obtain δγ.

$$[ \frac{\partial {R}_{j}}{\partial γ} ] ( δ {γ}^{r} ) = {g}_{j} - {R}_{j} ( {γ}^{r} )$$

The vastly underdetermined form of this system leads one to define the function d such that the following equation holds:

$$δ {γ}^{r} ≡ {[ \frac{\partial {R}_{j}}{\partial γ} ]}^{H}$$

Then the corresponding system or is:

$$[ {[ \frac{\partial {R}_{j}}{\partial γ} ] [ \frac{\partial {R}_{j}}{\partial γ} ]}^{H} ] d = {g}_{j} - {R}_{j} ( {γ}^{r} )$$

and the expression for δγ^{r} is given by:

$$δ {γ}^{r} ≡ {[ \frac{\partial {R}_{j}}{\partial γ} ]}^{H} d = {{[ \frac{\partial {R}_{j}}{\partial γ} ]}^{H} [ {[ \frac{\partial {R}_{j}}{\partial γ} ] [ \frac{\partial {R}_{j}}{\partial γ} ]}^{H} ]}^{- 1} ( {g}_{j} - {R}_{j} ( {γ}^{r} ) )$$

Now, the efficient method for the determination of δγ^{r}, will involve some form of approximation:

$${C}_{j} \approx {[ {[ \frac{\partial {R}_{j}}{\partial γ} ] [ \frac{\partial {R}_{j}}{\partial γ} ]}^{H} ]}^{- 1}$$

For example Cⱼ=Identity has been shown to work well (This is the approach taken by Natterer in “A Propagation-Backpropagation Method for Ultrasound Tomography”. Other choices include Cⱼ=some diagonal matrix. One could also use Paige-Saunders Least Squares Conjugate Gradient method to find the minimum norm solution to:

$$[ \frac{\partial {R}_{j}}{\partial γ} ] δ {γ}^{r} = {g}_{j} - {R}_{j} ( {γ}^{r} )$$

In any case, once the update δγ^{r} has been found. It is added to the previous guess with some multiplicative factor μ≤1 to obtain the new estimate for the γ.

$${γ}^{′ + 1} = {γ}^{′} + μ ( {δγ}^{′} )$$

This process is repeated for j=1, . . . N_{view}. This approach differs significantly from the approach described earlier in that a vastly underdetermined problem is solved for each direction, as opposed to solving an overdetermined problem for the update δγ.

Note that the forward problem can be updated after 1, 2, or any finite number of directions have been carried out. The tradeoffs are that it is much more difficult to calculate the forward problem each time for each direction. However, the speed up in the convergence may make it worth the computational effort.

Scientific Background and Detailed Description for Brightness Functional Approach to Phase Aberration Correction with Conjugate Gradient Methods

The scientific background to the phase aberration correction based upon the brightness functional is simply that the L₂ norm (functional) of the B-scan image intensity is maximized when the phase shifts (time delays) are such that the image is maximally focused [L. Nock and G. E. Trahey, “Phase aberration correction in medical ultrasound using speckle brightness as a quality factor,” Journ. Acoustical Society of America, 1989, 85, 1819-1833, herein included as reference].

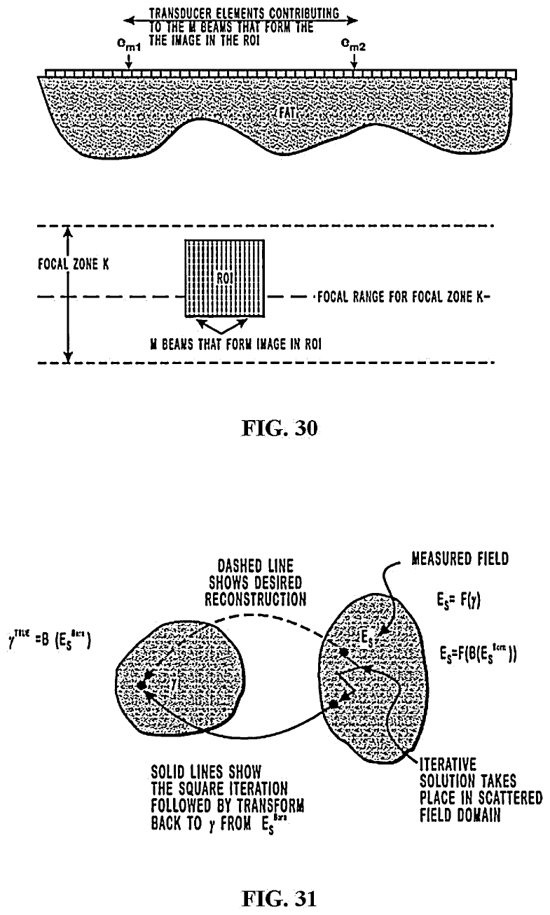

FIG. 30 shows the geometry of a linear B-scan acoustic transducer array illuminating an anatomical region through an aberrating layer of fat. We develop the algorithm here for the linear array for simplicity. Modification for convex, sector scanning arrays etc. is trivial. A region of interest (ROI), selected by the user, is also shown. The image in the ROI is formed by M beams produced by the beamformer hardware. The transducer elements that contribute to the formation of the M beams in the ROI are denoted eₘ₁ to eₘ₂.

The goal of the algorithm is to focus the image in the ROI by finding a set of time delays applied to the signals from each transducer element eₘ₁ to eₘ₂ such that the brightness functional in the ROI (square of the L2 norm of the image intensity, B(t), over the ROI) is maximized:

$$B ( t ) = {\sum }_{\{ n , m \} ∈ ROl} {I}_{n , m}^{2} , maximi {z}_{t} e \{ B ( t ) \}$$

where I_{n,m} is the image intensity at pixel (n,m) and the vector t, tₘ: m=m₁, . . . , m₂ is the vector of transducer element time delays. Our algorithm applies gradient based optimization methods (steepest descent, Fletcher-Reeves conjugate gradients, Ribere Polak conjugate gradients, etc.) to solve the maximization problem.

Clinical B-scanners operate by breaking the image up in range into a number of focal zones. The receiver beamformer then focuses the transmitter and receiver at a focal range equal to the center of the focal zone. Thus, each beam (laterally scanned) has one delay set (one delay for ech element contributing to the beam) over the focal zone range. If the ROI is contained entirely within one focal zone (as in FIG. 30), then the delay perturbations for focusing need to be added to only one beamformer delay set per beam. In the case that the ROI overlaps two of more focal zones, the time delay perturbations must be added to the beam delay sets for each focal zone.

The only hardware needed to implement this phase aberration correction algorithm is a B-scanner with a computer interface, allowing the image to be read from the B-scanner into the computer memory and allowing the beamformer hardware delays in the B-scanner to be reset from the computer.

Nearly all ultrasound clinical scanners use and display the envelope of the RF beam signals as the image intensity. This is done by low pass filtering the modulus of the analytic RF signal or, in some low cost systems, by low pass filtering the rectificated video. The enveloping process is often followed by further processing that includes time variable bandwidth filtering to suppress noise and by logarithmic compression to extend dynamic range. We emphasise that the proper use of the brightness phase aberration correction algorithm must de-emphasize the amount of, or eliminate entirely, logarithmic compression, else logarithmic emphasized excess brightness in the side lobes of the point response function will destroy the attempt to focus the image by maximizing the strength of the central lobe of the point response function of the image.

The following steps outline the algorithm for brightness based phase-aberration correction.

Choose δt=the time perturbation to be added to a selected element delay for derivative calculation.

Choose line search time increment, t, and number of line search steps, Nₛ.
1. Load the beamformer with precalculated delays based on 1540 m/s tissue average speed.
2. Acquire the initial image from the b-scanner.
3. Select the ROI (region of interest) comprising one or more receiver and transmit and one or more beam locations and focal ranges.

This selection determines a sub set of transducer elements that are used in the image formation of this ROI, eₘ₁, . . . , eₘ₂ where m1 is the first element and m₂ is the last. e.g., m₁, m₂∈[1, . . . , Nₜᵣₐₙ] for an Nₜᵣₐₙ element array.

Set itertype=‘SD’, ‘FR’, ‘RP’ or ‘RPP’ for steepest descents, Fletcher-Reeves, Ribere-Polack, or Ribere-Polak with the Powel modification.

Set hₘ=0, m=m₁, . . . ,m₂, set pₘ=0, m=m₁, . . . ,m₂.

Set r0=1.

Set tₘ=0, m=m₁, . . . , m₂ the element time delay perturbation vector.
4. For l=1, . . . .
5. Compute b₀=sum of squares of the image intensity on the ROI.

If l=0, bᵢₙᵢₜᵢₐₗ=b₀.
6. For m=m₁, . . . m₂
6.a. Calculate a new delay set with δt added to all delays for which element eₘ is the transducer element used.
6.b Load the new delay set into the beamformer.
6.c Acquire the new image from the B-scanner.
6.d Compute bₘ=sum of squares of the image intensity on the ROI.
6.e gₘ=(bₘ−b₀)/δt.
6.f Next m.
7. If (itertype=‘SD’) set pₘ=gₘ, m=m₁, . . . ,m₂, go to 13.
8. Compute

$${r}_{1} = {\sum }_{m = {m}_{1}}^{{m}_{2}} {g}_{m}^{2}$$

9. If(itertype=‘FR’) β=r₁/r₀.
10. If(itertype=‘RP’)

$$β = {\sum }_{m = {m}_{1}}^{{m}_{2}} {g}_{m} ( {g}_{m} - {h}_{m} ) / {r}_{0}$$

11. If (itertype=‘RPP’) then, If β<0, β→0.
12. Update the search direction and save the gradient in vector h:

$${p}_{m} \to {g}_{m} + β {p}_{m} , m = {m}_{1} , … , {m}_{2}$$

$${h}_{m} \to {g}_{m} , m = {m}_{1} , … , {m}_{2}$$

$${r}_{0} \to {r}_{1}$$

13. Compute

$$M = {max}_{m = {m}_{1} … , {m}_{2}} \vert {p}_{m} \vert .$$

14. Line search by Trial and Error. For n=1, . . . , Nₛ:
14.a fₘ=tₘ+nδσpₘ/M, m=m₁, . . . , m₂
(Note that this formula ensures that the maximum time perturbation is Nₛ.δτ.
14.b Compute the new beamformer delay set with element time delay perturbation vector f.
14.c Acquire the new B-scan image and compute the brightness bₙ over the ROI.
14.d Next n.
15. Determine n for which bₙ is maximum, then set: tₘ→tₘ+50nδτpₘ/M, m=m₁, . . . , m₂.

Steps 14 thru 15 can easily be replaced by a gradient, or quadratic, or Fibonacci search, or other line search for efficiency. The above trial and error method is included for concreteness only)
16. Load new delays into beamformer with vector t of element time delay perturbations added.
17. Display B-scan image.
18. Check convergence criteria such as the percent change in brightness functional is less than some small and arbitrary number such as 5%. Do another gradient convergence step? If yes, increase 1 by 1, (go to 5).

Clearly computing the gradient by perturbation of each time delay in sequence is one of many ways and is straightforward. It is possible to use a basis set for the gradient based upon the singular value decomposition of the Hessian of the Brightness functional (which is closely related to the Jacobian of the Brightness vector—ie the vector whose modulus squared is the Brightness functional)

The number of significant singular vectors will generally be somewhat less than m₂−m₁, so that using the singular vectors as a basis for finding the gradient will in general be much more efficient.

Scientific Background for Imaging with Diffusion Equation Models

Electric Conductivity Imaging by Frequency Domain, Nonlinear Inversion (the method we use for wave equation inversion, now modified for the Diffusion Equation)

We start with the receiver and media diffusion equations, modified to eliminate the Electric field E, internal to the image grid, which then becomes a non-linear expression for the conductivity (or its reciprocal, resistivity) in terms of the incident field and the measured field at a fixed frequency ω. Define the residual field R (note the frequency, source and receiver indices of R are suppressed, but under stood to be active) by the standard formula:

$$R = {E}_{m} - {E}_{b} + D [ ∖ γ ∖ ] ( [ I - {C [ ∖ γ ∖ ]}^{- 1} {E}_{b} = 0$$

Here, Eₘ(r) is the measured electric field, Eₛ=Eₘ−E_{b} is the measured scattered electric field, and E_{b}(r) is the incident field or response in the (homogeneous) background medium.

It should be noted that the definitions provided in this section are isomorphic to the wave equation definitions given in the previous examples and only the Green's functions have changed (but these are available from “Morse and Feschback, Methods of Theoretical Physics, Vols. 1, and 2, McGraw-Hill.”).

This provides the correct value for the measured field Electric field Eₘ when substituting the correct value for γ and E_{b} and on setting R=0. We solve for γ by finding the γ that minimizes R by minimizing the objective functional

$$F ( γ ) = ( \frac{1}{2} ) {‖ E - E b - {D [ ∖ γ ∖ ] [ I - C [ ∖ γ ∖ ] ]}^{- 1} Eb ‖}^{2} = ( \frac{1}{2} ) {‖ R ‖}^{2}$$

In general, we can define the norm of the residual vector R to be general enough to weight each frequency component of F(γ) to increase the convergence rate in some cases:

$$F ( γ ) = ( \frac{1}{2} ) {‖ R ‖}^{2} = {\sum }_{frequency ω} {\sum }_{sources s} {\sum }_{receivers m} {\vert {W}_{ω} {R}_{ω s m} \vert}^{2}$$

Define the Jacobian of the residual R with respect to γ by

$$( \partial / \partial γ ) R = - {D ( I - [ ∖ γ ∖ ] C )}^{- 1} [ ∖ E ∖ ] ]$$

Then the Gauss-Newton algorithm for finding the γ that minimizes F(γ) is isomorphic to the earlier wave equation case, and is given by:
(a) Select an initial guess γ⁽ⁿ⁾. Set n=0.
(b) Solve the forward problem for E⁽ⁿ⁾ by us of the biconjugate gradient (BCG) or stabilized biconjugate gradient (BiSTAB) algorithms.

$${E}^{( n )} = ( {[ I - C [ ∖ {γ}^{( n ) ∖} ] ]}^{- 1} {E}_{b}$$

(c) compute the receiver residuals

$${R}^{( n )} = {E}_{m} - {E}_{b} + D [ ∖ γ {∖}^{( n )} ] ( {[ I - C [ ∖ {γ}^{∖ ( n )} ] ]}^{- 1} {E}_{b}$$

(d) For some small number ϵ the size of the noise to signal ratio,
if

$$‖ {R}^{( n )} ‖ / ‖ {E}_{m} ‖ < ϵ ,$$

then stop. Else
(e) Minimize

$${‖ ( \partial / \partial γ ) {R}^{( n )} δ {γ}^{( n )} + {R}^{( n )} ‖}^{2}$$

for δγ⁽ⁿ⁾ by using the conjugate gradient algorithm.
(f) Update γ by γ⁽ⁿ⁺¹⁾=γ⁽ⁿ⁾+δγ⁽ⁿ⁾
(g) Set n=n+1, go to (b).

We note that for many problems that the Jacobian can be approximated by the first two terms of the Neumann series

$$( \partial / \partial γ ) R = - D ( I + [ \vert γ \vert C ) [ ∖ E ∖ ] ]$$

This is like a Born approximation to the Jacobian, but it is not the Born approximation to the scattering equations. We find that this Jacobian has a larger radius of convergence than the Born approximation itself.

This method tends to be self regularizing if the iterations are halted when the changes in

$$‖ {R}^{( n )} ‖ / ‖ {E}_{m} ‖$$

become small relative their early changes. Other regularization methods may also be used such as the “squaring” method described below:

### Scientific Background on Squaring an Overdetermined System to Apply Bistab:

The basic algorithm for reconstruction of γ is reviewed below: We start with the receiver and media propagation equations, modified to eliminate E, which then becomes a non linear expression for the object function in terms of the incident field and the measured field at a fixed frequency ω. Define the residual field R (note the frequency, source and receiver indices of R are suppressed) by

$${R - ( {E}_{m} - {E}_{b} ) - D [ ∖ γ ∖ ] ( [ I - C [ ∖ γ ∖ ] ] ]}^{- 1} {E}_{b} = 0$$

Here, Eₘ(r) is the measured field, Eₛ=Eₘ−E_{b} is the measured scattered field, and E_{b}(r) is the incident field or response in the (homogeneous) background medium. [\γ\] is the diagonal matrix formed from the vector γ.

This provides the correct value for the measured field Eₘ when substituting the correct value for γ and E_{b} and on setting R=0. We solve for γ by finding the γ that minimizes R by minimizing the objective functional

$${{F ( γ ) = ( \frac{1}{2} ) ‖ E - E b - D [ ∖ γ ∖ ] ] [ I - C [ ∖ γ ∖ ] ]}^{- 1} Eb ‖}^{2} = ( \frac{1}{2} ) {‖ R ‖}^{2}$$

In general, we can define the norm of R to be general enough to weight each frequency component of F(γ) to increase the convergence rate in some cases:

$$F ( γ ) = ( \frac{1}{2} ) {‖ R ‖}^{2} = {\sum }_{frequency ω} {\sum }_{sources s} {\sum }_{receivers m} {\vert {W}_{ω} {R}_{ω s m} \vert}^{2}$$

Define the Jacobian of the residual R with respect to γ by

$$( \partial / \partial γ ) R = - D ( I - [ \vert γ \vert C ) [ [ ∖ E ∖ ] ]$$

Then The Gauss-Newton algorithm for finding the γ that minimizes F(γ) is given by:
(a) Select an initial guess γ⁽ⁿ⁾. Set n=0.
(b) Solve the forward problem for E⁽ⁿ⁾ by us of the biconjugate gradient (BCG) or stabilized biconjugate gradient (BiSTAB) algorithms.

$${E}^{( n )} = ( {[ I - C [ ∖ {γ}^{( n )} ] ]}^{- 1} {E}_{b}$$

(c) compute the receiver residuals

$${R}^{( n )} = {E}_{m} - {E}_{b} + D [ ∖ {γ}^{( n )} ∖ ] ( {[ I - C [ ∖ {γ}^{( n )} ∖ ] ]}^{- 1} {E}_{b}$$

(d) For some small number
the size of the noise to signal ratio,
if

$$‖ {R}^{( n )} ‖ / ‖ {E}_{m} < ϵ$$

then stop. Else
(e) Minimize

$${‖ ( \partial / \partial γ ) {R}^{( n )} δ {γ}^{( n )} + {R}^{( n )} ‖}^{2}$$

for δγ⁽ⁿ⁾ by using the conjugate gradient algorithm.
(f) Update γ by γ⁽ⁿ⁺¹⁾=γ⁽ⁿ⁾+δγ⁽ⁿ⁾
(g) Set n=n+1, go to (b).

We note that for many problems that the Jacobian can be approximated by the first two terms of the Neumann series

$$( \partial / \partial γ ) R = - D ) I + [ ∖ γ ∖ ] C ) [ ∖ E ∖ ] ]$$

This is like a Born approximation to the Jacobian, but it is not the Born approximation to the scattering equations. We have found that this algorithm with approximate Jacobian has a larger radius of convergence than the Born approximation per se.

This method tends to be self regularizing if the iterations are halted when the changes in

$$‖ {R}^{( n )} ‖ / ‖ {E}_{m} ‖$$

become small relative to their early changes. Other regularization methods may also be used such as Singular Value Decomposition based methods. The difficulty with this algorithm is that it is based upon using some form of general conjugate gradients to solve an overdetermined system. The way to avoid this bottleneck is to create a square system from the overdetermined system and use BiSTAB on the square system. This can be done in several ways when the system is linear.

Investigation of a new inversion method based on finding square Jacobian.

It is known that solving the rectangular, over determined, linear system Ax=b can be accomplished by multiplying both sides of the equation by the adjoint AG, thereby giving the square system (A [symbol] A)x=A [symbol] b. This s stem is called the normal system and the corresponding equations the normal equations. The solution is the least squares solution to the original over determined system. However, the new system has singular values that are the square of the singular values if the original system (i.e., AGA is ill conditioned). This means the conjugate gradient (CG), one the most efficient of all methods, converges slowly in solving the normal equations. Although CG methods for solving the over determined system directly exist, they essential form products (A [symbol] ,A) implicitly. Thus, the speed advantages of CG are compromised. The system Ax=b in our example represents the system

$$( \partial / \partial γ ) {R}^{( n )} δ {γ}^{( n )} + {R}^{( n )} = 0$$

i.e., equation 4.2; 18(e), where (∂/∂γ)R⁽ⁿ⁾ is the Jacobian J⁽ⁿ⁾.

We note J⁽ⁿ⁾ [symbol] J⁽ⁿ⁾ is square but ill conditioned. In our example Ax=b, let x=By and let AB be square. Then on substitution, ABy=b. Now, y can be solved by BiCG and then x is found by a simple multiplication by B. Further, suppose that B can be chosen so that y≈b, then AB≈I, where I is the square identity matrix. In this case, solving ABy=b is very well conditioned and BiCG should converge in only a few iterations. We next generalize A and B to be nonlinear operators (not matrices). Then the Jacobian of the operator AB is square. We also may choose B to be an improved generalized Born approximation, the Born approximation, or backpropagation. These methods are discussed in the detailed description section below in conjunction with FIGS. 28 and 29.

### General Discussion of Conditioning

Suppose that the goal is to find

$${min}_{t} {‖ At - r ‖}_{2} ,$$

where A is m by n matrix (m<n), t is an n-dimensional vector, and r is m-dimensional “residual” vector. By making a change of variables Bs=t, to s, where B is n by in, the system to be solved is now

$$ABs = r$$

and AB is a square system. Therefore the BiConjugate gradient method or BiCGsquared algorithm can be applied to this system. BiCG squared has much better convergence behaviour than standard conjugate gradient methods for non-square systems. Consequently the convergence to a solution ŝ will generally be much quicker, whereupon \hat{t}≡Bŝ computes the answer to the original problem. Due to the position of the conditioning matrix B, after the original coefficient matrix A, this procedure is sometimes referred to as ‘postconditioning’

### Two Special Cases:

1. B=A^{T}, in which case s is the minimum norm solution to an underdetermined problem

$$A ( γ ) = {⊕}_{ω ϕ} {{G}_{ω}^{SC} ( I - {G}_{ω} γ )}^{- 1} {f}_{ω ϕ}^{i n c} ,$$

Where ⊕_{ωϕ} indicates the direct sum in the sense of vector spaces over all frequencies, ie concatenation.

In fact, for operators A₁, which act on vectors vⱼ: Aⱼvⱼ, the operator

$$[ \begin{matrix} \\ ⊕ {A}_{j} \\ j\end{matrix} ] = [ \begin{matrix}{A}_{1} & 0 & 0 & 0 \\ 0 & {A}_{2} & 0 & 0 \\ 0 & 0 & ⋱ & 0 \\ 0 & 0 & 0 & {A}_{N}\end{matrix} ]$$

acts on the total vector

[⊕ⱼvⱼ] to yield: [⊕ⱼAⱼ][⊕ⱼvⱼ],
where

$$[ {⊕}_{j} {A}_{j} ] [ {⊕}_{j} {v}_{j} ] = [ \begin{matrix}{A}_{1} & 0 & 0 & 0 \\ 0 & {A}_{2} & 0 & 0 \\ 0 & 0 & ⋱ & 0 \\ 0 & 0 & 0 & {A}_{N}\end{matrix} ] [ \begin{matrix}{v}_{1} \\ {v}_{2} \\ ⋮ \\ {v}_{N}\end{matrix} ] = [ \begin{matrix}{A}_{1} {v}_{1} \\ {A}_{2} {v}_{2} \\ ⋮ \\ {A}_{N} {v}_{N}\end{matrix} ]$$

Of course, this is a nonlinear operator, and the corresponding Jacobian is

$$\frac{\partial A B}{g} ≡ \frac{\partial A ( γ )}{\partial γ} \frac{\partial B}{\partial g} = ( \begin{matrix}b y \\ linearity\end{matrix} ) = \frac{\partial A ( γ )}{\partial γ} B \qquad 1$$

This Jacobian is evaluated at some iterated guess, and is an object that is used in the BiCG minimization routine. B in this case is the Born or Rytov reconstruction.

The general procedure for the solution of the Jacobian equation:

$$\frac{\partial A ( γ )}{\partial γ} B δ {g}^{SC} = - {r}^{( n )}$$

then involves the application of said Jacobian (1), and its conjugate transpose:

$${B}^{H} \frac{\partial {A ( γ )}^{H}}{\partial γ} {r}^{( n )}$$

In the above formula g^{sc} is the scattered field to which the Born reconstruction procedure is applied to yield γ,

$$γ = B {g}^{s c}$$

where the standard Born approximation is utilized:

$${g}^{s c} ( x ) = {\int }_{Ω} G ( x , {x}^{′} ) r ( {x}^{′} ) {f}^{i n c} ( {x}^{′} ) {dx}^{′}$$

which upon discretization reads:

$${g}^{s c} = G [ {f}^{i n c} ] γ$$

where the standard notation for the matrix whose diagonal elements are the components of the vector f^{inc} has been used:

$$[ {f}^{i n c} ] ≡ [ \begin{matrix}{f}_{1}^{inc} & & & \\ & {f}_{2}^{inc} & & \\ & & ⋱ & \\ & & & {f}_{N}^{inc}\end{matrix} ]$$

Consequently the operator B can be written explicitly:

$$B = [ 1 / {f}^{i n c} ] {G}^{- 1}$$

although it is important to realize that the actual inversion is never carried out: rather, the standard Born reconstruction based upon the Fourier Diffraction Theorem is used, for computational efficiency.

### Scientific Background for Calibration Techniques

The idea behind the use of nonlinear optimization for calibration is to collect scattering data from several known phantoms. A computational model of the scattering process, functionally dependent on the unknown calibration parameters, is then varied with respect to these parameters until a match with the data is obtained. If the objects are sufficiently well known and sufficient in number and location, then the resulting match should provide an accurate estimate of the unknown calibration parameters.

### Generic Gauss-Newton Minimization Algorithm for Calibration:

Suppose that a dataset, f^{s}_{θϕγ}, fⁱ_{θϕ}, of scattered and incident fields is measured for several view angles q, receiver positions f, and known test phantoms, g. A typical phantom used in practice is an agar cylinder. Note that the incident measurements fⁱ_{θϕ} are not dependent on the test object.

Assume further that a computational model for the scattering problem exists which predicts the measured data, given such calibration parameters as position, radius, and speed of sound in the agar cylinder, and position and orientation of the receiver array (e.g., the orientation defined by Euler angles with respect to a selected fixed coordinate system). This might be a general forward solver such as an integral equation solution or an FDTD algorithm. A simpler, less computationally involved method, in the event that the test phantoms are cylinders or collections of cylinders, is the use of the Bessel series analytic solution. Let this computational solution be denoted:

$${ϕ}_{θφ r}^{s} ( C ) , {ϕ}_{θ ϕ} i ( C )$$

for the M scattering parameters in the vector C

$$C ≡ ( \begin{matrix}{C}_{1} \\ {C}_{2} \\ ⋮ \\ {C}_{M}\end{matrix} )$$

The optimization approach is to minimize the L2 norm of the mismatch between theoretically predicted and experimentally measured fields by adjusting the values of the scattering parameters in vector C:

$${min}_{ε} F = {min}_{ε} [ {\vert {φ}_{θ ϕ γ}^{s} ( c ) - {f}_{θ ϕ γ}^{s} \vert}^{2} + {\vert {φ}_{θ ϕ}^{i} ( c ) - {f}_{θ ϕ}^{i} \vert}^{2} ]$$

It is also possible and desirable to enforce bounds on the scattering parameters:

$${L}_{j} ≦ {c}_{j} ≦ {U}_{j} , j = 1 , … , M$$

Minimization of the functional is computed by first explicitly deriving the elements of the Jacobian for the scattering equations:

$$\frac{\partial {f}_{θ ϕ γ}^{s} ( c )}{\partial {c}_{m}} \frac{\partial {f}_{θ ϕ}^{i} ( c )}{\partial {c}_{m}}$$

The Gauss-Newton method for the minimization of the functional

$$F ≡ [ {\vert {φ}_{θ ϕ y}^{s} ( c ) - {f}_{θ ϕ y}^{s} \vert}^{2} + {\vert {φ}_{θ ϕ}^{i} ( c ) - {f}_{θ ϕ}^{i} \vert}^{2} ]$$

is the following
STEP 1: Choose an initial guess for the parameter vector, Cₒ and put n=0.
STEP 2: Form the residual vectors

$${r}_{n}^{s} = {ϕ}_{n}^{s} - {f}_{n}^{s} , {r}_{n}^{i} = {ϕ}_{n}^{i} - {f}_{n}^{i}$$

where the vectors used in the definition of rⱼ are given by:

$${ϕ}_{n}^{s} = ( \begin{matrix}⋮ \\ {φ}_{θϕ y}^{s} ( {c}_{n} ) \\ ⋮\end{matrix} ) {f}_{n}^{s} = ( \begin{matrix}⋮ \\ {f}_{θϕ y}^{s} ( {c}_{n} ) \\ ⋮\end{matrix} )$$

STEP 3: Form the Jacobian matrices:

$$[ \begin{matrix}\frac{\partial {ϕ}_{θϕ y}^{s} ( {c}_{n} )}{\partial {c}_{m}} \\ \frac{\partial {ϕ}_{θϕ}^{i} ( {c}_{n} )}{\partial {c}_{m}}\end{matrix} ] = [ \begin{matrix}\frac{\partial {ϕ}_{n}^{s} ( {c}_{n} )}{\partial {c}_{m}} \\ \frac{\partial {ϕ}_{n}^{i} ( {c}_{n} )}{\partial {c}_{m}}\end{matrix} ] = [ \begin{matrix}J {ϕ}_{n}^{s} ( {c}_{n} ) \\ J {ϕ}_{n}^{i} ( {c}_{n} )\end{matrix} ]$$

and solve the system:

$$[ \begin{matrix}J {ϕ}_{n}^{s} ( c ) \\ J {ϕ}_{n}^{i} ( c )\end{matrix} ] {δC}_{n} = - [ \begin{matrix}{r}_{n}^{s} \\ {r}_{n}^{i}\end{matrix} ]$$

STEP 4: Update the parameter values:

$${C}_{n + 1} = {C}_{n} + δ {C}_{n}$$

and update n: n←n+1, and go to STEP 1 if a convergence criterion has not been met.

Modification of this algorithm to incorporate equality constraints can be achieved by the method of Lagrange multipliers.

Inequality constraints can be included by the use of slack variables, Sequential Quadratic Programming, Projected Gradient, Augmented Lagrangian or Generalized Reduced Gradient methods. Of course Penalty function methods can also be employed in theory, but straightforward application of this procedure is generally ill-posed in the numerical sense. Enforcement of these constraints tends to be very important for the present problem. For instance, receiver positions might be constrained to movements less than the assumed precision in their physical measurement (these physical measurements determine the starting values, c₀).

### Modifications

Perhaps the most essential modification to the basic Gauss-Newton algorithm above, for the present problem, is the incorporation of rescaling and truncation operators. An example of rescaling would be the case where one of the unknowns is an angle, a, (linear array angle offset).

In order to give this unknown angle the same weight as an unknown distance, we solve for δα′=rδα where r is a relevant distance which may be a function of receiver position. A need for truncation is illustrated by the fact that multiple test phantoms are needed to fully test the receiver transducer pattern. For a given phantom, only a certain range of receiver pattern parameters are tested. Also, the sensitivity of each receiver position to a given pattern parameter can be estimated geometrically. This information can be incorporated into the Jacobian with a combination of rescaling (emphasizing some receiver values and deemphasizing others) and truncation operators (removal of data not affected by a given parameter). Rescaling and truncation improve the conditioning of the Jacobian and avoid the emphasis of noise in data not relevant to a given parameter.

### Description of Calibration Procedure for Acoustic Imaging

This example gives a specific example of how the calibration procedure is employed for a particularly simple system as employed at TechniScan Inc. This example can easily be applied to electromagnetic waves with the appropriate changes in the notation.

In order to obtain a well calibrated inverse scattering image, the acoustic pressure field due to the transmitter and the sensitivity field of the receiver (equal to the pressure field produced by the receiver acting as a transmitter) must be known. Presently available transducers (PZT and PVDF) suffer from piezoelectric parameter variations, preventing the construction of a transducer with a field pattern prescribed to within better than 10%. Thus, transducer field measurements are needed to calibrate the transducer fields prior to (or concurrent with) optimization for the target acoustic parameters.

The inverse scattering algorithm also requires accurate measurement of geometrical parameters such as the physical location of the transmitter and receiver transducers relative to the region to be imaged. As a concrete example, suppose that multiple view data for the imaging of an object is obtained by a single receiver mechanically scanned to form a linear receiver array, a fixed transmitter location, and a rotated object holder: See FIG. 28 for a picture of the relevant geometry.

In FIG. 28, the object to be imaged is assumed to be fixed in the primed coordinates. The unprimed coordinates are defined by segment l connecting the transmitter center to the rotation axis of the object. dₜ is the unknown length of segment l, bₜ is the unknown transmitter misalignment angle, qₘ, m=1, . . . , M is the rotation angle of the object, dᵣ is the unknown distance from the rotation center to the receiver scan line (in the direction of segment l), a is the unknown misalignment angle of the receiver scan line with respect to the normal to l, bᵣ is the unknown misalignment angle of the receiver with respect to the scan line, D is the known receiver scan increment, yₙ, n=−N, . . . , N is the distance from the intersection of segment l with the receiver scan line to the nth receiver position which is known if y₀=yₛ and D are known. We assume that the rotation increment, d_{q}, is known. The exact placement of the object to be imaged in the primed coordinate system is not needed since its absolute shift and rotation will be imaged. The 6 geometrical unknowns are thus: dₜ, dᵣ, bₜ, bᵣ, a, yₛ. In this 2D example, the third dimension is ignored. In a real experiment, displacements and angle offsets in this dimension are also unknown. However, a basic assumption of our present approach is that, even if fields with 3D variation are produced by the transmitter and receiver, if the object is 2D (no variation in the third dimension) then there exist 2D transducers which will produce the same eikonal (total field measurement divided by the incident field measurement) data.

### Calibration by Optimization

In order to determine the transducer fields and geometry of the experiment, optimization techniques are used. Consider the case where the transducers fields are known and it is desired to calibrate for the geometry. Assume that a calibration phantom consisting of a saline filled plastic circular cylinder is placed in the sample holder. Assume that the thickness of the plastic is known but the exact speed of sound in the saline is unknown. Assume also that the cylinder is centered precisely on the rotation axis so that only one view is needed (if this is not so, one could rotate the object holder and include x,y displacements of the cylinder axis with respect to the rotation axis as unknowns). Adding the unknown scattering potentials of the saline and plastic to the list of unknowns gives the 8 values dₜ, dᵣ, bₜ, bᵣ, a, yₛ, gₛ, gₚ to be determined. As a further simplification, let us take the transducers to be line sources which are perfectly colinear with the cylinder axis. For these omnidirectional transducers, there are no bₜ, bᵣ, unknowns.

As a simulation example, data was generated by analytic cylinder solution for the following parameter values:

$$frequency = 312.5 KHz$$

$${C}_{0} = 1485 m / s ( speed of sound in water )$$

$$Outer diameter of plastic cylinder = {1}^{″}$$

$$Thickness of plastic = 0.73 mm$$

$$Number of receiver positions = 128$$

$$Receiver sample interval , D = 1.195 mm ( {y}_{n} = ( n - 64.5 ) D + {y}_{s} )$$

$${g}_{s} = - 0.12$$

$${g}_{p} = - 0.5$$

$${d}_{r} = 5 cm$$

$$a = 3 degrees$$

$${y}_{s} = 3 mm$$

$${d}_{t} 15. cm$$

The data used to optimize for the parameters is the log of the eikonal:

$${data}_{n} = ln ( {f}_{n} / {f}_{n}^{i} ) = ln ( \vert {f}_{n} / {f}_{n}^{i} \vert ) + i Phase ( {f}_{n} / {f}_{n}^{i} )$$

The optimization functional is then:

$$F ( {γ}_{s} , {γ}_{p} , α , {y}_{s} , {d}_{r} , {d}_{t} ) = {\sum }_{n} {\vert ln ( {f}_{n} / {f}_{n}^{i} ) ( {γ}_{s} , {γ}_{p} , α , {y}_{s} , {d}_{r} , {d}_{t} ) - {data}_{n} \vert}^{2}$$

The starting guess for the optimization is:

$${g}_{s} = - 0.1$$

$${g}_{p} = - 0.4$$

$${d}_{r} = 4.5 cm$$

$$a = 0. degrees$$

$${y}_{s} 0. mm$$

$${d}_{t} = 15.5 . cm$$

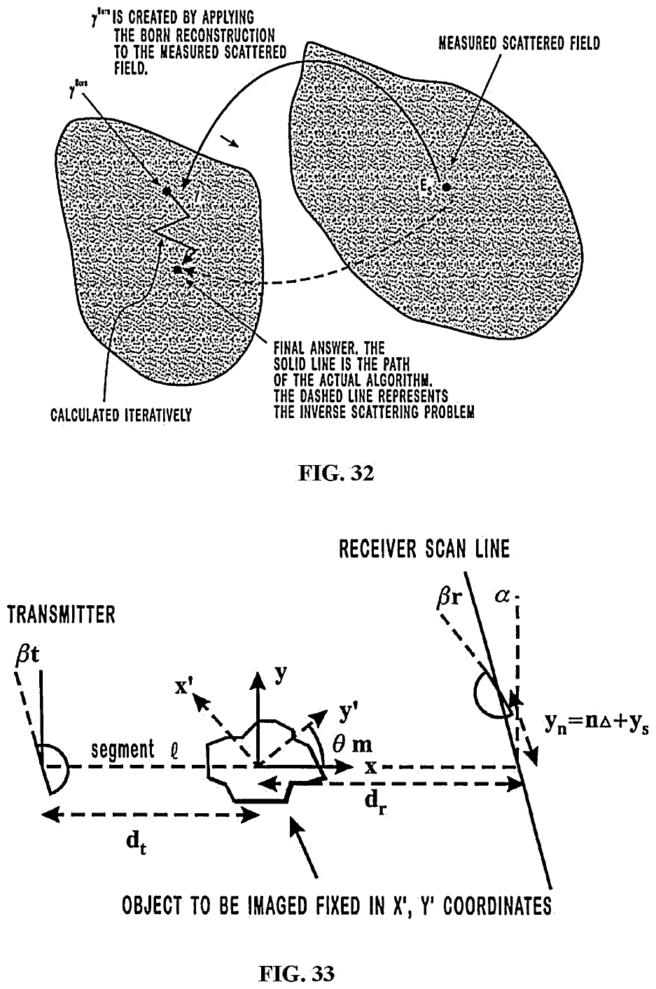

Computing the data for these two parameter sets gives the comparison shown in FIGS. 31 and 32.

Solution after 3 Ribere-Polack steps applied to the minimization of the functional gives the solution:

$$Normalized residual ≡ \sqrt{\frac{F ( final solution )}{{\sum }_{n} {\vert {data}_{n} \vert}^{2}}} = 0.1016598$$

$$Normalized gradient ≡ \sqrt{\frac{∇ F ( final solution )}{∇ F ( starting guess )}} = 0.2283959$$

| | | | |
|---|---|---|---|
| gₛ = | −0.116789, | 2.68% | error |
| gₚ = | −0.59277, | 18.6% | error |
| dᵣ = | 4.756 cm | 4.9% | error |
| a = | 2.29916 degrees | 23.4% | error |
| yₛ = | 2.18673 mm | 27.1% | error |
| dₜ = | 15.0 cm | 0% | error |

For 5 R-P steps:

$$Normalized residual = 5.11123 E - 02$$

$$Normalized gradient = 0.122296$$

| | | | |
|---|---|---|---|
| gₛ = | −0.117978, | 1.75% | error |
| gₚ = | −0.57, | 14.0% | error |
| dᵣ = | 4.87 cm | 2.6% | error |
| a = | 3.08706 degrees | 2.9% | error |
| yₛ = | 2.92 mm | 2.7% | error |
| dₜ = | 15.0 cm | 0% | error |

For 9 R-P steps:

$$Normalized residual = 1.0188 E - 02$$

$$Normalized gradient = 1.22823 E - 02$$

| | | | |
|---|---|---|---|
| gₛ = | −0.118573, | 1.17% | error |
| gₚ = | −0.518917, | 3.78% | error |
| dᵣ = | 5.0 cm | 0% | error |
| a = | 3.08706 degrees | 2.9% | error |
| yₛ = | 2.96672 mm | 1.1% | error |
| dₜ = | 15.0 cm | 0% | error |

### Overview of Calibration Steps for Acoustic 2D Geometry

First incident field, scattered field, the total field corresponding to the FIG. 30 are read into memory.

### Step 1:

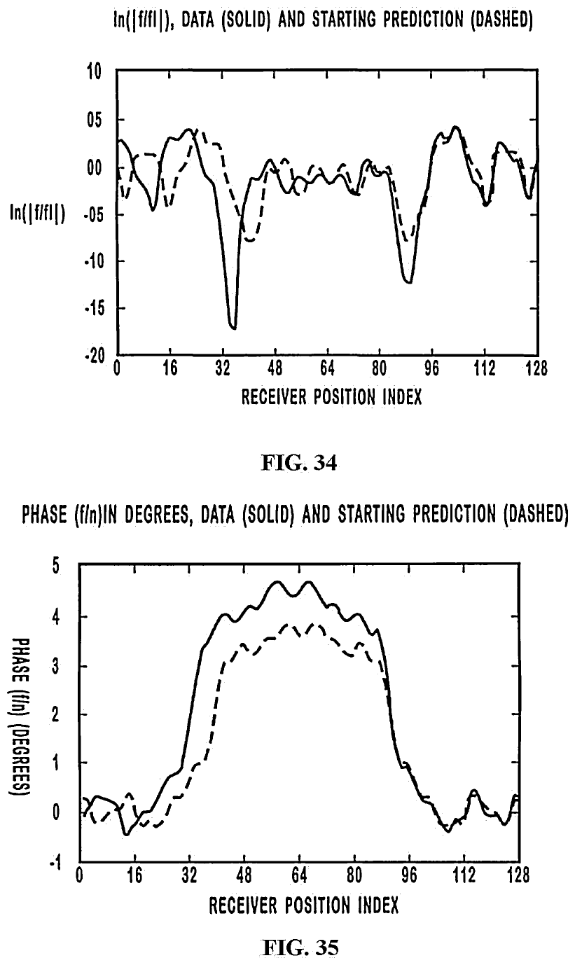

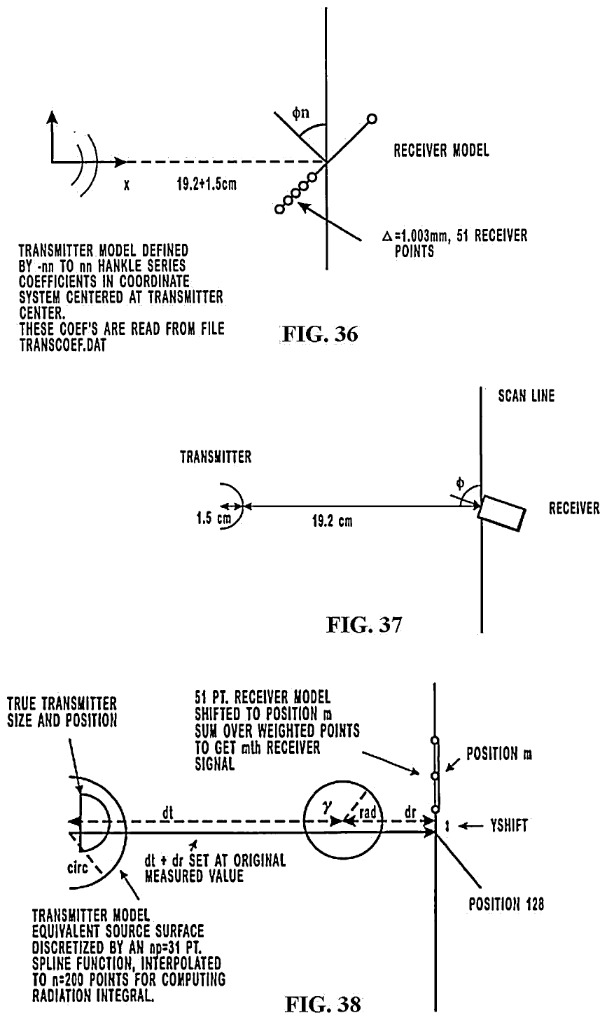

Creation of receiver model. For this purpose a procedure is carried out whereby the receiver is rotated in its center position with transmitter at some fixed position. The receiver is rotated at equi-angular intervals from −p/2 to p/2. For the 2D model the receiver is modelled as 51 weighted line receivers separated by some fixed distance drec, which is fixed, as shown in FIGS. 33 and 34.

### Step 2

Next a transmitter model is constructed which consists of an infinitely long, infinite impedance cylinder with a source function on its surface. The transmitter model equivalent source surface is discretized by an np=31 pt. spline function, interpolated to n>np points for the computation of the incident field.

The np node values on the surface of the non-physical infinite impedance cylinder are the unknowns which are optimized to give a best fit in the least squares sense of the incident field as measured on the receiver array, as shown in FIG. 33.

### Step 3

The next step is the use of the incident field generated by this impedance cylinder model, and exact Bessel solution models for scattering from a cylinder to create a scattered field, which is matched with the measured scattered field, using the receiver model calculated above. The setup is as shown in FIG. 38.

### Software Descriptions

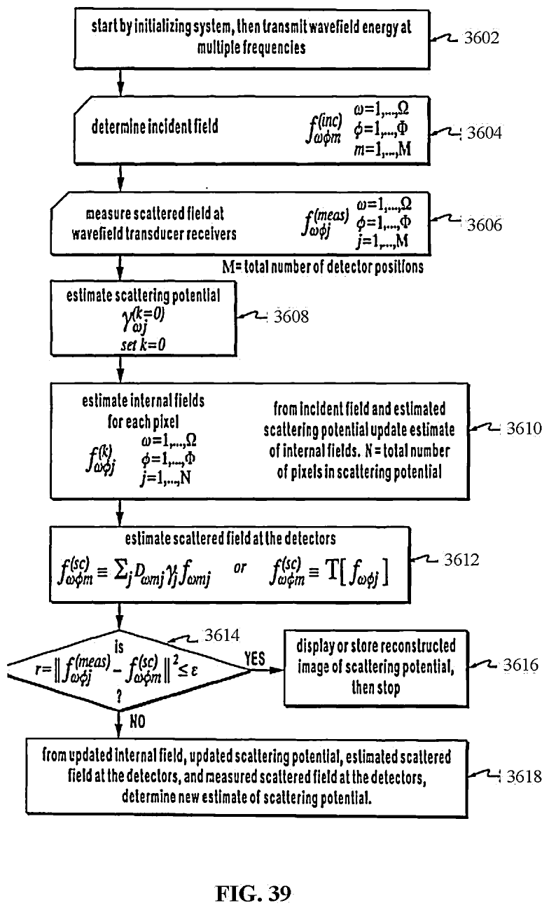

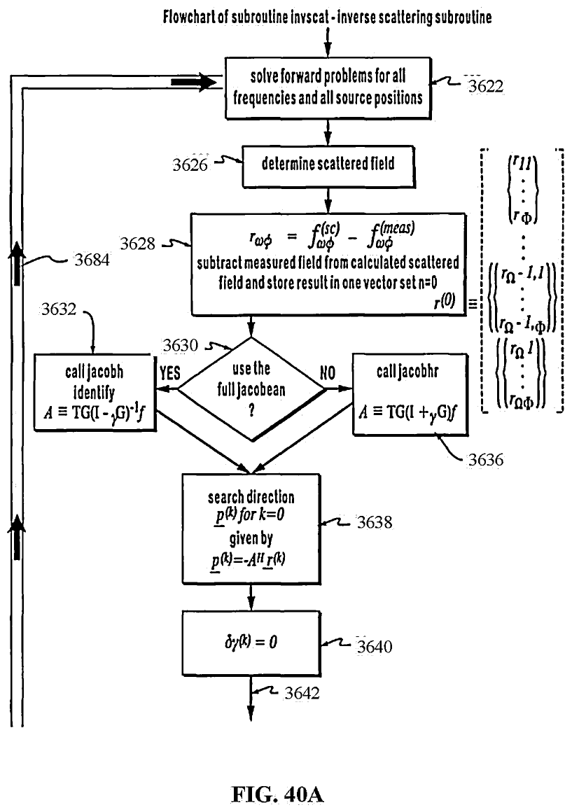

Having described some examples in the previous section which illustrate mathematical and physical models which may be used to model scattered wavefield energy and to process data so as to reconstruct an image of material parameters using inverse scattering theory, reference is next made to FIG. 39.

This figure schematically illustrates a flow diagram for a computing system carrying out an imaging process. The flow diagrams provided herein are merely illustrative of one implementation using the scalar Helmholtz wave equation or vector wave equation or similar wave equation for modeling scattered wavefield energy as described in Example 1.

As shown in FIG. 39, as schematically illustrated in Step 3602, for each transmitter from which a signal is sent, the system next causes a receiver multiplexer to sequence each receiver array so as to detect the scattered wavefield energy at each position.

Once the scattered wavefield energy has been detected, processed, and digitized, the system next determines the incident field f_{ωφ}^{inc}, as indicated at step 3604. The incident field is determined from the wavefield energy (e.g., frequency and amplitude components of the transmitted waveform) and transducer geometry from which the incident field is transmitted. The process of determining this incident field may involve direct measurement of the incident field or calculation of the incident field using the appropriate Green's function whether it be Biot, elastic, electromagnetic, scalar, vector, free space or some combination or permutation of these. The incident field is determined at each pixel in the imaged space for each source position φ=1, . . . , Φ and for each frequency ω, which varies from 1 to Ω. In step 3606, the scattered wavefield is measured, recorded and stored at the wavefield transducer receivers for each ω, varying from 1 to Ω each source position 1−Φ, and each pixel element at each wavefield transducer receiver position. In step 3608, the initial estimate for the scattering potential can be set at either 0 or at an average value which is derived from the average material parameters estimated a priori for the object. When applicable, a lower resolution image of γ, such as produced by a time of flight method as described in U.S. Pat. No. 4,105,018 (Johnson) may also be used to advantage (in the acoustic case) to help minimize the number of iterations. Similar approximations for other forms of wavefield energy may be used to advantage to help minimize the number of iterations.

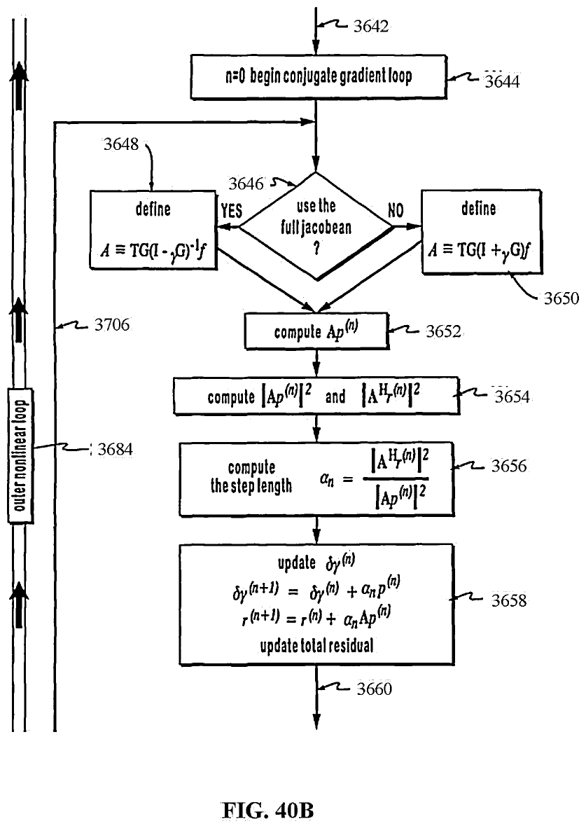

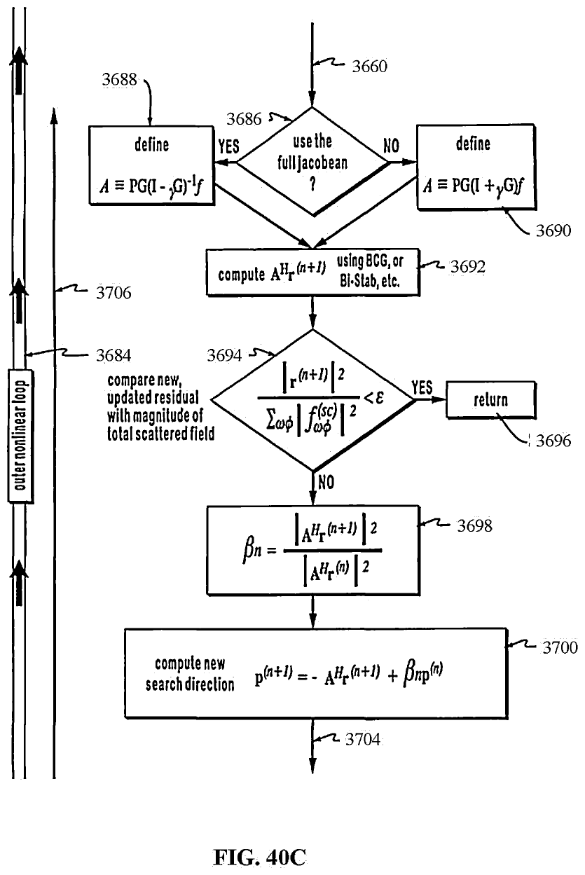

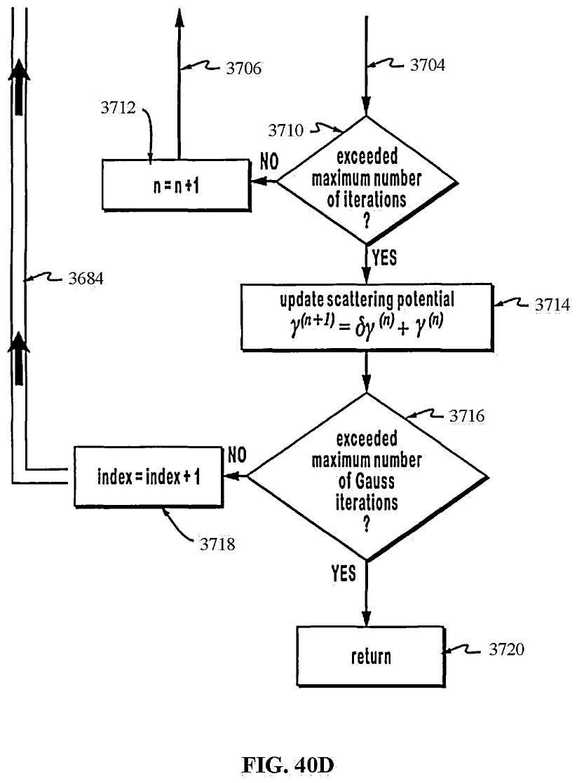

The system next determines in step 3610 an estimate of the internal fields for each pixel in the image space and for each frequency ω, and each source position Ω. This estimate is derived from the incident field and the estimated scattering potential. This step corresponds to step 3622 in FIG. 40A as well as to step 3610 in FIG. 39.

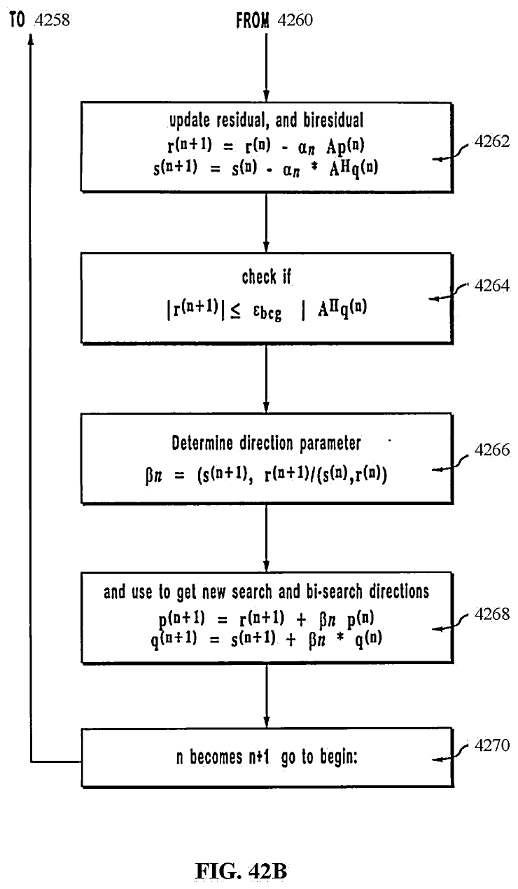

For a given and fixed frequency ω, and fixed source position φ, the forward problem consists of using equation 1 or 17 (provided in EXAMPLE 1) to determine the total field f_{Y} given the scattering potential γ and the incident field f^{inc}_{ωφ} and the appropriate Green's function. This Green's function could be the scalar Green's function which is essentially the Hankel function of the second kind of zero order, or it could be the elastic Green's functions which is derived from the scalar Helmholtz Green's function, or it could be the Biot Green's function, or it could be the electromagnetic vector or scalar Green's function as well as a layered Green's function derived from one of the preceding green's function in the manner described above. The process whereby this is done is elucidated in FIGS. 42A and 42B.

Before applying this algorithm, equation 1 or 17 is discretized so that it may be input into a processor. This process is described in detail in the Examples. The number of frequencies that are used in the inverse scattering problem is a function of the source receiver geometry, among other things.

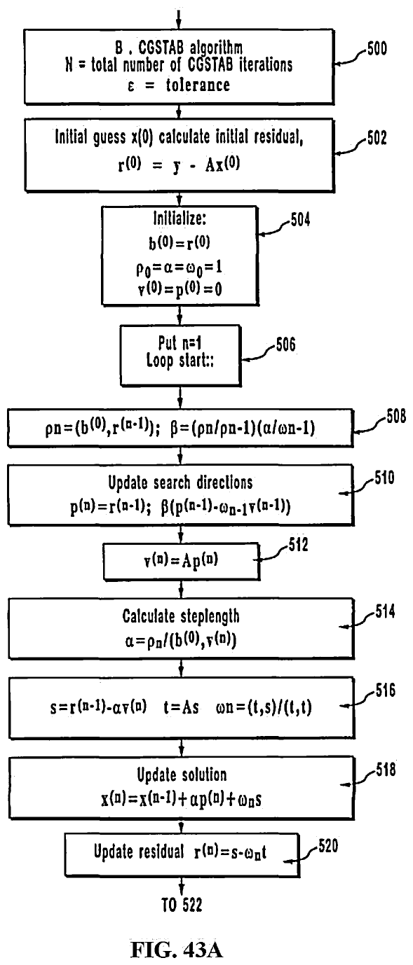

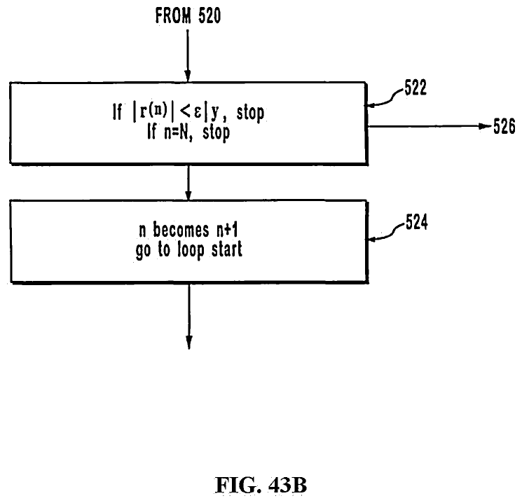

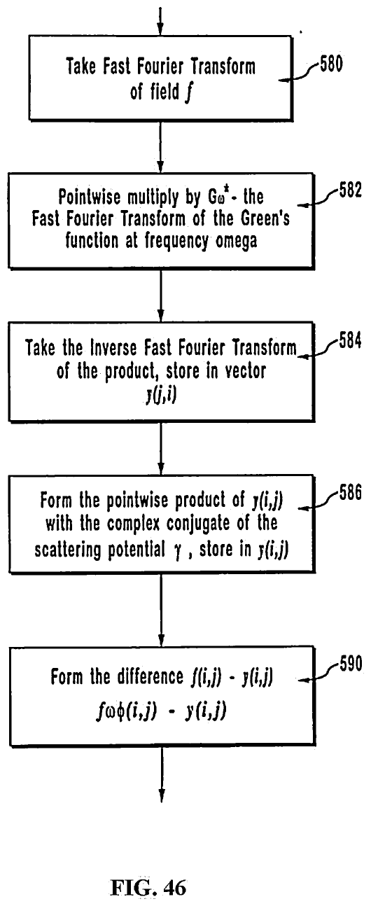

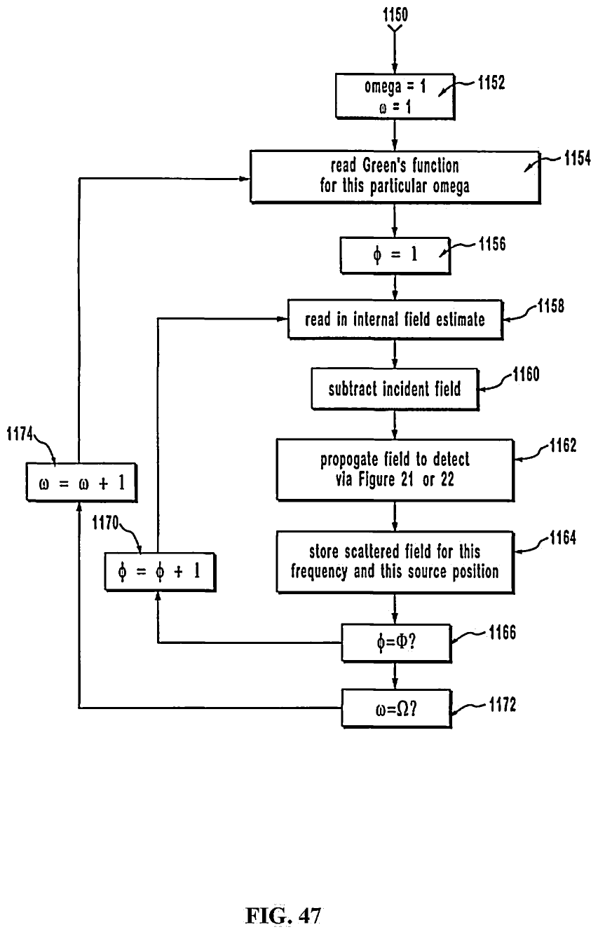

All forward problems for all possible frequencies, and all possible source positions can be solved, for example, for the two-dimensional case or for the three-dimensional case, whichever is relevant. FIG. 41 gives in detail the procedure for solving for the internal fields at all possible source positions and all possible frequencies. From step 3622 in FIG. 40A we enter step 4140 on FIG. 41. In step 4140 of FIG. 41, all of the files that contain all of the Green's functions for all possible frequencies and the files that contain the incident fields for all possible frequencies and all possible source positions are rewound. In step 4142 of FIG. 41, ω is set equal to 1, i.e., we indicate that we are about to calculate the forward problem for the first frequency. In step 4144 of FIG. 41, the Green's function for this particular frequency is read into memory. In step 4146 of FIG. 41, the source position index φ, is set equal to 1 indicating that we are solving the forward problem for the first source position. In step 4148, the incident field for this particular y, i.e., source position, and this particular ω, i.e., frequency, is read into memory. In step 4150, the forward problem for this particular frequency and this particular source position is solved using either biconjugate gradients or BiSTAB. This process is detailed in FIGS. 42A and 42B for the biconjugate gradient or BCG algorithm and FIGS. 43A and 43B for the BiSTAB or biconjugate gradient stabilized algorithm. In FIG. 42A, in step 450 which follows step 4150 in FIG. 41, the predetermined maximum number of biconjugate steps and tolerance are read into memory, and the forward problem, which in discrete form reads (I−G_{ω})f=f^{inc}, is solved. For purposes of generality, we consider the BCG or biconjugate gradient algorithm applied to the system Ax=y. A is a matrix which is square, x and y is a vector of suitable dimension. Step 4252 consists of reading in an initial guess X⁰ and calculating an initial residual which is calculated by applying A to the initial guess X⁰ and subtracting from the right-hand side Y. In step 4254, the initial search direction P⁰, bi-search direction Q, bi-residual S are calculated and stored. In step 4256, we put N equal to 0. In step 4258, we calculate the step length an by taking the inner product of the two vectors s and r, the bi-residual and residual respectively, and dividing by a quantity which is the inner product of two vectors. These two vectors are 1) q, the bi-search direction, and 2) the result of the application of the operator A to the present search direction P. In step 4260 of FIG. 42A, the guess for x is updated as per the equation that is shown there. In Step 4262, the residual R is updated as per the formula given, and the bi-residual S is updated as per the formula given. (H indicates Hermitian conjugate.) In particular, the bi-residual is given by the previous bi-residual Sⁿ minus a quantity which is given as α*n, which is the complex conjugate of an, multiplied by a vector, which is the result of the operation of the Hermitian conjugate of the operator A, applied to the vector q, the bi-search direction. This is carried out by a call to FIG. 46, which we discuss below. In step 4264, check to see if the stop condition has been satisfied, i.e., is the present updated residual less than or equal to E (which is given a priori) multiplied by the initial residual in absolute value. Step 4266 consists of the determination of β, the direction parameter, which is given as the ratio of two numbers. The first number in this ratio is the inner product of the new updated bi-residual with the updated residual and the denominator is the inner product of the previous residual with the previous bi-residual, which has already been calculated in box 4258. In step 4268, use the calculated β to update and get a new bi-search direction, and a new search direction given by the formulas shown. In step 4270, N is incremented by 1 and control is taken back to step 4258. In step 4258, a new step length is calculated, etc. This loop is repeated until the condition in step 4264 is satisfied, at which time control is returned to step 4150 in FIG. 41. The forward problem for this particular frequency and this particular source position has now been solved and the result, i.e., the internal field, is now stored.

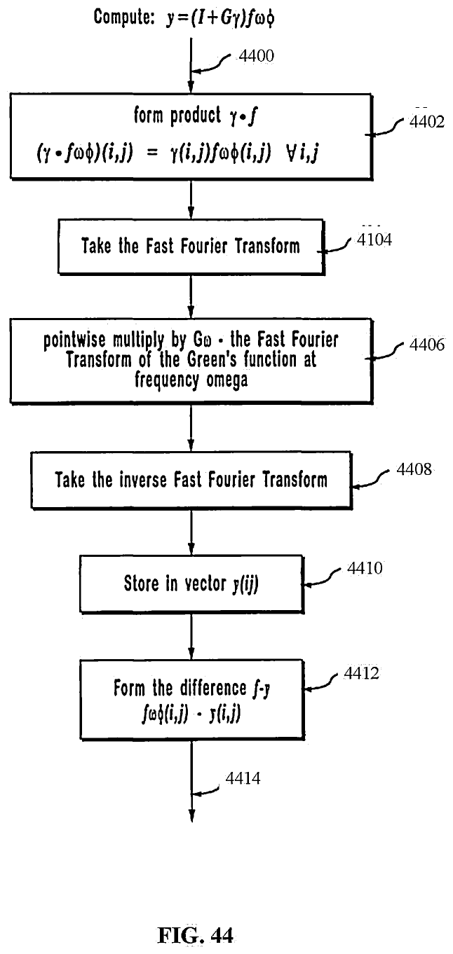

In step 4152, back in FIG. 41, it is determined if φ is equal to Φ, i.e., have we computed all the forward problems for this particular frequency for all possible source positions. If not, then φ is incremented by 1 in step 4154 and control is returned to step 4148 of FIG. 41. If so, we have calculated the forward problem for all possible source positions y and control is transferred to step 4156 where it is determined if ω is equal to Ω, i.e. have we calculated the forward problem for all possible frequencies ω?” If not, ω is incremented by 1 in box 4157 and control is returned to step 4144. If so, we have completed the computation of the internal fields for all possible source positions and all possible frequencies and have stored the results, i.e. we have finished the subroutine for the two-dimensional or three-dimensional problem, respectively. Control is now transferred to the main program for the two-dimensional case or for the three-dimensional case. This corresponds to step 3622 in FIG. 40A. This also corresponds to step 3612 of FIG. 39. FIGS. 40A, 40A, 40C, and 40D together comprise an expanded version of the inverse scattering algorithm in its entirety, which is given in compressed form by FIG. 39. Before proceeding further to describe in detail step 3612 of FIG. 39, i.e., also step 3626 of FIG. 40A, we mention that the BCG algorithm just described may be replaced by the BiSTAB (biconjugate gradient stabilized algorithm) of FIGS. 43A and 43B. Furthermore, in the calculation of both the BiSTAB algorithm or the biconjugate gradient algorithm, the implementation of the operation of the operator A acting on the vector x is carried out in a very specific manner which is described in FIG. 44. Furthermore, in FIG. 46, the Hermitian conjugate of the operator A, denoted A^{H}, is applied to vectors in its domain. Specifically, in step 4400 of FIG. 44, we come in from any step in FIGS. 43A and 43B or 42A and 42B, respectively, the conjugate gradient stabilized algorithm or the bi-conjugate gradient algorithm which requires the calculation of the operation of the operator A acting on the vector “x”. In step 4102, the pointwise product of the scattering potential γ and the present estimate of the internal field at frequency ω and source position φ is formed and stored. Step 4404, the Fast Fourier Transform (FFT) of this pointwise product is taken. In step 4406, the result of the Fast Fourier Transform (FFT) taken in step 4404 is pointwise multiplied by G, which is the Fast Fourier Transform (FFT) of the Green's function at frequency ω, which has been stored and retrieved. In step 4408, the inverse Fast Fourier Transform (FFT) is taken the pointwise multiplication that was formed in the previous step. In step 4410 this result is stored in vector y. In step 4412 the difference, taken pointwise, of the f_{ωφ}, the present value for the internal field at frequency ω, and source position φ, and the constructed vector y is taken. Step 4414 returns control to either the BCG or the BiSTAB algorithm, whichever has been called by step 4150 of FIG. 41. The application of the operator which is described in FIG. 44 is carried out multiple times for a given call of the biconjugate gradient or the BiSTAB algorithm, it is imperative, therefore, that this step be computed extremely quickly. This explains the presence of the fast Fourier transforms to compute the convolutions that are required. This operator, (I−Gγ) symbolically written, we refer to as the Lippmann-Schwinger operator. FIG. 46 calculates the adjoint or Hermitian conjugate of the given Lippman-Schwinger operator and applies this to a given vector. Step 580 of FIG. 46 takes control from the appropriate step in the biconjugate gradient algorithm. It is important to note that the BiSTAB algorithm does not require the computation of the adjoint of the Lippmann-Schwinger operator. Therefore, FIG. 46 is relevant only to FIGS. 42A and 42B, i.e., the biconjugate gradient algorithm. In step 580 of FIG. 46 the Fast Fourier Transform (FFT) of the field f to which the adjoint of the Lippmann-Schwinger operator is being applied, is taken. In step 582 of FIG. 46, the Fast Fourier Transform (FFT) computed above is pointwise multiplied by G*_{ω}, the complex conjugate of the Fast Fourier Transform (FFT) of the Green's function at frequency ω. In step 584, the inverse Fast Fourier Transform (FFT) of this pointwise product is taken and stored in vector γ. In step 586, the pointwise product of the complex conjugate of γ, the present estimate of the scattering potential is taken with the vector γ. The result is stored in vector y. In step 590, the difference is formed between the original field to which the adjoint of the Lippmann-Schwinger operator is being applied, and the vector y just calculated. Now (I−Gγ)^{Hf}_{ωφ} has been calculated and control is now returned to box 4262 of FIGS. 42A and 42B, the biconjugate gradient algorithm, where use is made of the newly created vector, A^{H}q. Next the stop condition is checked at step 4264.

We now return to step 3622 of FIG. 40A, i.e. step 3612 of FIG. 39 where we determine the scattered field at the detector locations for all possible source positions, i.e., incident fields and also all possible frequencies ω. This process is carried out in FIG. 47, to which we now turn.

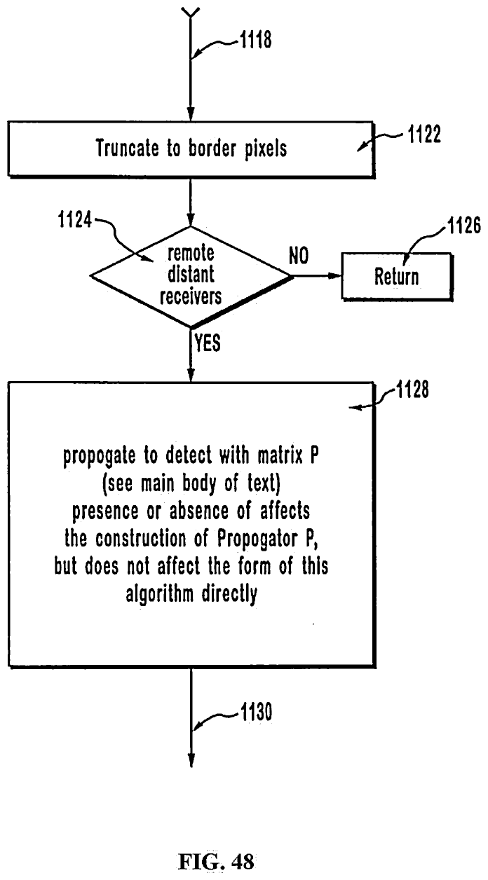

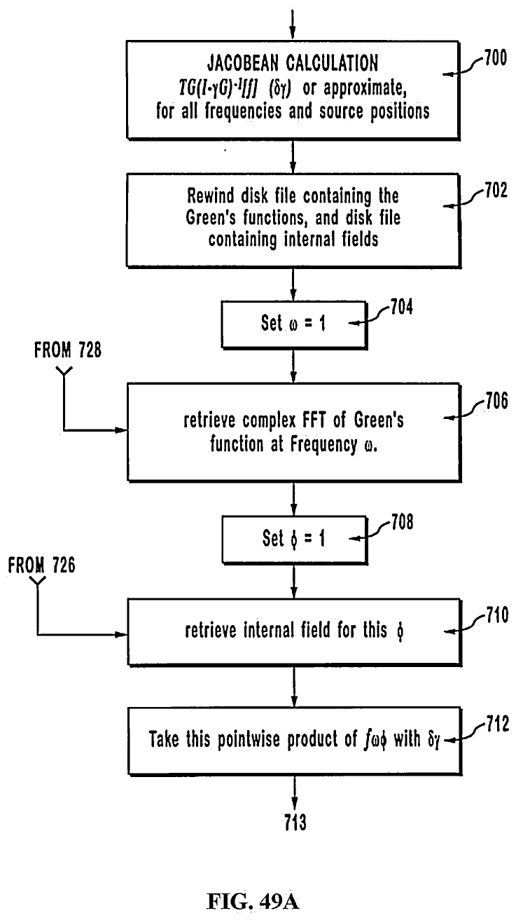

Step 1150 of FIG. 47 transfers control to step 1152 of FIG. 47 where ω is set equal to 1. Control is then transferred to step 1154 where the Green's function for this particular ω is read into memory. Control is then transferred to step 1156 where φ is set equal to 1. Control is then transferred to step 1158 where the internal estimate for this source and this frequency is read into memory. Control is then transferred to step 1160 where the incident field is subtracted from estimated total internal field. In step 1162 the truncation or propagation operator shown FIG. 48 is performed. When the truncation or propagation has been performed, control is transferred back to FIG. 47 and in step 1164 the calculated scattered field is stored. In step 1166 it is determined if φ is equal to Φ, if it is not, then φ is incremented by one, in step 1170, and control is returned to step 1158 of FIG. 47. If φ is equal to Φ, then control is transferred to step 1172 where it is determined if ω is equal to Ω. If it is not, ω is incremented by one in step 1174, and control is returned to step 1154 of FIG. 47. If ω is equal to Ω, then control is returned to step 3626 of FIG. 40A, the inverse scattering subroutine.

Now consider FIG. 48, which was called by FIG. 47 above.

Step 1118 of FIG. 48 transfers control to step 1122 where the input is truncated to the border pixels. Control is passed to step 1124 which determines if the receivers are distant from the image space or whether the receivers are juxtaposed to the image space. If the detectors are in fact juxtaposed to the image space, then control is transferred to step 1126 and from there to the calling subroutine. If the detectors are not juxtaposed to the image space, the fields are propagated to the detector positions. The details of this propagation are given in the “EXTENSION OF THE METHOD TO REMOTE RECEIVERS WITH ARBITRARY CHARACTERISTICS” section of this patent. Actually, there are several methods which could be used to accomplish this. For example, an application of Green's theorem is one possibility. The beauty and utility of the method shown in the “Extension of the method to remote receivers with arbitrary characteristics” section however, is that the normal derivatives of the Green's function, or the fields themselves, do not need to be known or estimated.

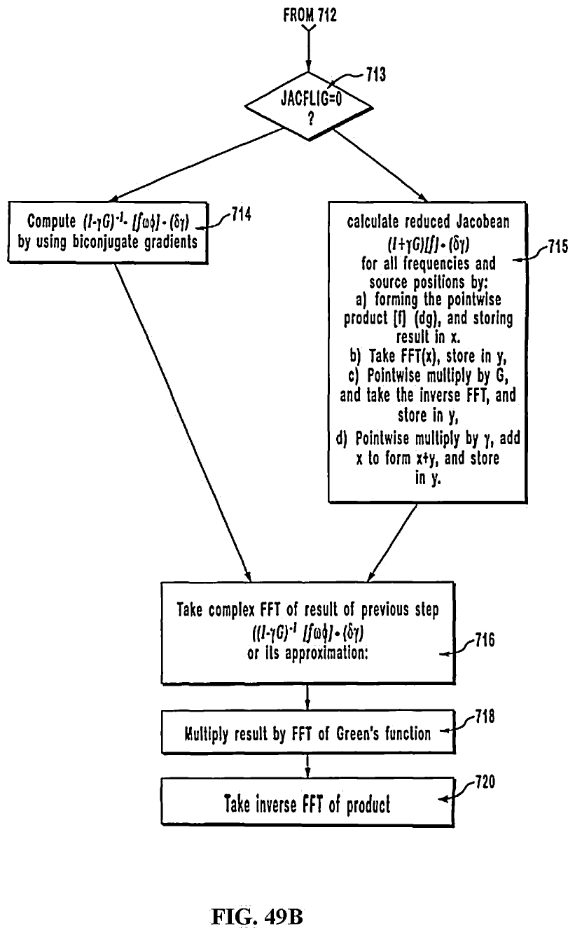

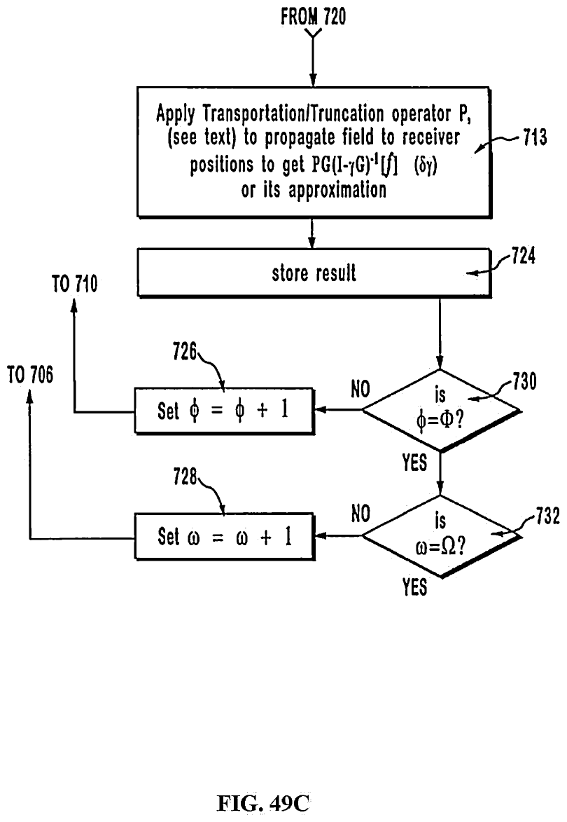

The input to FIG. 48 is a vector of length NₓN_{y}. FIG. 48 represents the subroutine that is called by the Jacobian calculation or following immediately after the calculation of the internal fields. The input in the first case of the Jacobian calculation consists of box 720 of FIG. 49B. The input in the second case is the scattered field calculated for a particular frequency ω and a particular source position cp. The purpose of FIG. 48 is to propagate the input (scattered field in the image space) to the detector positions. In step 1124 it is determined whether the receivers are distant. If the detector positions are located at some distance from the image space, the subroutine passes control from step 1124 to step 1128.

In step 1128, the matrix P, constructed earlier is used to propagate the field to remote detectors. In step 1130 of FIG. 48, control is returned to the program which called the subroutine. The matrix P will change when correlation is present; the fundamental form of the propagation algorithm remains unchanged, however.

These scattered fields are stored in vector d^{ωφ}. Control is then transferred to step 3628 in FIG. 40A which subtracts the measured scattered field from the calculated scattered field and stores the result in one vector r_{ωφ} which is the residual vector for frequency ω and source position φ. This step is repeated until all possible frequencies and all possible source positions have been accounted for, whereupon the residual vectors r^{ωφ} for all possible ω's and φ 's are stored in one large residual vector shown in FIG. 40A as step 3628. We now apply the Hermitian conjugate of the Jacobian to the residual vector r just calculated in order to establish the search direction Pⁿ where n=0, i.e., the initial search direction. The search direction is given explicitly by the negative of the action of the Hermitian conjugate of the Jacobian acting upon the residual vector r, in step 3638.

There are in effect at least two possibilities regarding the use of the Jacobian. First, the full Jacobian could be used (e.g., step 3632) or, secondly, an approximation to the Jacobian could be used (e.g., step 3636). The determinator of which path to follow is the programmer, who bases his decision on the magnitude of the γ which is to be solved in the full inverse scattering problem. If the γ is large, the exact Jacobian must be used. On the other hand, if the γ is reasonably small, although still larger than required for the Born or Rytov approximation, the approximation to the full exact Jacobian which is shown on FIG. 40A, step 3636 may be used. The application of the exact or approximate Jacobian will be discussed later.

Now we step through FIGS. 40A to 40D to get an overall view of how the algorithm works.

Step 3638 computes the initial search direction P⁽⁰⁾ Step 3640 of FIG. 40A zeros the initial guess for δγ, which is the Gauss-Newton (or Ribiere-Polak, or other) update to γ, the scattering potential. γ can be initially set=0. A large portion of the rest of FIG. 40A, 40A, 40C, 40D is concerned with the calculation of δγ. This is used in FIG. 39 also, of course.

Step 3612 of FIG. 39 has estimated the scattered field at the detector positions. Step 3614 of FIG. 39 now determines if the residual (which is given by the magnitude squared of the difference between the measured scattered field and the calculated scattered field, for each possible source position and each possible frequency) is less than some predetermined ε tolerance. If the condition is satisfied then control is transferred to Step 3616 where the reconstructed image of the scattering potential is either displayed or stored. If the condition is not met, then from the updated internal field calculated in Step 3610 of FIG. 39 and also from the most recent estimate of the scattering potential and also from the estimated scattered field at the detectors calculated in Step 3612 of FIG. 39 and also from the measured scattered field at the detectors, a new estimate of the scattering potential γ is calculated in a manner to be made explicit below. This updated scattering potential estimate is derived in Step 3618, and is now used in Step 3610 of FIG. 39 to calculate a new update of the estimate of the internal fields, for each possible frequency ω and each possible source position φ. Then in Step 3612 of FIG. 39 a new estimated scattered field at the detectors is calculated by means of Green's theorem as shown in FIG. 47. This new estimate of the scattered field is then used in Step 3614 as before, to estimate the magnitude of the residual vector between the measured scattered field and the calculated scattered field to determine if this quantity is less than E, the predetermined tolerance. As before if this tolerance is exceeded the loop just described, is repeated until such time as either the tolerance is satisfied or the maximum number of iterations of this loop has been exceeded. This maximum number is predetermined by the operator.

We have just concluded a general description of the overall inverse scattering algorithm leaving out many important details. We now look at each of the steps in more detail. First, we explain how the δγ⁽⁰⁾ shown in Step 3640 of FIG. 40A is related to Step 3618 of FIG. 39. δγ⁽⁰⁾ in FIG. 39 is the first estimate of δγ in Step 3714, which is the vector which is solved for, in Steps 3644 through to Step 3714 in FIGS. 40A-40D. This δγ is the correction term to the scattering potential which is applied to the present estimated scattering potential γ⁽ⁿ⁾ in Step 3714 of FIG. 40D in order to get the updated scattering potential γ⁽ⁿ⁺¹⁾. Once this new updated scattering potential has been determined, transfer to Step 3716 of FIG. 40D where it is determined if the maximum number of Gauss-Newton corrections have been exceeded. Each pass through Steps 3644 through 3714, to calculate δγ is one Gauss-Newton step. These Gauss-Newton steps are repeated until one of two things occurs. Either the maximum number of Gauss-Newton corrections is exceeded, or the tolerance condition of Step 3694 is satisfied. It is important to reiterate that which was noted earlier, namely, that the description of the Gauss-Newton algorithm here, does not preclude the use of a suitable Ribiere-Polak, or Fletcher-Reeves non-linear algorithm, or other non-linear system solver. The changes in detail do not change the overall character of the minimization algorithm.

It should be noted that the most general case is presented where layering as discussed in Example 2 can be accounted for. In addition, the Lippman-Schwinger operator described earlier incorporates a convolution and correlation part. If it is known a priori that there is negligible layering in the geophysical or non-destructive testing or other scenario envisioned then it is appropriate to use code covering the case of no correlation part in the Green's function. This code also computes the two-dimensional free space Green's function from scratch. This is in contradistinction to the two-dimensional case in the presence of layering, where three separate programs are used to solve the inverse scattering problem, the forward problem and the construction of the layered Green's function. The construction of this layered Green's function which is discussed in Example 2 for the acoustic scalar case, requires substantial amount of time for its computation. However, it is important to note that once the Green's function has been constructed for a given layered scenario, in a geophysical context, for example, it is not necessary to recalculate this Green's function iteratively. Rather the Green's function once it has been determined by methods discussed in the literature, see for example, [Johnson et al. 1992, or Wiskin et al, 1993],

As noted, Example 2 shows explicitly how the convolution and correlation are preserved even though the distribution of the layers above and below the layer containing the image space are arbitrarily distributed. The preservation of convolution and correlation is a critical element of the ability to image objects in a layered or Biot environment (Biot referring to the Biot theory of wave propagation in porous media) in real time. The reflection coefficients which are used in the construction of the layered Green's function in the acoustic, elastic and the electromagnetic case are well known in the literature. See, for example [Muller, 1985] or Aki and Richards, 1980. The incorporation of this Green's function for layered media in the acoustic, elastic and electromagnetic case for the express purpose of imaging objects buried within such a layered medium is novel. The ability to image in real time is critical to practical application of this technology in the medical, geophysical, non-destructive and testing microscopic environments.

We now elucidate the steps shown in FIG. 40A. We have already discussed in detail, steps up to 3640, Step 3642 of FIG. 40A begins the conjugate gradient loop. Step 3644 of FIG. 40A sets n equal to 0.

This n is the counter that keeps track of the number of conjugate, gradient steps that have been performed in the pursuit of δγ, the update to the scattering potential estimate. Step 3646 of FIG. 40A again determines if the full Jacobian will be used. As in previous step 3630 of FIG. 40A the answer depends on the magnitude of γ. In the case that the γ is large, the approximation given in Step 3650 is not appropriate. Instead step 3648 is performed. Furthermore, the actual implementation of the algorithm depends on whether there is correlation present. They are sufficiently different to warrant separate flow charts. FIGS. 49A/B/C deal with the free space case. Considering this case first, JACFLAG is set to one and Step 3652 is computed via FIGS. 49A/B/C. Step 700 of FIG. 49A transfers the algorithm control to Step 702 where the disk file containing the Green's functions and the disk file containing the internal fields for all possible ω and all possible φ source positions are rewound. Control is then transferred to Step 704 where ω, the index for the frequency, is set equal to 1. In Step 706 thereafter, the complex Fast Fourier Transform (FFT) of the Green's function; at this particular frequency, is retrieved. In Step 708, φ, the counter corresponding to the source positions, is set equal to 1. The Step 710 in FIGS. 49A/B/C retrieves the internal field for this particular source position φ, the Step 712 takes the point-wise product of this internal field which was retrieved in Step 710, with the δγ which is the input to FIGS. 49A/B/C and is also the same δγ which is being sought after in the main program in FIG. 40A-40D. In Step 3652 of FIG. 40A which is where we have come from in FIGS. 49A/B/C, the Jacobian is applied to p, which is the search direction for δγ. That is p is the input to FIGS. 49A/B/C, i.e. δγ in this call of FIGS. 49A/B/C is p of Step 3652 in FIG. 40A. In Step 714 of FIGS. 49A/B/C either the inverse of a certain operator or the first order binomial approximation of the inverse, is applied to the point-wise product produced in Step 712. This operator, (I−γG), is similar to, but not the same, as the Lippmann-Schwinger operator which is represented as (I−Gγ). Note, the determinator of which operator (I−Gγ)⁻¹ or (I+Gγ) is applied is the JACFLAG variable defined in FIG. 40A. JACFLAG=1 corresponds to application of (I−Gγ)⁻¹.

It is important to note that the inverse of this operator, (I−γG)⁻¹, is never explicitly calculated in Step 714. The actual computation of the inverse of this operator, which in discrete form is represented as a matrix would be computationally very expensive. Therefore, the inverse action of the operator (I−γG)⁻¹ acting on the point-wise product calculated in Step 712 is calculated by using bi-conjugate gradients or BiSTAB as indicated in Step 714 of FIG. 49A/B/C. This bi-conjugate gradient algorithm is the same bi-conjugate algorithm shown in FIGS. 42A and 42B and was used to calculate the application of the Lippman-Schwinger operator [I−Gγ] on a vector f. However, it is important to note that the operator represented by the matrix A in Step 4250 of FIG. 42A is not the Lippman-Schwinger operator, rather it is the above (I−γG) operator in this case. As before, the BiSTAB or Biconjugate gradient stabilized algorithm may be used in place of the bi-conjugate gradient algorithm. This BiSTAB or conjugate gradient stabilized algorithm is shown in FIGS. 43A and 43B. The application of the algorithm shown in FIG. 44 for the actual application of the operator A in BiSTAB, FIGS. 43A and 43B, or BCG, FIGS. 42A and 42B, will of course, differ, in that the point-wise multiplication by γ will be performed after the convolution with the Green's function G appropriate to this frequency ω, in this case. However, the changes that must be made to FIG. 44 are clear in this case. Namely, the point-wise product shown in Step 4402 is taken after the computation of the inverse Fast Fourier Transform (FFT) in Step 4408 instead of before. A similar comment applies to b where the Hermitian conjugate of I−γG is computed. In this case the Hermitian conjugate of the operator I−γG requires that the point-wise product of the conjugate of γ which is carried out in Step 586 of FIG. 46 be calculated before the Fast Fourier Transform (FFT) is taken in Step 580 of FIG. 46. Step 3654 of FIG. 40B now calculates the magnitude of the vector computed in Step 3652 namely, the application of the vector A to the search direction p. Step 3654 of FIG. 40B also computes the magnitude of the vector which is the result of the application of the Hermitian conjugate of the vector A to the residual vector r. Again, A is either the exact Jacobian or the approximation to the Jacobian shown in Step 3650. The Step 3656 computes the square of the ratio of the two magnitudes computed in Step 3654, this ratio is denoted by α. Step 3658 of FIG. 40B now updates δγ which is the Gauss-Newton correction sought after in the loop from Step 3644 to 3714 of FIGS. 40A-40D. The update is given explicitly in Step 3658 of FIG. 40B. Also at the same time the residual is updated via the formula shown explicitly in Step 3658 of FIG. 40B. Step 3660 transfers control to the Step 3686 in FIG. 40C which determines if the full, exact Jacobian will be used (e.g., step 3688) or the approximation shown explicitly in Step 3690. Depending upon which A is defined i.e., either the exact Jacobian or the approximation, JACFLAG is set to 1 or 0 and Step 3692 is carried out. It is the application of the Hermitian conjugate of the matrix A which is defined in either Step 3690 or 3688, depending upon whether the exact Jacobian is used. The Hermitian conjugate of A is applied to the updated residual vector r. Step 3694 calculates the ratio of two numbers. The first number is the magnitude of the updated residual calculated in Step 3692, the second number is the magnitude of the scattered field for all possible ω's, i.e. source positions, and φ's i.e., frequencies, and compares this ratio to ε the pre-determined tolerance. If the tolerance is larger than this calculated number, control is transferred to Step 3696 which effectively stores the image of the scattering potential and/or displays this image. If the tolerance is not satisfied, control is transferred to Step 3698 where β is calculated as shown explicitly in Step 3698.

Step 3700 calculates the new search direction which is given by the explicit formula shown. Note that the Hermitian conjugate applied to the residual vector r has already been calculated in previous Step 3698. Now transfer to Step 3704 of FIG. 40D and it is determined in Step 3710 if the maximum number of iterations allowed for the conjugate gradient solution have been exceeded. If no, then n, the counter for the conjugate gradient steps is incremented by one (e.g., step 3712), and control is transferred to Step 3646 of FIG. 40B. If, however, the maximum number of conjugate gradient iterations has been in fact exceeded, control is transferred to Step 3714 of FIG. 40D. This is the step that has been discussed previously in which the scattering potential is updated by the δγ vector which has been computed by the conjugate gradient iterations. Control is then transferred to Step 3716 of FIG. 40D where it is determined if the maximum number of Gauss-Newton corrections, specifically the δγ's, have been calculated. If the maximum number of Gauss-Newton (see earlier note regarding Gauss Newton vs Ribiere Polak) steps has been exceeded then the control is transferred to Step 3720 where the present scattering potential estimate is stored and/or displayed. If the maximum number of Gauss-Newton corrections has not been exceeded then the index parameter that counts the number of Gauss-Newton corrections that have been computed, is incremented by 1 (e.g., step 3718) and control is transferred to Step 3684 (arrow) which transfers control to Step 3622 of FIG. 40A. Again as before, in Step 3622 the forward problems for all possible it's and all possible φ's are calculated. More specifically control is transferred to Step 4140 of FIG. 41. This Figure has been discussed in detail above.

The loop just described and displayed in FIG. 40A-40D is now reiterated as many times as necessary to satisfy the tolerance condition shown in Step 3694 of FIG. 40C or until the maximum of Gauss-Newton iteration steps has been performed.

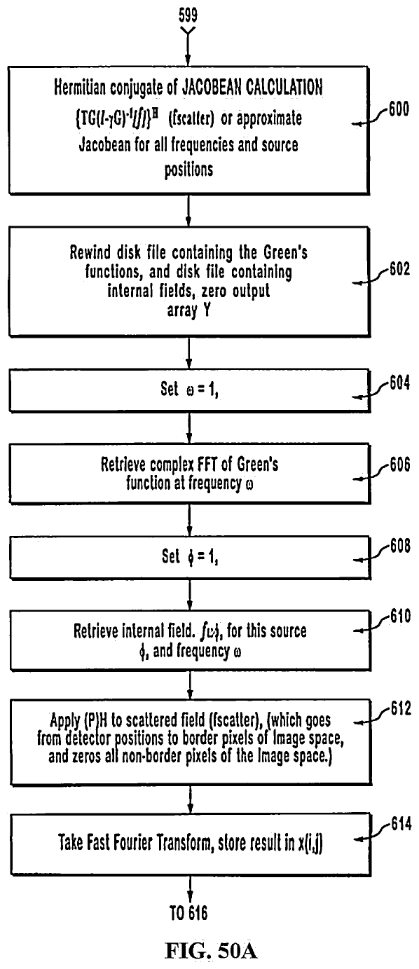

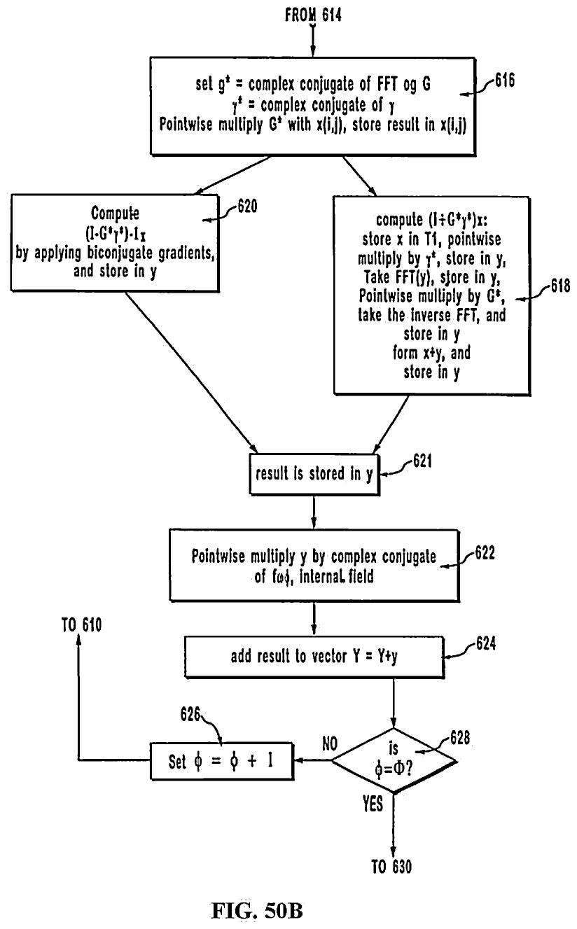

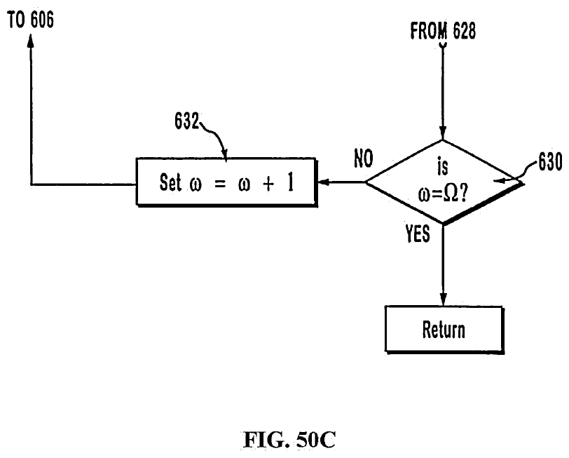

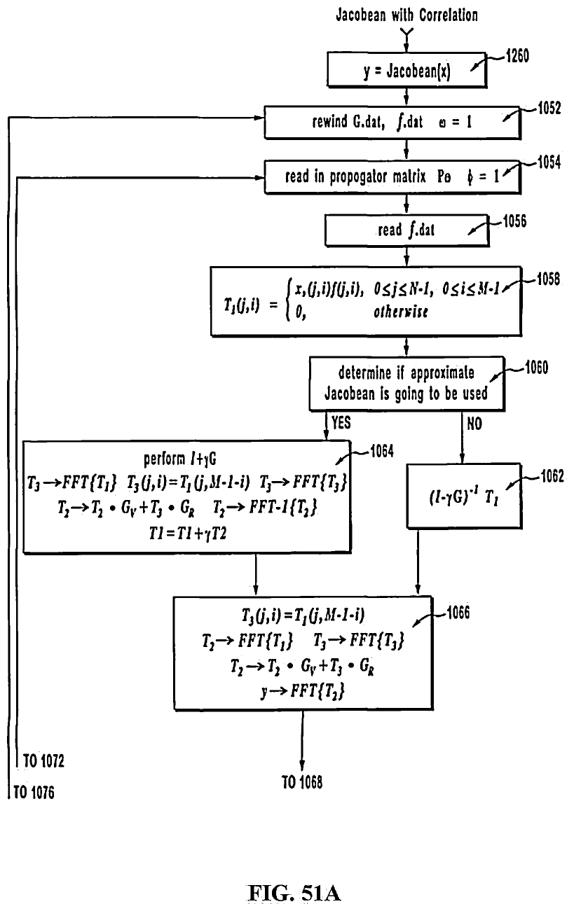

It is appropriate to study in detail the application of the Lippman-Schwinger operator and the Jacobian in the situation where layering is present. It is important to note that the discussion heretofore, has emphasized the special situation where layering is not present so that the free space or similar Green's function not involving any correlation calculation, has been used. This specialization is appropriate for pedagogical reasons, however, the importance of the layered Green's function makes it necessary to discuss in detail the application of the Lippman-Schwinger operator in this case. Before doing this however, we look at FIGS. 50A/B/C where the Hermitian conjugate of the Jacobian calculation is considered in the free space case. Just as in the calculation of the Jacobian itself (or its approximation) the variable JACFLAG determines whether BCG (or variant thereof) is applied or whether the binomial approximation shown is used.

In Step 600 the full Hermitian conjugate of the Jacobian calculation is undertaken with the disk files containing the Green's functions and the disk files containing the internal fields for each ω and each φ being rewound. In Step 604 ω is set equal to 1, i.e., the first frequency is considered. In Step 606 the complex Fast Fourier Transform (FFT) of the Green's function at this particular frequency ω is retrieved from disk where it has been stored previously. In Step 608 φ is set equal to 1 indicating we are dealing with the first source position. Step 610 retrieves the internal field foy, for this source position φ at frequency ω. Step 612 propagates the scattered field from the detector positions to the border pixels of the image space. Also in Step 612 the field values at the non-border pixels of the image space are equated to 0. In Step 614 the Fast Fourier Transform (FFT) of the field resulting from Step 612 is computed and stored in vector x. In Step 616 the complex conjugate of the Fast Fourier Transform (FFT) of the Green's function G is point-wise multiplied by x, the vector computed in Step 614. As before the actual computation of this inverse operator is computationally prohibitive. Therefore, the application of the inverse of I−G*γ* to the vector x is carried out by using bi-conjugate gradients or BiSTAB as discussed above. In Step 620 either the inverse of the operator I−G*γ* or the approximation (I+G*γ*) is applied to this result. The system [I−G*γ*]y=x is solved for γ using BCG. See FIGS. 42A and 42B. In Step 622 of FIG. 50B the point-wise multiplication by the complex conjugate of f_{ωφ}, the internal field for this frequency and source position, is carried out. This point-wise multiplication is the product with the result of Step 620. The Step 624 stores the result of this point-wise multiplication. Step 626 determines if φ is equal to Φ, the maximum number of source positions. If the maximum number of source positions has not yet been achieved, then the φ is incremented by 1 to indicate movement to the next source position. Transfer of control is now made to Step 610, where the internal field for this particular source position is retrieved. The loop from Step 610 to Step 626 is performed iteratively until the maximum number of source positions has been achieved. When this occurs, it is determined if ω is equal to Ω, the maximum number of frequencies, in Step 630. If the maximum number of frequencies has been achieved then control is transferred to Step 634 which transfers control back to FIG. 40A where the Hermitian conjugate of the Jacobian, or its approximation, was being applied. As shown in FIG. 50C, if, however, the maximum number of frequencies has not been achieved then transfer is made to Step 632 where the frequency index is incremented by 1 and control is transferred to Step 606 of FIG. 50A which is the retrieval of the complex Fast Fourier Transform (FFT) of the Green's function at new frequency o. This loop is now repeated until all possible frequencies and all possible source positions have been exhausted. At this time control is transferred to the inverse scattering routine in FIG. 40A-40D.

It is appropriate now to turn to that part of FIG. 49A/B/C which details the application of the reduced Jacobian calculation which is used by FIG. 40A-40D, i.e., the Gauss-Newton iteration in the situation where it is permissible to use the approximation to the Jacobian in place of the exact Jacobian. This calculation is called the reduced Jacobian calculation by virtue of the fact that no inverse operator is required to be calculated in this Figure. Therefore, the total computational complexity of this part of FIG. 49A/B/C (i.e., Step 715) is less than the corresponding Step 714 of FIG. 49A/B/C, in which the full exact Jacobian calculation is carried out. When the Jacobian calculation in FIG. 49A/B/C is being carried out to compute Step 3652 in FIG. 40A δγ is in fact pⁿ, the search direction for the conjugate gradient step in determining δγ, which in turn is the update to the scattering potential determined δγ this particular Gauss-Newton step. In Step 715a) the point-wise product is stored in vector x. In Step 715b) the Fast Fourier Transform (FFT) of the point-wise product is computed and in Step 715c) this Fast Fourier Transform (FFT) is taken with a point-wise product with the Fast Fourier Transform (FFT) of the Green's function for this particular frequency ω, and the inverse Fast Fourier Transform (FFT) of the point-wise product is taken to yield G convolved with the product f*δγ which result is stored in vector γ. In Step 715d) the point-wise product of vector y is taken with γ and the vector x computed and stored in Step 715a) is added to it. The Fast Fourier Transform (FFT) of this sum is calculated in Step 716 of FIG. 49A/B/C and the algorithm finishes exactly as described previously. Recall that transportation takes place in the scenario where the receivers exist at some finite distance from the image space in contradistinction to the truncation operation which takes place when the receivers exist at the border pixels of the image space. The truncation or transportation operator is discussed in FIG. 48 for the general case in which correlation, i.e. layering, is present or absent.

It is very important to note that the correlation that is calculated in the case of layering is, without exception, calculated as a convolution by a change of variables. It is the presence of the convolution which allows the introduction of the Fast Fourier Transform (FFT) and allows this algorithm to be completed in real time when the Fast Fourier Transform (FFT) is used in conjunction with the other specific algorithms and devices of this apparatus.

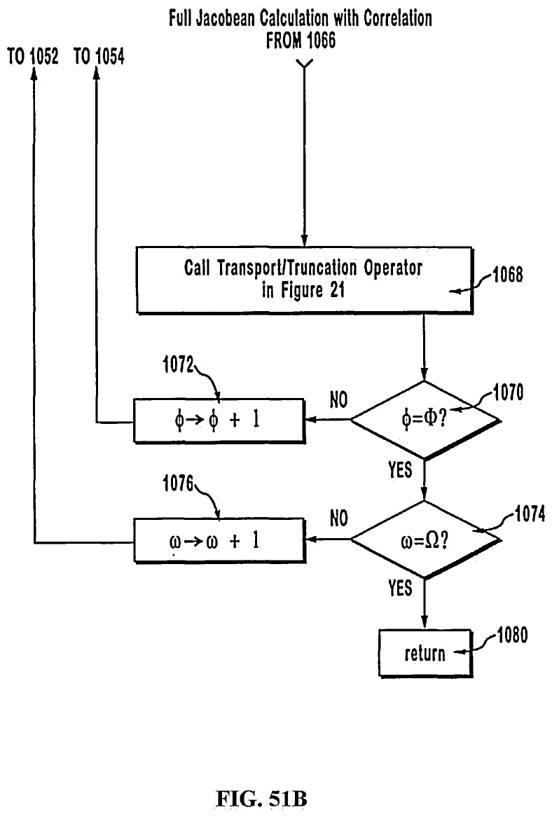

It is now appropriate to consider in detail the action of the Lippmann-Schwinger operator and the Jacobian in the case when layering is present. FIGS. 51A/B displays this case.

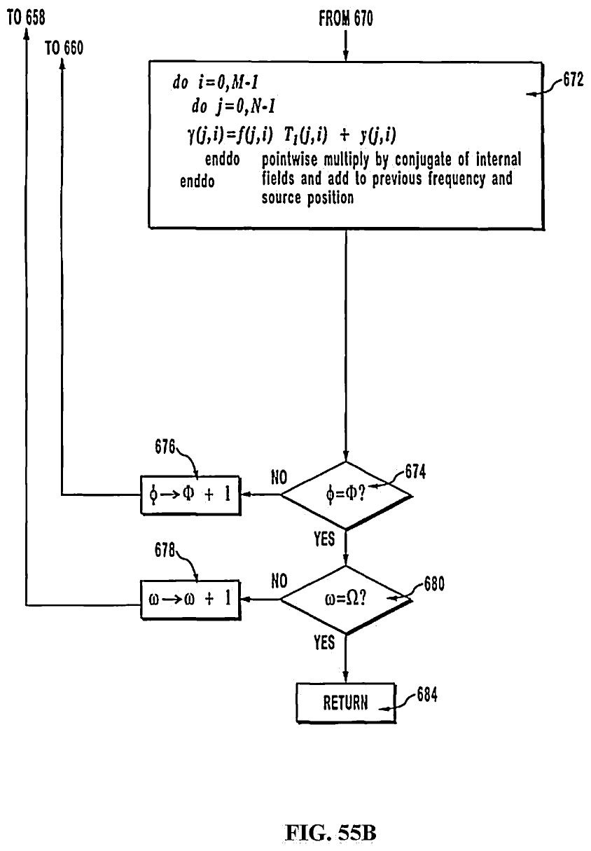

It is also important to note regarding the Jacobian calculation with correlation, that both the reduced and the exact form have their counterpart in the Hermitian conjugate. This is discussed in FIGS. 55A/B. In Step 654, the rewinding of the disk with the Green's function and the disk with the internal fields is carried out, and ω is set equal to 1. In Step 658 the complex Fast Fourier Transform (FFT) of the Green's function at this particular frequency ω is retrieved. Step 658 also sets φ (source position) equal to 1. Step 660 retrieves the internal field for this φ. Step 662 calculates the Hermitian conjugate of the truncation operator which puts values in border positions of the image space of the appropriate field and zeros everywhere else. Step 664 point-wise multiplies the result of 662 by complex conjugate of the Fast Fourier Transform (FFT) of the Green's function. Step 668 takes the inverse Fast Fourier Transform (FFT) and stores this in vector y. Now, in Step 670 depending upon the value for JACFLAG, either the full inverse (I−G*γ*)⁻¹ (via BCG or BiSTAB, etc.) or (I+G*γ*) is applied directly to y. The second alternative is carried out in the standard manner. That is, one takes the point-wise multiplication of the complex conjugate of γ, the scattering potential estimate at this time with the vector γ. The one takes the Fast Fourier Transform (FFT) of the result and takes the point-wise multiplication of the complex conjugate of the Fast Fourier Transform (FFT) with this result. Finally one adds the vector y and stores it in vector y, (overwriting y). The Step 672 multiplies the final result point-wise, with the complex conjugate of the internal field for this particular frequency and source position, and stores the result. Step 672 transfers control to 674 where it is determined if φ is equal to Φ. If no, then φ is incremented by 1 and returned to Step 660 in Step 676. If yes, control is transferred to 680 where it is determined if ω is equal to Ω; if yes, increment ω by 1 in Step 682, and transfer control to Step 656. If no, transfer control to Step 684 where control is transferred back to the inverse scattering main algorithm, FIGS. 40A-40D.

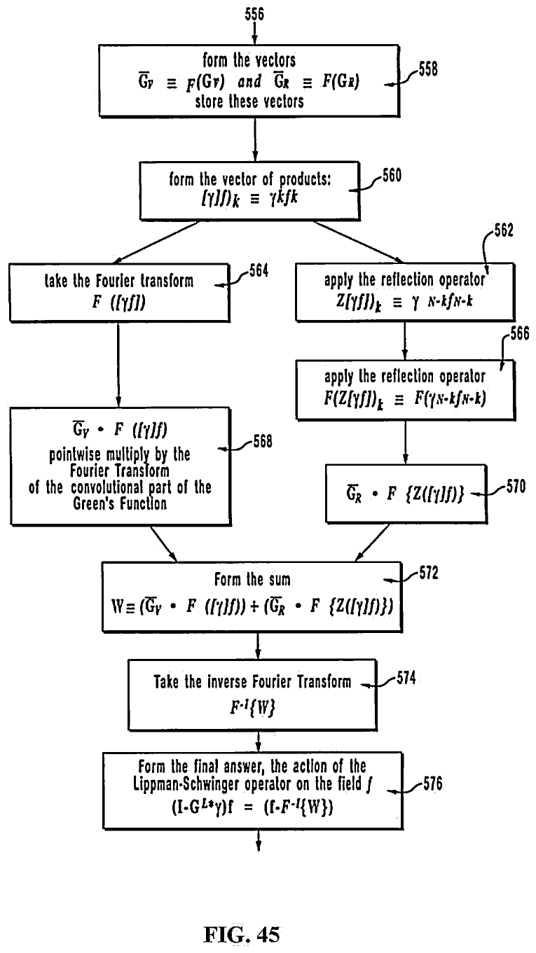

It is important to note at this point that when the operator I−Gγ is applied to the vector T₁, and also when the Lippmann-Schwinger operator I−Gγ is applied to any input vector in the layering scenario, FIG. 45 must be followed. Step 556 of FIG. 45 forms the vectors which store the Fast Fourier Transform (FFT) of the convolutional and the correlational parts of the layered Green's function separately. These convolutional and correlational parts are computed by appendices C and G for the two and three-dimensional cases, respectively, and are given explicitly by the formulas herein.

The step 558 passes control to step 560 where the vector of pointwise products shown explicitly there, is formed. This pointwise produce of γ, the scattering potential estimate, and F, the internal field, is Fourier transformed in step 564 and in parallel simultaneously in step 562, the reflection operator is applied to this pointwise product γF computed in step 560. In step 566, following step 562, the Fourier Transform of the reflected product γF is taken. In step 570 and in step 568, there is pointwise multiplication by the correlational part and the convolutional part of the layered Green's function, respectively. The correlational part of the layered Green's function is pointwise multiplied by the Fast Fourier Transform (FFT) computed in step 566, the convolutional part of layered Green's function fast Fourier transformed is pointwise multiplied by the Fast Fourier Transform (FFT) which was computed in step 564. In step 572, the vector W, which is the difference of the results of step 568 and 570, is computed and stored. In step 574, the inverse Fast Fourier Transform (FFT) of the vector W is computed. In step 576, the final answer which is the action of the Lippmann-Schwinger operator acting upon the field f is computed for the layered medium case, i.e., the case in which correlation exists. This process of applying a Lippmann-Schwinger operator in the presence of layering established in FIG. 45 is used explicitly in the calculation of the forward problems in step 3622 of FIG. 40A in the presence of layering. It is also carried out explicitly in Appendices B and P for the two and three-dimensional cases, respectively. It is also required in some of the calculations of the application of the Jacobian in the layered medium case.

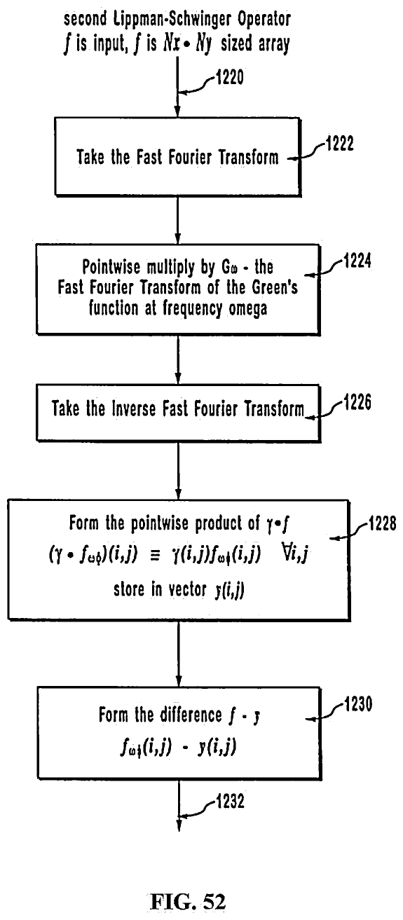

The operator described by FIG. 52, (I−γG), is called the “Second Lippmann-Schwinger operator”. This operator is in fact the transpose of the “Lippmann-Schwinger” operator so-called, i.e. (I−Gγ). Note that this is not the Hermitian adjoint of the Lippmann-Schwinger operator, precisely because no complex conjugate is taken.

The input to FIG. 52 is Fast Fourier Transformed in step 1222. The result is then pointwise multiplied by the Fast Fourier Transform (FFT) of the Green's function for this particular frequency. The result is inverse Fast Fourier Transformed and stored in vector y, in step 1226. The pointwise product γ·y is computed and stored in vector y. Finally the difference between input vector x, and y is formed in step 1230. Finally, the control is transferred back to the calling subroutine.

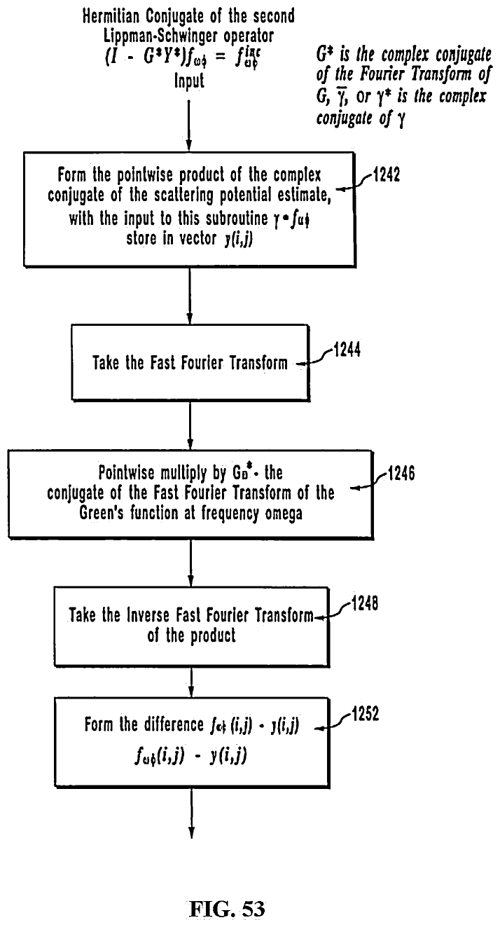

FIG. 53 details the application of the Hermitian conjugate of the “second Lippmann-Schwinger” operator In FIG. 53 control is transferred to step 1242, where the pointwise product is formed, between the complex conjugate of the scattering potential estimate, and the input to this subroutine, x. The Fast Fourier Transform (FFT) is then applied to this product in step 1244. In step 1246 the pointwise multiplication by G*, the complex conjugate of the Fast Fourier Transform (FFT) of the Green's function at frequency ω, is effected. In step 1248, the Inverse Fast Fourier Transform (FFT) of the product is taken, and finally the result is stored in y. In step 1252, the difference is formed between the input to this subroutine, x, and the computed vector y, and the result is stored in y.

FIG. 48 delineates the propagation operator that takes fields in the image space, and propagates them to the remote receivers at some distance from the image space. This propagator is discussed elsewhere in more detail.

In FIG. 48, the input is truncated to the border pixels, in step 1122. Next it is determined if the receivers are remote, or juxtaposed to the image space. If the detectors are in fact juxtaposed to the image space, the control is returned to the calling subroutine.

If the detectors are remote, the field is propagated with the aid of the propagator matrix P (described in section EXTENSION OF THE METHOD TO REMOTE RECEIVERS WITH ARBITRARY CHARACTERISTICS), in step 1128. Control is then transferred to the calling subroutine in the situation where the detectors are remote.

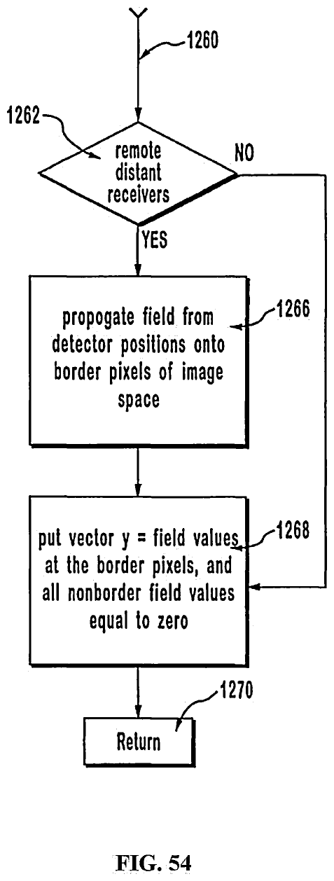

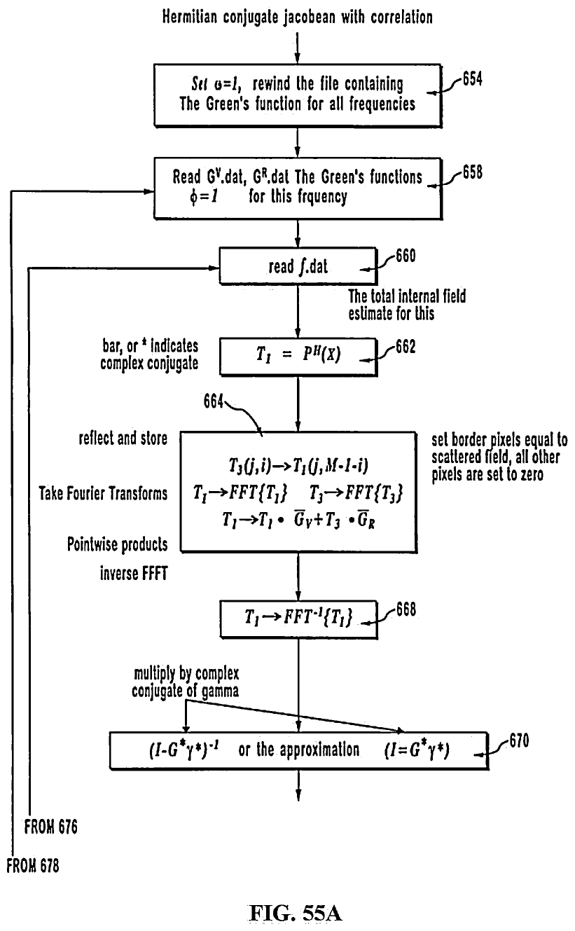

FIG. 54 details the Hermitian conjugate of the propagation operator discussed in FIG. 48:

In FIG. 54, the Hermitian conjugate of the Propagation operator P, it is first determined in step 1262, whether the receivers are at some remote location. If not, then control is transferred directly to step 1268. If the detectors are in fact remote, then control is passed to step 1266, where the fields are propagated via the propagation matrix to the border pixels of the image space. In either case, control is passed then to step 1270, and on to the calling subroutine.

FIGS. 51A/B determines the effect of the Jacobian on an input vector x in the presence of layering. Step 1050 transfers control to step 1052 where ω is set equal to 1. Also the files containing the Greens function for this frequency (both the correlations and the convolutional parts), and the internal field estimates, and the propagator matrix are rewound. Control is then transferred to step 1054 where the propagator matrix for this frequency is read in and also the Greens function for this particular frequency is read into memory. Also in step 1054 φ resource position index is set equal to 1. Control is then transferred to step 1056 where the file that contains the internal field estimate for this particular frequency and source position is read into memory. Control is then transferred to step 1058 where the pointwise product of the internal field estimate and the input vector x is computed. Next in step 1060 it is determined if the approximate Jacobian or the full Jacobian will be used. If the full Jacobian is to be used then control is transferred to step 1062 where biconjugate gradients or biconjugate gradients stabilized is used to solve for the action of the inverse matrix of I−γG acting on T₁. Recall T₁ is the temporary matrix which holds the pointwise product computed in step 1058. Control is then transferred to step 1066. If in step 1060 the determination is made to perform the approximate Jacobian operation then control is transferred to step 1064. In step 1064 the Fast Fourier Transform (FFT) is taken of the vector T₁ and it is stored in temporary vector T₂. Also the reflection operator is applied to the vector T₁ and the result is stored in vector T₃. Also the Fast Fourier Transform (FFT) is taken of the result of the planar reflection operator which is stored in T₃ and T₂ now contains the pointwise product of the convolutional part of the Greens function and the previous T₂ added to the pointwise product of T₃ with the correlational part of the Greens function for this particular frequency. The inverse Fast Fourier Transform (FFT) is applied to vector T₂ and the result is stored in vector T₂. Finally, the pointwise product between y and T₂ is formed and the result is added to vector T₁ and the result is stored in vector T₁. Regardless of whether step 1064 or 1062 has just been completed in step 1066 the reflection operator is applied to the result of either box 1062 or 1064 and the result is Fast Fourier transformed in vector T₃. Also the original vector T₁ is Fast Fourier transformed and the result is stored in Vector T₂. T₂ is the pointwise multiplied by the convolutional part of the layered Greens function at this particular frequency and the result is added to the pointwise product of T₃ with the correlational part of the layered Greens function at this frequency. The result is stored in vector T₂. Finally a Fast Fourier Transform (FFT) is applied to the resulting vector T₂ control is then transferred to step 1068 where the transportation or truncation operator given in FIG. 48 is applied to the result. In step 1070 it is determined if φ is equal to Φ if in fact φ is equal to Φ control is transferred to step 1074 where it is determined if ω is equal to ω. If φ is not equal to Φ control is transferred to step 1072 where φ is incremented by 1 and control is then transferred back to step 1056. If in step 1074 ω is determined to be equal to Ω control is transferred to step 1076 where φ is incremented by 1 and control is transferred back to step 1054. If ω is equal to Ω in step 1074 control is transferred step 1080 from there control is returned to the calling subroutine.

In FIG. 55A the Hermitian conjugate Jacobian with correlation present is applied to an input vector x in step 654 of FIG. 55A ω is set equal to 0 and the files containing the Greens functions for all frequencies are rewound in step 658. The Greens function both the convolutional and the correlational parts for this particular frequency are read into memory, also φ is set equal to 1. In step 660 the file containing the internal field estimate for this particular source position and frequency is read into memory in step 662 the Hermitian conjugate of the propagation operator is applied to the vector x the result is stored in vector T₁. In step 664 the reflection operator is applied to the vector T₁ and the result is stored in vector T₃ also Fast Fourier transforms are applied to both vector T₁ and vector T₃. Finally the pointwise product of the FFT of the complex conjugate of the convolution part of the layered Greens function is formed with T₁ and the result is added to the pointwise product of T₃ with the FFT of the correlational part of the layered Greens function with its complex conjugate taken. The result is stored in vector T₁. Control is then transferred to step 668 where the inverse Fast Fourier Transform (FFT) of T₁ is taken and the result is stored in vector T₁. In step 670 either the approximation or the full action of the inverse of the second Lippmann-Schwinger operator with Hermitian conjugate taken is applied as before. If the inverse operator is applied it is approximated by the solution to a system via biconjugate gradients or biconjugate gradients stabilized. In step 670 also, the approximation I+G* γ* is applied to the vector T₁ resulting from step 668, and the pointwise product with Gamma* i.e., the complex conjugate of γ is taken. The Fast Fourier Transform (FFT) of the result is then pointwise multiplied by G* the complex conjugate of the Greens function, with care being taken to insure that the convolution and the correlational parts are performed separately as before. This result is added to T₁, then stored in T₁ and control is then transferred to step 672 where the result is pointwise multiplied by the complex conjugate of the internal field estimates and the result is added to the previous frequency and source position result. Then control is transferred to step 674 where it is determined if φ is equal to Φ if not φ is incremented by 1 and control is transferred to step 660 if it is, control is transferred to step 680 where it is determined if ω is equal to Ω. If not the ω in incremented by 1 and control is transferred back to step 658. If it is, control is transferred back to the calling subroutine.

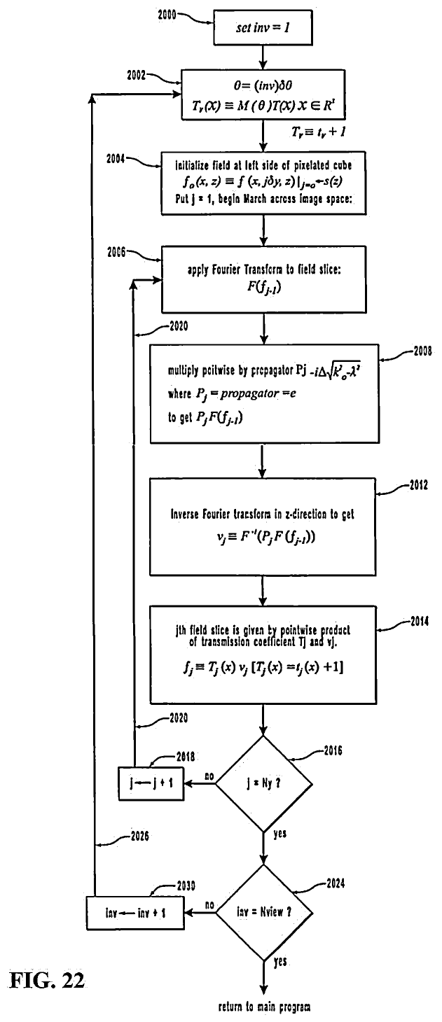

### Detailed Description of FIG. 22

Subroutine “scatter” is called from main program in FIG. 22, in order to effect the forward calculation of the fields for each view. Control is transferred to box 2000, where inν is set=1 (inv is the index of views). Next, in box 2002, is the calculation of rotation angle θ=(inν−1)δθ, and the setting up of rotation matrix M(θ), which is then applied to transmission coefficient matrix T to yield T_{y}(x)=M(θ)T(x)x∈R³

Next control is moved to box 2004, where the field for the first “slice” (i.e., j=1) is set to predetermined values: s(i,k), and the γ index, “j” is set=1, fₒ(x, z)≡f(x, jδy, z)|ⱼ₌₀←s(z).

Control is then transferred to box 2006, where the Fourier Transform is applied to field slice j−1 F(fⱼ₋₁)

Control is then transferred to box 2008, where the pointwise product is taken between the propagator Pⱼ, defined as

$${P}_{j} = propagator = e \sqrt[- Δ]{{λ}_{0}^{2} - {λ}^{2}} ,$$

and the result from box 2006, to get PⱼF(fⱼ₋₁). Now transfer to box 2012 where we take the Inverse Fourier transform in z-direction to get vⱼ≡F⁻¹(PⱼF(fⱼ₋₁)). Then move to box 2014, where j^{th} field slice is given by pointwise product of transmission coefficient Tⱼ and vⱼ. f_{j=Tj}(x)vⱼ. Then transfer control to decision box 2016, whereupon: if j is strictly less than N_{y}, then control moves to box 2018, where index j is increased by 1, and control is directed along arrow 2020 to box 2006. If j is exactly Nⱼ, control is transferred to decision box 2024. Then, if inν<N_{view}, control is transferred to box 2030 where inν is incremented by 1 to inν+1, and control is then transferred along arrow 2026 to box 2002. When inν=N_{view}, control is returned to the main program.

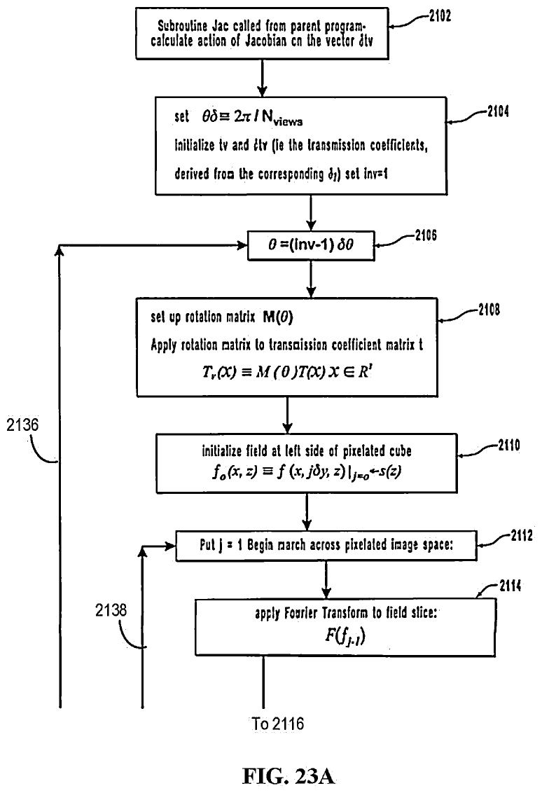

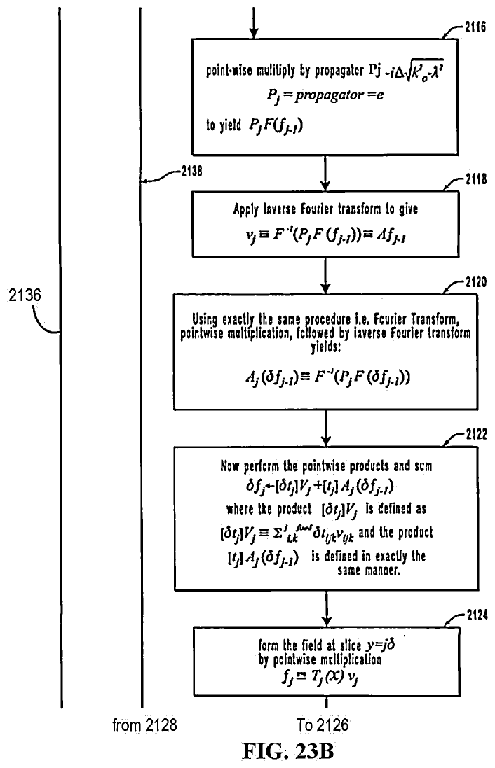

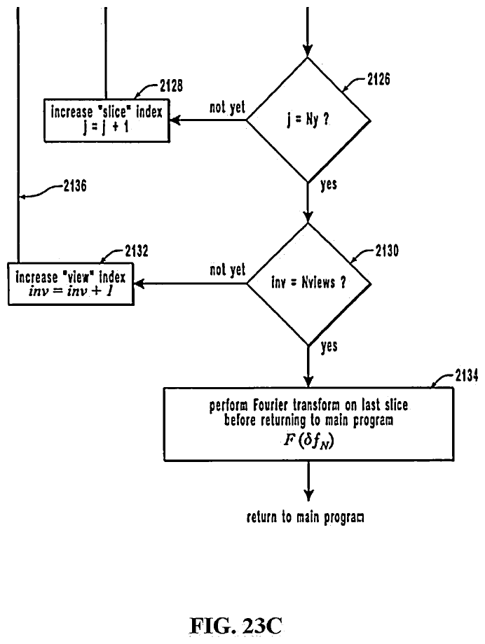

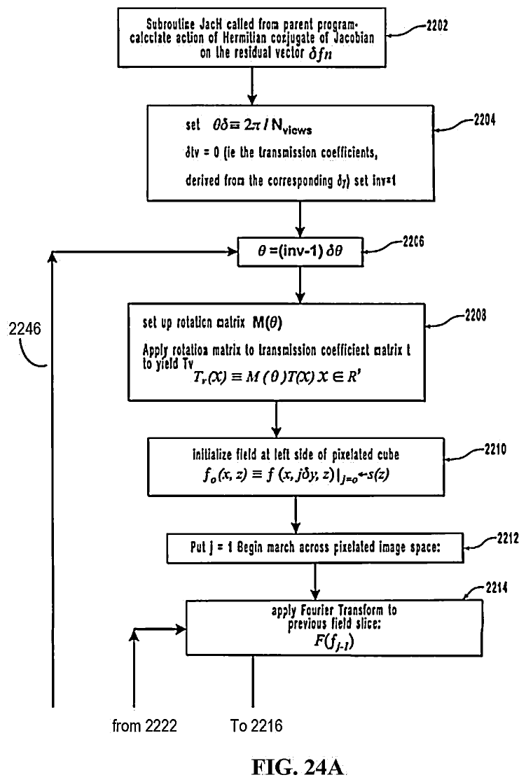

### Detailed Description of FIGS. 23A/B/C

Control is passed to subroutine Jac from the parent program in box 2102 It will calculate the action of the Jacobian of the forward problem on the vector δγ. Control is transferred to box 2104 where δθ is determined via the formula δθ≡2π/N_{views}, the spatially dependent transmission coefficient Tᵥ=tᵥ+1(Tᵥ=Tᵥ(x, y, z) and the input δt, or the corresponding δγ (object function) are also initialized, and inν is set=1, i.e., the index corresponding to view number Control is then transferred to box 2106 where one sets θ=(inν−1)δθ, and thus to box 2108 which sets up rotation matrix M(θ), and applies rotation matrix to transmission coefficient matrix T: Tᵥ(x)≡M(θ)T(x)x∈R³. Control is then transferred to box 2110 which initializes field at left side of pixelated cube using the formula fₒ(x, z)≡f(x, jδy, z)|ⱼ₌₀←s(z).

Now begin the march across the pixelated image space by transferring control to box 2112 where j (y index) is set=1. Then control moves to box 2114 where we apply Fourier Transform to field slice j−1: F(fⱼ₋₁). Then control is transferred to box 2116 of FIG. 20B where we multiply pointwise by propagator Pⱼ

$${P}_{j} = propagator = e \sqrt[- i Δ]{{k}_{0}^{2} - {λ}^{2}}$$

to get Pⱼ F(fⱼ₋₁). We now move to box 2118 where the Inverse Fourier transform in z-direction is applied to get vⱼ≡F⁻¹ (Pⱼ F(fⱼ₋₁))≡Afⱼ₋₁ (this slice vⱼ will be used later, and so is not overwritten). Control is then transferred to box 2120, where Aⱼ(δfⱼ₋₁)≡F⁻¹ (Pⱼ F(δfⱼ₋₁)) is computed in exactly the same manner, i.e. the Fourier transform is applied to δfⱼ₋₁, then the propagator is pointwise multiplied, and finally the inverse Fourier Transform is taken.

Control is then transferred to box 2122, where the pointwise product between the vⱼ (calculated above) and δtⱼ, [δtⱼ]vⱼ is formed. Also the pointwise product involving tⱼ: [tⱼ]Aⱼ(δfⱼ₋₁) is formed. The definition of the pointwise product is given explicitly by

$$[ δ {t}_{j} ] {v}_{j} ≡ {\sum }_{i , k}^{j fixed} δ {t}_{ijk} {v}_{ijk}$$

Finally form the sum of the resulting “vectors”, fⱼ←[δtⱼ]vⱼ+[tⱼ]Aⱼ(δfⱼ₋₁).

Control is then transferred to box 2124, where the field at slice γ=δj is formed to obviate the need to store/read the fields on disk, which is very time consuming. The field is given by the pointwise product of transmission coefficient Tⱼ and “slice” vⱼ. fⱼ≡Tⱼ(x)vⱼ

Control is now transferred to decision box 2126: If j=N_{γ} (ie the right hand side of the pixelated image space has been reached) then control is transferred to decision box 2130. If, however, the answer to query j=N_{γ} ? is no”, then control is transferred to 2128 where the “slice” index is incremented by +1, and control is transferred along arrow 2138 to box 2114 of FIG. 23A, where the Fourier transform is applied to the field slice fⱼ₋₁.

If control has been transferred to decision box 2130 of FIG. 23C, and if the index for “view”, inν=N_{view}, then control moves to box 2134, where the Fourier Transform is applied to the error on the detectors δf_{N}: fc←F(r≡δf_{N}), The resulting fc is pointwise multiplied by a scaling which in some cases, leads to more rapid convergence. This scaling is not critical to the implementation of the algorithm. The vector fc (scaled or not) is the output for the subroutine.

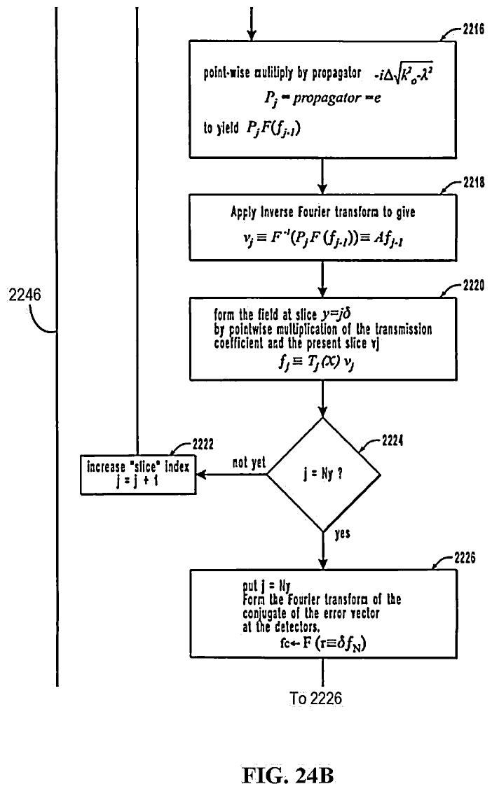

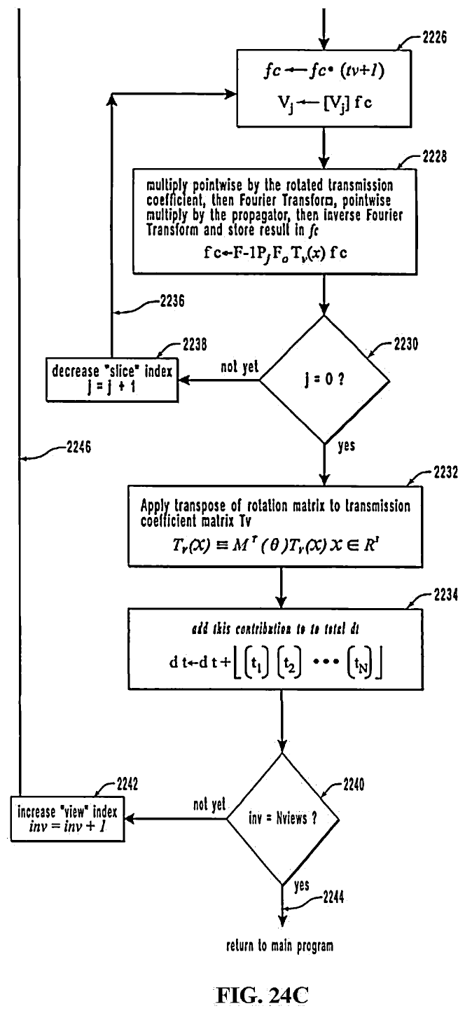

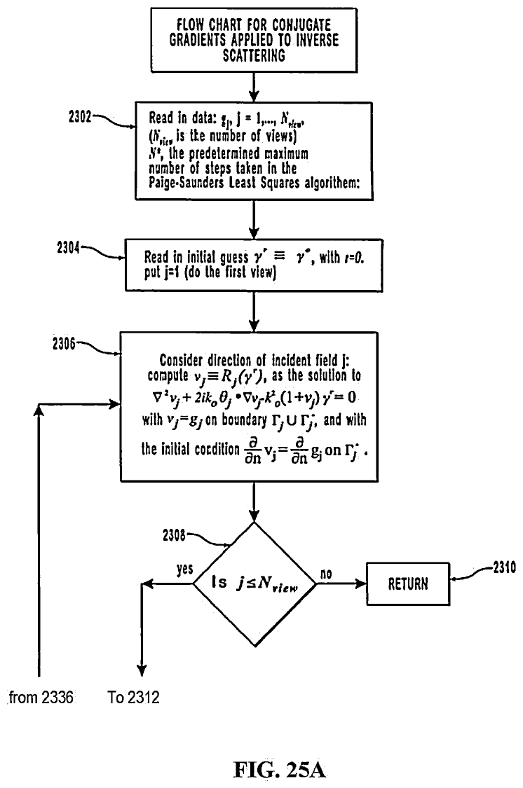

### Detailed Description of FIGS. 24A/B/C

Subroutine JacH(r) is called from parent program—it will calculate action of Hermitian conjugate of Jacobian on the residual vector r. Control is transferred from box 2202 to box 2204 where δθ is determined: δθ=2π/N_{views}, also δt is initialized=0 and inν is set=1. Control is transferred then, to box 2206 where θ is defined according to: θ=(inν−1)δθ. Control is transferred to box 2208, where the rotation matrix M(θ) is defined, and applied to transmission coefficient matrix T to yield Tᵥ. Tᵥ(x)≡M(θ)T(x)x∈R³. Then control is transferred to box 2210, where the field at the left side of the pixelated cube is initialized: fₒ(x, z)≡f(x, jδy, z)|ⱼ₌₀←s(z)

Next control is transferred to box 2212, where j is set=1, and the march across the pixelated image space is begun.

Next control is transferred to box 2214 the Fourier transform is applied to the field slice fⱼ₋₁:^{F}(fⱼ₋₁) and control is transferred to box 2216, where pointwise multiplication by the propagator Pⱼ is carried out, where Pⱼ=propagator=e⁻

$${P}_{j} = propagator = e \sqrt[- i Δ]{{k}_{0}^{2} - {λ}^{2}} .$$

The result, Pⱼ F(fⱼ₋₁) is then transferred to box 2218 where the inverse Fourier transform in the z (vertical) direction is applied, yielding vⱼ=F⁻¹ (Pⱼ F(fⱼ₋₁)). This vⱼ will be used below, and so is saved.

From this, control moves to box 2220, where the field slice at γ=jδ (recall that field values are recomputed to obviate the time consuming storage/retrieval of these on disk) is given by pointwise product of transmission coefficient Tⱼ and vⱼ. fⱼ ≡Tⱼ(x)vⱼ. Control now goes to decision box 2224, whereupon if j is strictly less than N_{γ}, control is switched to box 9292, where index j is increased by 1, and control reverts to box 2214, where the Fourier transform of the next field slice is taken. When j is equal to N_{γ}, control is transferred to box 2226 of FIG. 24B. This is the beginning of the backward moving loop (i.e., j decreases from N_{γ} to 1). Here we form the Fourier transform of the conjugate of the error vector at the detectors, and store result in C: fc←F(r≡δf_{N}).

Now use vⱼ (calculated in loop above) and fields fcⱼ to calculate action of Jacobian on r, the error in field at the detector (far side of image space) in the following manner:

Control then moves to box 2226 where fc is redefined as fc·(t_{ν}+1) and the pointwise product between the resulting fc, and vⱼ is taken, and stored in vⱼ←[vⱼ]fc. Control is then transferred to box 2228 where we pointwise multiply fc by rotated transmission coefficient Tν, Fourier Transform the result, pointwise multiply by propagator Pⱼ, then inverse Fourier Transform (in z direction) the result; finally store the resulting slice in fc (overwriting previous contents) fc←F⁻¹PⱼF o Tν(x)fc. Next, control is transferred to box 2230, in order to test whether j=0. If not, then control is transferred to box 2238, where the y index, “j”, is decreased by one (put j←j−1), and control is transferred along arrow 2236, back to box 2226. If, however, j does=0, then the loop is done, and control moves to box 2232, where the hermitian conjugate of rotation matrix is applied to matrix of transmission coefficients tv computed in the above loop, where:

$$tv = [ ( {v}_{1} ) ( {v}_{2} ) … ( {v}_{N} ) ]$$

in order to rotate (estimate of) object back to original orientation

$$[ ( {t}_{1} ) ( {t}_{2} ) … ( {t}_{N} ) ] ≡ {Rot}^{H} ( tv )$$

Then, control moves to box 2234, where we add contribution for this view to total transmission coefficient adjustment dₜ

$$dt ← dt + [ ( {t}_{1} ) ( {t}_{2} ) … ( {t}_{N} ) ]$$

Now control is transferred over to decision box 2240, where it is determined if index inν=N_{views}. If it is, then control is transferred over to box 2242, where index inν is increased by 1: inν←inν+1, and control is transferred up arrow 2242 to box 2246 to do the next view If it is determined that index inν does in fact=N_{views}, the control is passed along arrow 2244, to return to the calling program, with output→(δt)

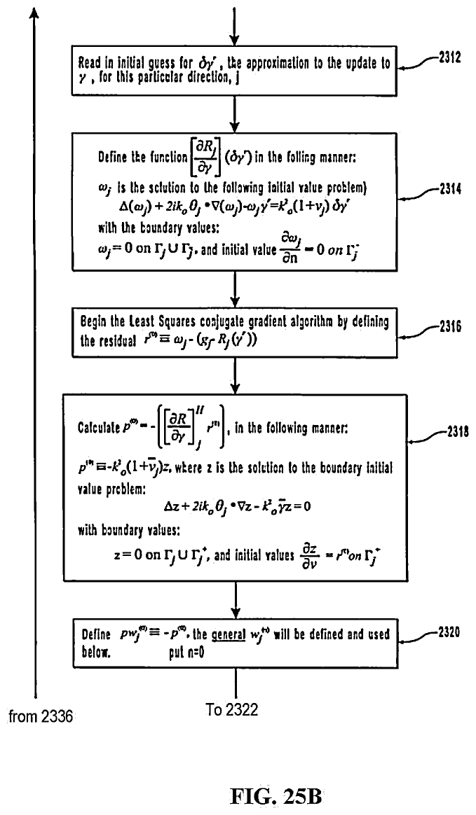

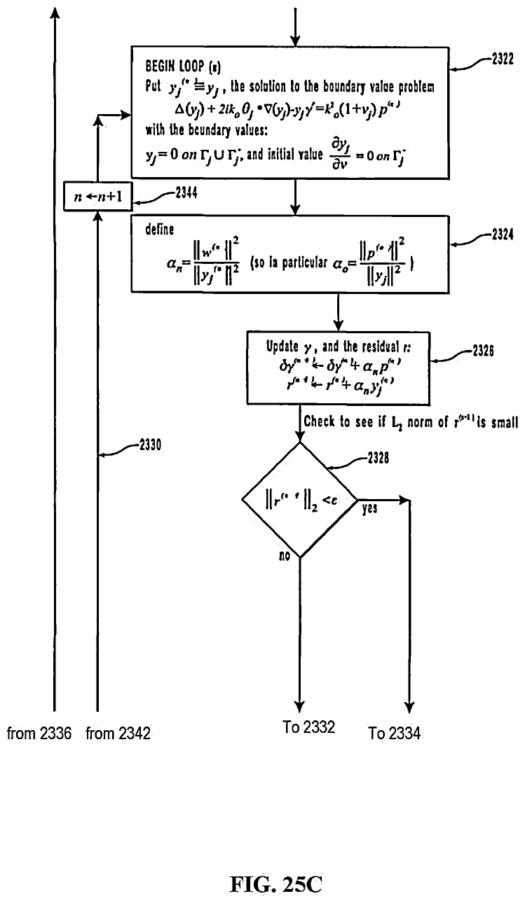

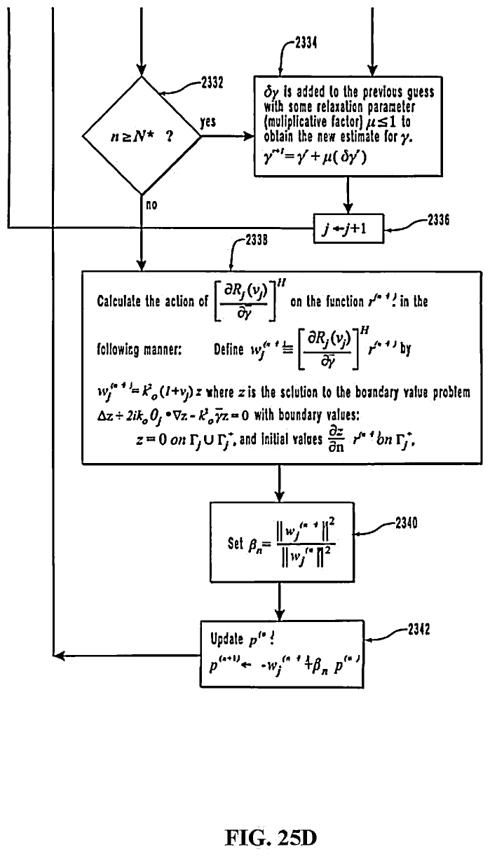

### Detailed Description of FIGS. 25A/B/C/D

Control is transferred from the calling program through arrow 2300 on FIG. 25A to 2302 where the data gⱼ, for j=1, . . . ,N_{view} is read in, and the predetermined maximum number of steps taken in the conjugate gradient method is read in as well. Then control is transferred to box 2304 of FIG. 25A where the initial guess for γ the object function, is read into memory. Also, in this box 2304, the index j is set=1 to indicate that the first view is being considered. Then control is transferred down to box 2306 where the forward problem for direction j is solved, that is, the partial differential equation for vⱼ is solved:

$${∇}^{2} {ν}_{f} + 2 i {k}_{o} {θ}_{j} \cdot ∇ {v}_{f} - {k}_{o}^{2} ( 1 + {ν}_{j} ) {γ}^{r}$$

with vⱼ=gⱼ on boundary

$${Γ}_{j} ⋃ {Γ}_{j}^{-} ,$$

and with the initial condition

$$\frac{\partial }{\partial n} {ν}_{j} = \frac{\partial }{\partial n} {g}_{j}$$

on

$${Γ}_{j}^{-} .$$

The notation ∂/∂n designates the interior normal derivative. The forward operator R is defined as follows:

$${R}_{j} ( {γ}^{r} ) ≡ {v}_{j} {\vert}_{{Γ}_{j}^{+}} ,$$

that is, Rⱼ(γ)≡vⱼ_{^{−}} restricted-to-

$${Γ}_{j}^{+} , {Γ}_{j}^{+}$$

being the forward scattering part of the boundary of Q.

Now the control is transferred over to decision box 2308, where it is determined if j≤N_{view}. If it is not, then the algorithm continues to box 2310, where control is returned to a calling subprogram, or the final image is stored and displayed. That is, if j>N_{view}, then the algorithm has updated the object function γ for all incident directions, and control is transferred to back to the calling program or the image is stored/displayed.

If j≤N_{view}, then control is transferred over to box 2312, where an initial guess for δγ^{r}, the update to γ for this particular direction j. is read in.

Control is then transferred to box 2314 (if j≤N_{view}) where the solution to the initial value problem

$$Δ ( {ω}_{j} ) + 2 i {k}_{o} {θ}_{j} \cdot ∇ ( {ω}_{j} ) - {ω}_{j} {γ}^{r} = {k}_{o}^{2} ( 1 + {ν}_{j} ) δ {γ}^{{r}_{1}}$$

with the boundary values:
ωⱼ=0 on

$${Γ}_{j} ⋃ {Γ}_{j}^{-} ,$$

and initial value

$$\frac{\partial {ω}_{j}}{\partial v} = 0$$

on

$${Γ}_{j}^{-}$$

is determined by the method of finite differences or the marching method described in this patent (F. Natterer and F. Wubbelling paper). The vⱼ is the same as in box 2306. The solution to this problem, the function ωⱼ, is the result of applying the Jacobian of Rⱼ to the current guess for the “object function correction,” δγ.

Next control is transferred to box 2316 where the residual r⁽⁰⁾≡−(gⱼ−Rⱼ(γ^{r})) is defined. Next control is transferred to box 2318, where the boundary initial value problem:

$$Δ z + 2 i {k}_{o} {θ}_{j} ∇ z - {k}_{o}^{2} γ z = 0$$

with boundary values: P
z=0 on

$${Γ}_{j} ⋃ {Γ}_{j}^{+} ,$$

and initial values

$$\frac{\partial z}{\partial v} = {r}^{( 0 )}$$

on

$${Γ}_{j}^{+}$$

is solved. Then, the function

$${p}^{( 0 )} = - ( {[ \frac{\partial R}{\partial γ} ]}_{j}^{H} {r}^{( 0 )} )$$

is defined as

$${ρ}^{( 0 )} ≡ - {k}_{o}^{2} ( 1 + {\overline{v}}_{j} ) z ,$$

where z is the solution to the above initial value problem. Note: in the above formula: v̅ⱼ is the complex conjugate of vⱼ.

Now, control is transferred to box 2320, where the definition wⱼ⁽⁰⁾≡−ρ⁽⁰⁾ is enforced—the general wⱼ⁽ⁿ⁾ will be defined and used later in this algorithm, also the loop index n is set=0. Then control is transferred to box 2322 where we define yⱼ⁽ⁿ⁾=yⱼ, with γⱼ the solution to the boundary value problem

$$Δ ( {y}_{j} ) + 2 i {k}_{o} {θ}_{j} \cdot ∇ ( {y}_{j} ) - {y}_{j} {γ}^{r} = {k}_{o}^{2} ( 1 + {v}_{j} ) {ρ}^{( n )}$$

with the boundary values:
   yⱼ=0 on Γⱼ∪Γⱼ, and initial value

$$\frac{\partial {y}_{j}}{\partial v} = 0$$

on Γⱼ³¹

Then control is transferred to box 2324, where an is defined as an appropriate ratio. Then control is transferred to box 2326 where the correction to the object function δγ and the residual r are updated. Control is then transferred to decision box 2328, where it is determined whether or not the magnitude of the vector of residuals is smaller than some predetermined and fixed E. If it is not then control is transferred to decision box 2332, where it is determined whether the index n is larger than N*. If either of these two criteria are met, then it is assumed that the subprocess that converges to δγⱼ is finished, and control is transferred to box 2334 where the object function γ is updated by the formula γ^{r+1}=γ^{r}+μ(δγ^{r}). Then in box 2336 the view index j is incremented by 1, and control is then transferred back to box 2306 to read in the next initial guess δγ·j for the next direction.

If neither of these conditions is met then control is transferred to box 2338, where the action of the Hermitian adjoint of the Jacobian

$$[ \frac{\partial {R}_{j}}{\partial γ} ]$$

on the function r⁽ⁿ⁺¹⁾,

$$( symbolically represented as {[ \frac{\partial {R}_{j}}{\partial γ} ]}^{H} {r}^{( n + 1 )} )$$

is calculated. We use the notation

$${w}_{j}^{( n + 1 )} ≡ {[ \frac{\partial {R}_{j} ( {v}_{j} )}{\partial γ} ]}^{H} {r}^{( n + 1 )}$$

This wⱼ⁽ⁿ⁺¹⁾ is explicitly calculated in two stages:
(1) Calculate z as the solution to the boundary value problem

$$Δ z + 2 i {k}_{o} {θ}_{j} \cdot ∇ z - {k}_{o}^{2} \overline{γ} z$$

with boundary values:
z=0 on

$${Γ}_{j} ⋃ {Γ}_{j}^{+} ,$$

and initial values

$$\frac{\partial z}{\partial v} = {r}^{( n + 1 )}$$

on

$${Γ}_{j}^{+} ,$$

(2)

$${w}_{j}^{( n + 1 )} = {k}_{o}^{2} ( 1 + {\overline{γ}}_{j} ) z$$

Control is then transferred to box 2340 where the constant βₙ is calculated via the formula:

$${β}_{n} = \frac{{‖ {w}_{j}^{( n + 1 )} ‖}^{2}}{{‖ {w}_{j}^{( n )} ‖}^{2}} .$$

Control is then transferred to box 2342 where the search direction ρ⁽ⁿ⁾ is updated via the formula

$${ρ}^{( n + 1 )} ← {w}_{j}^{( n + 1 )} + {β}_{n} {ρ}^{( n )} .$$

Now one loop of the conjugate gradient algorithm has been completed and control is transferred up to box 2344 where the loop index n is increased by 1 and control is then transferred to box 2322 where the next solution to the given boundary value problem is calculated. Note that the γ^{r}, and the vⱼ are both known. This loop is repeated until one of the criteria in box 2328 or 2332 is finally met, whereupon control is transferred to the box 2306 and the next view is dealt with.

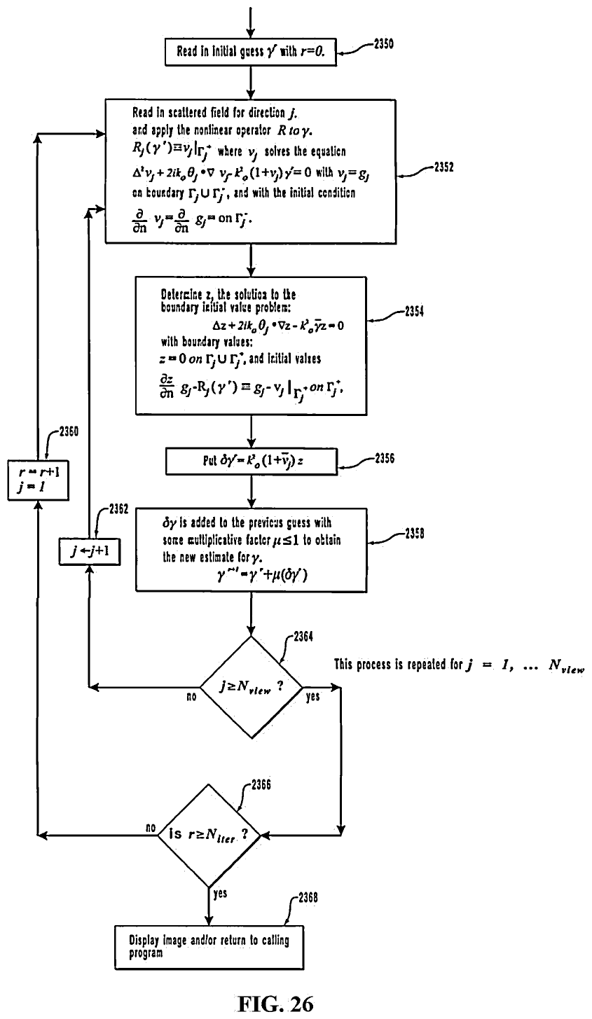

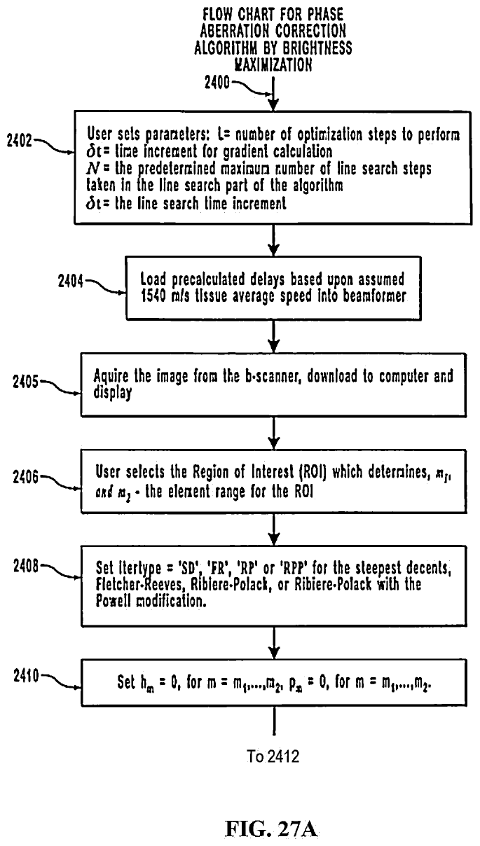

### Detailed Description of Flow Chart for Propagation-Backpropagation Method, FIG. 26

Control is transferred to box 2350 of FIG. 26, where the initial guess or estimate for γ. From there control is transferred to box 2352 where the data (scattered or total field) is read in. Also in this box the forward problem with an object function of γ^{r} is carried out for the j^{th} direction.

Next control is transferred to box 2354 where the initial value and boundary value problem shown in that box, is solved. This is part of the back-propagation stage of the process. Then control is transferred over to box 2356 where the back-propagation process is completed by the definition of δγ given there. Then control is transferred down to box 2358 where δ^{r} is updated with a multiplicative factor of δγ. Then control is transferred over to box 2364 where it is determined if j is greater than or equal to N_{view}. If so, then control is transferred over to box 2366 where it is determined if r is greater than or equal to Niter. If r is greater than or equal to Niter, then control is transferred down to box 2368 where control is transferred over to the calling program and/or the image of γ is displayed. If the criterion in box 2364 is not satisfied, control is transferred to 2362 where j is increased by 1. Then control is taken back to box 2352 where the data (scattered or total field) for new direction j is taken, and the corresponding forward problem is solved for direction j. If the r counter is not greater than Niter, control is transferred to box 2360, where j is reset to 1, since it has already been determined in box 2364 that all the views have been processed. Also the iteration counter r is increased by 1, and control is returned to box 2352, where the forward problem corresponding to the direction j=1 is performed. Note that one may wish to bypass the redoing of the forward problem after each iteration in the r variable. This would require the storing of each vⱼ for all of the directions, furthermore, since the computation of the forward problem is very fast, it is approximately as efficient to recompute the forward problem each iteration of r, as opposed to going through several iterations before recomputing the forward solutions.

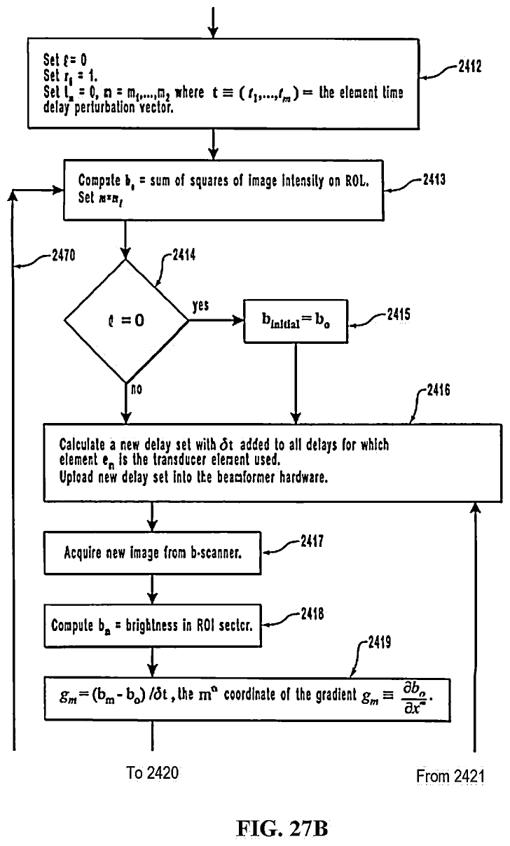

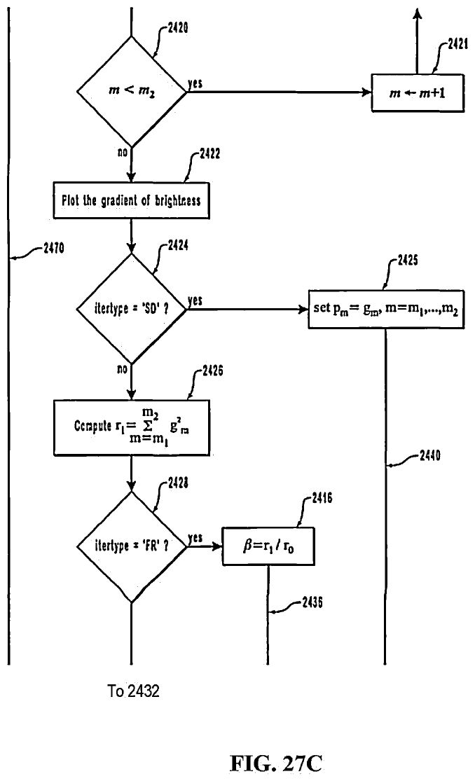

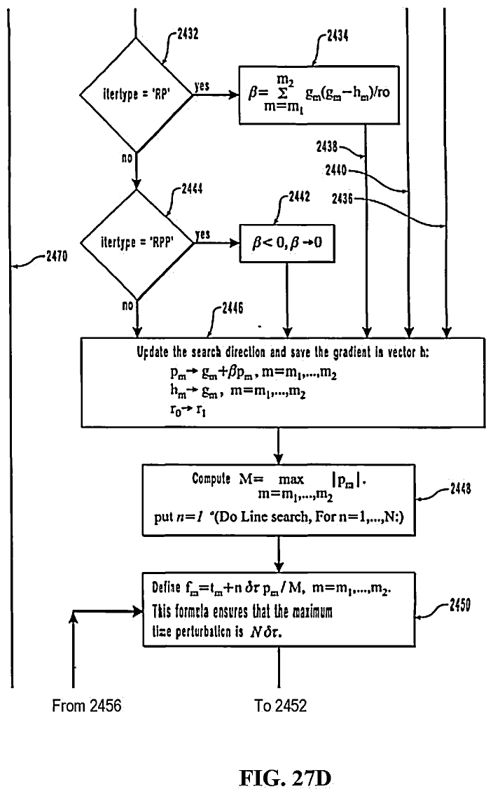

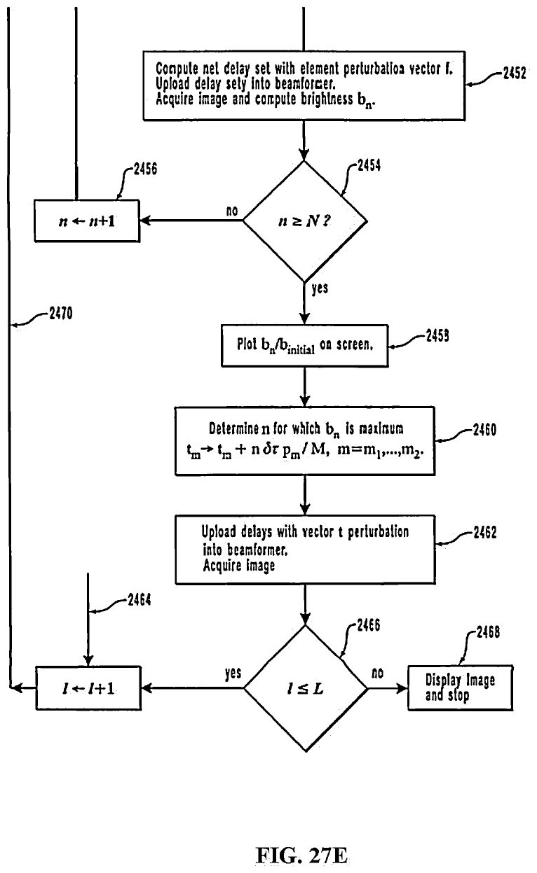

### Detailed Description of Flow Chart for Maximization of Brightness Phase Aberration Correction FIGS. 27A/B/C/D/E

First, flow is transferred down arrow 2400 from the calling program. Then in box 2402 δt, the time increment for gradient calculation, t, the line search time step, Nₛ the predetermined maximum number of line search steps, and L, the predetermined total number of CG iterations to be performed, are read in. Control is then transferred over to box 2404, where the precalculated beamformer delays based upon an assumed speed of sound in the tissue are loaded into the beamformer hardware. Then control is transferred to box 2405, where the b-scan image is acquired. Then control is transferred to box 2406 where the Region of Interest (ROI) is selected by the user. The element range used to form the image in the ROI, m₁ to m₂, are determined for this ROI. Then control is moved to box 2408 where the type of iteration is chosen: itertype=‘SD’ corresponds to steepest descents, itertype=‘FR’ corresponds to Fletcher Reeves', ‘RP’ corresponds to Ribiere-Polak, and finally ‘RPP’ corresponds to Ribiere-Polak with the Powell modification. Next control is transferred to box 2410 where the vector h with components hₘ for m=m₁, . . . , m₂ is set identically=0, similarly all of the components of the vector p, pₘ are set=0.

Next control is transferred to box 2412 of FIG. 27B, where the initial gradient magnitude, r₀, is set=1, and the vector t (with components tₘ=the time delay perturbation for element m) is set=0. Now control is transferred to box 2413 where the index 1 is set equal to zero, and the sum of squares of the image intensity in the ROI is computed and stored in b₀. Now control is transferred to decision box 2414 where it is determined if index l=0. If it is then control is transferred to box 2415, bₒ is stored in bᵢₙᵢₜᵢₐₗ, and control is transferred down to box 2416. If l≠0, then control is transferred to box 2416 where δt (NOT δτ) is added to all beamformer delays which correspond to transducer element eₘ. The new (perturbed) delay set is uploaded to the beamformer, and control goes to box 2417, where the new image is acquired form the b-scanner. Next control goes to box 2418, where the brightness in the ROI is computed and stored in bₘ. Now control is transferred over to box 2419, where the gradient component gₘ is approximated by a finite difference formula. Then control is transferred to box 2420, where it is determined if m<m₂, if it is then control goes to box 2421, where index m is increased by 1 and control goes back to box 2416. If not, then control is transferred down to box 2422, where the gradient of brightness is plotted. Next control is transferred to decision box 2424, if itertype=‘SD’, then the vector p is set equal to the gradient g and control is transferred directly down to box 2446 via arrow 2440. If itertype is not=‘SD’, then transfer goes to box 2426, where the gradient magnitude r₁ is computed as the sum of squares of the components of the vector g, the gradient. Next control is transferred down to decision box 2428, where itertype is compared to ‘FR’. If they are not equal then control goes to decision box 2432, where itertype is compared with ‘RP’. If they are in fact equal, then control is transferred to box 2430, where the constant β is defined as the ratio: r₁/r₀. Control is then transferred to box 2446 via arrow 2438.

At decision box 2432 it is determined if itertype=‘RP’. It is true then control is switched over to box 2434 where the constant β is calculated as shown. If itertype is not=‘RP’, then control is transferred down to box 2444 where it is determined if itertype=‘RPP’. If it is, then control is transferred over to box 2442, where R is checked to make sure that it is non-negative, if not, then p is set=0 and control is transferred over to box 2446. If it is, in fact non-negative, then control is transferred over to box 2446, where the search direction p is updated as shown in box 2446. Also in box 2446 the vector g with components, gₘ with m=m₁, . . . , m₂.is stored in vector h, and r_{ω} is redefined to be equal to r₁, that is, the value r₁ is stored in r₀. The control is then transferred over to box 2448, where the constant M is calculated as the maximum of the absolute values of the components of direction p. Also in this box, n the line search index is set=1. Then control is transferred down to box 2450, where the vector f with components fₘ is determined. Then control is transferred down to box 2452 where the net delay set with element perturbation vector f is computed and uploaded to the beamformer hardware. The corresponding image is acquired from the b-scanner and the resultant brightness in the ROL, bₙ, is computed (as the sum of squares mentioned before). Then control is transferred to box 2454 where it is determined if n is greater than or equal to Nₛ the predetermined number of steps in the line search. If so, then control is transferred down to box 2458 where the normalized values bₙ/bᵢₙᵢₜᵢₐₗ, are plotted. If not, then control is transferred over to box 2456, where the index n is increased by 1, and control is then transferred up to box 2450 where the perturbation vector f is recalculated with the new value of n.

Once control has been passed down to box 2458 and the normalized bₙ/bᵢₙᵢₜᵢₐₗ for n=1, 2, . . . Nₛ, has been plotted, then control is transferred to box 2460, where the n for which bₙ is maximal is determined. Then control moves to box 2462, where the delays tₘ (the components of vector t) are uploaded into the beamformer hardware. These delays are the ones which maximize the brightness of the image in the direction of the present search direction. Next control is transferred down to decision box 2466, where it is determined if l is strictly less than L, the predetermined number of iterations in the brightness maximizing subroutine. If it is true, then control is transferred to box 2464, where the index 1 is increased by 1, and control is transferred up to box 2413 where the image is acquired for the given beamformer delays. Also, the index m is reset to equal m₁, in order that the gradient can be calculated once more.

If the predetermined number of steps have been carried out, then control is then transferred over to box 2468, where the image is acquired from the b-scanner using the time delays in t and control reverts to the calling program, or else a “stop” is executed.

FIG. 31 shows the method for solving for the object function γ in the scattered field domain as discussed in METHOD 1 directly below. It uses an iterative procedure applied to a square non-linear operator to first determine an equivalent scattered field, and then transform to the object function solution. Since the nonlinear system is square, it is possible to use BiSTAB (BiConjugate Gradients Stabilized) in place of generic Conjugate Gradients in the solution algorithm, which is a great advantage.

FIG. 32 shows the method for solving for the object function γ iteratively in the object function domain. The method is discussed as METHOD 2 directly below. Again, since the system is square, it follows that BiSTAB is the proper algorithm to use.

### Method 1: Iteration in the Scattered Field Manifold

Solving for γ by inverting Eₛ=F(γ), where F(γ)=Dγ(I−Cγ)⁻¹E_{b}, requires inverting from a range to a domain that are not the same size. Define Eₛ^{Born}=DγE_{b}. It is now possible to solve for Eₛ^{Born} from the square system Eₛ=F(B(Eₛ^{Born})). After finding Eₛ^{Born} we find γ by the direct substitution γ=B(Eₛ^{Born}). This has the advantage of allowing use of square system solvers, such as biconjugate gradients (BCG) or stabilized biconjugate gradients (BiSTAB), which are faster and better conditioned than rectangular system solvers, e.g., conjugate gradients (CG). The function B can be any one-to-one mapping such as the Born approximation or backpropagation. Thus, we will also investigate and develop the potentially faster non linear, square system method described in FIG. 31.

Solving for γ by inverting Eₛ=F(γ), where F(γ)=Dγ(I−Cγ)⁻¹E_{b}, requires inverting from a range to a domain that are not the same size. However, we can solve for Eₛ^{Born} from the square system Eₛ=F(B(Eₛ^{Born})). After finding Eₛ^{Born} we find γ by the direct substitution γ=B(Eₛ^{Born}). This method has the advantage of allowing use of square system solvers, such as biconjugate gradients (BCG) or stabilized biconjugate gradients (BiSTAB), which are faster and better conditioned than rectangular system solvers, e.g., conjugate gradients (CG). The function B can be any one-to-one mapping such as the Born approximation or backpropagation.

### Method 2: Iteration in the Object Function Manifold

Use the Born approximation as an example.

$${E}_{s} = D {γ}^{B o r n} {E}_{b} = [ D {E}_{b} ] {γ}^{B o r n}$$

Then

$${γ}^{B o r n} = {[ D {E}_{b} ]}^{- 1} {E}_{s}$$

Where [DE_{b}]⁻¹ represents the Born reconstruction. Multiply both sides of

$${E}_{S} = D {γ ( I - C γ )}^{- 1} {E}_{b}$$

by [DE_{b}]⁻¹, that is, apply the Born reconstruction procedure to both sides.

Now, solve square nonlinear system

$${γ}^{Born} = {[ {DE}_{b} ]}^{- 1} D {γ ( I - C γ )}^{- 1} {E}_{b} ,$$

for γ, in the γ domain by means of BiSTAB. The Jacobian will be square. There may be less work than with Method 1.

Actually this will involve approximately the same workload as Method 1 at each iteration. This is because in Method 2, each iteration requires the application of the Born reconstruction procedure, whereas in Method 1), we are also required to calculate the Born approximation to the scattered field at each iteration, and then finish off with one application of the Born reconstruction procedure in order to get y.

METHOD 3:′ Use previous guess γ⁽ⁿ⁻¹⁾

To understand the third method and put all 3 methods into a consistent framework, consider the nonlinear operator “φ”, which maps “object functions” γ to “scattered fields”:

$$ϕ ( γ ) = D [ γ ] {( I - C [ γ ] )}^{- 1} {E}_{b}$$

In terms of this operator, the equation we wish to solve for γ, is

$$ϕ ( γ ) = {E}_{m}$$

where Eₘ is the measured field (as above). The standard approach is to solve the least squares minimization problem

$${min}_{y} {‖ {E}_{m} - ϕ ( γ ) ‖}^{2} ,$$

as discussed above. This involves the use of conjugate gradient methods. However, if we now define the “Born approximation operator”, B

$$B : {E}_{m} {γ}^{B}$$

which maps a measured field to the corresponding Born approximation, we can apply B to both sides of φ(γ)=Eₘ, to yield:

$$B ϕ ( γ ) = {γ}^{B}$$

This provides a nonlinear operator Bϕ acting on M_{γ}≡{manifold of all possible γ's}. This operator also has as its range this same My. As with any nonlinear operator we will solve it iteratively. Each iteration involves the solution of the linear system of equations:

$$[ {Jac}_{B ϕ} ] ( δγ ) = - ( B ϕ ( {γ}^{( n )} ) - {γ}^{B} )$$

where the “Jacobian” Jac_{Bφ}≡

$${Jac}_{B ϕ} ≡ \frac{\partial B ϕ}{\partial γ} = \frac{\partial ϕ}{\partial {γ}^{′}}$$

by virtue of the linearity of B.

Method 2) above, proposes the use of BisTAB to solve the system [Jac_{Bφ}](δγ)=−(Bφ(γ⁽ⁿ⁾)−γ^{B}). Then adjust γ via γ⁽ⁿ⁺¹⁾←γ⁽ⁿ⁾+δγ⁽ⁿ⁾.

Similarly method 1) above defines a different operator φB(Eₛ)=Eₘ, and solves this system for Eₛ, which is then used to give γ via: γ=B(Eₛ).

The advantage of having a square operator (Domain and Range of the operator are of equal dimension) is that the BiSTAB method is used to solve the linear equation for the update δγ.

Within this context method 3) can be explained as follows:

Suppose we have (as before):

$$ϕ ( γ ) = D [ γ ] {( I - C [ γ ] )}^{- 1} {E}_{b}$$

The system (nonlinear) we are required to solve for an estimate of γ is

$$ϕ ( γ ) = {E}_{m}$$

which in traditional methods involves some kind of minimization:

$${min}_{y} {‖ {E}_{m} - ϕ ( γ ) ‖}^{2}$$

using rectangular CG.

In Methods 1), and 2) we made particular use of the Born reconstruction operator:

$$B : {E}_{m} {γ}^{B}$$

to ameliorate the difficulties associated with ill-conditioning of the rectangular system by enabling use of BiCG. Now, however, imagine that we can define some nonlinear operator:

$$\hat{ϕ} : {E}_{m} γ ∈ {M}_{γ}$$

As yet, this operator is undefined, but a particular example might be the Born reconstruction operator. With this more general, and as yet undetermined \hat{φ} operator, Bφ(γ)=γ^{B} becomes \hat{ϕ}ϕ(γ)=\hat{γ}. Now suppose that \hat{φ} can vary with the iteration number \hat{ϕ}^{(κ)}ϕ(γ)=\hat{γ}

This provides a nonlinear operator \hat{ϕ}ϕ acting on M_{γ}≡{manifold of all possible γ's}. This operator also has as its range this same My. As with any nonlinear operator we will solve it iteratively. Each iteration involves the solution of the linear system of equations:

$$[ {Jac}_{\hat{ϕ} ϕ} ] ( δγ ) = - ( \hat{ϕ} ϕ ( {γ}^{( n )} ) - \hat{γ} )$$

where \hat{γ}≡\hat{φ}(Eₘ). Using \hat{γ}⁽ⁿ⁾ ≡\hat{ϕ}⁽ⁿ⁾(Eₘ)=γ⁽ⁿ⁻¹⁾ as the definition of ϕ⁽ⁿ⁾ means that each iteration solves (using BiSTAB) the system [Jac_{\hat{ϕ}}ϕ](δγ)=−(\hat{ϕ}ϕ(γ⁽ⁿ⁾)−γ⁽ⁿ⁻¹⁾). Then adjust γ⁽ⁿ⁺¹⁾←γ⁽ⁿ⁾+δγ⁽ⁿ⁾

Method 4). uses very similar ideas but instead of solving \hat{ϕ}(κ)ϕ(γ)=γ, it solves ϕ\hat{ϕ}(Eₛ)=Eₘ iteratively for Eₛ, and hence γ≡\hat{ϕ}(Eₛ). Both of these methods 3) and 4) are, in actual fact, finding fixed points of square (nonlinear) maps:

### Method 3) Schematic

Solve:

\hat{ϕ}ϕ(γ)=γ subject to \hat{ϕ}(Eₘ)=γ

Determine \hat{ϕ}^{j}→\hat{ϕ} iteratively. instead of using the Born reconstruction operator.

### Method 4) Schematic

Solve:

ϕ\hat{ϕ}(Eₘ)=Eₘ then determine γ=\vec{ϕ}(Eₘ)

Again \hat{ϕ}^{j}→\hat{ϕ} is determined iteratively in a similar manner to the method described for Method 3).

In one implementation using imaging algorithms as described above, generating, by a processor, a preliminary reconstruction image and/or reconstruction image comprises using a processing unit programmed to process data derived from incident wavefield energy that have been transmitted at one or more frequencies from one or more transmitter positions, each said transmitter position propagating wavefield energy at at least one transmitter orientation defined by Euler angles with respect to a selected fixed coordinate system, wherein said propagated wavefield energy is scattered by matter within a region and is detected by one or more receivers at one or more receiver positions and receiver orientations thereof, said receiver orientations defined by Euler angles with respect to said selected fixed coordinate system, by, at least: (a) choosing a forward scattering model which generates a total wavefield energy given said incident wavefield energy and one or more physical characteristics of said matter defined at selected points within said region; (b) propagating said incident wavefield energy toward said region from said one or more transmitter positions and transmitter orientations thereof, said incident wavefield energy having one or more frequencies; (c) detecting detected wavefield energy at each of said one or more receiver positions and respective receiver orientations thereof; (d) electronically processing said detected wavefield energy so as to transform said detected wavefield energy into detected scattered wavefield energy that is stored in a computer memory and that corresponds to said detected wavefield energy; (e) selecting a region characteristics estimate of selected physical characteristics at said selected points within the region and storing said region characteristics estimate in said computer memory; (f) said processing unit calculating a fixed target characteristics estimate from the detected wavefield energy, said fixed target characteristics estimate being defined such that when operated on by a selected fixed approximation of the forward scattering model, the detected wavefield energy results; (g) said processing unit performing a convergence step comprising the following steps: (1) preparing, for each of said one or more frequencies at each said transmitter position and respective transmitter orientations thereof, an estimate of said total wavefield energy at said selected points derived from said incident wavefield energy for said selected points stored in the computer memory and said region characteristics estimate for said selected points; (2) deriving, for each of said one or more frequencies at each said transmitter position and respective transmitter orientations thereof, a calculated scattered wavefield energy for one or more of said receiver positions and respective receiver orientations thereof from at least one of: (i) said region characteristics estimate at said selected points, and (ii) said estimate of said total wavefield energy at said selected points; (3) deriving from said calculated scattered field one or more variable region characteristics by solving a system whose right hand side is said calculated scattered wavefield energy, and whose left hand side is the result of applying said fixed approximation of the scattering model to said one or more variable region characteristics; (4) comparing said one or more variable region characteristics to said fixed target characteristics estimate to derive a comparator; (5) when said comparator is greater than a selected tolerance, said processing unit computing and storing in said computer memory an updated region characteristics estimate from: (i) said estimate of said internal field; (ii) said fixed target characteristics estimate; (iii) said one or more variable region characteristics; and (iv) the square Jacobian of the one or more variable region characteristics with respect to the region characteristics estimate, said square Jacobian utilization being implemented via a method of the family of gradient methods specifically designed for square systems, and then setting said region characteristics estimate equal to said updated region characteristics estimate; (h) repeating said processing unit convergence step until said comparator is less than or equal to said selected tolerance, said processing unit thereafter storing said updated region characteristics estimate in said computer memory.

FIG. 56 shows an example computing system through which the described methods may be carried out. In some implementations, the computing system may be embodied, at least in part, as a viewing station and/or picture archival and communication system (PACS). PACS refers to the software tools and environment providing storage, retrieval management, distribution, and presentation of medical images. PACS software provides an access point across multiple sites and platforms to the imaging (and imaging-related) data stored on one or more storage systems managed by the PACS software. The PACS' storage systems contain archives for storage and retrieval of images (using DICOM or other formats), documentation, and reports, which are generally accessed via secure networks and may reside on local hardware or in the cloud (AWS, Azure, or other hosting platform for example, but not limited to specific platforms). One or more secure networks (e.g., intranet, local area network, wide area network, etc.) are used to communicate between various devices and computing systems and the cloud. The PACS generally includes and/or communicates with one or more imaging systems such as ultrasound systems, magnetic resonance imaging (MRI) systems and computed tomography (CT) scan equipment; and one or more computing devices (including workstations and portable computing devices).

Referring to FIG. 56, the computing system 5600 can include a processor 5610 and a storage system 5620 in which a program 5630 may be stored. Examples of processor 5610 include general purpose central processing units, application specific processors, and logic devices, as well as any other type of processing device, combinations, or variations thereof. The processor 5610 processes data according to instructions of program 5630.

Storage system 5620 includes any computer readable storage media readable by the processor 5610 and capable of storing software, including program 5630. Storage system 5620 may include volatile and nonvolatile, removable and non-removable media implemented in any method or technology for storage of information, such as computer readable instructions, data structures, program modules, or other data. Examples of storage media include random access memory (RAM), read only memory (ROM), magnetic disks, optical disks, CDs, DVDs, flash memory, solid state memory, phase change memory, or any other suitable storage media. Certain implementations may involve either or both virtual memory and non-virtual memory. In no case do storage media consist of a propagated signal or carrier wave. In addition to storage media, in some implementations, storage system 5620 may also include communication media over which software may be communicated internally or externally.

Storage system 5620 may be implemented as a single storage device but may also be implemented across multiple storage devices or sub-systems co-located or distributed relative to each other. Storage system 5620 may include additional elements, such as a controller, capable of communicating with processor 5610.

Program 5630 includes the instructions for performing adaptive imaging such as described herein, including for a reconstruction manager 310 and the various reconstruction algorithms described herein.

A database 5640 storing speed of sound, reflection, and other imaging data (i.e., images—speed of sound, attenuation and/or reflection) from an imaging system can be coupled to the system via wired or wireless connections. In some cases, the database 5640 can be part of a PACS with which system 5600 communicates. In some cases, database 5640 is part of storage system 5620.

Database 5640 and/or storage system 5620 can also store reconstruction configurations such as described herein (e.g., configuration directory 370).

Visual output can be provided via a display 5650. Input/Output (I/O) devices (not shown) such as a keyboard, mouse, network card or other I/O device may also be included. It should be understood that any computing device implementing the described system may have additional features or functionality and is not limited to the configurations described herein.

Although the subject matter has been described in language specific to structural features and/or acts, it is to be understood that the subject matter defined in the appended claims is not necessarily limited to the specific features or acts described above. Rather, the specific features and acts described above are disclosed as examples of implementing the claims and other equivalent features and acts are intended to be within the scope of the claims.

What is claimed is:

1. A method comprising:
receiving, at a computing system, a reconstruction image of a breast, the reconstruction image comprising image data corresponding to a plurality of transmission frequencies used by a transmitter of an imaging system;
generating, by the computing system, an image of ductal tissue, glandular tissue, combination of ductal tissue and glandular tissue, or fibroglandular tissue from the reconstruction image of the breast; and
determining, by the computing system, a quantitative measure of topological complexity of the breast from the image.

2. The method of claim 1, wherein generating the image comprises:
performing, by the computing system, segmentation operations on the reconstruction image to generate a preliminary image of ductal tissue, glandular tissue, or a combination of ductal tissue and glandular tissue.

3. The method of claim 2, wherein generating the image further comprises:
applying, by the computing system, morphological operations to the preliminary image to generate the image.

4. The method of claim 3, wherein the morphological operations include erosion.

5. The method of claim 3, wherein determining the quantitative measure of topological complexity of the breast from the image comprises:
generating, by the computing system, a graph from the image; and
generating, by the computing system, the quantitative measure of topological complexity from the graph.

6. The method of claim 5, wherein the graph comprises a 3D graph.

7. The method of claim 5, wherein generating, by the computing system, the quantitative measure of topological complexity from the graph comprises:
evaluating, by the computing system, the graph using adjacency, incidence, distance, Laplacian matrices, or a combination thereof.

8. The method of claim 2, wherein the preliminary image provides a 2D manifold representation.

9. The method of claim 8, wherein determining the quantitative measure of topological complexity of the breast from the image comprises:
evaluating, by the computing system, the preliminary image based on algebraic topology.

10. The method of claim 9, wherein evaluating the preliminary image based on algebraic topology comprises applying a Morse function to the image, obtaining Betti numbers from the image, determining homology groups, cohomology ring, homotopy, or a combination thereof.

11. The method of claim 1, further comprising:
calculating a surface area and a volume of breast fibroglandular tissue.

12. The method of claim 11, wherein calculating the surface area and the volume of the breast fibroglandular tissue comprises evaluating glandular tissue, ductal tissue, a combination of glandular and ductal tissue, or fibroglandular tissue from a segmented image of the reconstruction image, wherein the segmented image of the reconstruction image for evaluating ductal tissue comprises a ductal image, the segmented image of the reconstruction image for evaluating glandular tissue comprises a glandular image, the segmented image of the reconstruction image for evaluating the combination of glandular and ductal image comprises a ductal and glandular image, the segmented image of the reconstruction image for evaluating fibroglandular tissue comprises a fibroglandular image.

13. The method of claim 11, further comprising:
generating a complexity value by calculating a ratio of the surface area to the volume of the breast fibroglandular tissue, calculating Betti numbers, calculating surface area and/or volume of glandular or ductal tissue separately or in combination, calculating a Morse Index, or a combination thereof.

14. The method of claim 13, further comprising providing a correction factor to a volumetric breast density estimate provided by mammography based software based on the complexity value.

15. The method of claim 13, further comprising providing the complexity value as an input to a risk assessment model.

16. The method of claim 1, further comprising providing a correction factor to a volumetric breast density estimate provided by mammography based software based on the quantitative measure of topological complexity of the breast.

17. The method of claim 1, further comprising providing the quantitative measure of topological complexity as an input to a risk assessment model.

18. The method of claim 17, wherein the risk assessment model comprises a Tyrer-Cuzick risk assessment model.

19. The method of claim 1, further comprising providing the quantitative measure of topological complexity as an input to a machine learning model for breast cancer risk assessment.

20. The method of claim 19, wherein the quantitative measure of topological complexity is one of a plurality of radiomic features extracted from the image.

21. The method of claim 20, further comprising:
tracking the plurality of radiomic features including the quantitative measure of topological complexity of the breast over a period of time from reconstruction images of the breast of a patient captured at different times over the period of time.

22. The method of claim 1, further comprising:
tracking the quantitative measure of topological complexity of the breast over a period of time from reconstruction images of the breast of a patient captured at different times over the period of time.

23. The method of claim 22, further comprising:
determining a presence of or likelihood of developing a disease using a model that includes a correlation of the quantitative measure of topological complexity of the breast over the period of time to the presence of or the likelihood of developing the disease.

24. The method of claim 1, wherein the quantitative measure of topological complexity is determined for a right breast and left breast of a patient.

25. The method of claim 24, the method further comprising:
providing, by the computing system, a visual indicator for a difference between the topological complexity of the right breast and the left breast of the patient.

26. The method of claim 1, further comprising:
generating, by the computing system, a visual representation of the quantitative measure of topological complexity of breasts for a plurality of patients having similar gene expressions associated with risk of breast cancer.

27. The method of claim 1, further comprising:
generating, by the computing system, a visual representation of the quantitative measure of topological complexity of breasts for a plurality of patients having one or more similar demographic characteristics, genetic characteristics, or a combination thereof.

28. The method of claim 1, further comprising displaying, at a graphical user interface associated with the computing system, the image.

29. A computer-readable storage medium having instructions stored thereon that when executed by a computing system direct the computing system to:
perform segmentation operations on a reconstructed image to generate a preliminary image of ductal tissue, glandular tissue, or a combination of ductal tissue and glandular tissue;
apply morphological operations to the preliminary image to generate a ductal and/or glandular image;
generate a graph from the ductal and/or glandular image; and
generate a quantitative measure of topological complexity from the graph.

30. The computer-readable storage medium of claim 29, wherein the morphological operations include erosion.

31. The computer-readable storage medium of claim 29, wherein the graph comprises a 3D graph.

32. The computer-readable storage medium of claim 29, wherein the instructions to generate the quantitative measure of topological complexity from the graph direct the computing system to:
evaluate the graph using adjacency, incidence, distance, Laplacian matrices, or a combination thereof.

33. A computing system comprising:
one or more processors; and
a computer-readable storage medium having instructions for adaptive image reconstruction stored thereon that when executed by the one or more processors of the computing system direct the computing system to:
generate a preliminary reconstruction image using a preliminary reconstruction configuration;
automatically adjust the preliminary reconstruction configuration to an updated reconstruction configuration, by, at least:
obtaining preliminary information from the preliminary reconstruction image;
accessing a database of reconstruction configurations, the database providing a mapping of characteristics of images and objects in the images to reconstruction configurations; and
performing a lookup operation to identify the updated reconstruction configuration based on the preliminary information;
generate a reconstruction image using the updated reconstruction configuration; and
obtain reconstruction information from the reconstruction image including determining topological complexity of a breast from the reconstruction image.

34. The computing system of claim 33, wherein the instructions to obtain reconstruction information from the reconstruction image including determining topological complexity of the breast from the reconstruction image direct the computing system to:
generate an image of ductal tissue, glandular tissue, combination of ductal tissue and glandular tissue, or fibroglandular tissue from the reconstruction image of the breast; and
determine a quantitative measure of topological complexity of the breast from the image.

35. The computing system of claim 33, wherein the instructions to obtain reconstruction information from the reconstruction image including determining topological complexity of the breast from the reconstruction image direct the computing system to:
segment, from the reconstruction image, ductal tissue from glandular tissue to generate a ductal image;
apply morphological operations to the ductal image to generate a final ductal image;
generate a 3D graph from the final ductal image; and
evaluate the 3D graph to generate a quantitative measure for the topological complexity of the breast from the reconstruction image.

36. The computing system of claim 35, wherein the instructions to evaluate the 3D graph to generate the quantitative measure comprises instructions to evaluate the 3D graph using adjacency, incidence, distance, Laplacian matrices, or a combination thereof.

37. The computing system of claim 33, wherein the instructions to obtain reconstruction information from the reconstruction image including determining topological complexity of the breast from the reconstruction image direct the computing system to:
segment, from the reconstruction image, ductal tissue from glandular tissue to generate a ductal image, the ductal image providing a 2D manifold representation; and
evaluate the ductal image based on algebraic topology.

38. The computing system of claim 37, wherein the instructions to evaluate the ductal image based on algebraic topology direct the computing system to apply a Morse function to the ductal image, obtain Betti numbers from the ductal image, determine homology groups, determine a cohomology ring, determine homotopy, or a combination thereof.

39. The computing system of claim 33, wherein the instructions to obtain reconstruction information from the reconstruction image including determining topological complexity of the breast from the reconstruction image direct the computing system to:
calculate a surface area and a volume of breast fibroglandular tissue.

40. The computing system of claim 39, wherein the instructions to calculate the surface area and the volume of the breast fibroglandular tissue directs the computing system to evaluate glandular tissue, ductal tissue, or a combination of glandular and ductal tissue.

41. The computing system of claim 39, wherein the instructions to obtain reconstruction information from the reconstruction image including determining topological complexity of the breast from the reconstruction image further direct the computing system to:
generate a complexity value by calculating a ratio of the surface area to the volume of the breast fibroglandular tissue, calculating Betti numbers, calculating surface area and/or volume of glandular or ductal tissue separately or in combination, calculating a Morse Index, or a combination thereof.

42. The computing system of claim 33, wherein the instructions to obtain reconstruction information from the reconstruction image include instructions to:
quantitatively determine a spectral behavior of speed of sound and attenuation images of the reconstruction image to identify tissue types based on a power law correlation of linear coefficients associated with attenuation, speed of sound or other tissue characteristic to a tissue type and/or abnormality.
